(19) United States
(12) Patent Application Publication
(10) Pub. No.: US 2006/0074243 A1
(43) Pub. Date: Apr. 6, 2006

(54) Hydroxamic acid and amide compounds and their use as protease inhibitors

(75) Inventors: Daniel P. Becker, Glenview, IL (US); Yiyuan Chen, Skokie, IL (US); John N. Freskos, Clayton, MO (US); Alan F. Gasiecki, Vernon Hills, IL (US); Margaret L. Grapperhaus, Troy, IL (US); Donald W. Hansen JR., Skokie, IL (US); Robert M. Heintz, Bourbon, MO (US); Darren J. Kassab, O'Fallon, MO (US); Ish K. Khanna, Libertyville, IL (US); Stephen A. Kolodziej, Ballwin, MO (US); Sergio Mantegani, Milan (IT); Mark A. Massa, Ballwin, MO (US); Joseph J. McDonald, Wildwood, MO (US); Deborah A. Mischke, Defiance, MO (US); Mark A. Nagy, Chesterfield, MO (US); Ettore Perrone, Milan (IT); Joseph G. Rico, O'Fallon, MO (US); Michelle A. Schmidt, Belleville, IL (US); Dale P. Spangler, San Diego, CA (US); John J. Talley, Somerville, MA (US); Mahima Trivedi, Danberry, CT (US); Thomas A. Wynn, Salem, MA (US)

Correspondence Address: WARNER-LAMBERT COMPANY, 2800 PLYMOUTH RD, ANN ARBOR, MI, 48105, US

(73) Assignees: Pharmacia Corporation; Pharmacia Italia SPA

(21) Appl. No.: 11/270,391

(22) Filed: Nov. 10, 2005

Related U.S. Application Data
(63) Continuation of application No. 10/603,441, filed on Jun. 25, 2003.
(60) Provisional application No. 60/391,329, filed on Jun. 25, 2002.

(51) Int. Cl. C07D 211/68

(52) U.S. Cl. 546/193

(57) ABSTRACT

This invention is directed generally to hydroxamic acid and amide compounds (including salts of such compounds), and, more particularly, to aryl- and heteroaryl-arylsulfonylmethyl hydroxamic acids and amides that, inter alia, inhibit protease activity, particularly matrix metalloproteinase (also known as “matrix metalloprotease” or “MMP”) activity and/or aggrecanase activity. These compounds generally correspond in structure to Formula I: 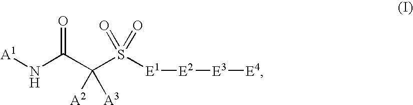
wherein A¹, A², A³, E¹, E², E³, and E⁴ are as defined in this patent. This invention also is directed to compositions of such compounds, intermediates for the syntheses of such compounds, methods for making such compounds, and methods for treating conditions associated with MMP activity and/or aggrecanase activity, particularly pathological conditions.

### PRIORITY CLAIM TO RELATED PATENT APPLICATIONS

This patent application is a continuation of U.S. patent application Ser. No. 10/603,441 filed Jun. 25, 2003, which claims priority to U.S. Provisional Patent Application Ser. No. 60/391,329 filed Jun. 25, 2002. The entire text of these applications are incorporated by reference into this patent.

### FIELD OF THE INVENTION

This invention is directed generally to hydroxamic acid and amide compounds (including salts of such compounds), and, more particularly, to aryl- and heteroaryl-arylsulfonylmethyl hydroxamic acids and amides that, inter alia, inhibit protease activity, particularly matrix metalloproteinase (also known as “matrix metalloprotease” or “MMP”) activity and/or aggrecanase activity. This invention also is directed to compositions of such compounds, intermediates for the syntheses of such compounds, methods for making such compounds, and methods for treating conditions associated with MMP activity and/or aggrecanase activity, particularly pathological conditions.

### BACKGROUND OF THE INVENTION

Connective tissue is a required component of all mammals. It provides rigidity, differentiation, attachments, and, in some cases, elasticity. Connective tissue components include, for example, collagen, elastin, proteoglycans, fibronectin, and laminin. These biochemicals make up (or are components of) structures, such as skin, bone, teeth, tendon, cartilage, basement membrane, blood vessels, cornea, and vitreous humor.

Under normal conditions, connective tissue turnover and/or repair processes are in equilibrium with connective tissue production. Degradation of connective tissue is carried out by the action of proteinases released from resident tissue cells and/or invading inflammatory or tumor cells.

Matrix metalloproteinases, a family of zinc-dependent proteinases, make up a major class of enzymes involved in degrading connective tissue. Matrix metalloproteinases are divided into classes, with some members having several different names in common use. Examples are: MMP-1 (also known as collagenase 1, fibroblast collagenase, or EC 3.4.24.3); MMP-2 (also known as gelatinase A, 72 kDa gelatinase, basement membrane collagenase, or EC 3.4.24.24), MMP-3 (also known as stromelysin 1 or EC 3.4.24.17), proteoglycanase, MMP-7 (also known as matrilysin), MMP-8 (also known as collagenase II, neutrophil collagenase, or EC 3.4.24.34), MMP-9 (also known as gelatinase B, 92kDa gelatinase, or EC 3.4.24.35), MMP-10 (also known as stromelysin 2 or EC 3.4.24.22), MMP-1 I (also known as stromelysin 3), MMP-12 (also known as metalloelastase, human macrophage elastase or HME), MMP-13 (also known as collagenase 111), and MMP-14 (also known as MT1-MMP or membrane MMP). See, generally, Woessner, J. F., “The Matrix Metalloprotease Family” in Matrix Metalloproteinases, pp. 1-14 (Edited by Parks, W. C. & Mecham, R. P., Academic Press, San Diego, Calif. 1998).

Excessive breakdown of connective tissue by MMPs is a feature of many pathological conditions. Inhibition of MMPs therefore provides a control mechanism for tissue decomposition to treat these pathological conditions. Such pathological conditions generally include, for example, tissue destruction, fibrotic diseases, pathological matrix weakening, defective injury repair, cardiovascular diseases, pulmonary diseases, kidney diseases, liver diseases, ophthalmologic diseases, and diseases of the central nervous system. Specific examples of such conditions include rheumatoid arthritis, osteoarthritis, septic arthritis, multiple sclerosis, a decubitis ulcer, corneal ulceration, epidermal ulceration, gastric ulceration, tumor metastasis, tumor invasion, tumor angiogenesis, periodontal disease, liver cirrhosis, fibrotic lung disease, emphysema, otosclerosis, atherosclerosis, proteinuria, coronary thrombosis, dilated cardiomyopathy, congestive heart failure, aortic aneurysm, epidermolysis bullosa, bone disease, Alzheimer's disease, defective injury repair (e.g., weak repairs, adhesions such as post-surgical adhesions, and scarring), post-myocardial infarction, bone disease, and chronic obstructive pulmonary disease.

Matrix metalloproteinases also are involved in the biosynthesis of tumor necrosis factors (TNFs). Tumor necrosis factors are implicated in many pathological conditions. TNF-α, for example, is a cytokine that is presently thought to be produced initially as a 28 kD cell-associated molecule. It is released as an active, 17 kD form that can mediate a large number of deleterious effects in vitro and in vivo. TNF-α can cause and/or contribute to the effects of inflammation (e.g., rheumatoid arthritis), autoimmune disease, graft rejection, multiple sclerosis, fibrotic diseases, cancer, infectious diseases (e.g., malaria, mycobacterial infection, meningitis, etc.), fever, psoriasis, cardiovascular diseases (e.g., post-ischemic reperfusion injury and congestive heart failure), pulmonary diseases, hemorrhage, coagulation, hyperoxic alveolar injury, radiation damage, and acute phase responses like those seen with infections and sepsis and during shock (e.g., septic shock and hemodynamic shock). Chronic release of active TNF-α can cause cachexia and anorexia. TNF-α also can be lethal.

Inhibiting TNF (and related compounds) production and action is an important clinical disease treatment. Matrix metalloproteinase inhibition is one mechanism that can be used. MMP (e.g., collagenase, stromelysin, and gelatinase) inhibitors, for example, have been reported to inhibit TNF-α release. See, e.g., Gearing et al. Nature 376, 555-557 (1994). See also, McGeehan et al. See also, Nature 376, 558-561 (1994). MMP inhibitors also have been reported to inhibit TNF-α convertase, a metalloproteinase involved in forming active TNF-α. See, e.g., WIPO Int'l Pub. No. WO 94/24140. See also, WIPO Int'l Pub. No. WO 94/02466. See also, WIPO Int'l Pub. No. WO 97/20824.

Matrix metalloproteinases also are involved in other biochemical processes in mammals. These include control of ovulation, post-partum uterine involution, possibly implantation, cleavage of APP (β-amyloid precursor protein) to the ainyloid plaque, and inactivation of (α_{I}-protease inhibitor (α_{I}-PI). Inhibiting MMPs therefore may be a mechanism that may be used to control of fertility. In addition, increasing and maintaining the levels of an endogenous or administered serine protease inhibitor (e.g., α_{I}-PI) supports the treatment of pathological conditions such as emphysema, pulmonary diseases, inflammatory diseases, and diseases of aging (e.g., loss of skin or organ stretch and resiliency).

Numerous metalloproteinase inhibitors are known. See, generally, Brown, P. D., “Synthetic Inhibitors of Matrix Metalloproteinases,” in Matrix Metalloproteinases, pp. 243-61 (Edited by Parks, W. C. & Mecham, R. P., Academic Press, San Diego, Calif. 1998).

Metalloproteinase inhibitors include, for example, natural biochemicals, such as tissue inhibitor of metalloproteinase (TIMP), α2-macroglobulin, and their analogs and derivatives. These are high-molecular-weight protein molecules that form inactive complexes with metalloproteinases.

A number of smaller peptide-like compounds also have been reported to inhibit metalloproteinases. Mercaptoamide peptidyl derivatives, for example, have been reported to inhibit angiotensin converting enzyme (also known as ACE) in vitro and in vivo. ACE aids in the production of angiotensin II, a potent pressor substance in mammals. Inhibiting ACE leads to lowering of blood pressure.

A wide variety of thiol compounds have been reported to inhibit MMPs. See, e.g., WO95/12389. See also, WO96/11209. See also, U.S. Pat. No. 4,595,700. See also, U.S. Pat. No. 6.013,649.

Various hydroxamic acid compounds also have been reported to inhibit MMPs. Such compounds reportedly include compounds having a carbon backbone. See, e.g., WIPO Int'l Pub. No. WO 95/29892. See also, WIPO Int'l Pub. No. WO 97/24117. See also, WIPO Int'l Pub. No. WO 97/49679. See also, European Patent No. EP 0 780 386. Such compounds also reportedly include compounds having peptidyl backbones or peptidomimetic backbones. See, e.g, WIPO Int'l Pub. No. WO 90/05719. See also, WIPO Int'l Pub. No. WO 93/20047. See also, WIPO Int'l Pub. No. WO 95/09841. See also, WIPO Int'l Pub. No. WO 96/06074. See also, Schwartz et al., Progr. Med. Chem., 29:271-334 (1992). See also, Rasmussen et al., PharmacoL Ther., 75(1): 69-75 (1997). See also, Denis et al., Invest New Drugs, 15(3): 175-185 (1997). Various piperazinylsulfonylmethyl and piperidinylsulfonylmethyl hydroxamic acid compounds also have been reported to inhibit MMPs. See, WIPO Int'l Pub. No. WO 00/46221. See also, U.S. Pat. Nos. 6,448,250; 6,372,758; and 6,492,367. And various aromatic sulfone compounds have been reported to inhibit MMPs. See, WIPO Int'l Pub. No. WO 99/25687 (which issued as U.S. Pat. No. 6,541,489 on Apr. 1, 2003). See also, WIPO Int'l Pub. No. WO 00/50396. See also, WIPO Int'l Pub. No. WO 00/69821. See also, WIPO Int'l Pub. No. WO 02/15257. See also, U.S. Appl. Publ. No. US-2003-0073718.

Various amide compounds also have been reported to inhibit MMPs. Such compounds reportedly include, for example, various aromatic sulfone compounds. See, WIPO Int'l Pub. No. WO/50396.

It is often advantageous for an MMP inhibitor drug to target a certain MMP(s) over another MMP(s). For example, it is typically preferred to inhibit MMP-2, MMP-3, MMP-9, and/or MMP-13 (particularly MMP-13) when treating cancer, inhibiting of metastasis, and inhibiting angiogenesis. It also is typically preferred to inhibit MMP-13 when treating osteoarthritis. See, e.g., Mitchell et al., J. Clin. Invest., 97:761-768 (1996). See also, Reboul et al., J. Clin. Invest., 97:2011-2019 (1996). Normally, however, it is preferred to use a drug that has little or no inhibitory effect on MMP-1 and MMP-14. This preference stems from the fact that both MMP-1 and MMP-14 are involved in several homeostatic processes, and inhibition of MMP-1 and/or MMP-14 consequently tends to interfere with such processes.

Many known MMP inhibitors exhibit the same or similar inhibitory effects against each of the MMPs. For example, batimastat (a peptidomimetic hydroxamic acid) has been reported to exhibit IC₅₀ values of from about 1 to about 20 nM against each of MMP-1, MMP-2, MMP-3, MMP-7, and MMP-9. Marimastat (another peptidomimetic hydroxamic acid) has been reported to be another broad-spectrum MMP inhibitor with an enzyme inhibitory spectrum similar to batimastat, except that Marimastat reportedly exhibited an IC₅₀ value against MMP-3 of 230 nM. See Rasmussen et al., Pharmacol. Ther., 75(1): 69-75 (1997).

Meta analysis of data from Phase I/II studies using Marimastat in patients with advanced, rapidly progressive, treatment-refractory solid tumor cancers (colorectal, pancreatic, ovarian, and prostate) indicated a dose-related reduction in the rise of cancer-specific antigens used as surrogate markers for biological activity. Although Marimastat exhibited some measure of efficacy via these markers, toxic side effects reportedly were observed. The most common drug-related toxicity of Marimastat in those clinical trials was musculoskeletal pain and stiffness, often commencing in the small joints in the hands, and then spreading to the arms and shoulder. A short dosing holiday of 1-3 weeks followed by dosage reduction reportedly permits treatment to continue. See Rasmussen et al., Pharmacol. Ther., 75(1): 69-75 (1997). It is thought that the lack of specificity of inhibitory effect among the MMPs may be the cause of that effect.

Another enzyme implicated in pathological conditions associated with excessive degradation of connective tissue is aggrecanase, particularly aggrecanase-1 (also known as ADAMTS-4). Specifically, articular cartilage contains large amounts of the proteoglycan aggrecan. Proteoglycan aggrecan provides mechanical properties that help articular cartilage in withstanding compressive deformation during joint articulation. The loss of aggrecan fragments and their release into synovial fluid caused by proteolytic cleavages is a central pathophysiological event in osteoarthritis and rheumatoid arthritis. It has been reported that two major cleavage sites exist in the proteolytically sensitive interglobular domains at the N-terminal region of the aggrecan core protein. One of those sites has been reported to be cleaved by several matrix metalloproteases. The other site, however, has been reported to be cleaved by aggrecanase-1. Thus, inhibiting excessive aggrecanase activity provides an additional and/or alternative treatment method for inflammatory conditions. See generally, Tang, B. L., “ADAMTS: A Novel Family of Extracellular Matrix Proteases,” Int'l Journal of Biochemistry & Cell Biology, 33, pp. 33-44 (2001). Such diseases reportedly include, for example, osteoarthritis, rheumatoid arthritis, joint injury, reactive arthritis, acute pyrophosphate arthritis, and psoriatic arthritis. See, e.g., European Patent Application Publ. No. EP 1 081 137 A1.

In addition to inflammatory conditions, there also is evidence that inhibiting aggrecanase may be used for treating cancer. For example, excessive levels of aggrecanase-1 reportedly have been observed with a ghoma cell line. It also has been postulated that the enzymatic nature of aggrecanase and its similarities with the MMPs would support tumor invasion, metastasis, and angiogenesis. See Tang, Int'l Journal of Biochemistry & Cell Biology, 33, pp. 33-44 (2001).

Various hydroxamic acid compounds have been reported to inhibit aggrecanase-1. Such compounds include, for example, those described in European Patent Application Publ. No. EP 1 081 137 A1. Such compounds also include, for example, those described in WIPO PCT Int'l Publ. No. WO 00/09000. Such compounds also include, for example, those described in WIPO PCT Int'l Publ. No. WO 00/59874. Such compounds also include, for example, those described in WIPO Int'l Pub. No. WO 02/007930. Such compounds also include, for example, those described in WIPO Int'l Pub. No. WO 02/092588. Such compounds also include, for example, those described in U.S. Appl. Publ. No. US-2003-0073718.

In view of the importance of hydroxamic acid and amide compounds in the treatment of several pathological conditions and the lack of enzyme specificity exhibited by two of the more potent MMP-inhibitor drugs that have been in clinical trials, there continues to be a need for hydroxamic acid and amide compounds having greater enzyme specificity (preferably toward MMP-2, MMP-9, MMP-13, and/or aggrecanase (particularly toward MMP-13 in some instances, toward both MMP-2 and MMP-9 in other instances, and aggrecanase in yet other instances), while exhibiting little or no inhibition of MMP-1 and/or MMP-14. The following disclosure describes hydroxamic acid and amide compounds that tend to exhibit such desirable activities.

### SUMMARY OF THE INVENTION

This invention is directed to hydroxamic acid and amide compounds (and salts thereof) that inhibit pathological protease activity (particularly compounds that inhibit MMP-2, MMP-9, MMP-13, and/or aggrecanase activity), while generally exhibiting relatively little or no inhibition against MMP-1 and/or MMP-14 activity. This invention also is directed to a method for inhibiting MMP activity and/or aggrecanase activity, particularly pathological MMP and/or aggrecanase activity. Such a method is particularly suitable to be used with mammals, such as humans, other primates (e.g., monkeys, chimpanzees. etc.), companion animals (e.g., dogs, cats, horses. etc.), farm animals (e.g., goats, sheep, pigs, cattle, etc.), laboratory animals (e.g., mice, rats, etc.), and wild and zoo animals (e.g., wolves, bears, deer, etc.).

Briefly, therefore, this invention is directed in part to a compound or salt thereof. The compound corresponds in structure to Formula I: 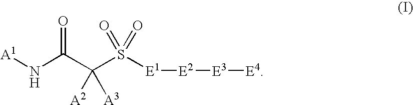
Here:

A¹ is hydrogen, hydroxy, carbocyclyloxy, or heterocyclyloxy.

A² and A³, together with the carbon to which they are bonded, form heterocyclyl or carbocyclyl. Here:
the heterocyclyl or carbocyclyl optionally is substituted with up to 3 independently selected R^{x} substituents, and/or
the heterocyclyl or carbocyclyl optionally is substituted with two substituents such that the two substituents, together with the atom(s) to which they are bonded, form a carbocyclyl or heterocyclyl, wherein the optional heterocyclyl or carbocyclyl is, in turn, optionally substituted with up to 3 independently selected R^{x} substituents.
   Alternatively, A² and A³ are independently selected from the group consisting of hydrogen, alkyl, alkoxyalkyl, alkylthioalkyl, alkenyl, alkynyl, carbocyclyl, carbocyclylalkyl, carbocyclylalkenyl, carbocyclylalkynyl, carbocyclyloxyalkyl, carbocyclylalkoxyalkyl, carbocyclylalkylthio, carbocyclylthioalkyl, carbocyclylalkylthioalkyl, heterocyclyl, heterocyclylalkyl, heterocyclylalkenyl, heterocyclylalkynyl, heterocyclyloxyalkyl, heterocyclylalkoxyalkyl, heterocyclylalkylthio, heterocyclylthioalkyl, and heterocyclylalkylthioalkyl. Any such substituent optionally is substituted with:
up to 3 independently selected R^{x} substituents, and/or
two substituents such that the two substituents, together with the atom(s) to which they are bonded, form a carbocyclyl or heterocyclyl, wherein the heterocyclyl and carbocyclyl, in turn, are optionally substituted with up to 3 independently selected R^{x} substituents.

E¹ is aryl (typically phenyl). In addition to being substituted with -E²-E³-E⁴, this aryl optionally is substituted with one or more independently selected R^{x} substituents.

In some embodiments, E² is aryl or heteroaryl. In addition to being bonded to -E³-E⁴, this aryl or heteroaryl optionally is substituted with one or more independently selected R^{x} substituents.

In alternative embodiments, E² is 2 rings fused together. In these embodiments, the ring bonded to E¹ is an unsaturated, 6-member ring. One or both of the rings comprise one or more independently selected heteroatoms (i.e., at least one ring atom in at least one of the rings is a heteroatom). In addition to being bonded to -E³-E⁴, one or both of the rings optionally are substituted with one or more independently selected R^{x} substituents.

E³ is —O—, —C(O)—, —C(O)—O—, —O—C(O)—, —N(R^{b})—, —C(O)—N(R^{b})—, —N(R^{b})—C(O)—, —C(O)—N(R^{b})—N(R^{b})—C(O)—N(R^{b})—, —S—, —S(O)—, —S(O)₂—, —N(R^{b})—S(O)₂—, —S(O)₂—N(R^{b})—, —O—S(O)₂—, —S(O)₂—O—, —C(NH)—, —C(NOH)—, —N(R^{b})—C(NH)—, —N(R^{b})—C(NOH)—, —C(NH)—N(R^{b})—, —C(NOH)—N(R^{b})—, alkyl, alkenyl, carbonylalkyl, alkylcarbonyl, or a bond. Any alkyl or alkenyl portion of any such substituent optionally is substituted with one or more independently selected R^{c} substituents. To the extent the alkyl or alkenyl is the portion of E³ that is bonded to E⁴, the E⁴ is bonded directly to the alkyl or alkenyl, and not to any optional R^{c} substituent of the alkyl or alkenyl.

E⁴ is hydrogen, alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, aminoalkyl, carbocyclyl, carbocyclylalkyl, carbocyclylalkoxyalkyl, heterocyclyl, heterocyclylalkyl, or heterocyclylalkoxyalkyl. Any such substituent optionally is substituted with one or more independently selected R^{d} substituents.

Each R^{x} is independently selected from the group consisting of halogen, cyano, hydroxy, nitro, nitroso, oxo, alkyl, alkenyl, alkynyl, alkoxy, alkoxyalkoxy, R^{b}-oxyalkyl, alkenyloxy, alkynyloxy, alkylthio, R^{b}R^{b}-amino, R^{b}R^{b}-aminoalkyl, R^{b}R^{b}-aminoalkoxy, R^{b}R^{b}-aminoalkyl(R^{b})amino, carbocyclyl, carbocyclylalkyl, carbocyclyloxy, carbocyclyloxyalkoxy, carbocyclylthio, heterocyclyl, heterocyclylalkyl, heterocyclyloxy, heterocyclyloxyalkoxy, heterocyclylthio, alkyliminocarbonyl, alkylthioalkyl, alkylsulfonylalkyl, alkylsulfoxidoalkyl, alkylthioalkenyl, alkylsulfoxidoalkenyl, alkylsulfonylalkenyl, carbocyclylalkoxyalkyl, carbocyclyliminocarbonyl, carbocyclylthioalkyl, carbocyclylsulfoxidoalkyl, carbocyclylsulfonylalkyl, carbocyclylthioalkenyl, carbocyclylsulfoxidoalkenyl, carbocyclylsulfonylalkenyl, heterocyclylalkoxyalkyl, heterocyclylthioalkyl, heterocyclylsulfoxidoalkyl, heterocyclylsulfonylalkyl, heterocyclylthioalkenyl, heterocyclylsulfoxidoalkenyl, heterocyclylsulfonylalkenyl, heterocyclyliminocarbonyl, aminosulfonylalkyl, and —R^{x1}—R^{x2}. Any such group optionally is substituted with one or more substituents independently selected from the group consisting of halogen, hydroxy, cyano, amino, carboxy, thiol, sulfo, nitro, nitroso, oxo, thioxo, imino, alkyl, alkoxy, alkoxyalkyl, and alkoxyalkoxy. With respect to these optional substituents:
the alkyl, alkoxy, alkoxyalkyl, and alkoxyalkoxy optionally are substituted with one or more substituents independently selected from the group consisting of halogen and hydroxy; and
the amino optionally is substituted with up to 2 independently selected alkyl.

Each R^{x1} is independently selected from the group consisting of —C(O)—, —C(S)—, —C(NR^{y})—, and —S(O)₂—.

Each R^{y} is independently selected from the group consisting of hydrogen and hydroxy.

Each R^{x2} is independently selected from the group consisting of hydrogen, hydroxy, alkyl, alkenyl, alkynyl, alkoxy, alkoxyalkyl, alkoxyalkoxy, R^{b}-oxyalkyl, alkenyloxy, alkynyloxy, R^{b}R^{b}-amino, R^{b}R^{b}-aminoalkyl, R^{b}R^{b}-aminoalkoxy, R^{b}R^{b}-aminoalkyl(R^{b})amino, carbocyclyl, carbocyclylalkyl, carbocyclyloxy, carbocyclyloxyalkoxy, heterocyclyl, heterocyclylalkyl, heterocyclyloxy, and heterocyclyloxyalkoxy. Any such substituent optionally is substituted with one or more substituents independently selected from the group consisting of halogen, hydroxy, cyano, carboxy, thiol, sulfo, nitro, nitroso, oxo, thioxo, imino, alkyl, alkoxy, alkoxyalkyl, and alkoxyalkoxy. Any such optional substituent is, in turn, optionally substituted with one or more substituents independently selected from the group consisting of halogen and hydroxy.

Each R^{b} is independently selected from the group consisting of hydrogen, hydroxy, alkyl, alkenyl, alkynyl, alkoxy, alkoxyalkyl, bisalkoxyalkyl, alkylthioalkyl, alkylthioalkenyl, alkylsulfoxidoalkyl, alkylsulfonyl, alkylsulfonylalkyl, carbocyclyl, carbocyclylalkyl, carbocyclyloxyalkyl, carbocyclylalkoxyalkyl, carbocyclylthioalkyl, carbocyclylthioalkenyl, carbocyclylsulfoxidoalkyl, carbocyclylsulfonyl, carbocyclylsulfonylalkyl, heterocyclyl, heterocyclylalkyl, heterocyclyloxyalkyl, heterocyclylalkoxyalkyl, heterocyclylthioalkyl, heterocyclylsulfoxidoalkyl, heterocyclylsulfonyl, heterocyclylsulfonylalkyl, aminoalkyl, aminosulfonyl, aminoalkylsulfonyl, and alkoxyalkylaminoalkyl. Any such substituent optionally is substituted with one or more substituents independently selected from the group consisting of halogen, hydroxy, cyano, carboxy, thiol, sulfo, nitro, nitroso, oxo, thioxo, imino, alkyl, alkylcarbonyl, carbocyclyl, and carbocyclylalkyl.

Each R^{c} is independently selected from the group consisting of halogen, hydroxy, cyano, carboxy, —C(H)(NH), —C(H)(NOH), thiol, sulfo, nitro, nitroso, oxo, thioxo, imino, amino, alkyl, alkoxy, alkenyl, alkynyl, alkoxyalkyl, mono-alkylamino, di-alkylamino, alkylthio, carbocyclyl, carbocyclylalkyl, heterocyclyl, and heterocyclylalkyl. Any such substituent optionally is substituted with one or more substituents independently selected from the group consisting of halogen, hydroxy, cyano, carboxy, thiol, sulfo, nitro, nitroso, oxo, thioxo, imino, aminocarbonyl, amino, alkyl, and carbocyclylalkyl.

Each R^{d} is independently selected from the group consisting of halogen, hydroxy, cyano, sulfo, nitro, nitroso, oxo, thioxo, imino, alkyl, alkoxy, alkoxyalkyl, —N(R^{e})(R^{e}), —C(O)(R^{g}), —S—R^{e}, —S(O)₂—R^{e}, carbocyclyl, alkylcarbocyclyl, carbocyclylalkyl, heterocyclyl, alkylheterocyclyl, and heterocyclylalkyl. Any such substituent optionally is substituted with one or more substituents independently selected from the group consisting of halogen, hydroxy, cyano, carboxy, thiol, sulfo, nitro, nitroso, oxo, thioxo, imino, aminocarbonyl, and amino.

Each R^{e} is independently selected from the group consisting of hydrogen alkyl, carbocyclyl, carbocyclylalkyl, heterocyclyl, and heterocyclylalkyl. Any such substituent optionally is substituted with one or more substituents independently selected from the group consisting of halogen, hydroxy, cyano, carboxy, thiol, sulfo, nitro, nitroso, oxo, thioxo, imino, aminocarbonyl, and amino.

Each R^{g} is independently selected from the group consisting of hydrogen, alkyl, —O—R^{h}, —N(R^{h})(R^{h}), carbocyclylalkyl, and heterocyclylalkyl. Any such substituent optionally is substituted with one or more substituents independently selected from the group consisting of halogen, hydroxy, cyano, carboxy, thiol, sulfo, nitro, nitroso, oxo, thioxo, imino, aminocarbonyl, and amino.

Each R^{h} is independently selected from the group consisting of hydrogen, alkyl, carbocyclyl, carbocyclylalkyl, heterocyclyl, and heterocyclylalkyl. Any such substituent optionally is substituted with one or more substituents independently selected from the group consisting of halogen, hydroxy, cyano, carboxy, thiol, sulfo, nitro, nitroso, oxo, thioxo, imino, aminocarbonyl, and amino.

This invention also is directed, in part, to a method for treating a condition associated with matrix metalloprotease activity (particularly pathologically excessive matrix metalloprotease activity) in a mammal. The method comprises administering an above-described compound or a pharmaceutically acceptable salt thereof to the mammal in an amount that is therapeutically-effective to treat the condition. In some preferred embodiments, the A¹ substituent of the compound or salt is hydrogen. In other preferred embodiments, the A¹ substituent of the compound or salt is hydroxy.

This invention also is directed, in part, to a method for treating a condition associated with TNF-α convertase activity (particularly pathologically excessive TNF-α convertase activity) in a mammal. The method comprises administering an above-described compound or a pharmaceutically acceptable salt thereof to the mammal in an amount that is therapeutically-effective to treat the condition.

This invention also is directed, in part, to a method for treating a condition associated with aggrecanase activity (particularly pathologically excessive aggrecanase activity) in a mammal. The method comprises administering an above-described compound or a pharmaceutically acceptable salt thereof to the mammal in an amount that is therapeutically-effective to treat the condition.

This invention also is directed, in part, to a method for treating a pathological condition in a mammal, wherein the pathological condition comprises tissue destruction, a fibrotic disease, pathological matrix weakening, defective injury repair, a cardiovascular disease, a pulmonary disease, a kidney disease, a liver disease, an ophthalmologic disease, and a central nervous system disease. The method comprises administering an above-described compound or a pharmaceutically acceptable salt thereof to the mammal in an amount that is therapeutically-effective to treat the condition.

This invention also is directed, in part, to a method for treating a pathological condition in a mammal, wherein the pathological condition comprises osteoarthritis, rheumatoid arthritis, septic arthritis, tumor invasion, tumor metastasis, tumor angiogenesis, a decubitis ulcer, a gastric ulcer, a corneal ulcer, periodontal disease, liver cirrhosis, fibrotic lung disease, otosclerosis, atherosclerosis, multiple sclerosis, dilated cardiomyopathy, epidermal ulceration, epidermolysis bullosa, aortic aneurysm, defective injury repair, an adhesion, scarring, congestive heart failure, post myocardial infarction, coronary thrombosis, emphysema, proteinuria, Alzheimer's disease, bone disease, and chronic obstructive pulmonary disease. The method comprises administering an above-described compound or a pharmaceutically acceptable salt thereof to the mammal in an amount that is therapeutically-effective to treat the condition.

This invention also is directed, in part, to pharmaceutical compositions comprising a therapeutically-effective amount of an above-described compound or a pharmaceutically-acceptable salt thereof.

This invention also is directed, in part, to a use of an above-described compound or a pharmaceutically acceptable salt thereof to prepare a medicament for treating a condition associated with matrix metalloprotease activity.

This invention also is directed, in part, to a use of an above-described compound or a pharmaceutically acceptable salt thereof to prepare a medicament for treating a condition associated with TNF-α convertase activity.

This invention also is directed, in part, to a use of an above-described compound or a pharmaceutically acceptable salt thereof to prepare a medicament for treating a condition associated with aggrecanase activity.

This invention also is directed, in part, to a use of an above-described compound or a pharmaceutically acceptable salt thereof to prepare a medicament for treating tissue destruction, a fibrotic disease, pathological matrix weakening, defective injury repair, a cardiovascular disease, a pulmonary disease, a kidney disease, a liver disease, an ophthalmologic disease, and a central nervous system disease. The method comprises administering an above-described compound or a pharmaceutically acceptable salt thereof to the mammal in an amount that is therapeutically-effective to treat the condition.

This invention also is directed, in part, to a use of an above-described compound or a pharmaceutically acceptable salt thereof to prepare a medicament for treating osteoarthritis, rheumatoid arthritis, septic arthritis, tumor invasion, tumor metastasis, tumor angiogenesis, a decubitis ulcer, a gastric ulcer, a corneal ulcer, periodontal disease, liver cirrhosis, fibrotic lung disease, otosclerosis, atherosclerosis, multiple sclerosis, dilated cardiomyopathy, epidermal ulceration, epidermolysis bullosa, aortic aneurysm, defective injury repair, an adhesion, scarring, congestive heart failure, post myocardial infarction, coronary thrombosis, emphysema, proteinuria, Alzheimer's disease, bone disease, and chronic obstructive pulmonary disease. The method comprises administering an above-described compound or a pharmaceutically acceptable salt thereof to the mammal in an amount that is therapeutically-effective to treat the condition.

Further benefits of Applicants' invention will be apparent to one skilled in the art from reading this patent.

### DETAILED DESCRIPTION OF THE PREFERRED EMBODIMENTS

This detailed description of preferred embodiments is intended only to acquaint others skilled in the art with Applicants' invention, its principles, and its practical application so that others skilled in the art may adapt and apply the invention in its numerous forms, as they may be best suited to the requirements of a particular use. This detailed description and its specific examples, while indicating preferred embodiments of this invention, are intended for purposes of illustration only. This invention, therefore, is not limited to the preferred embodiments described in this specification, and may be variously modified.

### A. Compounds of This Invention

In accordance with this invention, it has been found that certain piperidinyl- and piperazinyl-sulfonylmethyl hydroxamic acid compounds and salts thereof tend to be effective for inhibiting proteases, particularly those associated with excessive (or otherwise pathological) breakdown of connective tissue. Specifically, Applicants have found that these compounds and salts tend to be effective for inhibiting proteases (particularly MMP-2, MMP-9, MMP-13, other MMP's associated with pathological conditions, and/or aggrecanase) that are often particularly destructive to tissue if present or generated in abnormally excessive quantities or concentrations. Moreover, Applicants have discovered that these compounds and salts tend to be selective toward inhibiting pathological protease activity, while avoiding excessive inhibition of other proteases (particularly MMP-1 and/or MMP-14) that are typically essential to normal bodily function (e.g., tissue turnover and repair).

### A-1. Preferred Compound Structures

As noted above, the compounds of this invention generally correspond in structure to Formula I: 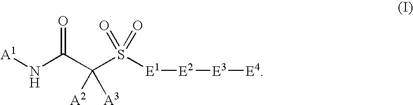
In these formulas, A¹, A², A³, E¹, E², E³, and E⁴ are defined as follows:

### General Description of Preferred A¹ Substituents

A¹ is hydrogen, hydroxy, carbocyclyloxy, or heterocyclyloxy.

In some preferred embodiments, A¹ is hydrogen. In such embodiments, the compound is an amide, and corresponds in structure to Formula (I-A):

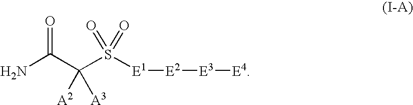

In some preferred embodiments, A¹ is tetrahydropyranyl. In such embodiments, the compound is a THP-hydroxamate and preferably corresponds in structure to Formula (I-B):

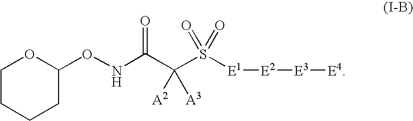

In some preferred embodiments, A¹ is hydroxy. In such embodiments, the compound is a hydroxamic acid and corresponds in structure to Formula (I-C):

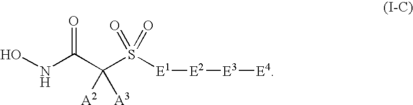

### General Description of Preferred A² and A³ Substituents

In some embodiments, A² and A³, together with the carbon to which they are bonded, form heterocyclyl or carbocyclyl. Here:
the heterocyclyl or carbocyclyl optionally is substituted with up to 3 independently selected R^{x} substituents, and/or
the heterocyclyl or carbocyclyl optionally is substituted with two substituents such that the two substituents, together with the atom(s) to which they are bonded, form a carbocyclyl or heterocyclyl, wherein the optional heterocyclyl or carbocyclyl is, in turn, optionally substituted with up to 3 independently selected R^{x} substituents.

In some preferred embodiments, the 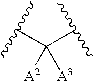
substituent corresponds in structure to one of the following:

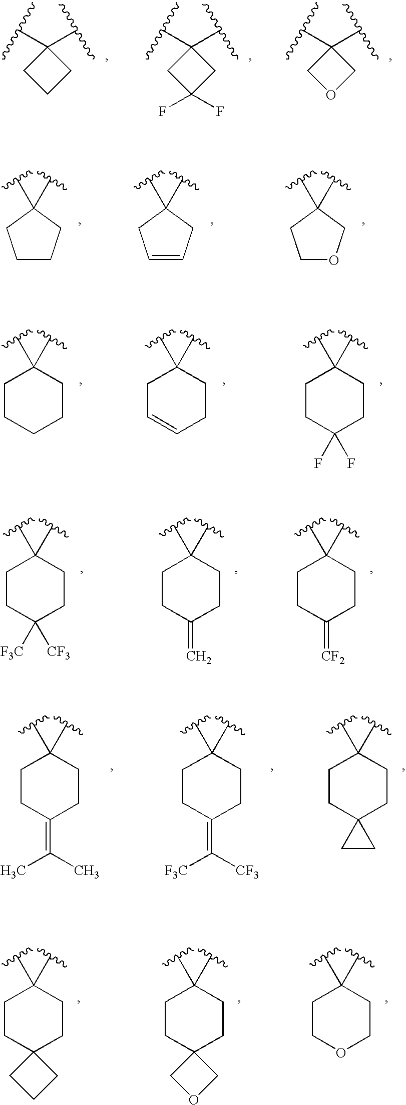

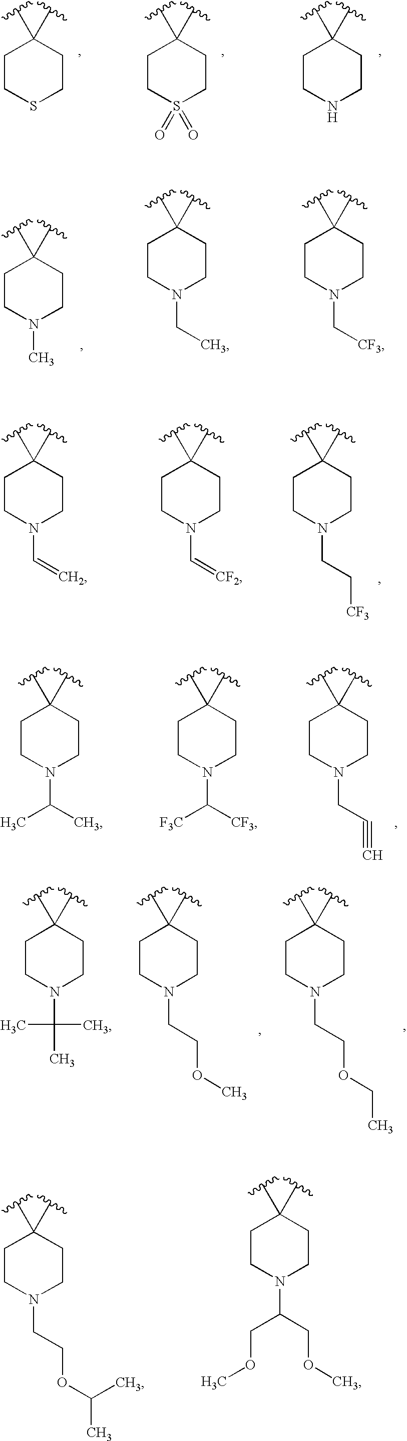

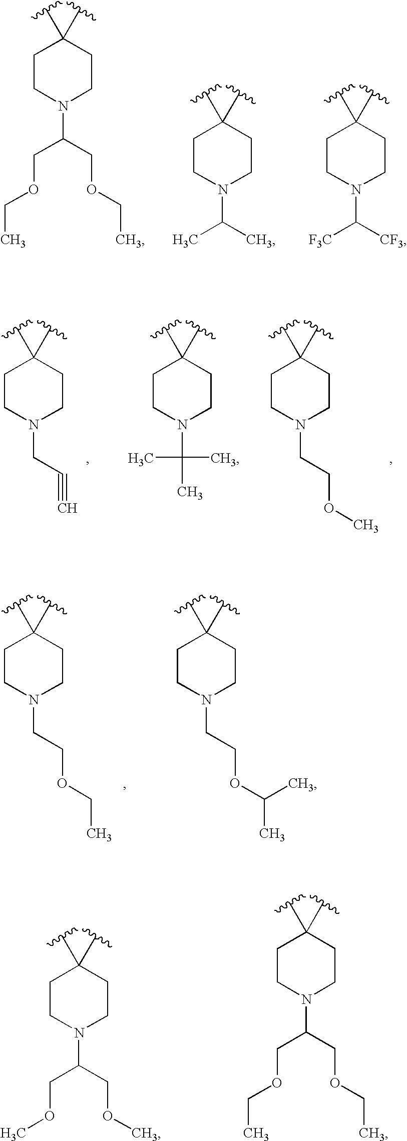

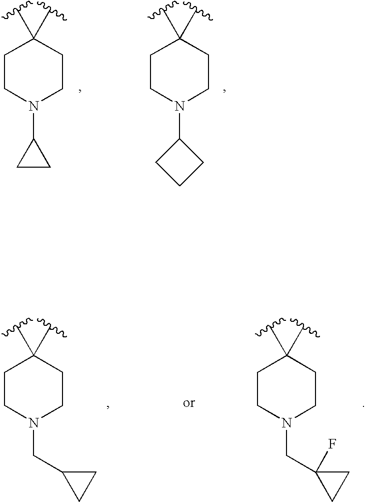

In some preferred embodiments, the 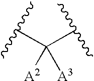
substituent corresponds in structure to one of the following:

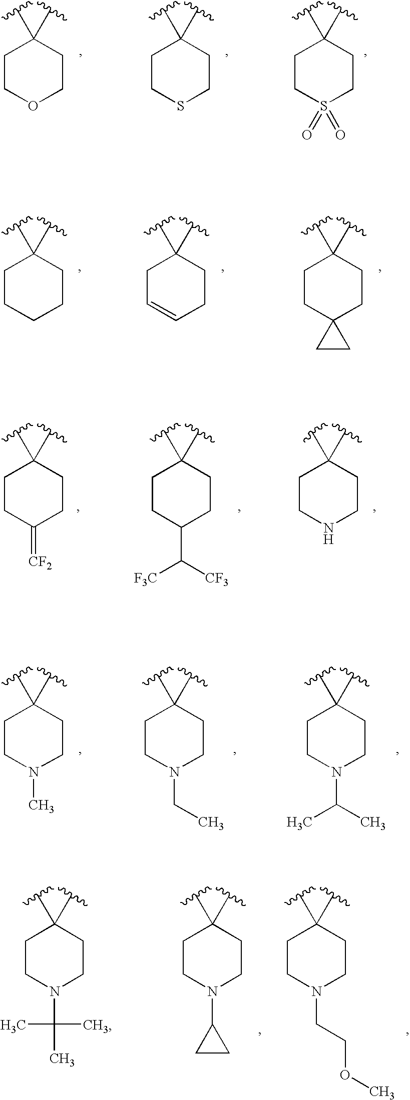

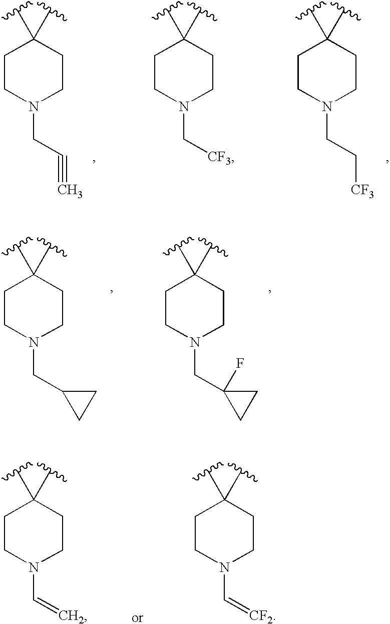

In some preferred embodiments, the compound corresponds in structure to Formula (I-D):

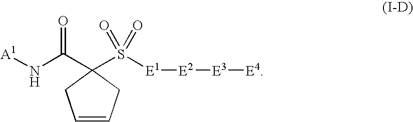

In some preferred embodiments, the compound corresponds in structure to Formula (I-E):

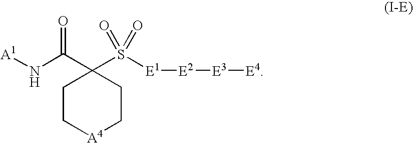

In some preferred embodiments, A⁴ is —O—, —N(H)—, —N(R^{x})—, —S—, —S(O)—, —S(O)₂—, —C(H)₂—, or —C(R^{x})₂—.

In some preferred embodiments, A⁴ is —N(H)—, —N(R^{x})—, —S—, —S(O)—, —S(O)₂—, —C(H)₂—, or —C(R^{x})₂—.

In some preferred embodiments, A⁴ is —C(H)₂— or —C(R^{x})₂— such that the compound corresponds in structure to Formula (I-F) or Formula (I-G): 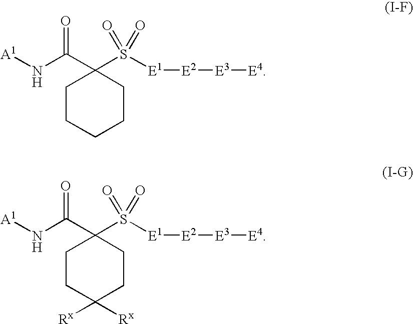
In some such embodiments, for example, the compound corresponds in structure to Formula (I-H): 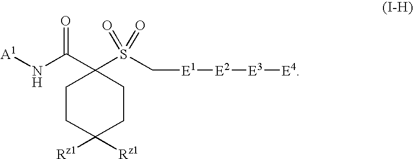
Here, each R^{z1} is independently selected from the group consisting of hydrogen, halogen, alkyl, haloalkyl, alkoxy, and alkoxyalkoxy. In some such embodiments, for example, the 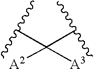
substituent corresponds in structure to one of the following formulas:

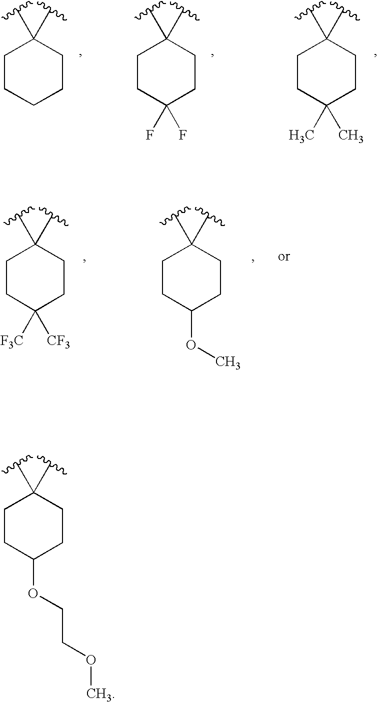

In some preferred embodiments, A⁴ is —O— such that the compound corresponds in structure to Formula (I-I):

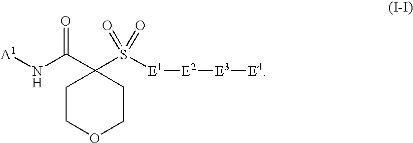

In some preferred embodiments, A⁴ is —S(O)₂— such that the compound corresponds in structure to Formula (I-J):

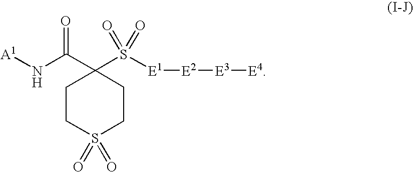

In some preferred embodiments, A⁴ is —N(R^{x})— such that the compound corresponds in structure to Formula (I-K):

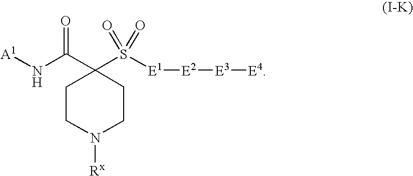

In some particularly preferred embodiments, the compound corresponds in structure to Formula (I-L): 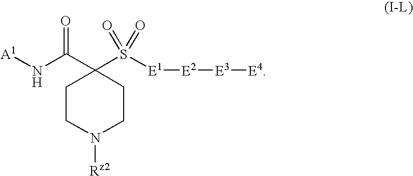
Here, R^{z2} is alkyl, alkoxyalkyl, cycloalkyl, formyl, heterocycloalkylcarbonyl, or dialkylaminocarbonyl. In some such embodiments, for example, the 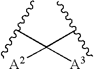
substituent corresponds in structure to one of the following formulas:

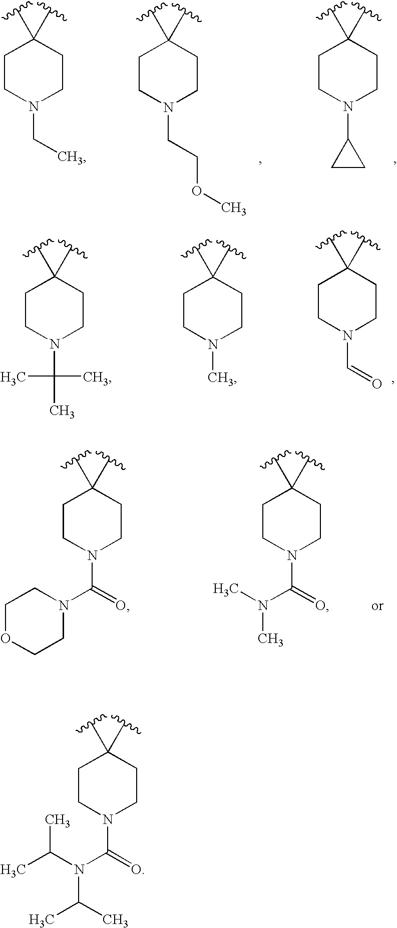

In some alternative embodiments, A² and A³ are independently selected from the group consisting of hydrogen, alkyl, alkoxyalkyl, alkylthioalkyl, alkenyl, alkynyl, carbocyclyl, carbocyclylalkyl, carbocyclylalkenyl, carbocyclylalkynyl, carbocyclyloxyalkyl, carbocyclylalkoxyalkyl, carbocyclylalkylthio, carbocyclylthioalkyl, carbocyclylalkylthioalkyl, heterocyclyl, heterocyclylalkyl, heterocyclylalkenyl, heterocyclylalkynyl, heterocyclyloxyalkyl, heterocyclylalkoxyalkyl, heterocyclylalkylthio, heterocyclylthioalkyl, and heterocyclylalkylthioalkyl. Any such substituent optionally is substituted with:
up to 3 independently selected R^{x} substituents, and/or
two substituents such that the two substituents, together with the atom(s) to which they are bonded, form a carbocyclyl or heterocyclyl, wherein the heterocyclyl and carbocyclyl, in turn, are optionally substituted with up to 3 independently selected R^{x} substituents.

In some preferred embodiments, A² is hydrogen.

In some preferred embodiments, A³ is alkoxyalkyl.

In some preferred embodiments, A² is hydrogen, and A³ is alkoxyalkyl.

### General Description of Preferred E¹ Substituents

E¹ is aryl. In addition to being substituted with -E²-E³-E⁴, this aryl optionally is substituted with one or more independently selected RX substituents.

In some preferred embodiments, E¹ is phenyl. Here, the compound corresponds in structure to Formula I-M: 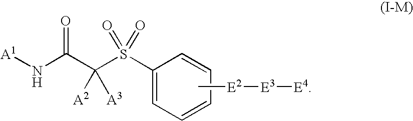
In some such embodiments, the compound corresponds in structure to Formula (I-N):

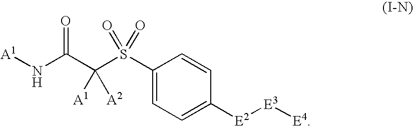

### General Description of Preferred E² Substituents

In some embodiments, E² is aryl or heteroaryl. In addition to being bonded to -E³-E⁴, the aryl or heteroaryl optionally is substituted with one or more independently selected R^{x} substituents.

In some preferred embodiments, E² is aryl or heteroaryl, wherein the aryl or heteroaryl is not substituted with any optional R^{x} substituents.

In some preferred embodiments, E² is aryl or heteroaryl, wherein the aryl or heteroaryl is:
substituted with one or more independently selected halogen, and
optionally substituted with one or more independently selected R^{x} substituents.

In some preferred embodiments, E² is aryl or heteroaryl, wherein the aryl or heteroaryl is substituted with one halogen.

In some preferred embodiments, E² is aryl or heteroaryl, wherein the aryl or heteroaryl is substituted with one fluoro.

In some preferred embodiments, E² is phenyl optionally substituted with one or more independently selected optional R^{x} substituents.

In some preferred embodiments, E² is phenyl that is not substituted with any optional R^{x} substituents.

In some preferred embodiments, E² is phenyl substituted with one or more substituents independently selected from the group consisting of halogen and haloalkyl.

In some preferred embodiments, E² is phenyl optionally substituted with one or more independently selected haloalkyl.

In some preferred embodiments, E² is phenyl optionally substituted with one or more independently selected halogen.

In some preferred embodiments, E² is phenyl substituted with one halogen.

In some preferred embodiments, E² is phenyl substituted with one fluoro.

In some preferred embodiments, the compound corresponds in structure to Formula (I-O): 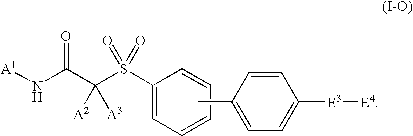
In some such embodiments, the compound corresponds in structure to Formula (I-P):

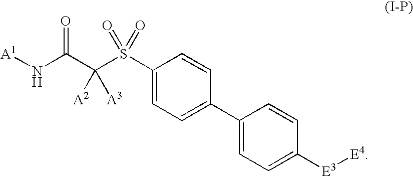

In some preferred embodiments, the compound corresponds in structure to Formula (I-Q): 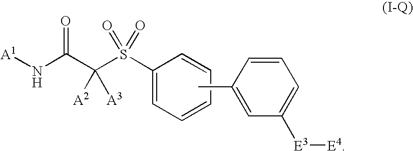
In some such embodiments, the compound corresponds in structure to Formula (I-R):

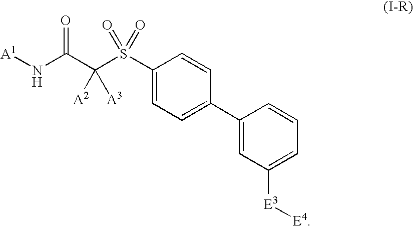

In some preferred embodiments, E² is naphthyl optionally substituted with one or more independently selected R^{x} substituents.

In some preferred embodiments, E² is naphthyl that is not substituted by any optional R^{x} substituents. In some such embodiments, the compound corresponds in structure to Formula (I-S): 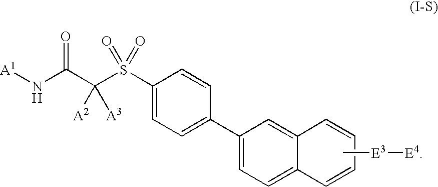
Those embodiments include, for example, compounds that correspond in structure to Formula (I-T):

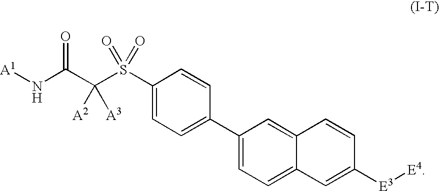

In some preferred embodiments, E² is heteroaryl substituted with one or more independently selected R^{x} substituents.

In some preferred embodiments, E² is heteroaryl, wherein the heteroaryl:
comprises at least two heteroatoms, and
is optionally substituted with one or more independently selected R^{x} substituents.

In some preferred embodiments, E² is heteroaryl not substituted with any optional R^{x} substituents.

In some preferred embodiments, E² is furanyl, thienyl, oxazolyl, isoxazolyl, thiazolyl, isothiazolyl, thiodiazolyl, oxadiazolyl, pyridinyl, pyrazinyl, pyrimidinyl, pyridazinyl, triazinyl, oxathiazinyl, oxepinyl, thiepinyl, benzofuranyl, isobenzofuranyl, benzoxazolyl, benzoisoxazolyl, benzothienyl, isobenzothienyl, benzothiazolyl, benzoisothiazolyl, benzothiadiazolyl, indolizinyl, pyranopyrrolyl, quinolinyl, isoquinolinyl, naphthyridinyl, phthalazinyl, quinoxalinyl, quinazolinyl, cinnolinyl, pteridinyl, or acridinyl.

In some preferred embodiments, E² is thienyl, oxadiazolyl, or pyridinyl.

In some preferred embodiments, E² is single-ring heteroaryl.

In some preferred embodiments, E² is 5-member heteroaryl. In some such embodiments, E² is thienyl or oxadiazolyl.

In some preferred embodiments, E² is 6-member heteroaryl. In some such embodiments, E² is pyrimidinyl. In other such embodiments, E² is pyrazinyl. In still other such embodiments, E² is pyridinyl.

In some preferred embodiments, E² is fused-ring heteroaryl.

In some preferred embodiments, E² is 9-member heteroaryl.

In some preferred embodiments, E² is 10-member heteroaryl.

In some alternative embodiments, E² is 2 rings fused together. In these embodiments, the ring bonded to E¹ is an unsaturated, 6-member ring. One or both of the rings comprise one or more independently selected heteroatoms. In addition to being bonded to E³-E⁴, one or both of the rings optionally are substituted with one or more independently selected R^{x} substituents.

In some preferred embodiments, E² is a 9-member heterocyclyl.

In some preferred embodiments, E² is a 10-member heterocyclyl.

### General Description of Preferred E³ and E⁴ Substituents

E³ is —O—, —C(O)—, —C(O)—O—, —O—C(O)—, —N(R^{b})—, —C(O)—N(R^{b})—, —N(R^{b})—C(O)—, —C(O)—N(R^{b})—N(R^{b})—C(O)—, —N(R^{b})—C(O)—N(R^{b})—, —S—, —S(O)—, —S(O)₂—, —N(R^{b})—S(O)₂—, —S(O)₂—N(R^{b})—, —O—S(O)₂—, —S(O)₂—O—, —C(NH)—, —C(NOH)—, —N(R^{b})—C(NH)—, —N(R^{b})—C(NOH)—, —C(NH)—N(R^{b})—, —C(NOH)—N(R^{b})—, alkyl, alkenyl, carbonylalkyl, alkylcarbonyl, or a bond. Any alkyl or alkenyl portion of any such substituent optionally is substituted with one or more independently selected R^{c} substituents. To the extent that the alkyl or alkenyl is the portion of E³ that is bonded to E⁴, the E⁴ is bonded directly to the alkyl or alkenyl, and not to any optional R^{c} substituent of the alkyl or alkenyl.

In some preferred embodiments, E³ is —O—, —C(O)—, —C(O)—O—, —O—C(O)—, —N(R^{b})—, —C(O)—N(R^{b})—, —N(R^{b})—C(O)—, —C(O)—N(R^{b})—N(R^{b})—C(O)—, —N(R^{b})—C(O)—N(R^{b})—, —S—, —S(O)—, —S(O)₂—, —N(R^{b})—S(O)₂—, —S(O)₂—N(R^{b})—, —O—S(O)₂—, —S(O)₂—O—, —C(NH)—, —C(NOH)—, —N(R^{b})—C(NH)—, —N(R^{b})—C(NOH)—, —C(NH)—N(R^{b})—, —C(NOH)—N(R^{b})—, alkyl, alkenyl, carbonylalkyl, or alkylcarbonyl. Any alkyl or alkenyl portion of such substituent optionally is substituted with one or more independently selected R^{c} substituents.

In some preferred embodiments, E³ is —O—, —C(O)—O—, —O—C(O)—, —N(R^{b})—, —C(O)—N(R^{b})—, —N(R^{b})—C(O)—, —C(O)—N(R^{b})—N(R^{b})—C(O)—, —N(R^{b})—C(O)—N(R^{b})—, —S—, —S(O)—, —S(O)₂—, —N(R^{b})—S(O)₂—, —S(O)₂—N(R^{b})—, —O—S(O)₂—, —S(O)₂—O—, —C(NH)—, —C(NOH)—, —N(R^{b})—C(NH)—, —N(R^{b})—C(NOH)—, —C(NH)—N(R^{b})—, —C(NOH)—N(R^{b})—, alkenyl, carbonylalkyl, alkylcarbonyl, or a bond. Any alkyl or alkenyl portion of a substituent in such group optionally is substituted with one or more independently selected R^{c} substituents.

In some preferred embodiments, E³ is —O—, —C(O)—, —C(O)—O—, —O—C(O)—, —N(R^{b})—, —C(O)—N(R^{b})—, —N(R^{b})—C(O)—, —C(O)—N(R^{b})—N(R^{b})—C(O)—, —N(R^{b})—C(O)—N(R^{b})—, —S—, —S(O)—, —S(O)₂—, —N(R^{b})—S(O)₂—, —S(O)₂—N(R^{b})—, —O—S(O)₂—, —S(O)₂—O—, —C(NH)—, —C(NOH)—, —N(R^{b})—C(NH)—, —N(R^{b})—C(NOH)—, —C(NH)—N(R^{b})—, —C(NOH)—N(R^{b})—, alkenyl, carbonylalkyl, alkylcarbonyl, or a bond. Any alkyl or alkenyl portion of a substituent in such group optionally is substituted with one or more independently selected R^{c} substituents.

In some preferred embodiments, E³ is a bond.

In some preferred embodiments, E³ is a —O—.

In some preferred embodiments, E³ is —C(O)—N(CH₃)—.

In some preferred embodiments, E³ is —C(O)—N(H)—.

In some preferred embodiments, E³ is —N(H)—.

In some preferred embodiments, E³ is carbonylalkyl.

In some preferred embodiments, E is —C(O)— or —C(O)—N(R^{b})—.

E⁴ is hydrogen, alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, aminoalkyl, carbocyclyl, carbocyclylalkyl, carbocyclylalkoxyalkyl, heterocyclyl, heterocyclylalkyl, or heterocyclylalkoxyalkyl. Any such substituent optionally is substituted with one or more independently selected R^{d} substituents.

In some preferred embodiments, E⁴ is alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, aminoalkyl, carbocyclyl, carbocyclylalkyl, carbocyclylalkoxyalkyl, heterocyclyl, heterocyclylalkyl, or heterocyclylalkoxyalkyl. Any such substituent optionally is substituted with one or more independently selected R^{d} substituents.

In some preferred embodiments, E⁴ is alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, aminoalkyl, carbocyclyl, carbocyclylalkyl, carbocyclylalkoxyalkyl, heterocyclyl, heterocyclylalkyl, or heterocyclylalkoxyalkyl. Any such substituent optionally is substituted with one or more independently selected R^{d} substituents.

In some preferred embodiments, E⁴ is alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, aminoalkyl, carbocyclyl, carbocyclylalkyl, carbocyclylalkoxyalkyl, heterocyclyl, heterocyclylalkyl, or heterocyclylalkoxyalkyl. In these embodiments, any such substituent:
comprises at least two carbon atoms, and
is substituted with one or more independently-selected halogen, and
is optionally substituted with one or more independently selected R^{d} substituents.

In some preferred embodiments, E⁴ is alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, aminoalkyl, carbocyclyl, carbocyclylalkyl, carbocyclylalkoxyalkyl, heterocyclyl, heterocyclylalkyl, or heterocyclylalkoxyalkyl. In these embodiments, any such substituent:
comprises at least two carbon atoms, and
is substituted with one or more independently selected halogen, and
is optionally substituted with one or more independently selected R^{d} substituents.

In some preferred embodiments, E⁴ is alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, aminoalkyl, carbocyclyl, carbocyclylalkyl, carbocyclylalkoxyalkyl, heterocyclyl, heterocyclylalkyl, or heterocyclylalkoxyalkyl. In these embodiments, any such substituent:
comprises at least two carbon atoms, and
is substituted with one or more fluoro, and
is optionally substituted with one or more independently selected R^{d} substituents.

In some preferred embodiments, E⁴ is alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, aminoalkyl, carbocyclyl, carbocyclylalkyl, carbocyclylalkoxyalkyl, heterocyclyl, heterocyclylalkyl, or heterocyclylalkoxyalkyl. In these embodiments, any such substituent:
comprises at least two carbon atoms, and
is substituted with one or more chloro, and
is optionally substituted with one or more independently selected R^{d} substituents.

In some preferred embodiments, E⁴ is alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, aminoalkyl, carbocyclyl, carbocyclylalkyl, carbocyclylalkoxyalkyl, heterocyclyl, heterocyclylalkyl, or heterocyclylalkoxyalkyl. In these embodiments, any such substituent:
comprises at least two carbon atoms, and
is substituted with one or more fluoro, and
is substituted with one or more chloro, and
is optionally substituted with one or more independently selected R^{d} substituents.

In some preferred embodiments, E⁴ is trifluoromethylmethyl, trifluoromethylethyl, trifluoromethylpropyl,

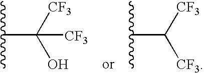

In some preferred embodiments, E⁴ is trifluoromethylmethyl, trifluoromethylethyl, trifluoromethylpropyl,

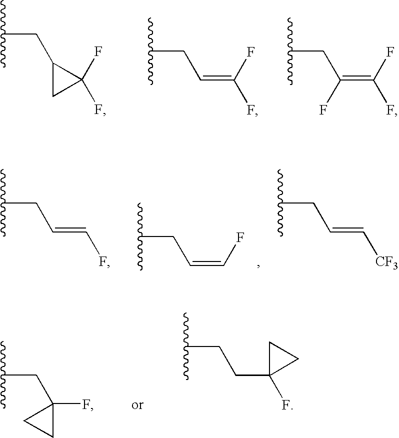

In some preferred embodiments, E⁴ is halo-C₂-C₆-alkyl.

In some preferred embodiments, E⁴ is C₂-C₆-alkyl substituted with one or more fluoro.

In some preferred embodiments, E⁴ is C₂-C₆-alkyl partially substituted with one or more independently selected halogen.

In some preferred embodiments, E⁴ is C₁-C₅-alkyl substituted with trifluoromethyl.

In some preferred embodiments, E⁴ is —(CH₂)₂—CF₃ or —(CH₂)₃—CF₃.

In some preferred embodiments, E⁴ is —CF₂—CH₃, or E⁴ is C₁-C₄-alkyl substituted with —CF₂—CH₃.

In some preferred embodiments, E⁴ is —CH₂—CF₂—CH₃ or —(CH₂)₂—CF₂—CH₃.

In some preferred embodiments, E⁴is —CF₂—CF₃, or E⁴ is C₁-C₄-alkyl substituted with —CF₂—CF₃.

In some preferred embodiments, E⁴ is —CH₂‘3CF₂—CF₃ or —(CH₂)₂—CF₂—CF₃.

In some preferred embodiments, E⁴is C₂-C₆-alkyl comprising a carbon atom bonded to at least one hydrogen and at least one halogen.

In some preferred embodiments, E⁴ is C₂-C₆-alkyl comprising a carbon atom bonded to at least one hydrogen and at least one fluoro.

In some preferred embodiments, E⁴ is C₁-C₅-alkyl substituted with —CF₂H.

In some preferred embodiments, E⁴ is —(CH₂)₃—CF₂H.

In some preferred embodiments, E⁴ is C₁-C₅-alkyl substituted with —CH₂F.

In some preferred embodiments, E⁴ is —(CH₂)₃—CH₂F.

In some preferred embodiments, E⁴ is —CF₂—CF₂H, or C₁-C₄-alkyl substituted with —CF₂—CF₂H.

In some preferred embodiments, E⁴is —CF₂—CF₂H or —CH₂—CF₂—CF₂H.

In some preferred embodiments, E⁴ is halo-C₂-C₄-alkyl.

In some preferred embodiments, E⁴is halo-C₃-C₄-alkyl.

In some preferred embodiments, E⁴ is —(CH₂)₂—CF₃, —(CH₂)₃—CH₂F, —(CH₂)₃—CF₂H, —(CH₂)₂—CF₂—CH₃, —(CH₂)₃—CF₃, —(CH₂)₂—CF₂—CF₃, or —(CH₂)₂—C(CF₃)₂F.

In some preferred embodiments, E⁴is —CF₂—CF₂H, —(CH₂)₃—CF₃, —CH₂—CF₂—CH₃, —CH₂—CF₂—CF₂H, or —CH₂—CF₂—CF₃.

In some preferred embodiments, E⁴ is phenyl substituted with one or more substituents selected from the group consisting of halogen, haloalkyl, and haloalkoxy.

In some preferred embodiments, E⁴is alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, aminoalkyl, carbocyclyl, carbocyclylalkyl, carbocyclylalkoxyalkyl, heterocyclyl, heterocyclylalkyl, or heterocyclylalkoxyalkyl. In these embodiments, any such substituent is:
substituted with one or more independently-selected halogen, and
optionally substituted with one or more independently selected R^{d} substituents.

In some preferred embodiments, E⁴ is hydroxyalkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, carbocyclyl, carbocyclylalkyl, carbocyclylalkoxyalkyl, heterocyclyl, or heterocyclylalkoxyalkyl. In these embodiments, any such group optionally is substituted with one or more independently selected R^{d} substituents.

In some preferred embodiments, E⁴ is alkynyl optionally substituted with alkoxy.

In some preferred embodiments, E⁴ is carbocyclyl or carbocyclylalkyl, wherein the carbocyclyl or carbocyclylalkyl optionally is substituted with one or more substituents independently selected from alkoxy and oxo.

In some preferred embodiments, E⁴ is heterocyclyl optionally substituted with alkyl.

In some preferred embodiments, E⁴ is heterocyclyl.

In some preferred embodiments, E⁴ is hydroxyalkyl or alkoxyalkyl, wherein the hydroxyalkyl or alkoxyalkyl optionally is substituted with oxo.

In some preferred embodiments, E⁴ is hydroxyalkyl, alkoxyalkyl, carbocyclyl, or carbocyclylalkyl.

In some preferred embodiments, E⁴ is carbocyclylalkyl or alkylheterocyclyl.

In some preferred embodiments, E⁴ is carbocyclylalkyl.

In some preferred embodiments, E⁴ is carbocyclyl.

In some preferred embodiments, E⁴ is alkyl, wherein the alkyl:
comprises a carbon chain of at least 4 carbon atoms, and
is optionally substituted with one or more independently selected R^{d} substituents.

In some preferred embodiments, E⁴ is —(CH₂)₃—CH₃.

In some preferred embodiments, E⁴ is —(CH₂)₄—CH₃.

In some preferred embodiments, E⁴ is —CH₂—CH₃.

In some preferred embodiments, E⁴ is —(CH₂)₂—CH₃.

In some preferred embodiments, E⁴ is —C(CH₃)₂H.

In some preferred embodiments, E⁴ is alkynyl.

In some preferred embodiments, -E³-E⁴ is —CH₂—CH₃, —(CH₂)₂—CH₃, —C(CH₃)₂H, or —O—CH₂-CH₃. In these embodiments, any member of such group optionally is substituted with one or more substituents independently selected from the group consisting of halogen, hydroxy, cyano, sulfo, nitro, nitroso, oxo, thioxo, imino, alkoxy, alkoxyalkyl, —N(R^{e})(R^{e}), —C(O)(R^{g}), —S—R^{e}, —S(O)₂—R^{e}, carbocyclyl, alkylcarbocyclyl, carbocyclylalkyl, heterocyclyl, alkylheterocyclyl, and heterocyclylalkyl. Any such optional substituent, in turn, is optionally substituted with one or more substituents independently selected from the group consisting of halogen, hydroxy, cyano, carboxy, thiol, sulfo, nitro, nitroso, oxo, thioxo, imino, aminocarbonyl, and amino.

In some preferred embodiments, -E³-E⁴ comprises at least 2 non-hydrogen atoms.

In some preferred embodiments, -E³-E⁴ is halo-C₁-C₆-alkyl.

In some preferred embodiments, -E³-E⁴ is trifluoromethyl.

In some preferred embodiments, -E³-E⁴ is —CH₂—CH₃ substituted with alkylheterocyclyl.

In some preferred embodiments, -E³-E⁴ is —CH₂—CH₃.

In some preferred embodiments, -E³-E⁴ is —(CH₂)₂—CH₃ substituted with heterocyclyl and oxo.

In some preferred embodiments, -E³-E⁴ is —(CH₂)₂—CH₃.

In some preferred embodiments, -E³-E⁴ is —C(CH₃)₂H.

In some preferred embodiments, -E³-E⁴ is C₁-C₆-alkoxy.

In some preferred embodiments, -E³-E⁴ is ethoxy.

In some preferred embodiments, -E³-E⁴ is methoxy.

In some preferred embodiments, -E³-E⁴ is hydrogen.

### General Description of Preferred R^{x} Substituents

Each R^{x} is independently selected from the group consisting of halogen, cyano, hydroxy, nitro, nitroso, oxo, alkyl, alkenyl, alkynyl, alkoxy, alkoxyalkoxy, R^{b}-oxyalkyl, alkenyloxy, alkynyloxy, alkylthio, R^{b}R^{b}-amino, R^{b}R^{b}-aminoalkyl, R^{b}R^{b}-aminoalkoxy, R^{b}R^{b}-aminoalkyl(R^{b})amino, carbocyclyl, carbocyclylalkyl, carbocyclyloxy, carbocyclyloxyalkoxy, carbocyclylthio, heterocyclyl, heterocyclylalkyl, heterocyclyloxy, heterocyclyloxyalkoxy, heterocyclylthio, alkyliminocarbonyl, alkylthioalkyl, alkylsulfonylalkyl, alkylsulfoxidoalkyl, alkylthioalkenyl, alkylsulfoxidoalkenyl, alkylsulfonylalkenyl, carbocyclylalkoxyalkyl, carbocyclyliminocarbonyl, carbocyclylthioalkyl, carbocyclylsulfoxidoalkyl, carbocyclylsulfonylalkyl, carbocyclylthioalkenyl, carbocyclylsulfoxidoalkenyl, carbocyclylsulfonylalkenyl, heterocyclylalkoxyalkyl, heterocyclylthioalkyl, heterocyclylsulfoxidoalkyl, heterocyclylsulfonylalkyl, heterocyclylthioalkenyl, heterocyclylsulfoxidoalkenyl, heterocyclylsulfonylalkenyl, heterocyclyliminocarbonyl, aminosulfonylalkyl, and —R^{x1}—R^{x2}. Any such group optionally is substituted with one or more substituents independently selected from the group consisting of halogen, hydroxy, cyano, amino, carboxy, thiol, sulfo, nitro, nitroso, oxo, thioxo, imino, alkyl alkoxy, alkoxyalkyl, and alkoxyalkoxy. With respect to these optional substituents:
the alkyl, alkoxy, alkoxyalkyl, and alkoxyalkoxy optionally are substituted with one or more substituents independently selected from the group consisting of halogen and hydroxy; and
the amino optionally is substituted with up to 2 independently selected alkyl.

Each R^{x1} is independently selected from the group consisting of —C(O)—, —C(S)—, —C(NR^{y})—, and —S(O)₂—.

Each R^{y} is independently selected from the group consisting of hydrogen and hydroxy.

Each R^{x2} is independently selected from the group consisting of hydrogen, hydroxy, alkyl, alkenyl, alkynyl, alkoxy, alkoxyalkyl, alkoxyalkoxy, R^{b}-oxyalkyl, alkenyloxy, alkynyloxy, R^{b}R^{b}-amino, R^{b}R^{b}-aminoalkyl, R^{b}R^{b}-aminoalkoxy, R^{b}R^{b}-aminoalkyl(R^{b})amino, carbocyclyl, carbocyclylalkyl, carbocyclyloxy, carbocyclyloxyalkoxy, heterocyclyl, heterocyclylalkyl, heterocyclyloxy, and heterocyclyloxyalkoxy. Any such substituent optionally is substituted with one or more substituents independently selected from the group consisting of halogen, hydroxy, cyano, carboxy, thiol, sulfo, nitro, nitroso, oxo, thioxo, imino, alkyl, alkoxy, alkoxyalkyl, and alkoxyalkoxy. Any such optional substituent is, in turn, optionally substituted with one or more substituents independently selected from the group consisting of halogen and hydroxy.

### General Description of Preferred R^{b}, R^{c}, R^{d}, R^{e}, R^{g}, and R^{h} Substituents

Each R^{b} is independently selected from the group consisting of hydrogen, hydroxy, alkyl, alkenyl, alkynyl, alkoxy, alkoxyalkyl, bisalkoxyalkyl, alkylthioalkyl, alkylthioalkenyl, alkylsulfoxidoalkyl, alkylsulfonyl, alkylsulfonylalkyl, carbocyclyl, carbocyclylalkyl, carbocyclyloxyalkyl, carbocyclylalkoxyalkyl, carbocyclylthioalkyl, carbocyclylthioalkenyl, carbocyclylsulfoxidoalkyl, carbocyclylsulfonyl, carbocyclylsulfonylalkyl, heterocyclyl, heterocyclylalkyl, heterocyclyloxyalkyl, heterocyclylalkoxyalkyl, heterocyclylthioalkyl, heterocyclylsulfoxidoalkyl, heterocyclylsulfonyl, heterocyclylsulfonylalkyl, aminoalkyl, aminosulfonyl, aminoalkylsulfonyl, and alkoxyalkylaminoalkyl. Any such substituent optionally is substituted with one or more substituents independently selected from the group consisting of halogen, hydroxy, cyano, carboxy, thiol, sulfo, nitro, nitroso, oxo, thioxo, imino, alkyl, alkylcarbonyl, carbocyclyl, and carbocyclylalkyl.

In some preferred embodiments, R^{b} is alkyl.

In some preferred embodiments, R^{b} is methyl.

In some preferred embodiments, R^{b} is hydrogen.

Each R^{c} is independently selected from the group consisting of halogen, hydroxy, cyano, carboxy, —C(H)(NH), —C(H)(NOH), thiol, sulfo, nitro, nitroso, oxo, thioxo, imino, amino, alkyl, alkoxy, alkenyl, alkynyl, alkoxyalkyl, mono-alkylamino, di-alkylamino, alkylthio, carbocyclyl, carbocyclylalkyl, heterocyclyl, and heterocyclylalkyl. Any such substituent optionally is substituted with one or more substituents independently selected from the group consisting of halogen, hydroxy, cyano, carboxy, thiol, sulfo, nitro, nitroso, oxo, thioxo, imino, aminocarbonyl, amino, alkyl, and carbocyclylalkyl.

Each R^{d} is independently selected from the group consisting of halogen, hydroxy, cyano, sulfo, nitro, nitroso, oxo, thioxo, imino, alkyl, alkoxy, alkoxyalkyl, —N(R^{e})(R^{e}), —C(O)(R^{g}), —S—R^{e}, —S(O)₂—R^{e}, carbocyclyl, alkylcarbocyclyl, carbocyclylalkyl, heterocyclyl, alkylheterocyclyl, and heterocyclylalkyl. Any such substituent optionally is substituted with one or more substituents independently selected from the group consisting of halogen, hydroxy, cyano, carboxy, thiol, sulfo, nitro, nitroso, oxo, thioxo, imino, aminocarbonyl, and amino.

Each R^{e} is independently selected from the group consisting of hydrogen alkyl, carbocyclyl, carbocyclylalkyl, heterocyclyl, and heterocyclylalkyl. Any such substituent optionally is substituted with one or more substituents independently selected from the group consisting of halogen, hydroxy, cyano, carboxy, thiol, sulfo, nitro, nitroso, oxo, thioxo, imino, aminocarbonyl, and amino.

Each R^{g} is independently selected from the group consisting of hydrogen, alkyl, —O—R^{h}, —N(R^{h})(R^{h}), carbocyclylalkyl, and heterocyclylalkyl. Any such substituent optionally is substituted with one or more substituents independently selected from the group consisting of halogen, hydroxy, cyano, carboxy, thiol, sulfo, nitro, nitroso, oxo, thioxo, imino, aminocarbonyl, and amino.

Each R^{h} is independently selected from the group consisting of hydrogen, alkyl, carbocyclyl, carbocyclylalkyl, heterocyclyl, and heterocyclylalkyl. Any such substituent optionally is substituted with one or more substituents independently selected from the group consisting of halogen, hydroxy, cyano, carboxy, thiol, sulfo, nitro, nitroso, oxo, thioxo, imino, aminocarbonyl, and amino.

### General Description of Preferred Structures

In some preferred embodiments, the compound of this invention corresponds in structure to Formula (I-U): 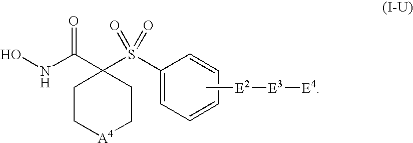
Here, A⁴ is —O—, —N(H)—, —N(R^{x})—, —S—, —S(O)—, —S(O)₂—, —C(H)₂—, or —C(R^{x})₂—. In some such embodiments, the compound of this invention corresponds in structure to Formula (I-V):

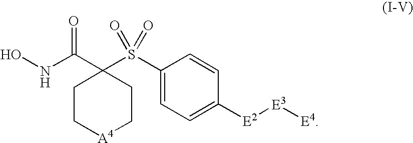

In some preferred embodiments, the compound corresponds in structure to Formula (I-W) or Formula (I-X):

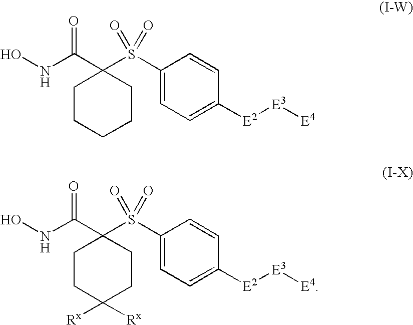

In some preferred embodiments, the compound corresponds in structure to Formula (I-Y): 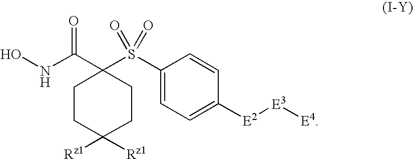
Here, each R^{z1} is independently selected from the group consisting of hydrogen, halogen, alkyl, haloalkyl, alkoxy, and alkoxyalkoxy.

In some particularly preferred embodiments, the compound corresponds in structure to Formula (I-Z):

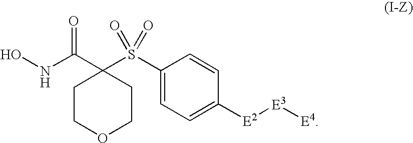

In some particularly preferred embodiments, the compound corresponds in structure to Formula (I-AA):

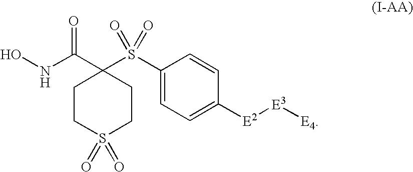

In some particularly preferred embodiments, the compound corresponds in structure to Formula (I-BB):

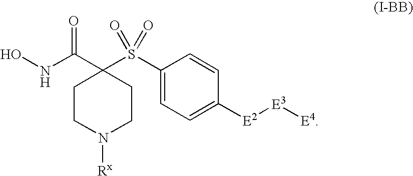

In some particularly preferred embodiments, the compound corresponds in structure to Formula (I-CC): 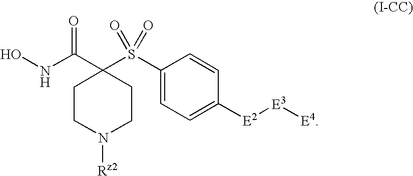
Here, R^{z2} is alkyl, alkoxyalkyl, cycloalkyl, formyl, heterocycloalkylcarbonyl, or dialkylaminocarbonyl.

### Detailed Description of Several Preferred Embodiments

The above discussion describes the compounds and salts of this invention in general terms. The following discussion, in turn, describes in detail several preferred embodiments.

### Preferred Embodiment No. 1

In some preferred embodiments, E⁴ is alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, aminoalkyl, carbocyclyl, carbocyclylalkyl, carbocyclylalkoxyalkyl, heterocyclyl, heterocyclylalkyl, or heterocyclylalkoxyalkyl. Here, any such substituent:
comprises at least two carbon atoms, and
is substituted with one or more independently-selected halogen, and
is optionally substituted with one or more independently selected R^{d} substituents.

### Particularly Preferred Embodiments of Embodiment No. 1

In some particularly preferred embodiments, A² is hydrogen.

In some particularly preferred embodiments, A³ is alkoxyalkyl.

In some particularly preferred embodiments, A² is hydrogen, and A³ is alkoxyalkyl. Examples of such compounds include the following:

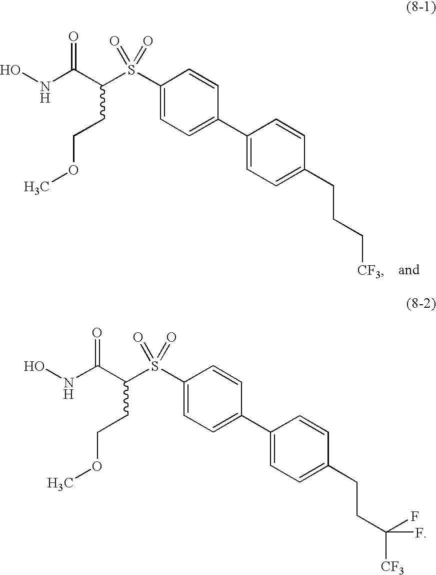

In some particularly preferred embodiments, the compound corresponds in structure to Formula (9-1): 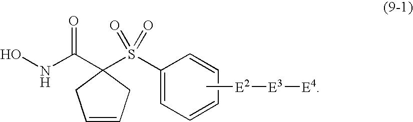
An example of such a compound includes the following:

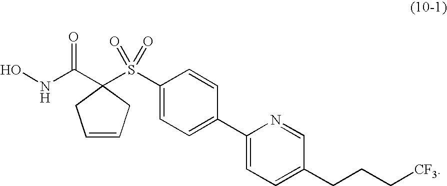

In some particularly preferred embodiments, the compound corresponds in structure to Formula (Error! Reference source not found.-1): 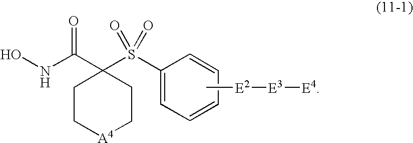
Here, A⁴ is —O—, —N(H)—, —N(R^{x})—, —S—, —S(O)—, —S(O)₂—, —C(H)₂—, or —C(R^{x})₂—.

In some particularly preferred embodiments, the compound corresponds in structure to Formula (12-1):

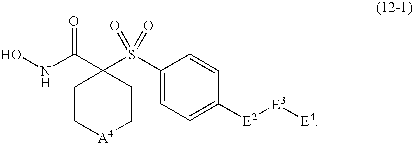

In some particularly preferred embodiments, the compound corresponds in structure to Formula (13-1): 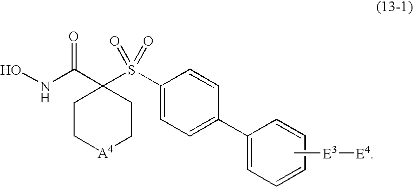
In some such embodiments, the compound corresponds in structure to Formula (14-1): 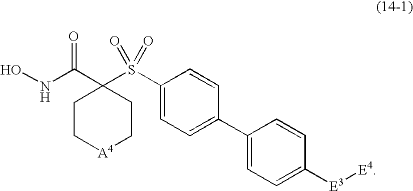
In other such embodiments, the compound corresponds in structure to Formula (15-1):

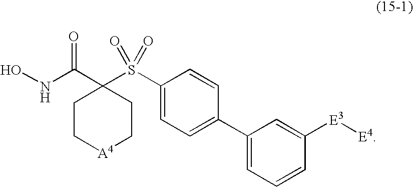

In some particularly preferred embodiments, the compound corresponds in structure to one of the following formulas:

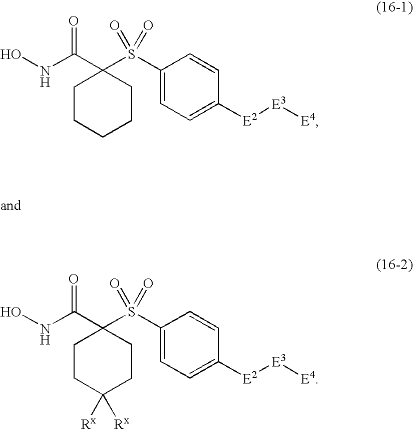

In some particularly preferred embodiments, the compound corresponds in structure to Formula (I-C-1): 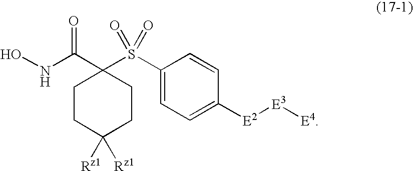
Here, each R^{z1} is independently selected from the group consisting of hydrogen, halogen, alkyl, haloalkyl, alkoxy, and alkoxyalkoxy.

In some particularly preferred embodiments, the compound corresponds in structure to Formula (18-1):

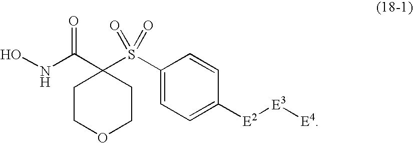

In some particularly preferred embodiments, the compound corresponds in structure to Formula (19-1):

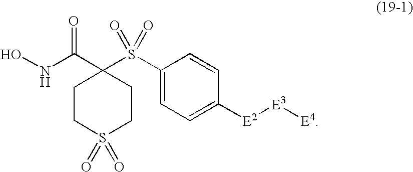

In some particularly preferred embodiments, the compound corresponds in structure to Formula (20-1):

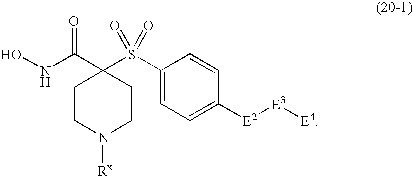

In some particularly preferred embodiments, the compound corresponds in structure to Formula (21-1): 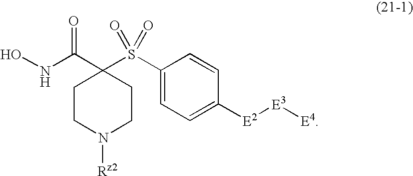
Here, R^{x2} is alkyl, alkoxyalkyl, cycloalkyl, formyl, heterocycloalkylcarbonyl, or dialkylaminocarbonyl.

In some particularly preferred embodiments, E² is phenyl substituted with one or more independently selected R^{x} substituents.

In some particularly preferred embodiments, E² is phenyl optionally substituted with one or more substituents independently selected from the group consisting of halogen and haloalkyl.

In some particularly preferred embodiments, E² is phenyl.

In some particularly preferred embodiments, E² is heteroaryl optionally substituted with one or more independently selected R^{x} substituents.

In some particularly preferred embodiments, E² is heteroaryl that is not substituted with any optional R^{x} substituents.

In some particularly preferred embodiments, E² is furanyl, thienyl, oxazolyl, isoxazolyl, thiazolyl, isothiazolyl, thiodiazolyl, oxadiazolyl, pyridinyl, pyrazinyl, pyrimidinyl, pyridazinyl, triazinyl, oxathiazinyl, oxepinyl, thiepinyl, benzofuranyl, isobenzofuranyl, benzoxazolyl, benzoisoxazolyl, benzothienyl, isobenzothienyl, benzothiazolyl, benzoisothiazolyl, benzothiadiazolyl, indolizinyl, pyranopyrrolyl, quinolinyl, isoquinolinyl, naphthyridinyl, phthalazinyl, quinoxalinyl, quinazolinyl, cinnolinyl, pteridinyl, or acridinyl.

In some particularly preferred embodiments, E² is thienyl, oxadiazolyl, or pyridinyl.

In some particularly preferred embodiments, E² is 5-member heteroaryl. In some such embodiments, E² is thienyl or oxadiazolyl.

In some particularly preferred embodiments, E² is 6-member heteroaryl. In some such embodiments, E² is pyridinyl, pyrazinyl, or pyrimidinyl.

In some particularly preferred embodiments, E³ is a bond.

In some particularly preferred embodiments, E³ is a —O—.

In some particularly preferred embodiments, E³ is —C(O)—N(H)—.

In some particularly preferred embodiments, E⁴ is alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, aminoalkyl, carbocyclyl, carbocyclylalkyl, carbocyclylalkoxyalkyl, heterocyclyl, heterocyclylalkyl, or heterocyclylalkoxyalkyl. In these embodiments, any such substituent:
comprises at least two carbon atoms, and
is substituted with one or more fluoro, and
is optionally substituted with one or more independently selected R^{d} substituents.

In some particularly preferred embodiments, E⁴ is alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, aminoalkyl, carbocyclyl, carbocyclylalkyl, carbocyclylalkoxyalkyl, heterocyclyl, heterocyclylalkyl, or heterocyclylalkoxyalkyl. In these embodiments, any such substituent:
comprises at least two carbon atoms, and
is substituted with one or more chloro, and
is optionally substituted with one or more independently selected Rd substituents.

In some particularly preferred embodiments, E⁴ is alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, aminoalkyl, carbocyclyl, carbocyclylalkyl, carbocyclylalkoxyalkyl, heterocyclyl, heterocyclylalkyl, or heterocyclylalkoxyalkyl. In these embodiments, any such substituent:
comprises at least two carbon atoms, and
is substituted with one or more independently selected halogen, and
is optionally substituted with one or more independently selected R^{d} substituents.

In some particularly preferred embodiments, E⁴ is trifluoromethylmethyl, trifluoromethylethyl, trifluoromethylpropyl,

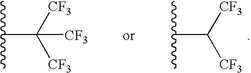

In some particularly preferred embodiments, E⁴ is trifluoromethylmethyl, trifluoromethylethyl, trifluoromethylpropyl,

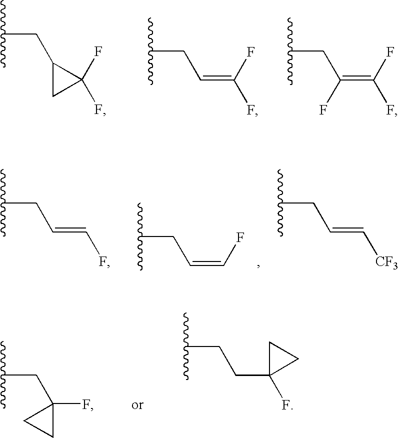

In some particularly preferred embodiments, E⁴ is halo-C₂-C₆-alkyl.

In some particularly preferred embodiments, E⁴ is C₂-C₆-alkyl substituted with one or more fluoro.

In some particularly preferred embodiments, E⁴ is C₂-C₆-alkyl partially substituted with one or more independently selected halogen.

In some particularly preferred embodiments, E⁴ is C₁-C₅-alkyl substituted with trifluoromethyl.

In some particularly preferred embodiments, E⁴ is —(CH₂)₂—CF₃ or —(CH₂)₃—CF₃.

In some particularly preferred embodiments, E⁴ is —CF₂—CH₃, or E⁴ is C₁-C₄-alkyl substituted with —CF₂—CH₃.

In some particularly preferred embodiments, E⁴ is —CH₂—CF₂—CH₃ or —(CH₂)₂—CF₂—CH₃.

In some particularly preferred embodiments, E⁴ is —CF₂—CF₃, or E⁴ is C₁-C₄-alkyl substituted with —CF₂—CF₃.

In some particularly preferred embodiments, E⁴ is —CH₂—CF₂—CF₃ or —(CH₂)₂—CF₂—CF₃.

In some particularly preferred embodiments, E⁴ is C₂-C₆-alkyl comprising a carbon atom bonded to at least one hydrogen and at least one halogen.

In some particularly preferred embodiments, E⁴ is C₂-C₆-alkyl comprising a carbon atom bonded to at least one hydrogen and at least one fluoro.

In some particularly preferred embodiments, E⁴ is C₁-C₅-alkyl substituted with —CF₂H.

In some particularly preferred embodiments, E⁴ is —(CH₂)₃—CF₂H.

In some particularly preferred embodiments, E⁴ is C₁-C₅-alkyl substituted with —CH₂F.

In some particularly preferred embodiments, E⁴ is —(CH₂)₃—CH₂F.

In some particularly preferred embodiments, E⁴ is —CF₂—CF₂H, or C₁-C₄-alkyl substituted with —CF₂—CF₂H.

In some particularly preferred embodiments, E⁴ is —CF₂—CF₂H or —CH₂—CF₂—CF₂H.

In some particularly preferred embodiments, E⁴ is halo-C₂-C₄-alkyl.

In some particularly preferred embodiments, E⁴ is halo-C₃-C₄-alkyl.

In some particularly preferred embodiments, E⁴ is —(CH₂)₂—CF₃, —(CH₂)₃—CH₂F, —(CH₂)₃—CF₂H, —(CH₂)₂—CF₂—CH₃, —(CH₂)₃—CF₃, —(CH₂)₂—C(CF₃)₂F.

In some particularly preferred embodiments, E⁴ is —CF₂—CF₂H, —(CH₂)₃—CF₃, —CH₂—CF₂—CH₃, —CH₂—CF₂—CF₂H, or —CH₂—CF₂—CF₃.

In some particularly preferred embodiments, E⁴ is phenyl substituted with one or more substituents selected from the group consisting of halogen, haloalkyl, and haloalkoxy.

In some particularly preferred embodiments, E² is phenyl optionally substituted with one or more substituents independently selected from the group consisting of halogen and haloalkyl; E³ is a bond; and E⁴ is —(CH₂)₂—CF₃, —(CH₂)₃—CH₂F, —(CH₂)₃—CF₂H, —(CH₂)₂—CF₂—CH₃, —(CH₂)₃—CF₃, —(CH₂)₂—CF₂—CF₃, or —(CH₂)₂—C(CF₃)₂F. Examples of compounds falling within these embodiments include:

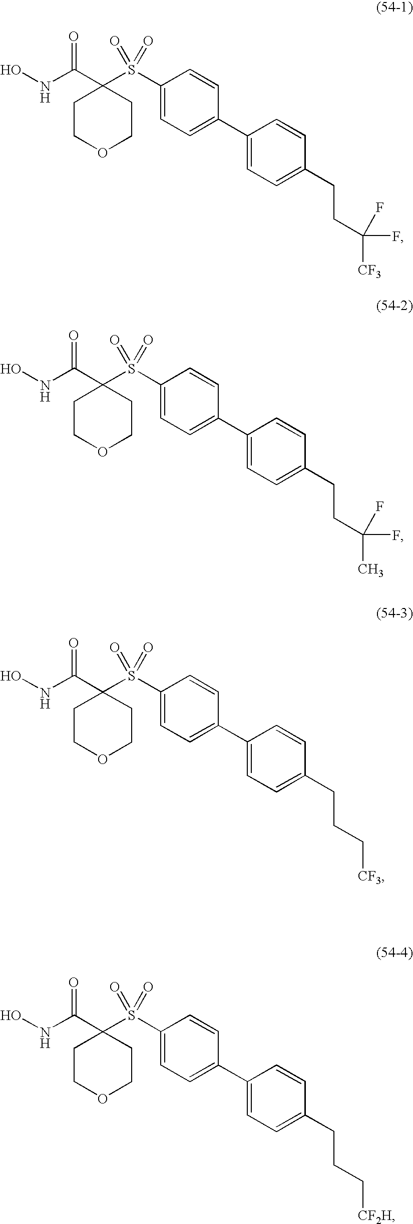

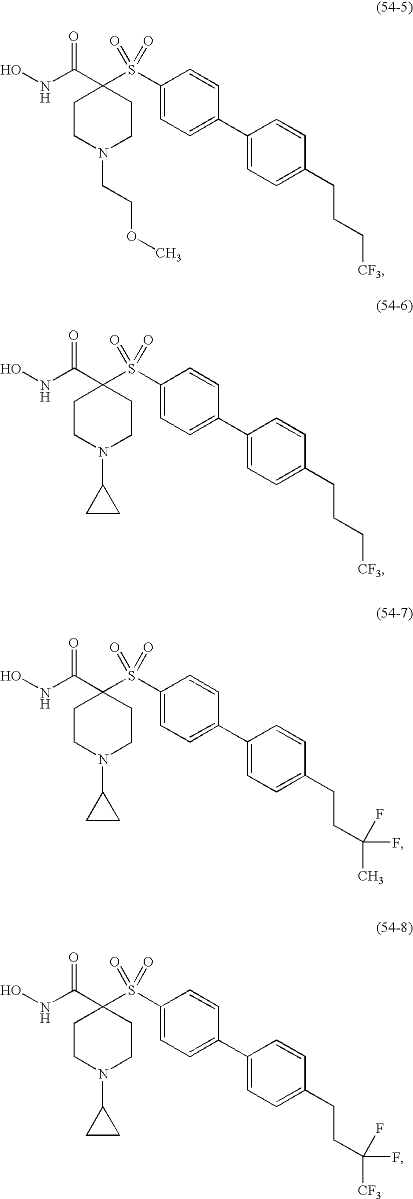

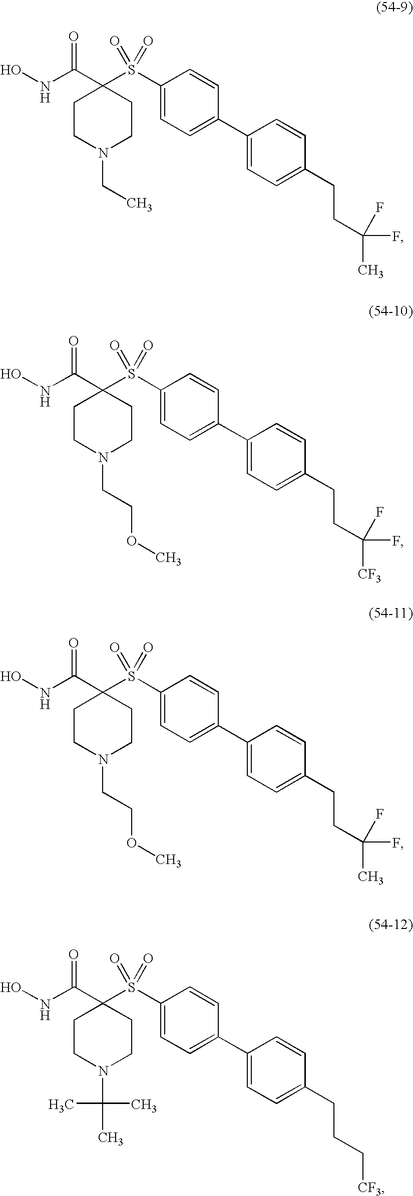

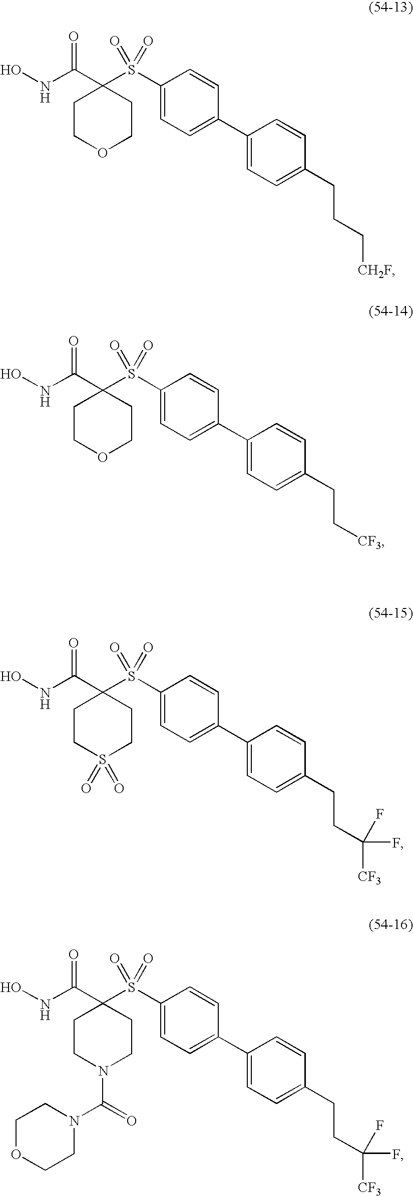

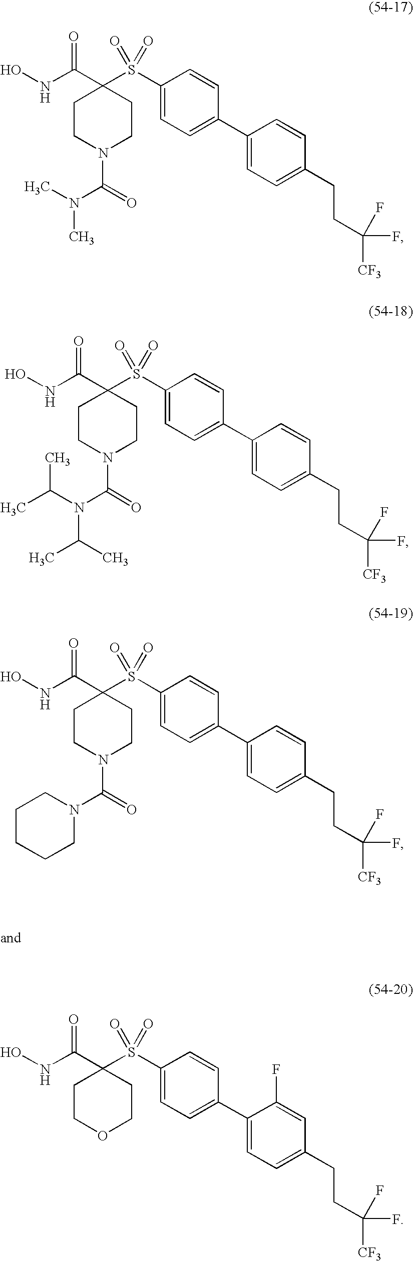

In some particularly preferred embodiments, E² is pyridinyl, pyrazinyl, or pyrimidinyl; E³ is a bond; and E⁴ is —(CH₂)₂—CF₃, —(CH₂)₃—CH₂F, —(CH₂)₃—CF₂H, —(CH₂)₂—CF₂—CH₃, —(CH₂)₃—CF₃, —(CH₂)₂—CF₂—CF₃, or —(CH₂)₂—C(CF₃)₂F. Examples of compounds falling within these embodiments include:

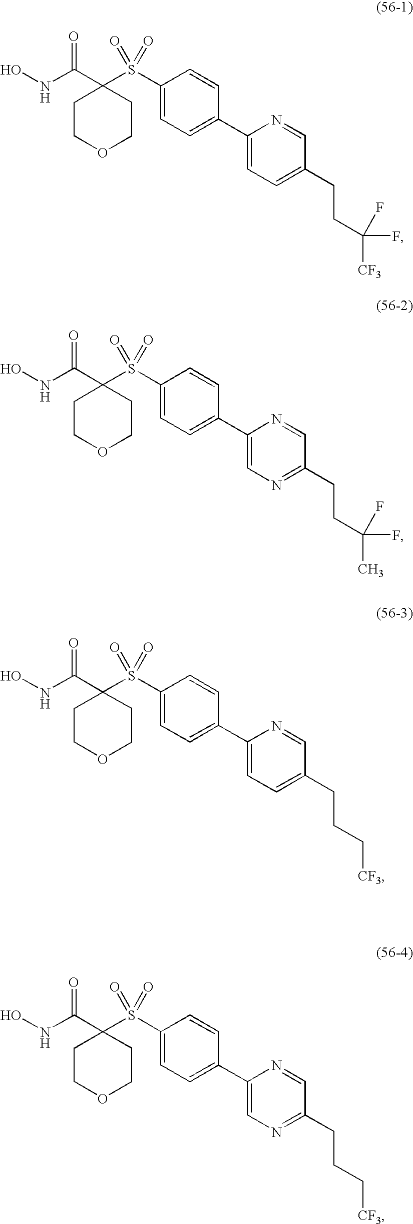

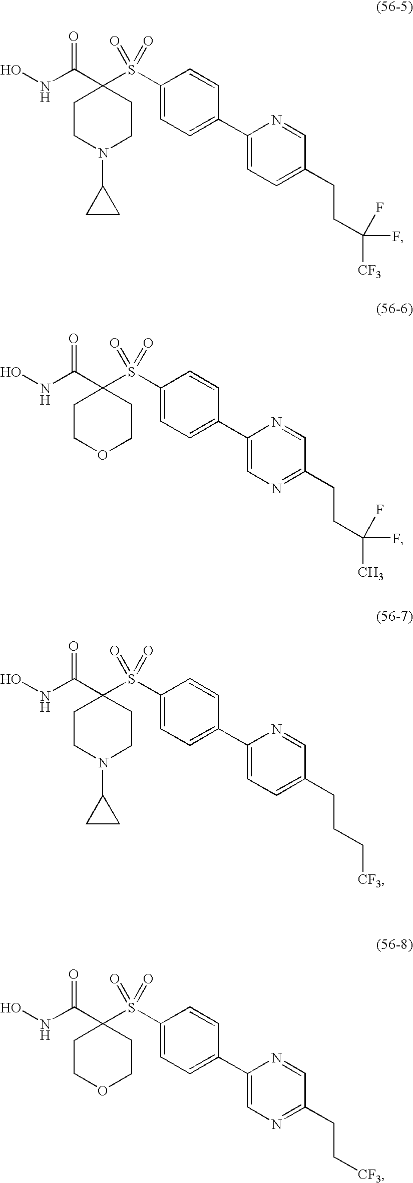

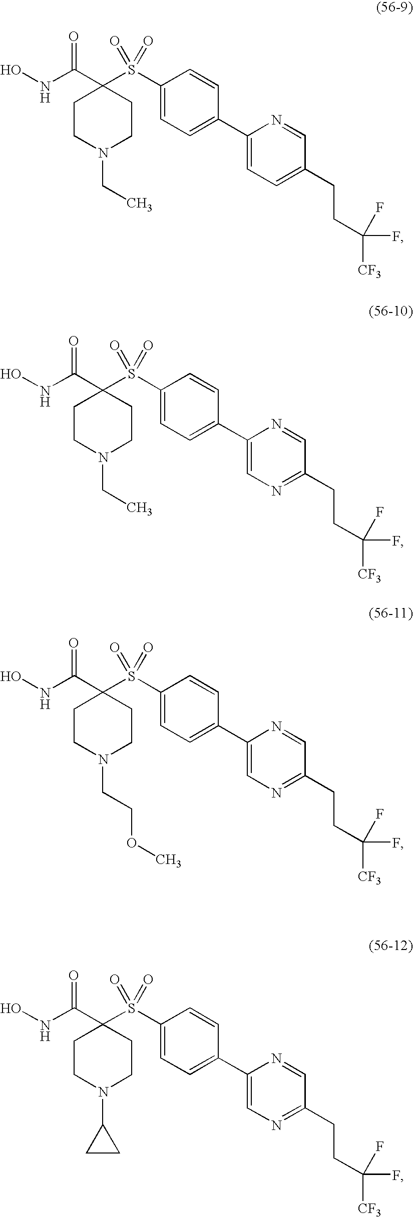

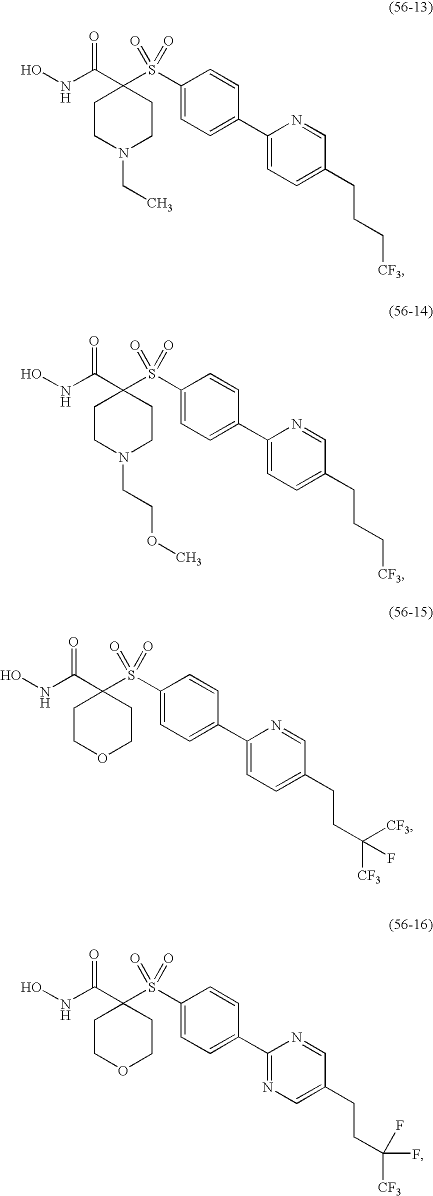

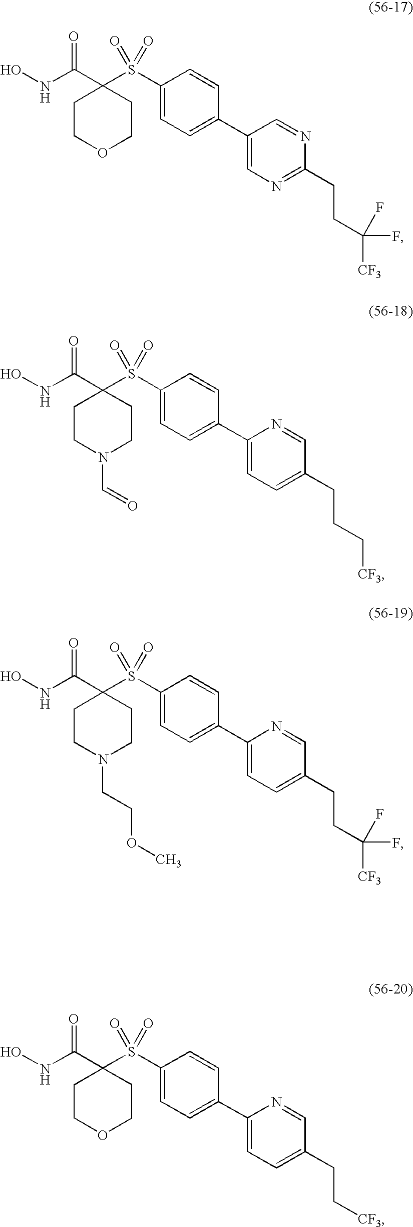

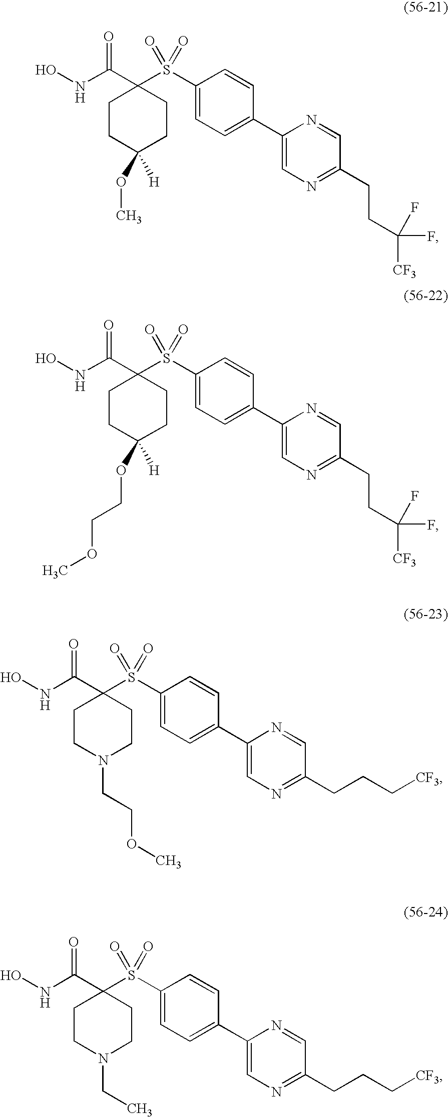

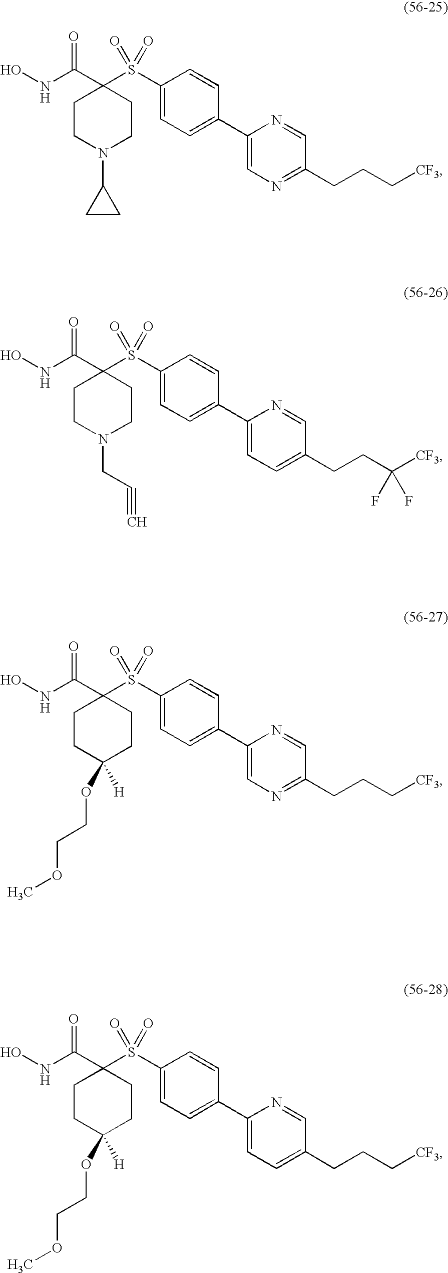

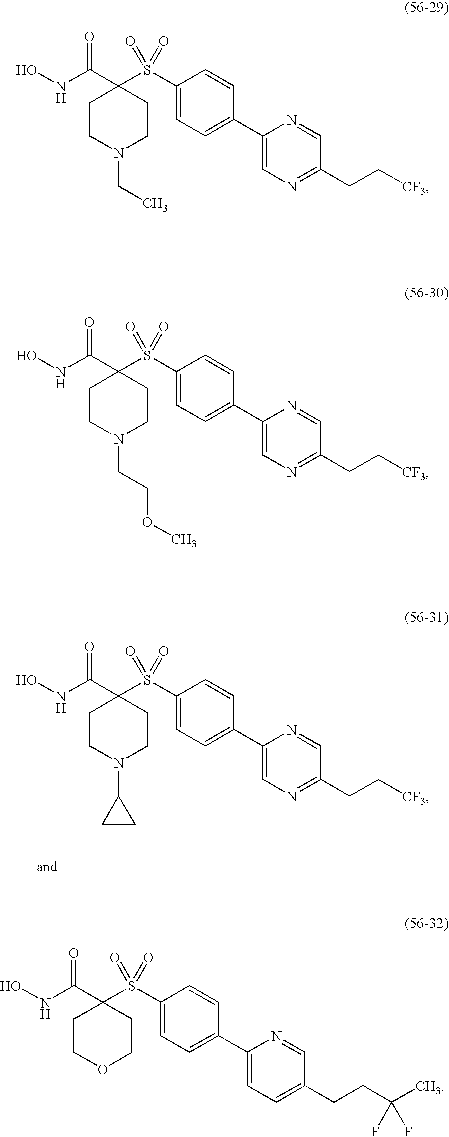

In some particularly preferred embodiments, E² is phenyl; E³ is —O—; and E⁴ is —CF₂—CF₂H, —(CH₂)₃—CF₃, —CH₂—CF₂—CH₃, —CH₂—CF₂—CF₂H, or —CH₂—CF₂—CF₃. Examples of compounds falling within these embodiments include:

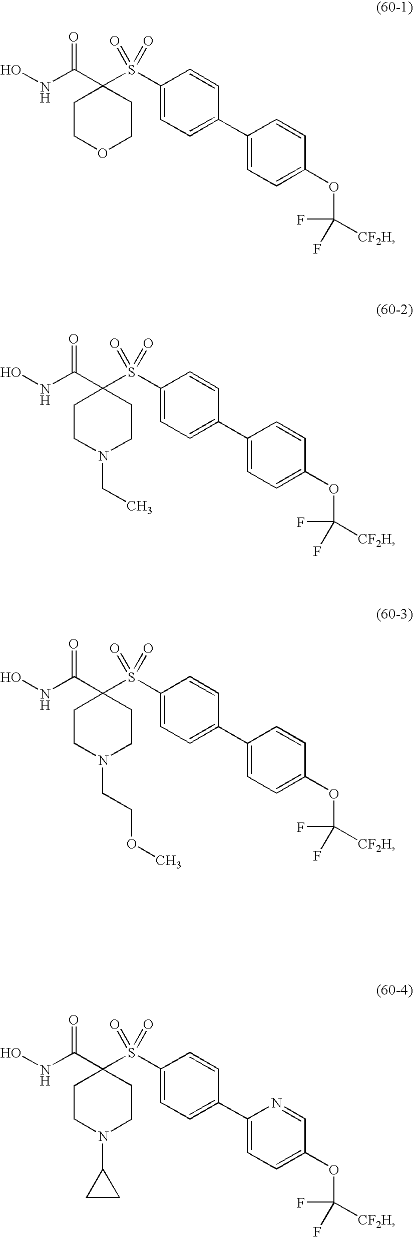

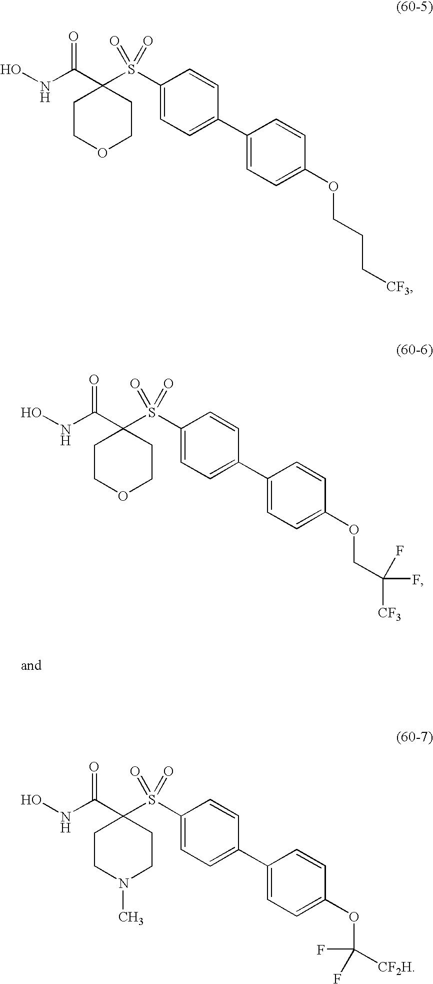

In some particularly preferred embodiments, E² is phenyl substituted with substituted with one or more substituents independently selected from the group consisting of halogen and haloalkyl; E³ is —O—; and E⁴ is —CF₂—CF₂H, —(CH₂)₃—CF₃, —CH₂—CF₂—CH₃, —CH₂—CF₂—CF₂H, or —CH₂—CF₂—CF₃. Examples of compounds falling within these embodiments include:

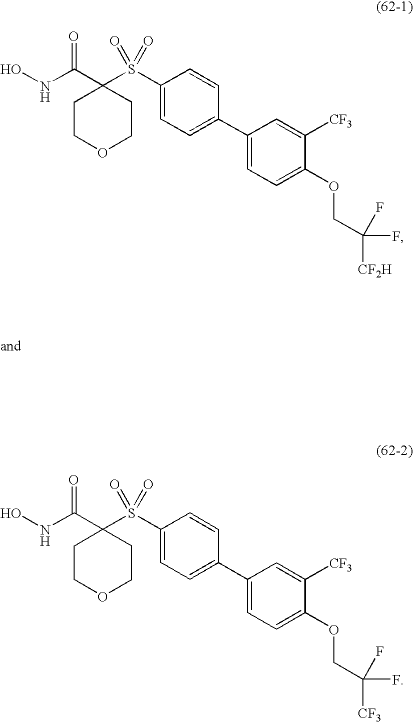

In some particularly preferred embodiments, E² is selected from the group consisting of pyridinyl, pyrazinyl, or pyrimidinyl; E³ is —O—; and E⁴ is —CF₂—CF₂H, —(CH₂)₃—CF₃, —CH₂—CF₂—CH₃, —CH₂—CF₂—CF₂H, or —CH₂—CF₂—CF₃. Examples of compounds within these embodiments include:

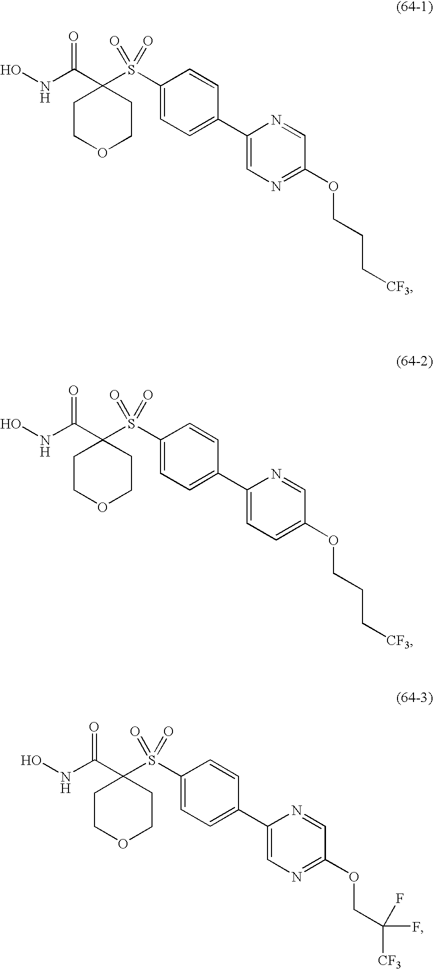

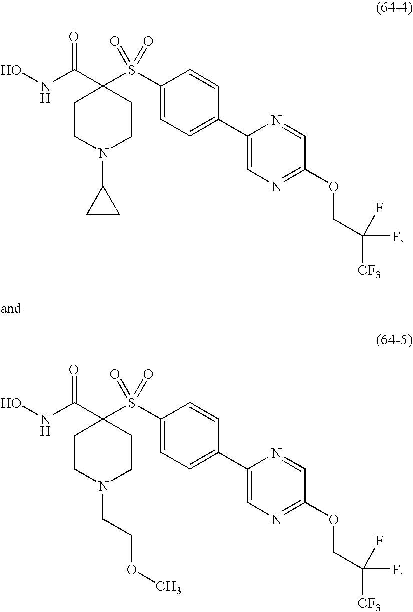

In some particularly preferred embodiments, E³ is —C(O)—N(H)—, and E⁴ is halo-C₂-C₄-alkyl. An example of a compound falling within these embodiments includes:

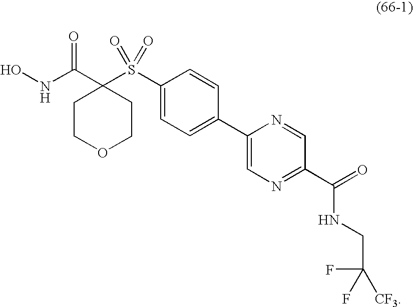

In some particularly preferred embodiments, E³ is a bond; and E⁴ is alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, aminoalkyl, carbocyclyl, carbocyclylalkyl, carbocyclylalkoxyalkyl, heterocyclyl, heterocyclylalkyl, or heterocyclylalkoxyalkyl. In these embodiments, any such substituent:
comprises at least two carbon atoms, and
is substituted with one or more independently selected halogen, and
is optionally substituted with one or more independently selected R^{d} substituents.
   An example of a compound falling within these embodiments includes:

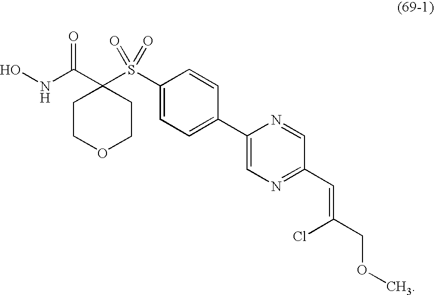

In some particularly preferred embodiments, E² is selected from the group consisting of oxadiazolyl, thienyl, and pyridinyl; E³ is a bond; and E⁴ is phenyl substituted with one or more substituents selected from the group consisting of halogen, haloalkyl, and haloalkoxy. Examples of compounds falling within these embodiments include:

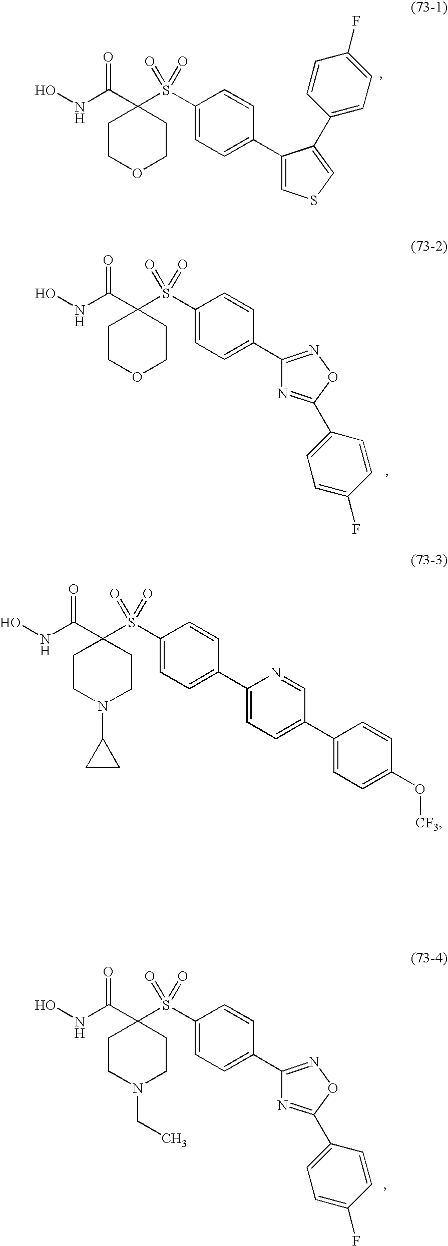

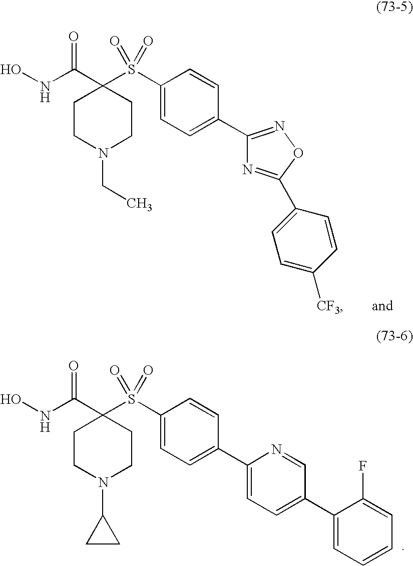

### Preferred Embodiment No. 2

In some preferred embodiments:

E³ is —O—, —C(O)—, —C(O)—O—, —O—C(O)—, —N(R^{b})—, —C(O)—N(R^{b})—, —N(R^{b})—C(O)—, —C(O)—N(R^{b})—N(R^{b})—C(O)—, —N(R^{b})—C(O)—N(R^{b})—, —S—, —S(O)—, —S(O)₂—, —N(R^{b})—S(O)₂—, —S(O)₂—N(R^{b})—, —O—S(O)₂—, —S(O)₂—O—, —C(NH)—, —C(NOH)—, —N(R^{b})—C(NH)—, —N(R^{b})—C(NOH)—, —C(NH)—N(R^{b})—, —C(NOH)—N(R^{b})—, alkyl, alkenyl, carbonylalkyl, or alkylcarbonyl, wherein:
any alkyl or alkenyl portion of such substituent optionally is substituted with one or more independently selected R^{c} substituents; and

E⁴ is alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, aminoalkyl, carbocyclyl, carbocyclylalkyl, carbocyclylalkoxyalkyl, heterocyclyl, heterocyclylalkyl, or heterocyclylalkoxyalkyl, wherein any such substituent is:
substituted with one or more independently-selected halogen, and
optionally substituted with one or more independently selected R^{d} substituents.

### Particularly Preferred Embodiments of Embodiment No. 2

In some particularly preferred embodiments, the compound corresponds in structure to Formula (78-1): 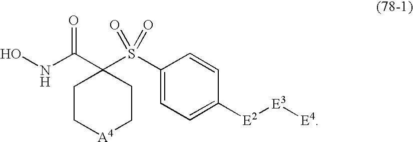
Here, A⁴ is —O—, —N(H)—, —N(R^{x})—, —S—, —S(O)—, —S(O)₂—, —C(H)₂—, or —C(R^{x})₂—. An example of a compound falling within such embodiments includes:

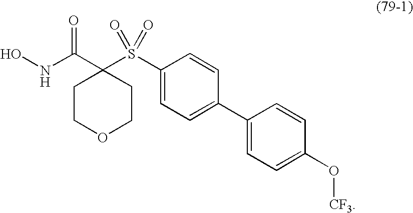

### Preferred Embodiment No. 3

In some preferred embodiments:

E² is aryl or heteroaryl, wherein the aryl or heteroaryl is:
substituted with one or more independently selected halogen, and
optionally substituted with one or more independently selected Rx substituents; and

E⁴ is alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, aminoalkyl, carbocyclyl, carbocyclylalkyl, carbocyclylalkoxyalkyl, heterocyclyl, heterocyclylalkyl, or heterocyclylalkoxyalkyl, wherein:
any such group optionally is substituted with one or more independently selected R^{d} substituents; and

-E³-E⁴ comprises at least 2 non-hydrogen atoms.

### Particularly Preferred Embodiments of Embodiment No. 3

In some particularly preferred embodiments, the compound corresponds in structure to Formula (83-1): 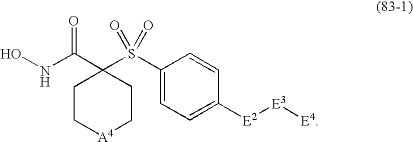
Here, A⁴ is —O—, —N(H)—, —N(R^{x})—, —S—, —S(O)—, —S(O)₂—, —C(H)₂—, or —C(R^{x})₂—.

In some particularly preferred embodiments, E² is aryl or heteroaryl, wherein the aryl or heteroaryl is substituted with one halogen.

In some particularly preferred embodiments, E² is aryl or heteroaryl, wherein the aryl or heteroaryl is substituted with one fluoro.

In some particularly preferred embodiments, E² is phenyl substituted with one halogen.

In some particularly preferred embodiments, E² is phenyl substituted with one fluoro.

In some particularly preferred embodiments, -E³-E⁴ is halo-C₁-C₆-alkyl.

In some particularly preferred embodiments, -E³-E⁴ is trifluoromethyl. Examples of compounds falling within these embodiments include:

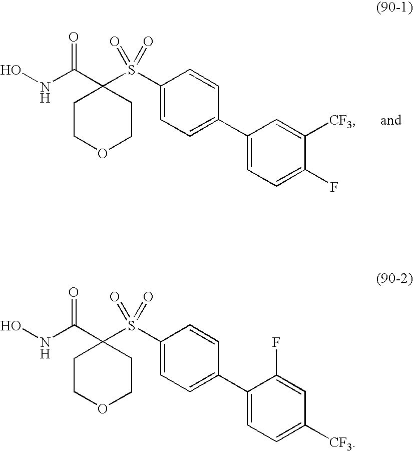

In some particularly preferred embodiments, -E³-E⁴ is C₁-C₆-alkoxy. In some such embodiments, -E³-E⁴ is methoxy. An example of a compound falling within these embodiments include:

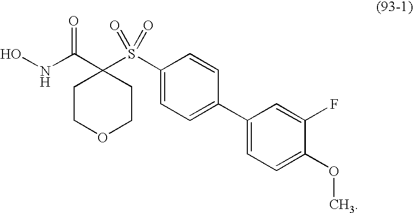

### Preferred Embodiment No. 4

In some preferred embodiments:

E³ is —O—, —C(O)—O—, —O—C(O)—, —N(R^{b})—, —C(O)—N(R^{b})—, —N(R^{b})—C(O)—, —C(O)—N(R^{b})—N(R^{b})—C(O)—, —N(R^{b})—C(O)—N(R^{b})—, —S—, —S(O)—, —S(O)₂—, —N(R^{b})—S(O)₂—, —S(O)₂—N(R^{b})—, —O—S(O)₂—, —S(O)₂—O—, —C(NH)—, —C(NOH)—, —N(R^{b})—C(NH)—, —N(R^{b})—C(NOH)—, —C(NH)—N(R^{b})—, —C(NOH)—N(R^{b})—, alkenyl, carbonylalkyl, alkylcarbonyl, or a bond, wherein:
any alkyl or alkenyl portion of a substituent in such group optionally is substituted with one or more independently selected RC substituents; and

E⁴ is hydroxyalkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, carbocyclyl, carbocyclylalkyl, carbocyclylalkoxyalkyl, heterocyclyl, or heterocyclylalkoxyalkyl, wherein:
any such group optionally is substituted with one or more independently selected R^{d} substituents.

### Particularly Preferred Embodiments of Embodiment No. 4

In some particularly preferred embodiments, the compound corresponds in structure to Formula (97-1): 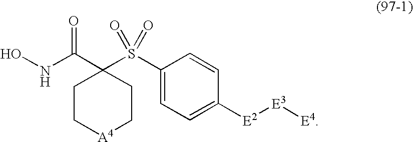
Here, A⁴ is —O—, —N(H)—, —N(R^{x})—, —S—, —S(O)—, —S(O)₂—, —C(H)₂—, or —C(R^{x})₂—.

In some particularly preferred embodiments, E² is phenyl.

In some particularly preferred embodiments, E² is naphthyl.

In some particularly preferred embodiments, E² is heteroaryl.

In some particularly preferred embodiments, E³ is a bond.

In some particularly preferred embodiments, E³ is —O—.

In some particularly preferred embodiments, E³ is —N(H)—.

In some particularly preferred embodiments, E³ is —C(O)—N(H)— or C(O)—N(CH₃)—.

In some particularly preferred embodiments, E³ is carbonylalkyl.

In some particularly preferred embodiments, E⁴ is alkynyl optionally substituted with alkoxy.

In some particularly preferred embodiments, E⁴ is carbocyclyl or carbocyclylalkyl, wherein the carbocyclyl or carbocyclylalkyl is optionally substituted with one or more substituents independently selected from alkoxy and oxo.

In some particularly preferred embodiments, E⁴ is heterocyclyl optionally substituted with alkyl.

In some particularly preferred embodiments, E⁴ is heterocyclyl.

In some particularly preferred embodiments, E⁴ is hydroxyalkyl or alkoxyalkyl, wherein hydroxyalkyl or alkoxyalkyl optionally is substituted with oxo.

In some particularly preferred embodiments, E⁴ is carbocyclylalkyl or alkylheterocyclyl.

In some particularly preferred embodiments, E⁴ is hydroxyalkyl, alkoxyalkyl, carbocyclyl, or carbocyclylalkyl.

In some particularly preferred embodiments, E⁴ is carbocyclylalkyl.

In some particularly preferred embodiments, E⁴ is carbocyclyl.

In some particularly preferred embodiments, E⁴ is alkynyl.

In some particularly preferred embodiments, E³ is a bond, and E⁴ is alkynyl optionally substituted with alkoxy. Examples of compounds falling within these embodiments include:

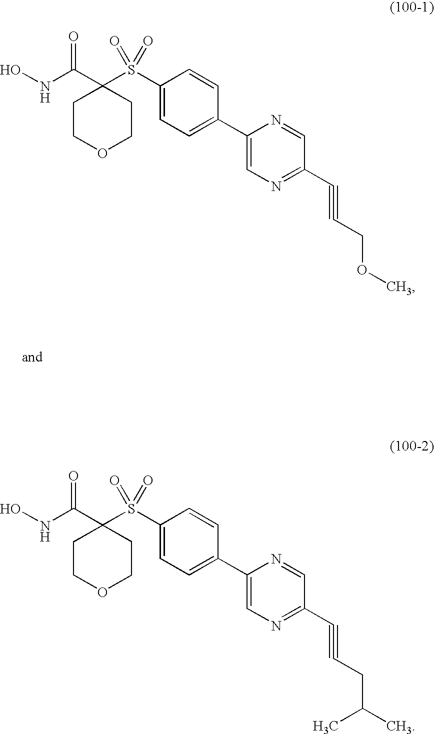

In some particularly preferred embodiments, E is phenyl; E³ is a bond; and E⁴ is carbocyclyl or carbocyclylalkyl, wherein the carbocyclyl or carbocyclylalkyl optionally is substituted with one or more substituents independently selected from alkoxy and oxo. Examples of compounds falling within these embodiments include:

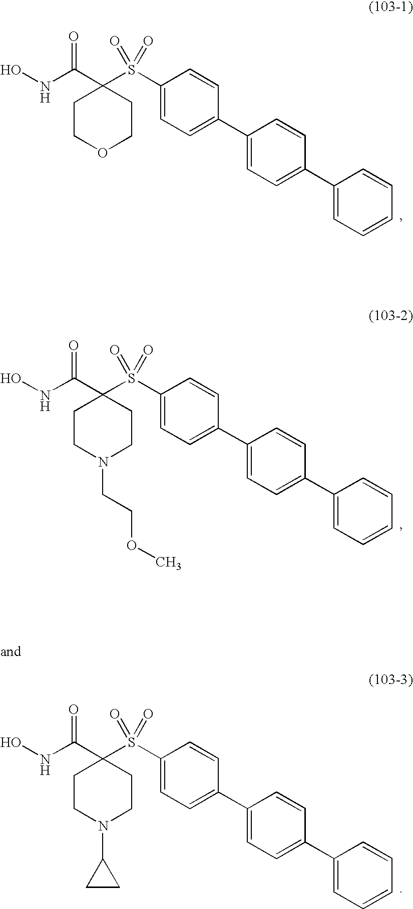

In some particularly preferred embodiments, E² is heteroaryl; E³ is a bond; and E⁴ is carbocyclyl or carbocyclylalkyl, wherein the carbocyclyl or carbocyclylalkyl optionally is substituted with one or more substituents independently selected from alkoxy and oxo. Examples of compounds falling within these embodiments include:

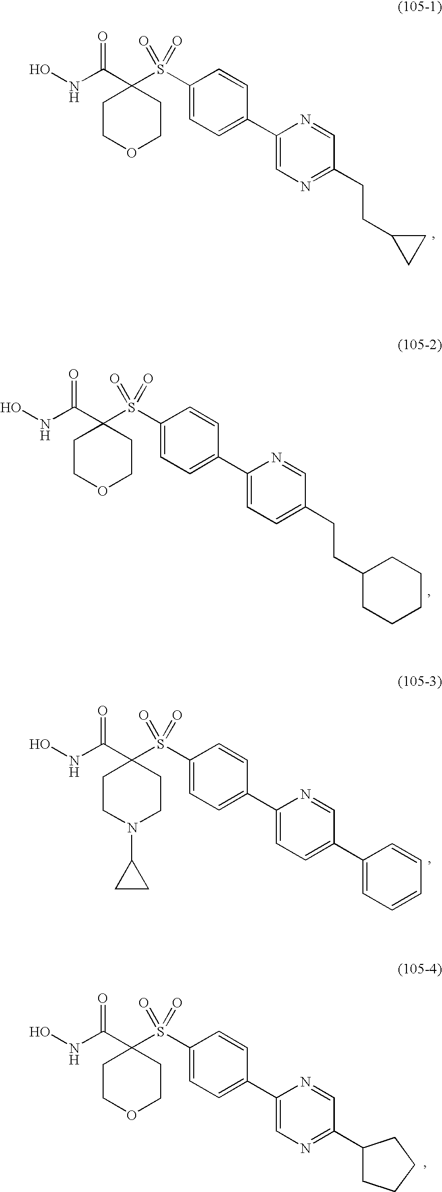

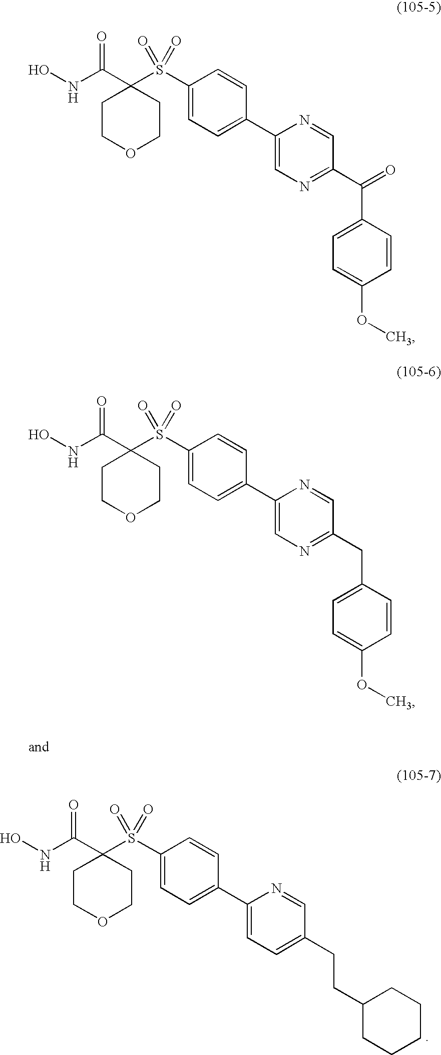

In some particularly preferred embodiments, E² is phenyl, E³ is a bond, and E⁴ is heterocyclyl optionally substituted with alkyl. Examples of compounds falling within these embodiments include:

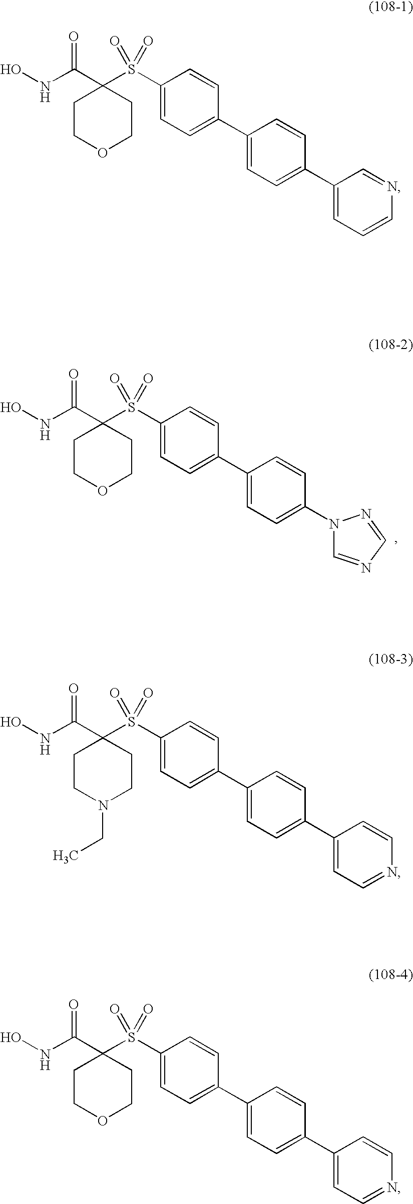

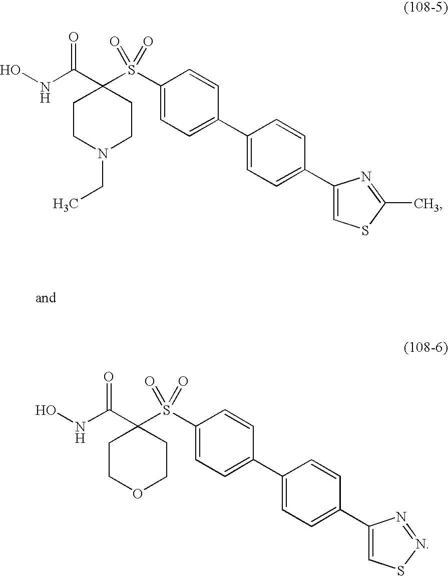

In some particularly preferred embodiments, E² is heteroaryl, E³ is a bond, and E⁴ is heterocyclyl optionally substituted with alkyl. Examples of compounds falling within these embodiments include:

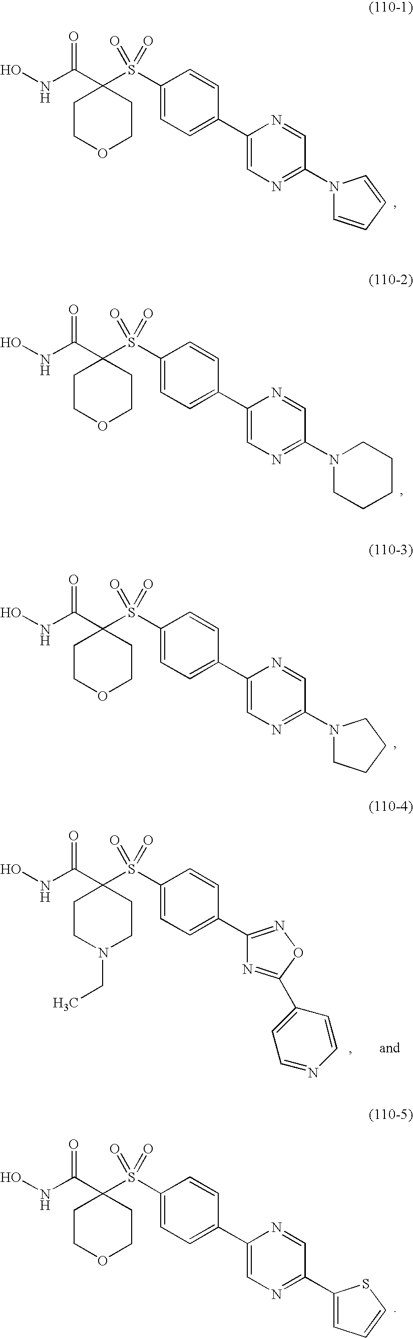

In some particularly preferred embodiments, E² is phenyl; E³ is a bond; and E⁴ is hydroxyalkyl or alkoxyalkyl, wherein the hydroxyalkyl or alkoxyalkyl optionally is substituted with oxo. An example of a compound falling within such embodiments includes: 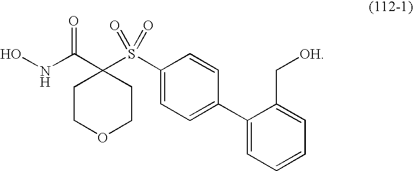
A generally more preferred (particularly if used as an MMP inhibitor) compound falling within such embodiments includes:

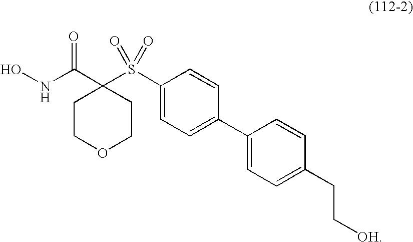

In some particularly preferred embodiments, E² is naphthyl; E³ is a bond; and E⁴ is hydroxyalkyl or alkoxyalkyl, wherein the hydroxyalkyl or alkoxyalkyl optionally is substituted with oxo. An example of a compound falling within such embodiments includes:

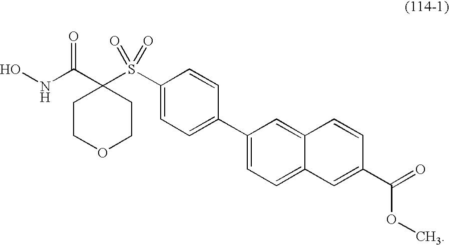

In some particularly preferred embodiments, E² is phenyl optionally substituted with one or more substituents independently selected from the group consisting of halogen and haloalkyl; E³ is —O—; and E⁴ is hydroxyalkyl, alkoxyalkyl, carbocyclyl, or carbocyclylalkyl. Examples of compounds falling within these embodiments include:

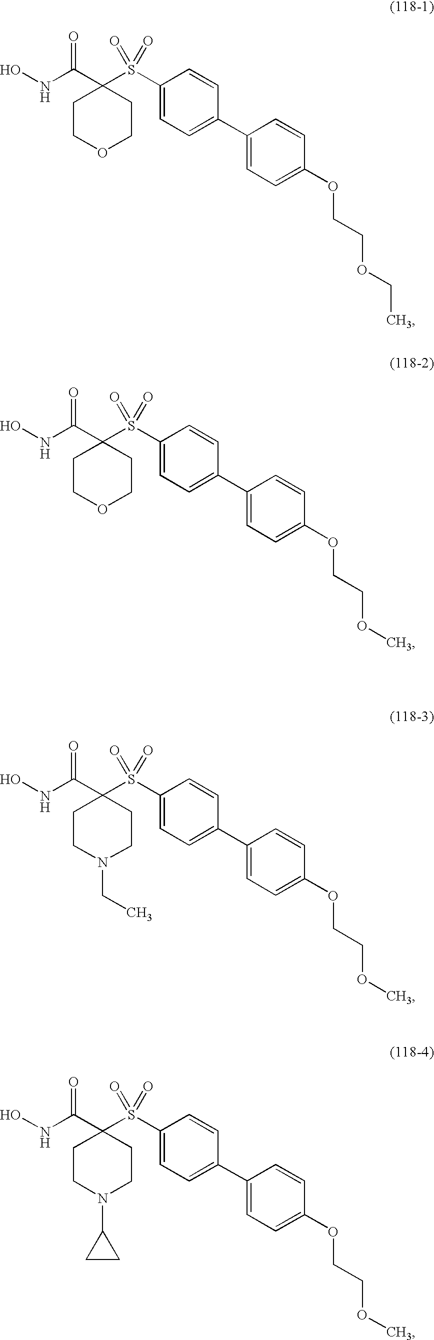

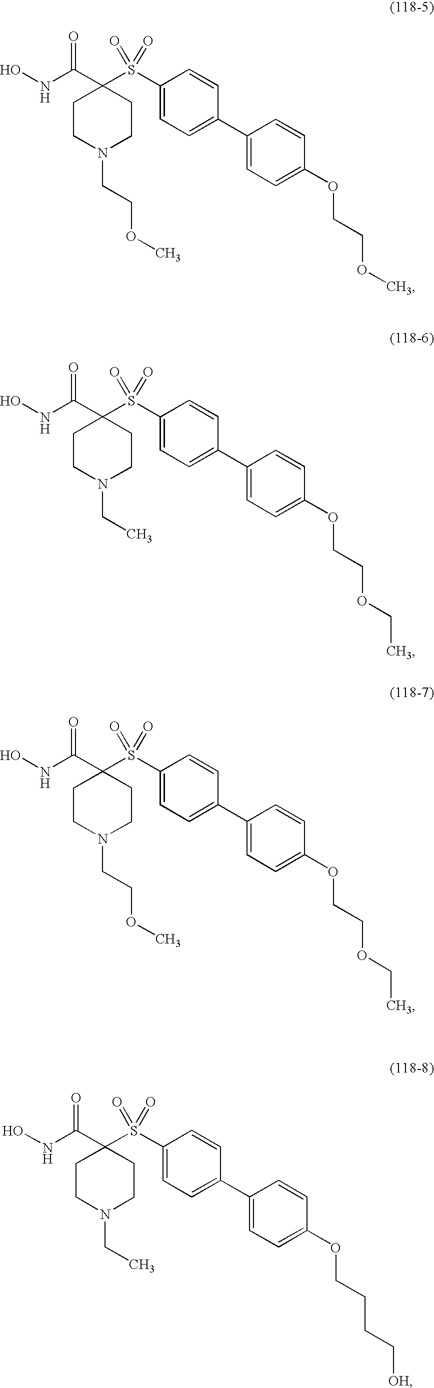

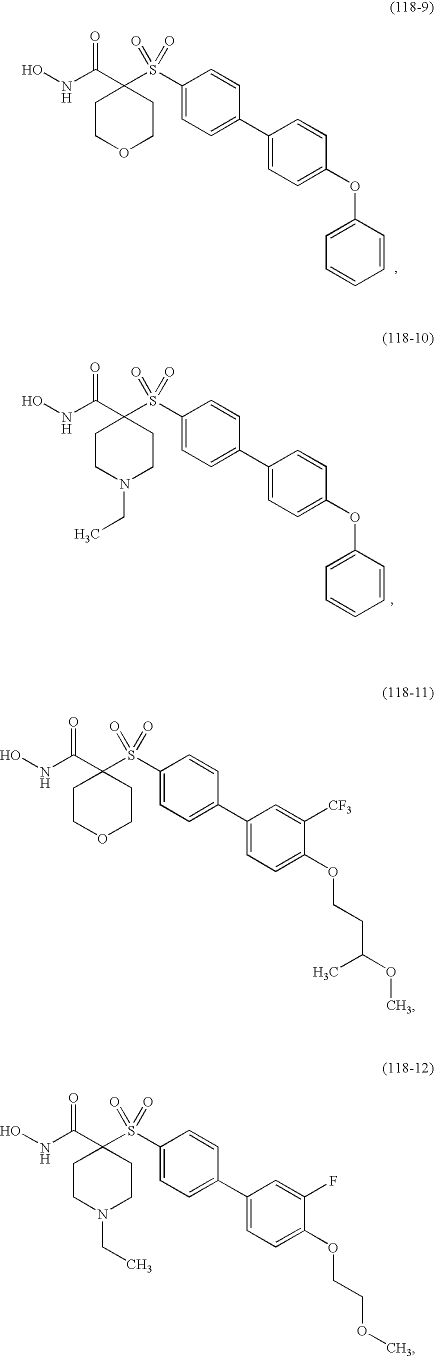

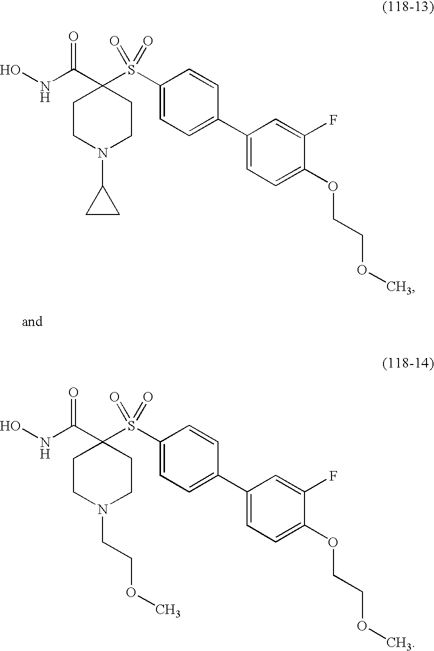

In some particularly preferred embodiments, E² is heteroaryl; E³ is —O—; and E⁴ is hydroxyalkyl, alkoxyalkyl, carbocyclyl, or carbocyclylalkyl. Examples of compounds falling within these embodiments include:

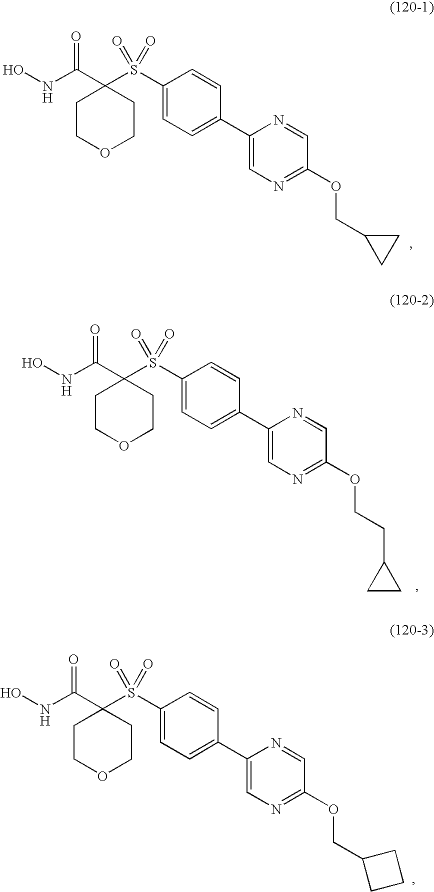

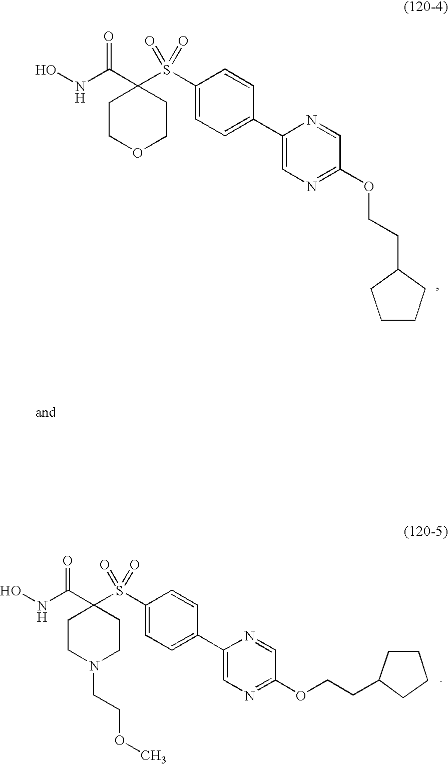

In some particularly preferred embodiments, E³ is —N(H)—, and E⁴ is carbocyclylalkyl or alkylheterocyclyl. Examples of compounds falling within these embodiments include:

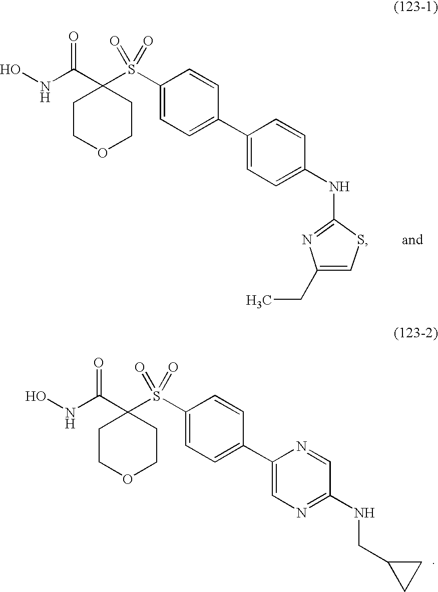

In some particularly preferred embodiments, E³ is —C(O)—N(H)— or —C(O)—N(CH₃)—, and E⁴ is alkynyl. An example of a compound falling within these embodiments includes:

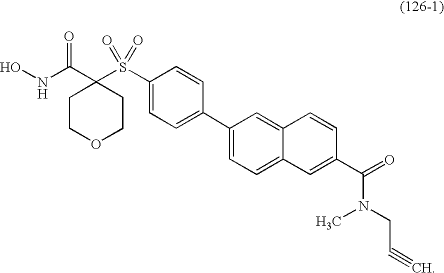

In some particularly preferred embodiments, E² is aryl, E³ is —C(O)—N(H)— or —C(O)—N(CH₃)—, and E⁴ is carbocyclyl or carbocyclylalkyl. An example of a compound falling within these embodiments includes:

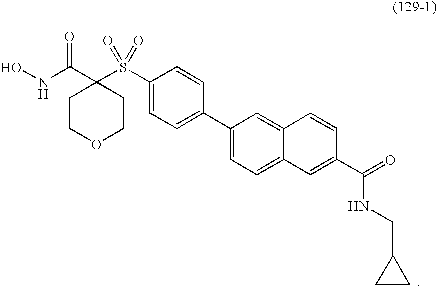

In some particularly preferred embodiments, E² is heteroaryl, E³ is —C(O)—N(H)— or —C(O)—N(CH₃)—, and E⁴ is carbocyclyl or carbocyclylalkyl. Examples of compounds falling within these embodiments include:

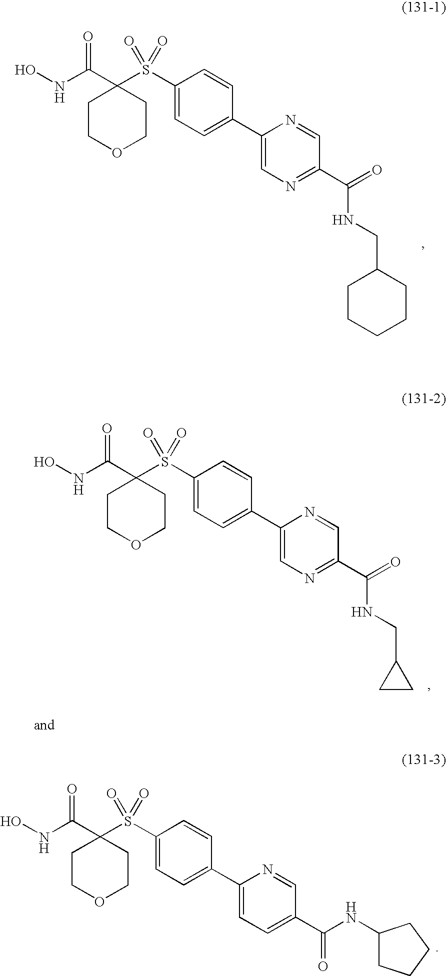

In some particularly preferred embodiments, E³ is carbonylalkyl, and E⁴ is heterocyclyl. An example of a compound falling within these embodiments includes:

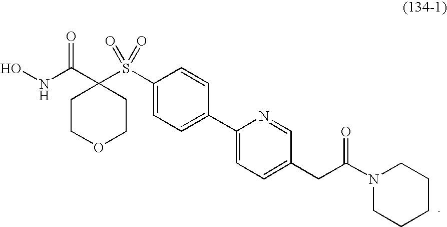

### Preferred Embodiment No. 5

In some preferred embodiments:

E³ is —O—, —C(O)—, —C(O)—O—, —O—C(O)—, —N(R^{b})—, —C(O)—N(R^{b})—, —N(R^{b})—C(O)—, —C(O)—N(R^{b})—N(R^{b})—C(O)—, —N(R^{b})—C(O)—N(R^{b})—, —S —, —S(O)—, —S(O)₂—, —N(R^{b})—S(O)₂—, —S(O)₂—N(R^{b})—, —O—S(O)₂—, —S(O)₂—O—, —C(NH)—, —C(NOH)—, —N(R^{b})—C(NH)—, —N(R^{b})—C(NOH)—, —C(NH)—N(R^{b})—, —C(NOH)—N(R^{b})—, alkenyl, carbonylalkyl, alkylcarbonyl, or a bond, wherein:
any alkyl or alkenyl portion of a substituent in such group optionally is substituted with one or more independently selected R^{c} substituents; and

E⁴ is alkyl, wherein the alkyl:
comprises a carbon chain of at least 4 carbon atoms (i.e., a chain of at least 4 carbon atoms bonded sequentially), and
is optionally substituted with one or more independently selected R^{d} substituents.

### Particularly Preferred Embodiments of Embodiment No. 5

In some particularly preferred embodiments, the compound corresponds in structure to Formula (138-1): 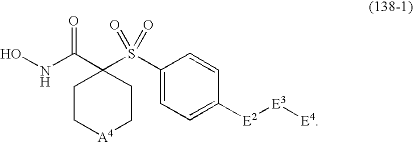
Here, A⁴ is —O—, —N(H)—, —N(R^{x})—, —S—, —S(O)—, —S(O)₂—, —C(H)₂—, or —C(R^{X})₂—.

In some particularly preferred embodiments, E² is phenyl optionally substituted with one or more independently selected halogen.

In some particularly preferred embodiments, E² is phenyl optionally substituted with one or more independently selected haloalkyl.

In some particularly preferred embodiments, E³ is a bond.

In some particularly preferred embodiments, E³ is —O—.

In some particularly preferred embodiments, E³ is —N(H)—.

In some particularly preferred embodiments, E³ is —C(O)—N(H)—

In some particularly preferred embodiments, E⁴ is —(CH₂)₃—CH₃.

In some particularly preferred embodiments, E⁴ is —(CH₂)₄—CH₃.

In some particularly preferred embodiments, E² is phenyl optionally substituted with one or more independently selected halogen, and E³ is a bond. Examples of compounds falling within these embodiments include:

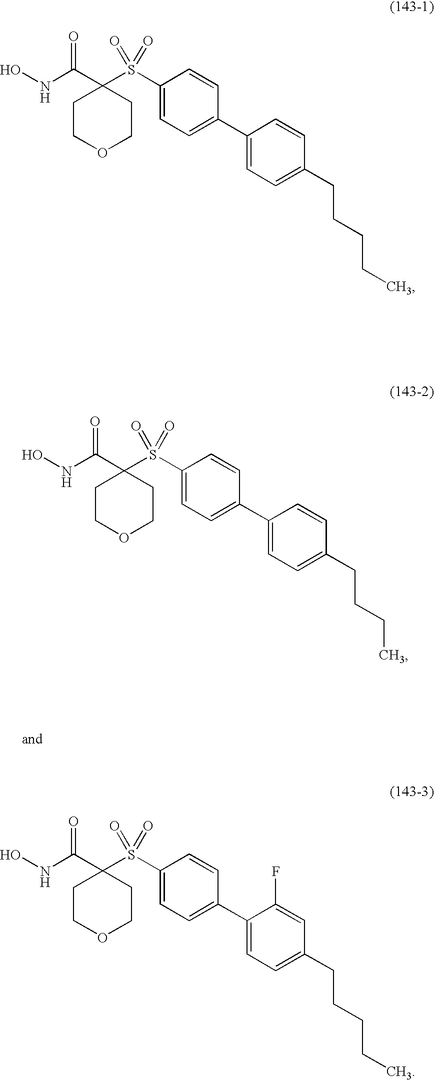

In some particularly preferred embodiments, E² is heteroaryl, and E³ is a bond. Examples of compounds falling within these embodiments include:

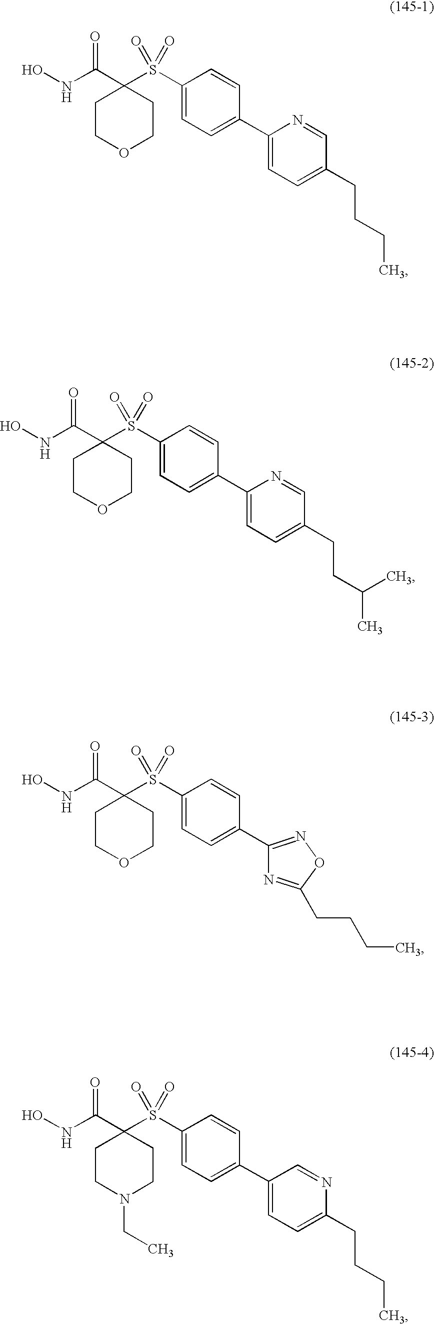

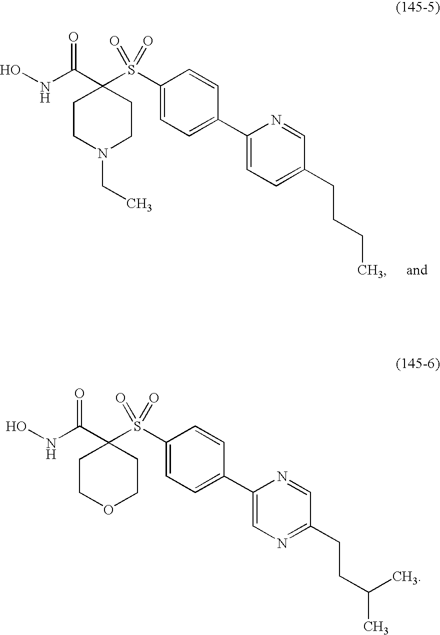

In some particularly preferred embodiments, E² is phenyl optionally substituted with one or more independently selected haloalkyl, and E³ is —O—. Examples of compounds falling within these embodiments include:

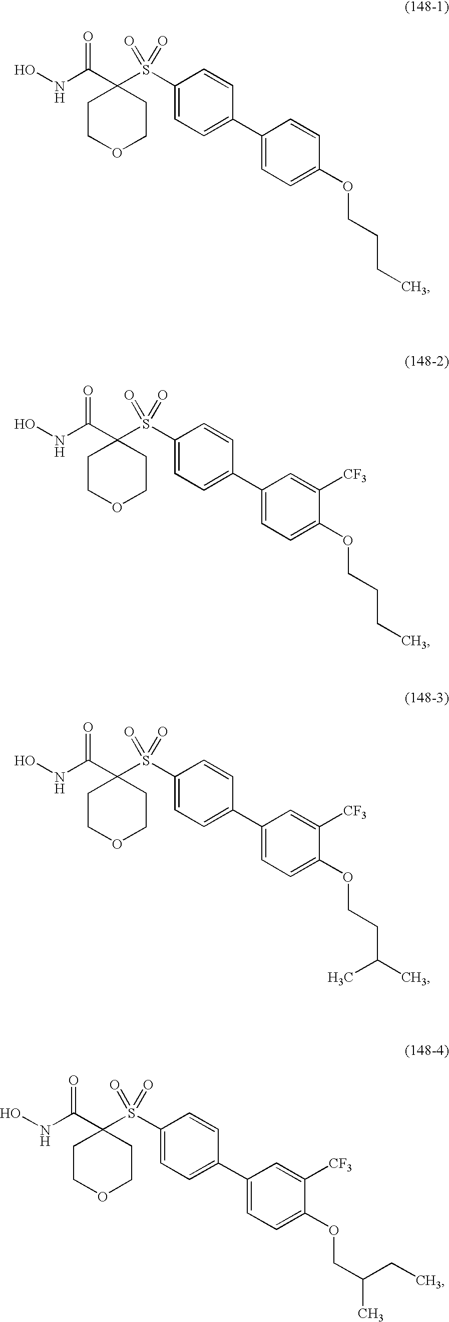

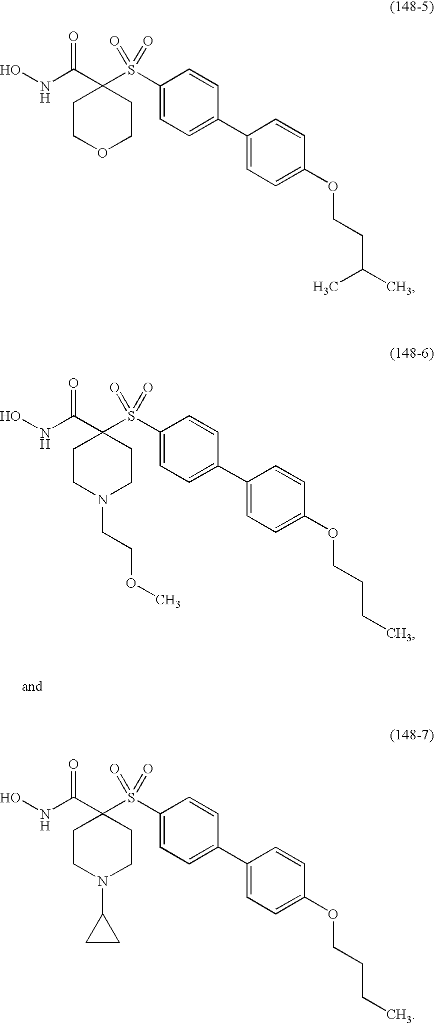

In some particularly preferred embodiments, E² is heteroaryl, and E³ is —O—. An example of a compound falling within these embodiments includes:

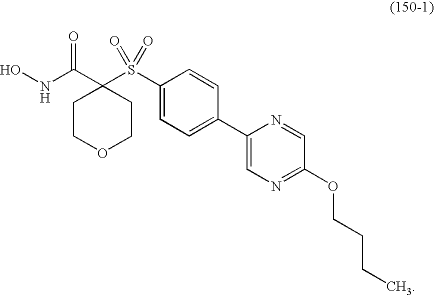

In some particularly preferred embodiments, E² is heteroaryl, and E³ is —N(H)—. Examples of compounds falling within these embodiments include:

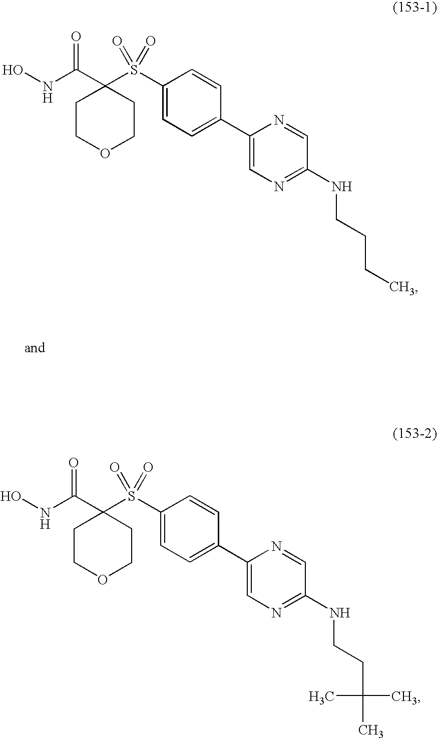

In some particularly preferred embodiments, E² is heteroaryl, E³ is —C(O)—N(H)—. Examples of compounds falling within these embodiments include:

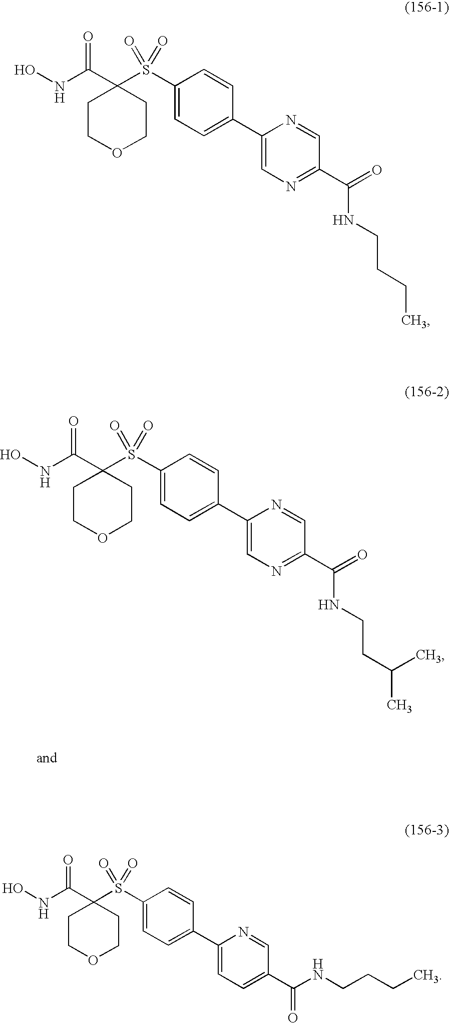

### Preferred Embodiment No. 6

In some preferred embodiments:

E² is heteroaryl optionally substituted with one or more independently selected R^{x} substituents; and

E⁴ is alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, aminoalkyl, carbocyclyl, carbocyclylalkyl, carbocyclylalkoxyalkyl, heterocyclyl, heterocyclylalkyl, or heterocyclylalkoxyalkyl, wherein:
any such group optionally is substituted with one or more independently selected R^{d} substituents.

### Particularly Preferred Embodiments of Embodiment No. 6

In some particularly preferred embodiments, the compound corresponds in structure to Formula (160-1): 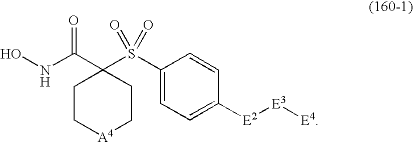
Here, A⁴ is —O—, —N(H)—, —N(R^{x})—, —S—, —S(O)—, —S(O)₂—, —C(H)₂, or —C(R^{X})₂—.

In some particularly preferred embodiments, E² is 5-member heteroaryl.

In some particularly preferred embodiments, E² is 6-member heteroaryl.

In some particularly preferred embodiments, E² is pyridinyl. Examples of compounds falling within these embodiments include:

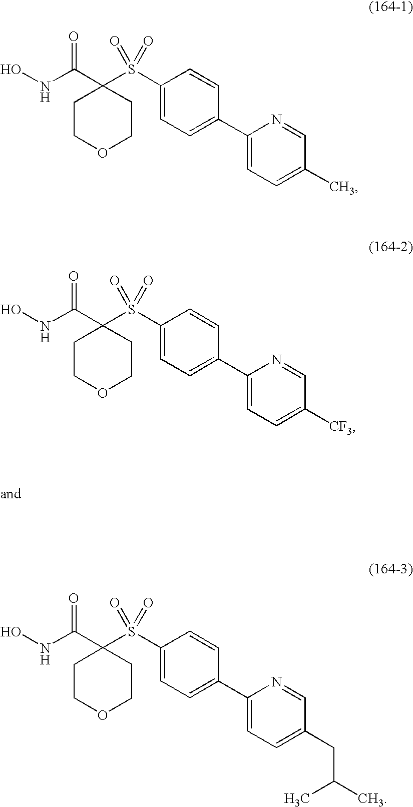

In some particularly preferred embodiments, E² is pyridinyl, and E³ is —C(O)—N(H)—. Examples of compounds falling within these embodiments include:

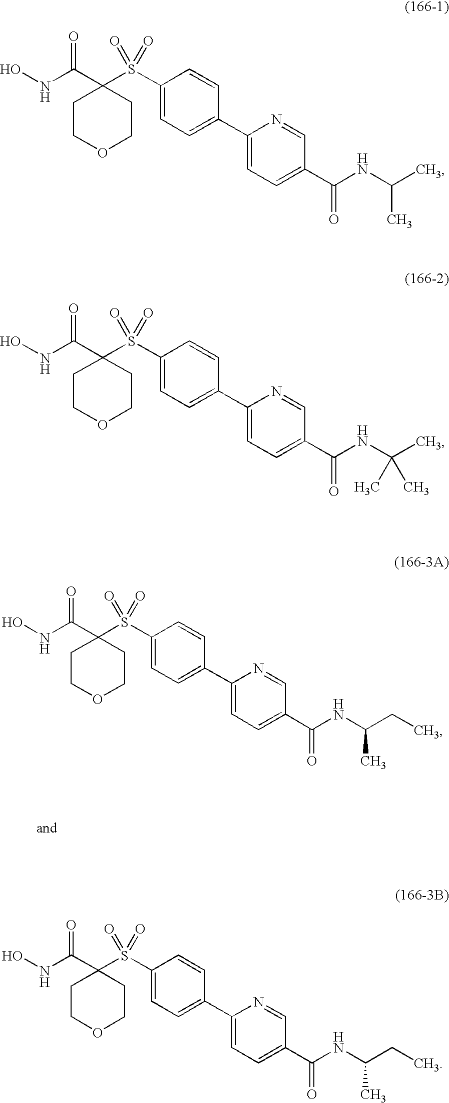

In some particularly preferred embodiments, E² is pyrazinyl. Examples of compounds falling within these embodiments include:

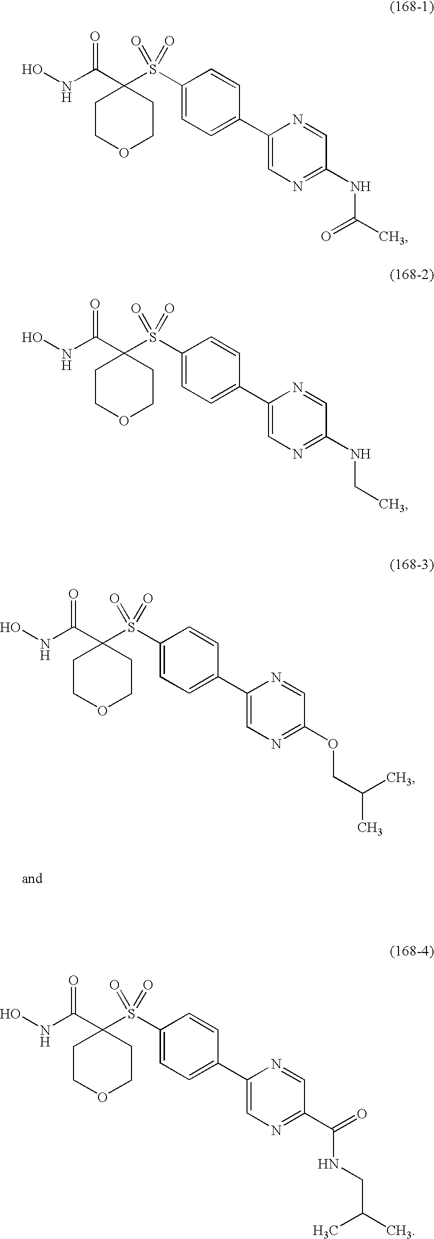

In some particularly preferred embodiments, E² is pyrimidinyl. An example of a compound falling within these embodiments is:

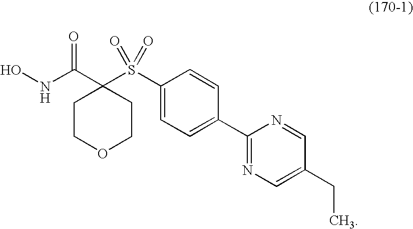

### Preferred Embodiment No. 7

In some preferred embodiments, E² is heteroaryl, wherein the heteroaryl:
comprises at least two heteroatoms, and
is optionally substituted with one or more independently selected R^{x} substituents.

### Particularly Preferred Embodiments of Embodiment No. 7

In some particularly preferred embodiments, the compound corresponds in structure to Formula (174-1): 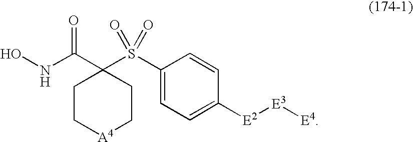
Here, A⁴ is —O—, —N(H)—, —N(R^{x})—, —S—, —S(O)—, —S(O)₂—, —C(H)₂—, or —C(R^{X})₂—.

In some particularly preferred embodiments, -E³-E⁴ is hydrogen.

In some particularly preferred embodiments, E² is single-ring heteroaryl.

In some particularly preferred embodiments, E² is pyrimidinyl or pyrazinyl.

In some particularly preferred embodiments, E² is pyrimidinyl or pyrazinyl, and -E³-E⁴ is hydrogen. Examples of compounds falling within these embodiments include:

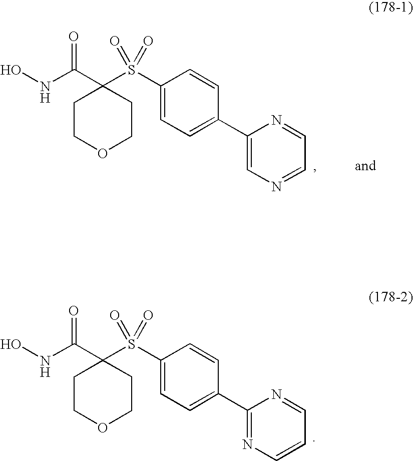

In some particularly preferred embodiments, E² is fused-ring heteroaryl.

In some particularly preferred embodiments, E² is 9-member heteroaryl.

In some particularly preferred embodiments, E² is 9-member heteroaryl, and -E³-E⁴ is hydrogen. Examples of compounds falling within these embodiments include:

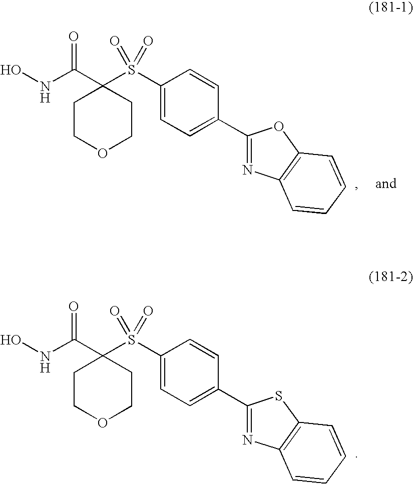

In some particularly preferred embodiments, E² is 10-member heteroaryl.

In some particularly preferred embodiments, E² is 10-member heteroaryl, and -E³-E⁴is hydrogen. An example of a compound falling within these embodiments is:

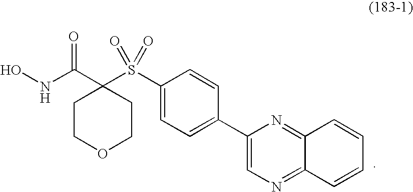

### Preferred Embodiment No. 8

In some preferred embodiments:

the compound corresponds in structure to Formula (184-1):

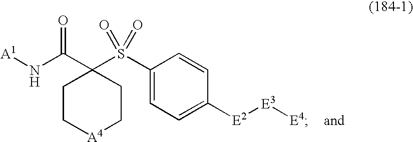

A⁴ is —N(H)—, —N(R^{x})—, —S—, —S(O)—, —S(O)₂—, —C(H)₂—, or —C(R^{X})₂—; and

E³ is —O—, —C(O)—, —C(O)—O—, —O—C(O)—, —N(R^{b})—, —C(O)—N(R^{b})—, —N(R^{b})—C(O)—, —C(O)—N(R^{b})—N(R^{b})—C(O)—, —N(R^{b})—C(O)—N(R^{b})—, —S—, —S(O)—, —S(O)₂—, —N(R^{b})—S(O)₂—, —S(O)₂—N(R^{b})—, —O—S(O)₂—, —S(O)₂—O—, —C(NH)—, —C(NOH)—, —N(R^{b})—C(NH)—, —N(R^{b})—C(NOH)—, —C(NH)—N(R^{b})—, —C(NOH)—N(R^{b})—, alkyl, alkenyl, carbonylalkyl, or alkylcarbonyl, wherein:
any alkyl or alkenyl portion of a substituent in such group optionally is substituted with one or more independently selected R^{c} substituents; and

E⁴ is alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, aminoalkyl, carbocyclyl, carbocyclylalkyl, carbocyclylalkoxyalkyl, heterocyclyl, heterocyclylalkyl, or heterocyclylalkoxyalkyl, wherein:
any such substituent optionally is substituted with one or more independently selected R^{d} substituents.

An example of a compound falling within these embodiments is:

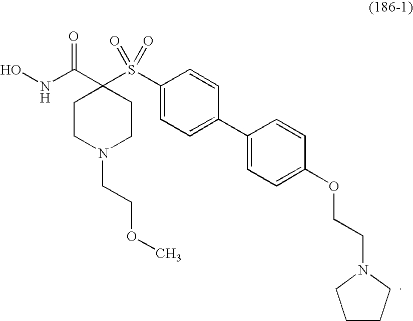

### Preferred Embodiment No. 9

In some preferred embodiments:

the compound corresponds in structure to Formula (187-1):

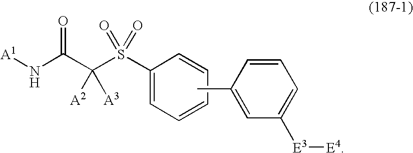

E³ is —O—, —C(O)—, —C(O)—O—, —O—C(O)—, —N(R^{b})—, —C(O)—N(R^{b})—, —N(R^{b})—C(O)—, —C(O)—N(R^{b})—N(R^{b})—C(O)—, —N(R^{b})—C(O)—N(R^{b})—, —S—, —S(O)—, —S(O)₂—, —N(R^{b})—S(O)₂—, —S(O)₂—N(R^{b})—, —O—S(O)₂—, —S(O)₂—O—, —C(NH)—, —C(NOH)—, —N(R^{b})—C(NH)—, —N(R^{b})—C(NOH)—, —C(NH)—N(R^{b})—, —C(NOH)—N(R^{b})—, alkyl, alkenyl, carbonylalkyl, or alkylcarbonyl, wherein:
any alkyl or alkenyl portion of a substituent in such group optionally is substituted with one or more independently selected R^{c} substituents; and

E⁴ is alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, aminoalkyl, carbocyclyl, carbocyclylalkyl, carbocyclylalkoxyalkyl, heterocyclyl, heterocyclylalkyl, or heterocyclylalkoxyalkyl, wherein:
any such group optionally is substituted with one or more independently selected R^{d} substituents.

### Particularly Preferred Embodiments of Embodiment No. 9

In some particularly preferred embodiments, the compound corresponds in structure to Formula (189-1): 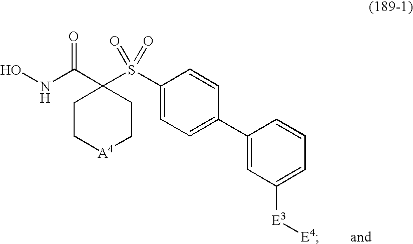
Here, A⁴ is —O—, —N(H)—, —N(R^{x})—, —S—, —S(O)—, —S(O)₂—, —C(H)₂, or —C(R^{X})₂—. An example of a compound falling within these embodiments is:

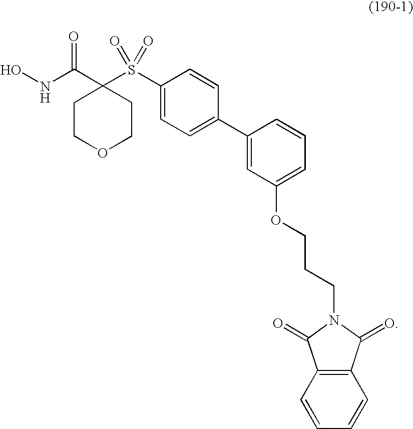

### Preferred Embodiment No. 10

In some preferred embodiments, E² is 2 rings fused together. In these embodiments, the ring bonded to E¹ is an unsaturated, 6-member ring. One or both of the rings comprise one or more independently selected heteroatoms. And one or both of the rings optionally are substituted with one or more independently selected R^{x} substituents.

### Particularly Preferred Embodiments of Embodiment No. 10

In some particularly preferred embodiments, the compound corresponds in structure to Formula (194-1): 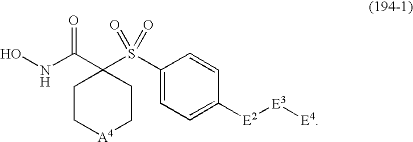
Here, A⁴ is —O—, —N(H)—, —N(R^{x})—, —S—, —S(O)—, —S(O)2—, —C(H)₂—, or —C(R^{X})₂—.

In some particularly preferred embodiments, E² is 10-member heterocyclyl.

In some particularly preferred embodiments, E² is 9-member heterocyclyl.

In some particularly preferred embodiments, E²is -E³-E⁴ is hydrogen.

In some particularly preferred embodiments, E is 9-member heteroaryl, and -E³-E⁴ is hydrogen. Examples of compounds falling within these embodiments include:

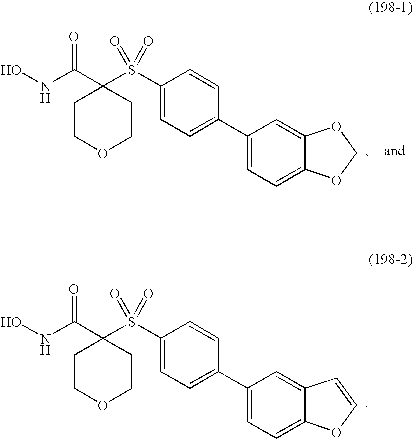

### Preferred Embodiment No. 11

In some preferred embodiments, -E³-E⁴ is —CH₂—CH₃, —(CH₂)₂—CH₃, —C(CH₃)₂H, or —O—CH₂—CH₃. In these embodiments, any member of such group optionally is substituted with one or more substituents independently selected from the group consisting of halogen, hydroxy, cyano, sulfo, nitro, nitroso, oxo, thioxo, imino, alkoxy, alkoxyalkyl, —N(R^{e})(R^{e}), —C(O)(R^{g}), —S—R^{e}, —S(O)₂—R^{e}, carbocyclyl, alkylcarbocyclyl, carbocyclylalkyl, heterocyclyl, alkylheterocyclyl, and heterocyclylalkyl. Any such optional substituent is, in turn, optionally substituted with one or more substituents independently selected from the group consisting of halogen, hydroxy, cyano, carboxy, thiol, sulfo, nitro, nitroso, oxo, thioxo, imino, aminocarbonyl, and amino.

### Particularly Preferred Embodiments of Embodiment No. 11

In some particularly preferred embodiments, the compound corresponds in structure to Formula (202-1): 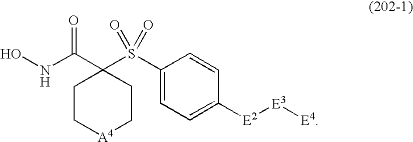
Here, A⁴ is —O—, —N(H)—, —N(R^{x})—, —S—, —S(O)—, —S(O)₂—, —C(H)₂—, or —C(R^{X})₂—.

In some particularly preferred embodiments, -E³-E⁴ is —CH₂—CH₃. An example of a compound falling within these embodiments is:

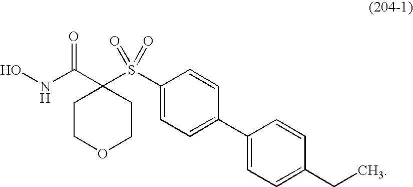

In some particularly preferred embodiments, -E³-E⁴ is —CH₂—CH₃ substituted with alkylheterocyclyl. An example of a compound falling within these embodiments is:

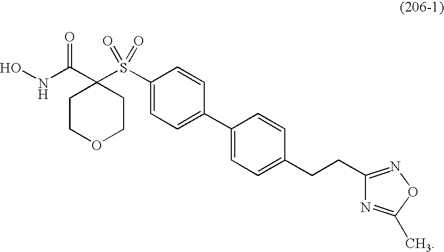

In some particularly preferred embodiments, -E³-E⁴ is —(CH₂)₂—CH₃. An example of a compound falling within these embodiments is:

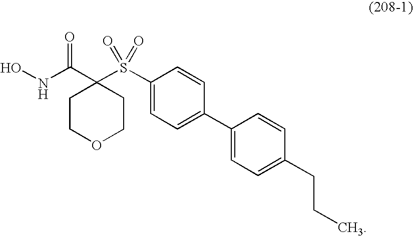

In some particularly preferred embodiments, -E³-E⁴ is —(CH₂)₂—CH₃ substituted with heterocyclyl and oxo. An example of a compound falling within these embodiments is:

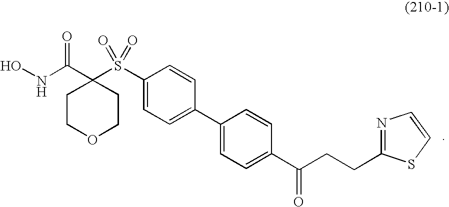

In some particularly preferred embodiments, -E³-E⁴ is —C(CH₃)₂H. An example of a compound falling within these embodiments is:

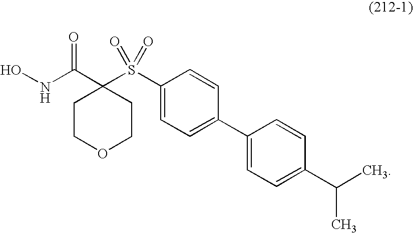

In some particularly preferred embodiments, -E³-E⁴ is —O—CH₂—CH₃. An example of a compound falling within these embodiments is:

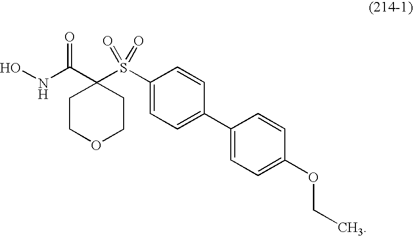

### Preferred Embodiment No. 12

In some preferred embodiments:

E² is naphthyl optionally substituted with one or more independently selected R^{x} substituents; and

E⁴ is selected from the group consisting of alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, aminoalkyl, carbocyclyl, carbocyclylalkyl, carbocyclylalkoxyalkyl, heterocyclyl, heterocyclylalkyl, and heterocyclylalkoxyalkyl, wherein:
any such group optionally is substituted with one or more independently selected R^{d} substituents.

### Particularly Preferred Embodiments of Embodiment No. 12

In some particularly preferred embodiments, the compound corresponds in structure to Formula (218-1): 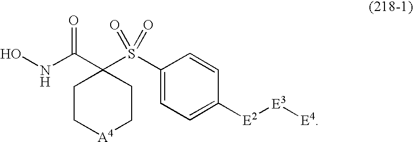
Here, A⁴ is —O—, —N(H)—, —N(R^{x})—, —S—, —S(O)—, —S(O)₂—, 'C(H)₂—, or —C(R^{X})₂—.

In some particularly preferred embodiments, the compound corresponds in structure to Formula (219-1):

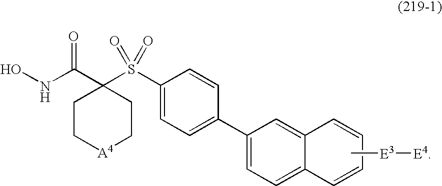

In some particularly preferred embodiments, the compound corresponds in structure to Formula (220-1):

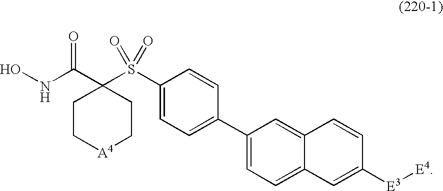

In some particularly preferred embodiments, E³ is —C(O)— or —C(O)—N(R^{b})—. Examples of compounds falling within these embodiments include the following:

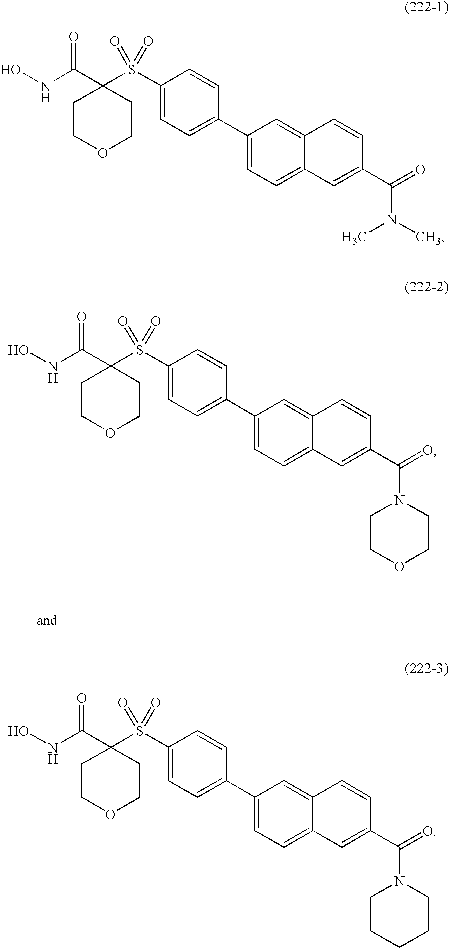

### A-2. Preferred Selectivities

When a compound or salt of this invention are used to treat conditions associated with MMP activity, the compound or salt preferably has an inhibitory activity against MMP-1 or MMP-14 that is substantially less than its inhibitory activity against MMP-2, MMP-9, or MMP-13. In other words, the compound or salt preferably has an in inhibition constant (Kᵢ) against at least one of MMP-2, MMP-9, and MMP-13 that is no greater than about 0.1 times its inhibition constant(s) against at least one of MMP-1 and MMP-14. The inhibition constant of a compound or salt may be determined using an in vitro inhibition assay, such as the Kᵢ assay described in the Examples below.

In some particularly preferred embodiments, the compound or salt preferably has a Kᵢ against MMP-2 that is no greater than about 0.1 (more preferably no greater than about 0.01, even more preferably no greater than about 0.001, still more preferably no greater than about 0.0001, and still even more preferably no greater than about 0.00001) times its Kᵢ(s) against one or both of MMP-1 and MMP-14.

In some particularly preferred embodiments, the compound or salt preferably has a Kᵢ against MMP-9 that is no greater than about 0.1 (more preferably no greater than about 0.01, even more preferably no greater than about 0.001, still more preferably no greater than about 0.0001, and still even more preferably no greater than about 0.00001) times its Kᵢ(s) against one or both of MMP-1 and MMP-14. It is believed that such a selectivity profile is often particularly preferred when treating, for example, a pathological condition of the central nervous system associated with nitrosative or oxidative stress. Such a pathological condition may be, for example, cerebral ischemia, stroke, or other neurodegenerative disease.

In some particularly preferred embodiments, the compound or salt preferably has a Kᵢ against MMP-13 that is no greater than about 0.1 (more preferably no greater than about 0.01, even more preferably no greater than about 0.001, still more preferably no greater than about 0.0001, and still even more preferably no greater than about 0.00001) times its Kᵢ(s) against one or both of MMP-1 and MMP-14. It is believed that such a selectivity profile is often particularly preferred when treating, for example, a cardiovascular condition or arthritis.

In some particularly preferred embodiments, the compound or salt preferably has Kᵢ's against both MMP-2 and MMP-9 that are no greater than about 0.1 (more preferably no greater than about 0.01, even more preferably no greater than about 0.001, still more preferably no greater than about 0.0001, and still even more preferably no greater than about 0.00001) times its Kᵢ(s) against one or both of MMP-1 and MMP-14. It is believed that such a selectivity profile is often particularly preferred when treating, for example, cancer, a cardiovascular condition, or an ophthalmologic condition.

In some particularly preferred embodiments, the compound or salt preferably has Kᵢ's against all of MMP-2, MMP-9, and MMP-13 that are no greater than about 0.1 (more preferably no greater than about 0.01, even more preferably no greater than about 0.001, still more preferably no greater than about 0.0001, and still even more preferably no greater than about 0.00001) times its Kᵢ(s) against one or both of MMP-1 and MMP-14. It is believed that such a selectivity profile is often particularly preferred when treating, for example, cancer, a cardiovascular condition, arthritis, or an ophthalmologic condition.

In some particularly preferred embodiments, the compound or salt preferably has a Kᵢ against MMP-2 that is no greater than about 0.1 (more preferably no greater than about 0.01, even more preferably no greater than about 0.001, still more preferably no greater than about 0.0001, and still even more preferably no greater than about 0.00001) times its Kᵢ's against both MMP-1 and MMP-14.

In some particularly preferred embodiments, the compound or salt preferably has a Kᵢ against MMP-9 that is no greater than about 0.1 (more preferably no greater than about 0.01, even more preferably no greater than about 0.001, still more preferably no greater than about 0.0001, and still even more preferably no greater than about 0.00001) times its Kᵢ's against both MMP-1 and MMP-14. It is believed that such a selectivity profile is often particularly preferred when treating, for example, a pathological condition of the central nervous system associated with nitrosative or oxidative stress. Such a pathological condition may be, for example, cerebral ischemia, stroke, or other neurodegenerative disease.

In some particularly preferred embodiments, the compound or salt preferably has a Kᵢ against MMP-13 that is no greater than about 0.1 (more preferably no greater than about 0.01, even more preferably no greater than about 0.001, still more preferably no greater than about 0.0001, and still even more preferably no greater than about 0.00001) times its Kᵢ's against both MMP-1 and MMP-14. It is believed that such a selectivity profile is often particularly preferred when treating, for example, a cardiovascular condition or arthritis.

In some particularly preferred embodiments, the compound or salt preferably has Kᵢ's against both MMP-2 and MMP-9 that are no greater than about 0.1 (more preferably no greater than about 0.01, even more preferably no greater than about 0.001, still more preferably no greater than about 0.0001, and still even more preferably no greater than about 0.00001) times its Kᵢ's against both of MMP-1 and MMP-14. It is believed that such a selectivity profile is often particularly preferred when treating, for example, cancer, a cardiovascular condition, or an ophthalmologic condition.

In some particularly preferred embodiments, the compound or salt preferably has Kᵢ's against all of MMP-2, MMP-9, and MMP-13 that are no greater than about 0.1 (more preferably no greater than about 0.01, even more preferably no greater than about 0.001, still more preferably no greater than about 0.0001, and still even more preferably no greater than about 0.00001) times its Kᵢ's against both of MMP-1 and MMP-14. It is believed that such a selectivity profile is often particularly preferred when treating, for example, cancer, a cardiovascular condition, arthritis, or an ophthalmologic condition.

The activity and selectivity of a compound or salt of this invention may alternatively be determined using an in vitro IC₅₀ assay, such as the IC₅₀ assay described in WIPO Publ. No. WO 02/092588 (Appl. No. PCT/US02/15257, filed May 10, 2002, published Nov. 21, 2002) (incorporated by reference into this patent). In that instance, the compound or salt preferably has an IC₅₀ value against at least one of MMP-2, MMP-9, and MMP-13 that is no greater than about 0.1 times its IC₅₀ value(s) against at least one of MMP-1 and MMP-14.

In some particularly preferred embodiments, the compound or salt preferably has an IC₅₀ value against MMP-2 that is no greater than about 0.1 (more preferably no greater than about 0.01, even more preferably no greater than about 0.001, still more preferably no greater than about 0.0001, and still even more preferably no greater than about 0.00001) times its IC₅₀ value(s) against one or both of MMP-1 and MMP-14.

In some particularly preferred embodiments, the compound or salt preferably has an IC₅₀ value against MMP-9 that is no greater than about 0.1 (more preferably no greater than about 0.01, even more preferably no greater than about 0.001, still more preferably no greater than about 0.0001, and still even more preferably no greater than about 0.00001) times its IC₅₀ value(s) against one or both of MMP-1 and MMP-14. It is believed that such a selectivity profile is often particularly preferred when treating, for example, a pathological condition of the central nervous system associated with nitrosative or oxidative stress. Such a pathological condition may be, for example, cerebral ischemia, stroke, or other neurodegenerative disease.

In some particularly preferred embodiments, the compound or salt preferably has an IC₅₀ value against MMP-13 that is no greater than about 0.1 (more preferably no greater than about 0.01, even more preferably no greater than about 0.001, still more preferably no greater than about 0.0001, and still even more preferably no greater than about 0.00001) times its IC₅₀ value(s) against one or both of MMP-1 and MMP-14. It is believed that such a selectivity profile is often particularly preferred when treating, for example, a cardiovascular condition or arthritis.

In some particularly preferred embodiments, the compound or salt preferably has IC₅₀ values against both MMP-2 and MMP-9 that are no greater than about 0.1 (more preferably no greater than about 0.01, even more preferably no greater than about 0.001, still more preferably no greater than about 0.0001, and still even more preferably no greater than about 0.00001) times its IC₅₀ value(s) against one or both of MMP-1 and MMP-14. It is believed that such a selectivity profile is often particularly preferred when treating, for example, cancer, a cardiovascular condition, or an ophthalmologic condition.

In some particularly preferred embodiments, the compound or salt preferably has IC₅₀ values against all of MMP-2, MMP-9, and MMP-13 that are no greater than about 0.1 (more preferably no greater than about 0.01, even more preferably no greater than about 0.001, still more preferably no greater than about 0.0001, and still even more preferably no greater than about 0.00001) times its IC₅₀ value(s) against one or both of MMP-1 and MMP-14. It is believed that such a selectivity profile is often particularly preferred when treating, for example, cancer, a cardiovascular condition, arthritis, or an ophthalmologic condition.

In some particularly preferred embodiments, the compound or salt preferably has an IC₅₀ value against MMP-2 that is no greater than about 0.1 (more preferably no greater than about 0.01, even more preferably no greater than about 0.001, still more preferably no greater than about 0.0001, and still even more preferably no greater than about 0.00001) times its IC₅₀ values against both MMP-1 and MMP-14.

In some particularly preferred embodiments, the compound or salt preferably has an IC₅₀ value against MMP-9 that is no greater than about 0.1 (more preferably no greater than about 0.01, even more preferably no greater than about 0.001, still more preferably no greater than about 0.0001, and still even more preferably no greater than about 0.00001) times its IC₅₀ values against both MMP-1 and MMP-14. It is believed that such a selectivity profile is often particularly preferred when treating, for example, a pathological condition of the central nervous system associated with nitrosative or oxidative stress. Such a pathological condition may be, for example, cerebral ischemia, stroke, or other neurodegenerative disease.

In some particularly preferred embodiments, the compound or salt preferably has an IC₅₀ value against MMP-13 that is no greater than about 0.1 (more preferably no greater than about 0.01, even more preferably no greater than about 0.001, still more preferably no greater than about 0.0001, and still even more preferably no greater than about 0.00001) times its IC₅₀ values against both MMP-1 and MMP-14. It is believed that such a selectivity profile is often particularly preferred when treating, for example, a cardiovascular condition or arthritis.

In some particularly preferred embodiments, the compound or salt preferably has IC₅₀ values against both MMP-2 and MMP-9 that are no greater than about 0.1 (more preferably no greater than about 0.01, even more preferably no greater than about 0.001, still more preferably no greater than about 0.0001, and still even more preferably no greater than about 0.00001) times its IC₅₀ values against both of MMP-1 and MMP-14. It is believed that such a selectivity profile is often particularly preferred when treating, for example, cancer, a cardiovascular condition, or an ophthalmologic condition.

In some particularly preferred embodiments, the compound or salt preferably has IC₅₀ values against all of MMP-2, MMP-9, and MMP-13 that are no greater than about 0.1 (more preferably no greater than about 0.01, even more preferably no greater than about 0.001, still more preferably no greater than about 0.0001, and still even more preferably no greater than about 0.00001) times its IC₅₀ values against both of MMP-1 and MMP-14. It is believed that such a selectivity profile is often particularly preferred when treating, for example, cancer, a cardiovascular condition, arthritis, or an ophthalmologic condition.

### B. Salts of the Compounds of this Invention

The compounds of this invention can be used in the form of salts derived from inorganic or organic acids. Depending on the particular compound, a salt of the compound may be advantageous due to one or more of the salt's physical properties, such as enhanced pharmaceutical stability in differing temperatures and humidities, or a desirable solubility in water or oil. In some instances, a salt of a compound also may be used as an aid in the isolation, purification, and/or resolution of the compound.

Where a salt is intended to be administered to a patient (as opposed to, for example, being used in an in vitro context), the salt preferably is pharmaceutically acceptable. Pharmaceutically acceptable salts include salts commonly used to form alkali metal salts and to form addition salts of free acids or free bases. In general, these salts typically may be prepared by conventional means with a compound of this invention by reacting, for example, the appropriate acid or base with the compound.

Pharmaceutically-acceptable acid addition salts of the compounds of this invention may be prepared from an inorganic or organic acid. Examples of suitable inorganic acids include hydrochloric, hydrobromic acid, hydroionic, nitric, carbonic, sulfuric, and phosphoric acid. Suitable organic acids generally include, for example, aliphatic, cycloaliphatic, aromatic, araliphatic, heterocyclyl, carboxylic, and sulfonic classes of organic acids. Specific examples of suitable organic acids include acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, mesylate, stearate, salicylate, p-hydroxybenzoate, phenylacetate, mandelate, embonate (pamoate), methanesulfonate, ethanesulfonate, benzenesulfonate, pantothenate, toluenesulfonate, 2-hydroxyethanesulfonate, sufanilate, cyclohexylaminosulfonate, algenic acid, b-hydroxybutyric acid, galactarate, galacturonate, adipate, alginate, bisulfate, butyrate, camphorate, camphorsulfonate, cyclopentanepropionate, dodecylsulfate, glycoheptanoate, glycerophosphate, hemisulfate, heptanoate, hexanoate, nicotinate, 2-naphthalesulfonate, oxalate, palmoate, pectinate, persulfate, 3-phenylpropionate, picrate, pivalate, thiocyanate, tosylate, and undecanoate.

Pharmaceutically-acceptable base addition salts of the compounds of this invention include, for example, metallic salts and organic salts. Preferred metallic salts include alkali metal (group Ia) salts, alkaline earth metal (group IIa) salts, and other physiological acceptable metal salts. Such salts may be made from aluminum, calcium, lithium, magnesium, potassium, sodium, and zinc. Preferred organic salts can be made from tertiary amines and quaternary amine salts, such as tromethamine, diethylamine, N,N′-dibenzylethylenediamine, chloroprocaine, choline, diethanolamine, ethylenediamine, meglumine (N-methylglucamine), and procaine. Basic nitrogen-containing groups can be quaternized with agents such as lower alkyl (C₁-C₆) halides (e.g., methyl, ethyl, propyl, and butyl chlorides, bromides, and iodides), dialkyl sulfates (e.g., dimethyl, diethyl, dibuytl, and diamyl sulfates), long chain halides (e.g., decyl, lauryl, myristyl, and stearyl chlorides, bromides, and iodides), aralkyl halides (e.g., benzyl and phenethyl bromides), and others.

Particularly preferred salts of the compounds of this invention include hydrochloric acid (HCl) salts and trifluoroacetate (CF₃COOH or TFA) salts.

### C. Treating Conditions Using the Compounds and Salts of this Invention

One embodiment of this invention is directed to a process for treating a pathological condition associated with MMP activity in a mammal (e.g., a human, companion animal, farm animal, laboratory animal, zoo animal, or wild animal) having or disposed to having such a condition. Such a condition may be, for example, tissue destruction, a fibrotic disease, pathological matrix weakening, defective injury repair, a cardiovascular disease, a pulmonary disease, a kidney disease, a liver disease, an ophthalmologic disease, or a central nervous system disease. Specific examples of such conditions include osteoarthritis, rheumatoid arthritis, septic arthritis, tumor invasion, tumor metastasis, tumor angiogenesis, a decubitis ulcer, a gastric ulcer, a corneal ulcer, periodontal disease, liver cirrhosis, fibrotic lung disease, otosclerosis, atherosclerosis, multiple sclerosis, dilated cardiomyopathy, epidermal ulceration, epidermolysis bullosa, aortic aneurysm, weak injury repair, an adhesion, scarring, congestive heart failure, post myocardial infarction, coronary thrombosis, emphysema, proteinuria, bone disease, chronic obstructive pulmonary diseases, Alzheimer's disease, and diseases of the central nervous system associated with nitrosative or oxidative stress (e.g., stroke, cerebral ischemia, and other neurodegenerative diseases).

In some particularly contemplated embodiments, the condition comprises arthritis.

In some particularly contemplated embodiments, the condition comprises tumor invasion, tumor metastasis, or tumor angiogenesis.

In some particularly contemplated embodiments, the condition comprises periodontal disease.

In some particularly contemplated embodiments, the condition comprises atherosclerosis.

In some particularly contemplated embodiments, the condition comprises multiple sclerosis.

In some particularly contemplated embodiments, the condition comprises dilated cardiomyopathy.

In some particularly contemplated embodiments, the condition comprises post myocardial infarction.

In some particularly contemplated embodiments, the condition comprises congestive heart failure.

In some particularly contemplated embodiments, the condition comprises chronic obstructive pulmonary disease.

In some particularly contemplated embodiments, the condition comprises a disease of the central nervous system associated with nitrosative or oxidative stress. Such a disease may be, for example, stroke, cerebral ischemia, and other neurodegenerative diseases.

The condition may alternatively (or additionally) be associated with TNF-α convertase activity. Examples of such a condition include inflammation (e.g., rheumatoid arthritis), autoimmune disease, graft rejection, multiple sclerosis, a fibrotic disease, cancer, an infectious disease (e.g., malaria, mycobacterial infection, meningitis, etc.), fever, psoriasis, a cardiovascular disease (e.g., post-ischemic reperfusion injury and congestive heart failure), a pulmonary disease, hemorrhage, coagulation, hyperoxic alveolar injury, radiation damage, acute phase responses like those seen with infections and sepsis and during shock (e.g., septic shock, hemodynamic shock, etc.), cachexia, and anorexia.

The condition may alternatively (or additionally) be associated with aggrecanase activity. Examples of such a condition include inflammation diseases (e.g., osteoarthritis, rheumatoid arthritis, joint injury, reactive arthritis, acute pyrophosphate arthritis, and psoriatic arthritis) and cancer.

In this specification, the phrase “treating a condition” means ameliorating, suppressing, eradicating, preventing, reducing the risk of, or delaying the onset of the condition. The pathological condition may be (a) the result of pathological aggrecanase and/or MMP activity itself, and/or (b) affected by aggrecanase and/or MMP activity (e.g., diseases associated with TNF-α).

A wide variety of methods may be used alone or in combination to administer the compounds and salt thereof described above. For example, the compounds or salts thereof may be administered orally, parenterally, by inhalation spray, rectally, or topically.

Typically, a compound (or pharmaceutically acceptable salt thereof) described in this patent is administered in an amount effective to inhibit a target MMP(s). The target MMP is/are typically MMP-2, MMP-9, and/or MMP-13, with MMP-13 often being a particularly preferred target.

In some preferred embodiments, the Al substituent of the compound or salt is hydrogen, i.e., the compound is an amide. In other preferred embodiments, the A¹ substituent of the compound or salt is hydroxy, i.e., the compound is a hydroxamic acid.

The preferred total daily dose of the compound or salt (administered in single or divided doses) is typically from about 0.001 to about 100 mg/kg, more preferably from about 0.001 to about 30 mg/kg, and even more preferably from about 0.01 to about 10 mg/kg (i.e., mg of compound or salt of this invention per kg body weight). Dosage unit compositions can contain such amounts or submultiples thereof to make up the daily dose. In many instances, the administration of the compound or salt will be repeated a plurality of times. Multiple doses per day typically may be used to increase the total daily dose, if desired.

Factors affecting the preferred dosage regimen include the type, age, weight, sex, diet, and condition of the patient; the severity of the pathological condition; the route of administration; pharmacological considerations, such as the activity, efficacy, pharmacokinetic, and toxicology profiles of the particular compound or salt used; whether a drug delivery system is utilized; and whether the compound or salt is administered as part of a drug combination. Thus, the dosage regimen actually employed can vary widely, and, therefore, can deviate from the preferred dosage regimen set forth above.

### D. Pharmaceutical Compositions Containing the Compounds and Salts of this Invention

This invention also is directed to pharmaceutical compositions comprising a compound or salt thereof described above, and to methods for making pharmaceutical compositions (or medicaments) comprising a compound or salt thereof described above. In some preferred embodiments, the A¹ substituent of the compound or salt is hydrogen. In other preferred embodiments, the A¹ substituent of the compound or salt is hydroxy.

The preferred composition depends on the method of administration, and typically comprises one or more conventional pharmaceutically acceptable carriers, adjuvants, and/or vehicles. Formulation of drugs is generally discussed in, for example, Hoover, John E., Remington's Pharmaceutical Sciences (Mack Publishing Co., Easton, Pa.: 1975). See also, Liberman, H. A. See also, Lachman, L., eds., Pharmaceutical Dosage Forms (Marcel Decker, New York, N.Y., 1980).

Solid dosage forms for oral administration include, for example, capsules, tablets, pills, powders, and granules. In such solid dosage forms, the compounds or salts are ordinarily combined with one or more adjuvants. If administered per os, the compounds or salts can be mixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation, as can be provided in a dispersion of the compound or salt in hydroxypropylmethyl cellulose. In the case of capsules, tablets, and pills, the dosage forms also can comprise buffering agents, such as sodium citrate, or magnesium or calcium carbonate or bicarbonate. Tablets and pills additionally can be prepared with enteric coatings.

Liquid dosage forms for oral administration include, for example, pharmaceutically acceptable emulsions, solutions, suspensions, syrups, and elixirs containing inert diluents commonly used in the art (e.g., water). Such compositions also can comprise adjuvants, such as wetting, emulsifying, suspending, flavoring (e.g., sweetening), and/or perfuming agents.

“Parenteral administration” includes subcutaneous injections, intravenous injections, intramuscular injections, intrasternal injections, and infusion. Injectable preparations (e.g., sterile injectable aqueous or oleaginous suspensions) can be formulated according to the known art using suitable dispersing, wetting agents, and/or suspending agents. Acceptable vehicles and solvents include, for example, water, 1,3-butanediol, Ringer's solution, isotonic sodium chloride solution, bland fixed oils (e.g., synthetic mono- or diglycerides), fatty acids (e.g., oleic acid), dimethyl acetamide, surfactants (e.g., ionic and non-ionic detergents), and/or polyethylene glycols.

Formulations for parenteral administration may, for example, be prepared from sterile powders or granules having one or more of the carriers or diluents mentioned for use in the formulations for oral administration. The compounds or salts of this invention can be dissolved in water, polyethylene glycol, propylene glycol, ethanol, com oil, cottonseed oil, peanut oil, sesame oil, benzyl alcohol, sodium chloride, and/or various buffers.

Suppositories for rectal administration can be prepared by, for example, mixing the drug with a suitable nonirritating excipient that is solid at ordinary temperatures, but liquid at the rectal temperature and will therefore melt in the rectum to release the drug. Suitable excipients include, for example, such as cocoa butter; synthetic mono-, di-, or triglycerides; fatty acids; and/or polyethylene glycols

“Topical administration” includes the use of transdermal administration, such as transdermal patches or iontophoresis devices.

Other adjuvants and modes of administration well-known in the pharmaceutical art may also be used.

### E. Definitions

The term “alkyl” (alone or in combination with another term(s)) means a straight-or branched-chain saturated hydrocarbyl substituent typically containing from 1 to about 20 carbon atoms, more typically from 1 to about 8 carbon atoms, and even more typically from 1 to about 6 carbon atoms. Examples of such substituents include methyl, ethyl, n-propyl, isopropyl, n-butyl, isobutyl, sec-butyl, tert-butyl, pentyl, iso-amyl, hexyl, octyl, and the like.

The term “alkenyl” (alone or in combination with another term(s)) means a straight- or branched-chain hydrocarbyl substituent containing one or more double bonds and typically from 2 to about 20 carbon atoms, more typically from about 2 to about 8 carbon atoms, and even more typically from about 2 to about 6 carbon atoms. Examples of such substituents include ethenyl (vinyl); 2-propenyl; 3-propenyl; 1,4-pentadienyl; 1,4-butadienyl; 1-butenyl; 2-butenyl; 3-butenyl; decenyl; and the like.

The term “alkynyl” (alone or in combination with another term(s)) means a straight- or branched-chain hydrocarbyl substituent containing one or more triple bonds and typically from 2 to about 20 carbon atoms, more typically from about 2 to about 8 carbon atoms, and even more typically from about 2 to about 6 carbon atoms. Examples of such substituents include ethynyl, 2-propynyl, 3-propynyl, decynyl, 1-butynyl, 2-butynyl, 3-butynyl, and the like.

The term “carbocyclyl” (alone or in combination with another term(s)) means a saturated cyclic (i.e., “cycloalkyl”), partially saturated cyclic (i.e., “cycloalkenyl”), or completely unsaturated (i.e., “aryl”) hydrocarbyl substituent containing from 3 to 14 carbon ring atoms (“ring atoms” are the atoms bound together to form the ring or rings of a cyclic substituent). A carbocyclyl may be a single ring, which typically contains from 3 to 6 ring atoms. Examples of such single-ring carbocyclyls include cyclopropanyl, cyclobutanyl, cyclopentyl, cyclopentenyl, cyclopentadienyl, cyclohexyl, cyclohexenyl, cyclohexadienyl, and phenyl. A carbocyclyl alternatively may be 2 or 3 rings fused together, such as naphthalenyl, tetrahydronaphthalenyl (also known as “tetralinyl”), indenyl, isoindenyl, indanyl, bicyclodecanyl, anthracenyl, phenanthrene, benzonaphthenyl (also known as “phenalenyl”), fluoreneyl, decalinyl, and norpinanyl.

The term “cycloalkyl” (alone or in combination with another term(s)) means a saturated cyclic hydrocarbyl substituent containing from 3 to 14 carbon ring atoms. A cycloalkyl may be a single carbon ring, which typically contains from 3 to 6 carbon ring atoms. Examples of single-ring cycloalkyls include cyclopropyl (or “cyclopropanyl”), cyclobutyl (or “cyclobutanyl”), cyclopentyl (or “cyclopentanyl”), and cyclohexyl (or “cyclohexanyl”). A cycloalkyl alternatively may be 2 or 3 carbon rings fused together, such as, decalinyl or norpinanyl.

The term “aryl” (alone or in combination with another term(s)) means an aromatic carbocyclyl containing from 6 to 14 carbon ring atoms. Examples of aryls include phenyl, naphthalenyl, and indenyl.

In some instances, the number of carbon atoms in a hydrocarbyl substituent (e.g., alkyl, alkenyl, alkynyl, or cycloalkyl) is indicated by the prefix “Cₓ-C_{y-}”, wherein x is the minimum and y is the maximum number of carbon atoms in the substituent. Thus, for example, “C₁-C₆-alkyl” refers to an alkyl substituent containing from 1 to 6 carbon atoms. Illustrating further, C₃-C₆-cycloalkyl means a saturated hydrocarbyl ring containing from 3 to 6 carbon ring atoms.

The term “hydrogen” (alone or in combination with another term(s)) means a hydrogen radical, and may be depicted as —H.

The term “hydroxy” (alone or in combination with another term(s)) means —OH.

The term “nitro” (alone or in combination with another term(s)) means —NO₂.

The term “cyano” (alone or in combination with another term(s)) means —CN, which also may be depicted:

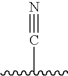

The term “keto” (alone or in combination with another term(s)) means an oxo radical, and may be depicted as ═O.

The term “carboxy” (alone or in combination with another term(s)) means —C(O)—OH, which also may be depicted as:

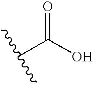

The term “amino” (alone or in combination with another term(s)) means —NH₂. The term “monosubstituted amino” (alone or in combination with another term(s)) means an amino substituent wherein one of the hydrogen radicals is replaced by a non-hydrogen substituent. The term “disubstituted amino” (alone or in combination with another term(s)) means an amino substituent wherein both of the hydrogen atoms are replaced by non-hydrogen substituents, which may be identical or different.

The term “halogen” (alone or in combination with another term(s)) means a fluorine radical (which may be depicted as —F), chlorine radical (which may be depicted as —Cl), bromine radical (which may be depicted as —Br), or iodine radical (which may be depicted as —I). Typically, a fluorine radical or chlorine radical is preferred, with a fluorine radical often being particularly preferred.

A substituent is “substitutable” if it comprises at least one carbon or nitrogen atom that is bonded to one or more hydrogen atoms. Thus, for example, hydrogen, halogen, and cyano do not fall within this definition.

If a substituent is described as being “substituted”, a non-hydrogen radical is in the place of a hydrogen radical on a carbon or nitrogen of the substituent. Thus, for example, a substituted alkyl substituent is an alkyl substituent wherein at least one non-hydrogen radical is in the place of a hydrogen radical on the alkyl substituent. To illustrate, monofluoroalkyl is alkyl substituted with a fluoro radical, and difluoroalkyl is alkyl substituted with two fluoro radicals. It should be recognized that if there are more than one substitutions on a substituent, each non-hydrogen radical may be identical or different (unless otherwise stated).

If a substituent is described as being “optionally substituted”, the substituent may be either (1) not substituted or (2) substituted. If a substituent is described as being optionally substituted with up to a particular number of non-hydrogen radicals, that substituent may be either (1) not substituted; or (2) substituted by up to that particular number of non-hydrogen radicals or by up to the maximum number of substitutable positions on the substituent, whichever is less. Thus, for example, if a substituent is described as a heteroaryl optionally substituted with up to 3 non-hydrogen radicals, then any heteroaryl with less than 3 substitutable positions would be optionally substituted by up to only as many non-hydrogen radicals as the heteroaryl has substitutable positions. To illustrate, tetrazolyl (which has only one substitutable position) would be optionally substituted with up to one non-hydrogen radical. To illustrate further, if an amino nitrogen is described as being optionally substituted with up to 2 non-hydrogen radicals, then a primary amino nitrogen will be optionally substituted with up to 2 non-hydrogen radicals, whereas a secondary amino nitrogen will be optionally substituted with up to only 1 non-hydrogen radical. Further illustrations of this definition may be found above at, for example, the sub-section entitled “General Description of Preferred A¹ and A² Substituents.”

This specification uses the terms “substituent” and “radical” interchangeably.

The prefix “halo” indicates that the substituent to which the prefix is attached is substituted with one or more independently selected halogen radicals. For example, haloalkyl means an alkyl substituent wherein at least one hydrogen radical is replaced with a halogen radical. Examples of haloalkyls include chloromethyl, 1-bromoethyl, fluoromethyl, difluoromethyl, trifluoromethyl, 1,1,1-trifluoroethyl, and the like. Illustrating further, “haloalkoxy” means an alkoxy substituent wherein at least one hydrogen radical is replaced by a halogen radical. Examples of haloalkoxy substituents include chloromethoxy, 1-bromoethoxy, fluoromethoxy, difluoromethoxy, trifluoromethoxy (also known as “perfluoromethyloxy”), 1,1,1,-trifluoroethoxy, and the like. It should be recognized that if a substituent is substituted by more than one halogen radical, those halogen radicals may be identical or different (unless otherwise stated).

The prefix “perhalo” indicates that every hydrogen radical on the substituent to which the prefix is attached is replaced with independently selected halogen radicals, i.e., each hydrogen radical on the substituent is replaced with a halogen radical. If all the halogen radicals are identical, the prefix typically will identify the halogen radical. Thus, for example, the term “perfluoro” means that every hydrogen radical on the substituent to which the prefix is attached is substituted with a fluorine radical. To illustrate, the term “perfluoroalkyl” means an alkyl substituent wherein a fluorine radical is in the place of each hydrogen radical. Examples of perfluoroalkyl substituents include trifluoromethyl (—CF₃), perfluorobutyl, perfluoroisopropyl, perfluorododecyl, perfluorodecyl, and the like. To illustrate further, the term “perfluoroalkoxy” means an alkoxy substituent wherein each hydrogen radical is replaced with a fluorine radical. Examples of perfluoroalkoxy substituents include trifluoromethoxy (—O—CF₃), perfluorobutoxy, perfluoroisopropoxy, perfluorododecoxy, perfluorodecoxy, and the like.

The term “carbonyl” (alone or in combination with another term(s)) means —C(O)—, which also may be depicted as: 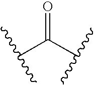
This term also is intended to encompass a hydrated carbonyl substituent, i.e., —C(OH)₂—.

The term “aminocarbonyl” (alone or in combination with another term(s)) means —C(O)—NH₂, which also may be depicted as:

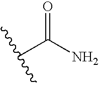

The term “oxy” (alone or in combination with another term(s)) means an ether substituent, and may be depicted as —O—.

The term “alkoxy” (alone or in combination with another term(s)) means an alkylether substituent, i.e., —O-alkyl. Examples of such a substituent include methoxy (—O—CH₃), ethoxy, n-propoxy, isopropoxy, n-butoxy, iso-butoxy, sec-butoxy, tert-butoxy, and the like.

The term “alkylcarbonyl” (alone or in combination with another term(s)) means —C(O)-alkyl. For example, “ethylcarbonyl” may be depicted as:

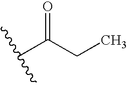

The term “aminoalkylcarbonyl” (alone or in combination with another term(s)) means —C(O)-alkyl-NH₂. For example, “aminomethylcarbonyl” may be depicted as:

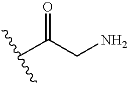

The term “alkoxycarbonyl” (alone or in combination with another term(s)) means —C(O)—O-alkyl. For example, “ethoxycarbonyl” may be depicted as:

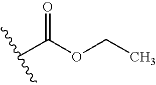

The term “carbocyclylcarbonyl” (alone or in combination with another term(s)) means —C(O)-carbocyclyl. For example, “phenylcarbonyl” may be depicted as: 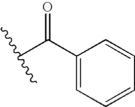
Similarly, the term “heterocyclylcarbonyl” (alone or in combination with another term(s)) means —C(O)-heterocyclyl.

The term “carbocyclylalkylcarbonyl” (alone or in combination with another term(s)) means —C(O)-alkyl-carbocyclyl. For example, “phenylethylcarbonyl” may be depicted as: 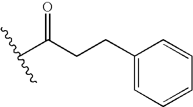
Similarly, the term “heterocyclylalkylcarbonyl” (alone or in combination with another term(s)) means —C(O)-alkyl-heterocyclyl.

The term “carbocyclyloxycarbonyl” (alone or in combination with another term(s)) means —C(O)-O-carbocyclyl. For example, “phenyloxycarbonyl” may be depicted as:

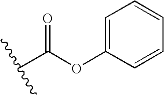

The term “carbocyclylalkoxycarbonyl” (alone or in combination with another term(s)) means —C(O)—O-alkyl-carbocyclyl. For example, “phenylethoxycarbonyl” may be depicted as:

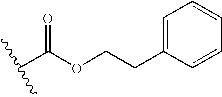

The term “thio” or “thia” (alone or in combination with another term(s)) means a thiaether substituent, i.e., an ether substituent wherein a divalent sulfur atom is in the place of the ether oxygen atom. Such a substituent may be depicted as —S—. This, for example, “alkyl-thio-alkyl” means alkyl-S-alkyl.

The term “thiol” or “sulfhydryl” (alone or in combination with another term(s)) means a sulfhydryl substituent, and may be depicted as —SH.

The term “(thiocarbonyl)” (alone or in combination with another term(s)) means a carbonyl wherein the oxygen atom has been replaced with a sulfur. Such a substituent may be depicted as —C(S)—, and also may be depicted as:

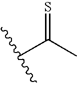

The term “sulfonyl” (alone or in combination with another term(s)) means —S(O)₂—, which also may be depicted as: 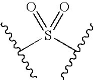
Thus, for example, “alkyl-sulfonyl-alkyl” means alkyl-S(O)₂-alkyl.

The term “aminosulfonyl” (alone or in combination with another term(s)) means —S(O)₂—NH₂, which also may be depicted as:

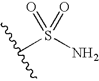

The term “sulfoxido” (alone or in combination with another tern(s)) means —S(O)—, which also may be depicted as: 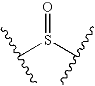
Thus, for example, “alkyl-sulfoxido-alkyl” means alkyl-S(O)-alkyl.

The term “heterocyclyl” (alone or in combination with another term(s)) means a saturated (i.e., “heterocycloalkyl”), partially saturated (i.e., “heterocycloalkenyl”), or completely unsaturated (i.e., “heteroaryl”) ring structure containing a total of 3 to 14 ring atoms. At least one of the ring atoms is a heteroatom (i.e., oxygen, nitrogen, or sulfur), with the remaining ring atoms being independently selected from the group consisting of carbon, oxygen, nitrogen, and sulfur.

A heterocyclyl may be a single ring, which typically contains from 3 to 7 ring atoms, more typically from 3 to 6 ring atoms, and even more typically 5 to 6 ring atoms. Examples of single-ring heterocyclyls include furanyl, dihydrofurnayl, tetradydrofurnayl, thiophenyl (also known as “thiofuranyl”), dihydrothiophenyl, tetrahydrothiophenyl, pyrrolyl, isopyrrolyl, pyrrolinyl, pyrrolidinyl, imidazolyl, isoimidazolyl, imidazolinyl, imidazolidinyl, pyrazolyl, pyrazolinyl, pyrazolidinyl, triazolyl, tetrazolyl, dithiolyl, oxathiolyl, oxazolyl, isoxazolyl, thiazolyl, isothiazolyl, thiazolinyl, isothiazolinyl, thiazolidinyl, isothiazolidinyl, thiodiazolyl, oxathiazolyl, oxadiazolyl (including 1,2,3-oxadiazolyl, 1,2,4-oxadiazolyl (also known as “azoximyl”), 1,2,5-oxadiazolyl (also known as “furazanyl”), or 1,3,4-oxadiazolyl), oxatriazolyl (including 1,2,3,4-oxatriazolyl or 1,2,3,5-oxatriazolyl), dioxazolyl (including 1,2,3-dioxazolyl, 1,2,4-dioxazolyl, 1,3,2-dioxazolyl, or 1,3,4-dioxazolyl), oxathiazolyl, oxathiolyl, oxathiolanyl, pyranyl (including 1,2-pyranyl or 1,4-pyranyl), dihydropyranyl, pyridinyl (also known as “azinyl”), piperidinyl, diazinyl (including pyridazinyl (also known as “1,2-diazinyl”), pyrimidinyl (also known as “1,3-diazinyl”), or pyrazinyl (also known as “1,4-diazinyl”)), piperazinyl, triazinyl (including s-triazinyl (also known as “1,3,5-triazinyl”), as-triazinyl (also known 1,2,4-triazinyl), and v-triazinyl (also known as “1,2,3-triazinyl”)), oxazinyl (including 1,2,3-oxazinyl, 1,3,2-oxazinyl, 1,3,6-oxazinyl (also known as “pentoxazolyl”), 1,2,6-oxazinyl, or 1,4-oxazinyl), isoxazinyl (including o-isoxazinyl or p-isoxazinyl), oxazolidinyl, isoxazolidinyl, oxathiazinyl (including 1,2,5-oxathiazinyl or 1,2,6-oxathiazinyl), oxadiazinyl (including 1,4,2-oxadiazinyl or 1,3,5,2-oxadiazinyl), morpholinyl, azepinyl, oxepinyl, thiepinyl, and diazepinyl.

A heterocyclyl alternatively may be 2 or 3 rings fused together, such as, for example, indolizinyl, pyrindinyl, pyranopyrrolyl, 4H-quinolizinyl, purinyl, naphthyridinyl, pyridopyridinyl (including pyrido[3,4-b]-pyridinyl, pyrido[3,2-b]-pyridinyl, or pyrido[4,3-b]-pyridinyl), and pteridinyl. Other examples of fused-ring heterocyclyls include benzo-fused heterocyclyls, such as indolyl, isoindolyl (also known as “isobenzazolyl” or “pseudoisoindolyl”), indoleninyl (also known as “pseudoindolyl”), isoindazolyl (also known as “benzpyrazolyl”), benzazinyl (including quinolinyl (also known as “1-benzazinyl”) or isoquinolinyl (also known as “2-benzazinyl”)), phthalazinyl, quinoxalinyl, quinazolinyl, benzodiazinyl (including cinnolinyl (also known as “1,2-benzodiazinyl”) or quinazolinyl (also known as “1,3-benzodiazinyl”)), benzopyranyl (including “chromanyl” or “isochromanyl”), benzothiopyranyl (also known as “thiochromanyl”), benzoxazolyl, indoxazinyl (also known as “benzisoxazolyl”), anthranilyl, benzodioxolyl, benzodioxanyl, benzoxadiazolyl, benzofuranyl (also known as “coumaronyl”), isobenzofuranyl, benzothienyl (also known as “benzothiophenyl”, “thionaphthenyl”, or “benzothiofuranyl”), isobenzothienyl (also known as “isobenzothiophenyl”, “isothionaphthenyl”, or “isobenzothiofuranyl”), benzothiazolyl, benzothiadiazolyl, benzimidazolyl, benzotriazolyl, benzoxazinyl (including 1,3,2-benzoxazinyl, 1,4,2-benzoxazinyl , 2,3,1-benzoxazinyl , or 3,1,4-benzoxazinyl), benzisoxazinyl (including 1,2-benzisoxazinyl or 1,4-benzisoxazinyl), tetrahydroisoquinolinyl , carbazolyl, xanthenyl, and acridinyl.

The term “2-fused'ring” heterocyclyl (alone or in combination with another term(s)) means a saturated, partially saturated, or aryl heterocyclyl containing 2 fused rings. Examples of 2-fused-ring heterocyclyls include indolizinyl, pyrindinyl, pyranopyrrolyl, 4H-quinolizinyl, purinyl, naphthyridinyl, pyridopyridinyl, pteridinyl, indolyl, isoindolyl, indoleninyl, isoindazolyl, benzazinyl, phthalazinyl, quinoxalinyl, quinazolinyl, benzodiazinyl, benzopyranyl, benzothiopyranyl, benzoxazolyl, indoxazinyl, anthranilyl, benzodioxolyl, benzodioxanyl, benzoxadiazolyl, benzofuranyl, isobenzofuranyl, benzothienyl, isobenzothienyl, benzothiazolyl, benzothiadiazolyl, benzimidazolyl, benzotriazolyl, benzoxazinyl, benzisoxazinyl, and tetrahydroisoquinolinyl.

The term “heteroaryl” (alone or in combination with another term(s)) means an aromatic heterocyclyl containing from 5 to 14 ring atoms. A heteroaryl may be a single ring or 2 or 3 fused rings. Examples of heteroaryl substituents include 6-membered ring substituents such as pyridyl, pyrazyl, pyrimidinyl, and pyridazinyl; 5-membered ring substituents such as 1,3,5-, 1,2,4- or 1,2,3-triazinyl, imidazyl, furanyl, thiophenyl, pyrazolyl, oxazolyl, isoxazolyl, thiazolyl, 1,2,3-, 1,2,4-, 1,2,5-, or 1,3,4-oxadiazolyl and isothiazolyl; 6/5-membered fused ring substituents such as benzothiofuranyl, isobenzothiofuranyl, benzisoxazolyl, benzoxazolyl, purinyl, and anthranilyl; and 6/6-membered fused rings such as 1,2-, 1,4-, 2,3- and 2,1-benzopyronyl, quinolinyl, isoquinolinyl, cinnolinyl, quinazolinyl, and 1,4-benzoxazinyl.

A carbocyclyl or heterocyclyl can optionally be substituted with, for example, one or more substituents independently selected from the group consisting of halogen, hydroxy, carboxy, keto, alkyl, alkoxy, alkoxyalkyl, alkylcarbonyl (also known as “alkanoyl”), aryl, arylalkyl, arylalkoxy, arylalkoxyalkyl, arylalkoxycarbonyl, cycloalkyl, cycloalkylalkyl, cycloalkylalkoxy, cycloalkylalkoxyalkyl, and cycloalkylalkoxycarbonyl. More typically, a carbocyclyl or heterocyclyl may optionally be substituted with, for example, one or more substituents independently selected from the group consisting of halogen, —OH, —C(O)—OH, keto, C₁-C₆-alkyl, C₁-C₆-alkoxy, C₁-C₆-alkoxy-C₁-C₆-alkyl, C₁-C₆-alkylcarbonyl, aryl, aryl-C₁-C₆-alkyl, aryl-C₁-C₆-alkoxy, aryl-C₁-C₆-alkoxy-C₁-C₆-alkyl, aryl-C₁-C₆-alkoxycarbonyl, cycloalkyl, cycloalkyl-C₁-C₆-alkyl, cycloalkyl-C₁-C₆-alkoxy, cycloalkyl-C₁-C₆-alkoxy-C₁-C₆-alkyl, and cycloalkyl-C₁-C₆-alkoxycarbonyl. The alkyl, alkoxy, alkoxyalkyl, alkylcarbonyl, aryl, arylalkyl, arylalkoxy, arylalkoxyalkyl, or arylalkoxycarbonyl substituent(s) may further be substituted with, for example, one or more halogen. The aryls or cycloalkyls are typically single-ring substituents containing from 3 to 6 ring atoms, and more typically from 5 to 6 ring atoms.

An aryl or heteroaryl can optionally be substituted with, for example, one or more substituents independently selected from the group consisting of halogen, —OH, —CN, —NO₂, —SH, —C(O)—OH, amino, aminocarbonyl, aminoalkyl, alkyl, alkylthio, carboxyalkylthio, alkylcarbonyl, alkylcarbonyloxy, alkoxy, alkoxyalkyl, alkoxycarbonyl, alkoxycarbonylalkoxy, alkoxyalkylthio, alkoxycarbonylalkylthio, carboxyalkoxy, alkoxycarbonylalkoxy, carbocyclyl, carbocyclylalkyl, carbocyclyloxy, carbocyclylthio, carbocyclylalkylthio, carbocyclylamino, carbocyclylalkylamino, carbocyclylcarbonylamino, carbocyclylcarbonyl, carbocyclylalkyl, carbonyl, carbocyclylcarbonyloxy, carbocyclyloxycarbonyl, carbocyclylalkoxycarbonyl, carbocyclyloxyalkoxycarbocyclyl, carbocyclylthioalkylthiocarbocyclyl, carbocyclylthioalkoxycarbocyclyl, carbocyclyloxyalkylthiocarbocyclyl, heterocyclyl, heterocyclylalkyl, heterocyclyloxy, heterocyclylthio, heterocyclylalkylthio, heterocyclylamino, heterocyclylalkylamino, heterocyclylcarbonylamino, heterocyclylcarbonyl, heterocyclylalkylcarbonyl, heterocyclyloxycarbonyl, heterocyclylcarbonyloxy, heterocyclylalkoxycarbonyl, heterocyclyloxyalkoxyheterocyclyl, heterocyclylthioalkylthioheterocyclyl, heterocyclylthioalkoxyheterocyclyl, and heterocyclyloxyalkylthioheterocyclyl. More typically, an aryl or heteroaryl may, for example, optionally be substituted with one or more substituents independently selected from the group consisting of halogen, —OH, —CN, —NO₂, —SH, —C(O)—OH, amino, aminocarbonyl, amino-C₁-C₆-alkyl, C₁-C₆-alkyl, C₁-C₆-alkylthio, carboxy-C₁-C₆-alkylthio, C₁-C₆-alkylcarbonyl, C₁-C₆-alkylcarbonyloxy, C₁-C₆-alkoxy, C₁-C₆-alkoxy-C₁-C₆-alkyl, C₁-C₆-alkoxycarbonyl, C₁-C₆-alkoxycarbonyl-C₁-C₆-alkoxy, C₁-C₆-alkoxy-C₁-C₆-alkylthio, C₁-C₆-alkoxycarbonyl-C₁-C₆-alkylthio, carboxy-C₁-C₆-alkoxy, C₁-C₆-alkoxycarbonyl-C₁-C₆-alkoxy, aryl, aryl-C₁-C₆-alkyl, aryloxy, arylthio, aryl-C₁-C₆-alkylthio, arylamino, aryl-C₁-C₆-alkylamino, arylcarbonylamino, arylcarbonyl, aryl-C₁-C₆-alkylcarbonyl, arylcarbonyloxy, aryloxycarbonyl, aryl-C₁-C₆-alkoxycarbonyl, aryloxy-C₁-C₆-alkoxyaryl, arylthio-C₁-C₆-alkylthioaryl, arylthio-C₁-C₆-alkoxyaryl, aryloxy-C₁-C₆-alkylthioaryl, cycloalkyl, cycloalkyl-C₁-C₆-alkyl, cycloalkyloxy, cycloalkylthio, cycloalkyl-C₁-C₆-alkylthio, cycloalkylamino, cycloalkyl-C₁-C₆-alkylamino, cycloalkylcarbonylamino, cycloalkylcarbonyl, cycloalkyl-C₁-C₆-alkylcarbonyl, cycloalkylcarbonyloxy, cycloalkyloxycarbonyl, cycloalkyl-C₁-C₆-alkoxycarbonyl, heteroaryl, heteroaryl-C₁-C₆-alkyl, heteroaryloxy, heteroarylthio, heteroaryl-C₁-C₆-alkylthio, heteroarylamino, heteroaryl-C₁-C₆-alkylamino, heteroarylcarbonylamino, heteroarylcarbonyl, heteroaryl-C₁-C₆-alkylcarbonyl, heteroaryloxycarbonyl, heteroarylcarbonyloxy, and heteroaryl-C₁-C₆-alkoxycarbonyl. Here, one or more hydrogen bound to a carbon in any such substituent may, for example, optionally be replaced with halogen. In addition, the cycloalkyl, aryl, and heteroaryl are typically single-ring substituents containing 3 to 6 ring atoms, and more typically 5 or 6 ring atoms.

A prefix attached to a multi-component substituent only applies to the first component. To illustrate, the term “alkylcycloalkyl” contains two components: alkyl and cycloalkyl. Thus, the C₁-C₆-prefix on C₁-C₆-alkylcycloalkyl means that the alkyl component of the alkylcycloalkyl contains from 1 to 6 carbon atoms; the C₁-C₆-prefix does not describe the cycloalkyl component. To illustrate further, the prefix “halo” on haloalkoxyalkyl indicates that only the alkoxy component of the alkoxyalkyl substituent is substituted with one or more halogen radicals. If halogen substitution may alternatively or additionally occur on the alkyl component, the substituent would instead be described as “halogen-substituted alkoxyalkyl” rather than “haloalkoxyalkyl.” And finally, if the halogen substitution may only occur on the alkyl component, the substituent would instead be described as “alkoxyhaloalkyl.”

If substituents are described as being “independently selected” from a group, each substituent is selected independent of the other. Each substituent therefore may be identical to or different from the other substituent(s).

When words are used to describe a substituent, the rightmost-described component of the substituent is the component that has the free valence. To illustrate, benzene substituted with methoxyethyl has the following structure: 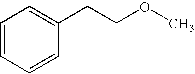
As can be seen, the ethyl is bound to the benzene, and the methoxy is the component of the substituent that is the component furthest from the benzene. As further illustration, benzene substituted with cyclohexanylthiobutoxy has the following structure:

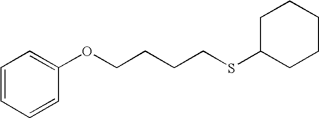

When words are used to describe a linking element between two other elements of a depicted chemical structure, the rightmost-described component of the substituent is the component that is bound to the left element in the depicted structure. To illustrate, if the chemical structure is X-L-Y and L is described as methylcyclohexanylethyl, then the chemical would be X-ethyl-cyclohexanyl-methyl-Y.

When a chemical formula is used to describe a substituent, the dash on the left side of the formula indicates the portion of the substituent that has the free valence. To illustrate, benzene substituted with —C(O)—OH has the following structure:

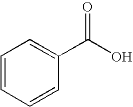

When a chemical formula is used to describe a linking element between two other elements of a depicted chemical structure, the leftmost dash of the substituent indicates the portion of the substituent that is bound to the left element in the depicted structure. The rightmost dash, on the other hand, indicates the portion of the substituent that is bound to the right element in the depicted structure. To illustrate, if the depicted chemical structure is X-L-Y and L is described as —C(O)—N(H)—, then the chemical would be:

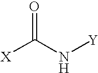

The term “pharmaceutically acceptable” is used adjectivally in this patent to mean that the modified noun is appropriate for use as a pharmaceutical product or as a part of a pharmaceutical product.

With reference to the use of the words “comprise” or “comprises” or “comprising” in this patent (including the claims), Applicants note that unless the context requires otherwise, those words are used on the basis and clear understanding that they are to be interpreted inclusively, rather than exclusively, and that Applicants intend each of those words to be so interpreted in construing this patent, including the claims below.

### F. Compound Preparation

The detailed examples below illustrate preparation of compounds and salts of this invention. Other compounds and salts of this invention may be prepared using the methods illustrated in these examples (either alone or in combination with techniques generally known in the art). Such known techniques include, for example, those disclosed in Int'l Publ. No. WO 99/25687 (PCT Patent Application No. PCT/US98/23242 published on May 27, 1999), which issued as U.S. Pat. No. 6,541,489 on Apr. 1, 2003 (incorporated herein by reference). Such known techniques also include, for example, those disclosed in Int'l Publ. No. WO 00/50396 (PCT Patent Application No. PCT/US00/02518 published on Aug. 31, 2000) (incorporated herein by reference). Such known techniques further include, for example, those disclosed in Int'l Publ. No. WO 00/69821 (PCT Patent Application No. PCT/US00/06719 published on Nov. 23, 2000) (incorporated herein by reference). Such known techniques also include, for example, those disclosed in Int'l Publ. No. WO 02/092588 (PCT Application No. PCT/US02/15257 published Nov. 21, 2002) (incorporated herein by reference).

### EXAMPLES

The following examples are merely illustrative, and not limiting to the remainder of this disclosure in any way.

### Example 1

### Preparation of tert-butyl 4-{[4-(4,4,5,5-tetramethyl-1,3,2-dioxaborolan-2-yl)phenyl]sulfonyl}perhydro-2H-pyran-4-carboxylate

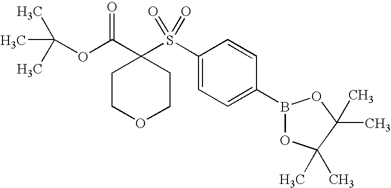

### Part A. Preparation of tert-butyl 2-[(4-bromophenyl)sulfonyl]acetate (2)

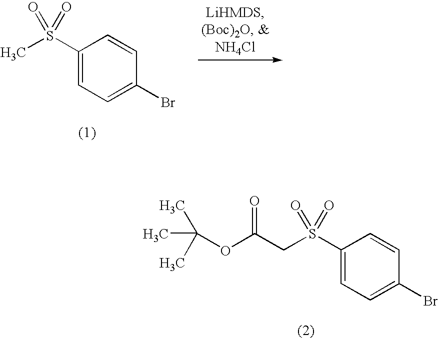

To a −78° C. mixture of 4-bromo-1-(methylsulfonyl)benzene (1) (58 g, 0.25 mol) and di-tert-butyldicarbonate (“(Boc)₂O”) (59 g, 0.27 mol) in 800 mL anhydrous tetrahydrofuran (“THF”) was added lithium hexamethyldisilazide (“LiHMDS”) (738 mL of 1.0M solution in THF, 0.74 mol). The resulting mixture was warmed to 0° C. and stirred for 1 hr, after which no starting material (1) was detected by HPLC. The mixture was quenched with saturated ammonium chloride (“NH₄Cl”) (700 mL) and warmed to room temperature. The organic layer was collected, and the aqueous layer was extracted with ethyl acetate (2×500 mL). The combined organic layers were washed with water (500 mL) and brine (500 mL), dried over MgSO₄, filtered, and concentrated to produce a yellow solid. LCMS: [M+Na]=358.95.

### Part B. Preparation of tert-butyl 4-[(4-bromophenyl)sulfonyl]perhydro-2H-pyran-4-carboxylate (3)

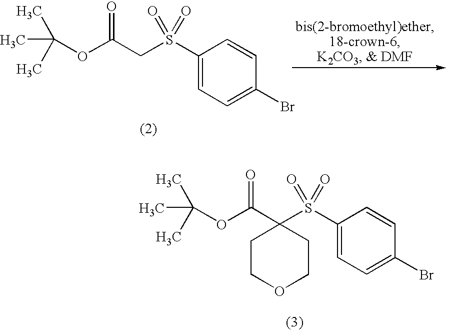

To a room temperature mixture of the tert-butyl 2-[(4-bromophenyl)sulfonyl]acetate (2) from Part A (0.25 mol) in 100 mL dimethyl formamide (“DMF”) was added 18-crown-6 (19.4 g, 0.07 mol), potassium carbonate (“K₂CO₃”) (169 g, 1.22 mol), and bis(2-bromoethyl)ether (62.5 g, 0.27 mol). The mixture was stirred at room temperature for 18 hr, after which time no starting material (2) was detected by HPLC. The resulting mixture was concentrated, diluted in 500 mL ethylacetate (“CH₃COOC₂H₅” or “EtOAc”), and filtered. The resulting filtrate was concentrated to produce a yellow oil that solidified upon standing to afford the desire product (3). LCMS: [M+Na]=427.05.

### Part C. Preparation of tert-butyl 4-{[4-(4,4,5,5-tetramethyl-1,3,2-dioxaborolan-2-yl)phenyl]sulfonyl}perhydro-2H-pyran-4-carboxylate (4)

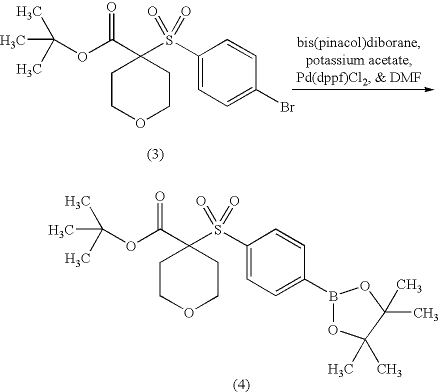

In a 2 L, 3-neck flask (equipped with an overhead stirring apparatus, an air-cooled condenser, and an N₂ inlet) were combined the tert-butyl 4-[(4-bromophenyl)sulfonyl]perhydro-2H-pyran-4-carboxylate (3) from Part B (0.25 mol), bis(pinacol)diborane (62 g, 0.25 mol), [1,1′-bis(diphenylphosphino)ferrocene]dichloropalladium(II) (“Pd(dppf)Cl₂”) (6.02 g, 7.38 mmol, 3 mol %), and potassium acetate (72 g, 0.74 mol). DMF (700 mL) was added, and the resulting mixture was stirred at 80° C. for 18 hr, after which time no starting material (3) was detected by HPLC. The resulting mixture was concentrated, diluted in 800 mL EtOAc, and washed with water (600 mL). The aqueous layer was extracted with EtOAc (2×400 mL). Afterward, the organic layers were combined, washed with brine (500 mL), dried over MgSO₄, filtered, and concentrated to form a dark oil. The crude material was purified by plug filtration silica (eluting with 4 L of 1:4 ethyl acetate:hexane, followed by 1:1 ethyl acetate:hexane), concentrated, and triturated with cold ether to afford 56 g (59% yield) of desired product (4) as a white solid. ¹H NMR (CDCl₃) δ: 1.35 (s, 12H), 1.45 (s, 9H), 2.16 (bs, 4H), 3.27 (m, 2H), 3.95 (bd, 2H), 7.79 (d, 2H), 7.94(d, 2H).

### Example 2

### Preparation of N-hydroxy-4-({4-[5-(3,3,4,4,4-pentafluorobutyl)pyridin-2-yl]phenyl}sulfonyl)tetrahydro-2H-pyran-4-carboxamide hydrochloride

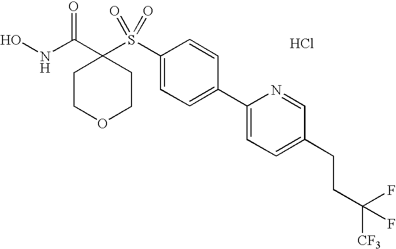

### Part A. Preparation of tert-butyl 4-{[4-(5-bromo-2-pyridyl)phenyl]sulfonyl}perhydro-2H-pyran-4-carboxylate (2)

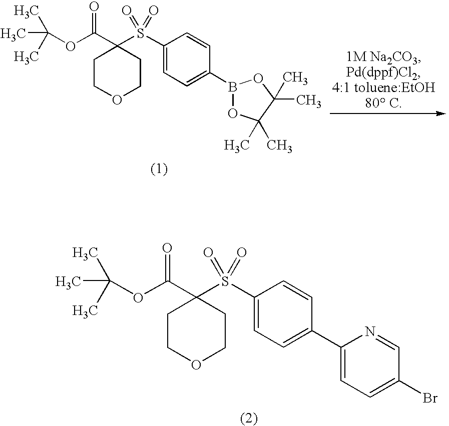

To a mixture of tert-butyl 4-{[4-(4,4,5,5-tetramethyl-1,3,2-dioxaborolan-2-yl)phenyl]sulfonyl}perhydro-2H-pyran-4-carboxylate (1) from Example 1 (10.0 g, 22.2 mmol) in toluene (40 mL), ethanol (10 mL), and 1M sodium carbonate (“Na₂CO₃”) (40 mL) under N₂ were added 2,5-dibromopyridine (6.54 g, 27.6 mmol) and [1,1′-bis(diphenylphosphino)ferrocene]dichloropalladium(II) (“Pd(dppf)Cl₂”) (0.90 g, 1.12 mmol). The mixture was heated at 80° C. under N₂ overnight. Afterward, the mixture was cooled to room temperature and diluted with ethyl acetate and water. The mixture was then filtered through a pad of Celite. The layers of the filtrate were separated, and the organic layer was washed with water (2 times) and saturated sodium chloride (1 time) before drying over anhydrous sodium sulfate. Filtration and evaporation of the solvent under reduced pressure produced a dark oil. The residue was dissolved in dichloromethane and purified on SiO₂ using 25% ethyl acetate/hexane. Some mixed fractions with the other regioisomer impurity were obtained, but only the clean, product-containing fractions were combined to afford 2.6 g of white solid (25% yield). ¹H NMR and mass spectrometry (MH⁺=482) were consistent with the desired compound (2).

### Part B. Preparation of tert-butyl 4-({4-[5-(3,3,4,4,4-pentafluorobutyl)-2-pyridyl]phenyl}sulfonyl)perhydro-2H-pyran-4-carboxylate (3)

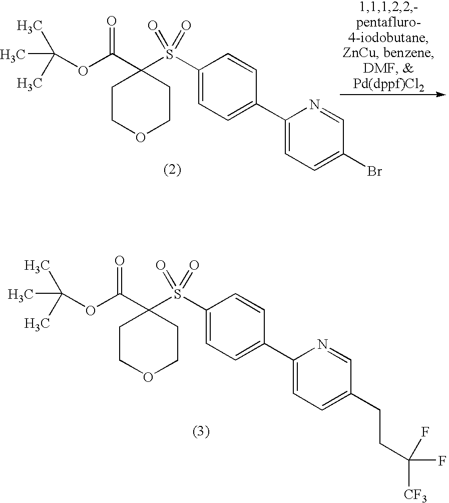

To a slurry of ZnCu couple (1.55 g, 23.9 mmol) in benzene (33 mL) and DMF (1.6 mL) was added 1,1,1,2,2-pentafluoro-4-iodobutane (4.29 g, 15.6 mmol). The mixture was heated at 60° C. under N₂ for 3 hr. A mixture of the tert-butyl 4-{[4-(5-bromo-2-pyridyl)phenyl]sulfonyl}perhydro-2H-pyran-4-carboxylate product (2) from Part A (2.5 g, 5.2 mmol) in benzene (8 mL) and DMF (2 mL) was subsequently added, followed by [1,1′-bis(diphenylphosphino)ferrocene]dichloropalladium(II) (0.21 g, 0.26 mmol). The temperature was increased to 75° C., and the reaction was continued overnight, after which time no starting material (2) was detected by HPLC. The mixture was cooled to room temperature and diluted with ethyl acetate and saturated ammonium chloride. The mixture was then filtered through a pad of Celite. The layers of the filtrate were separated, and the organic layer was washed with saturated ammonium chloride (2 times) and saturated sodium chloride (1 time) before drying over anhydrous sodium sulfate. Filtration and evaporation of the solvent under reduced pressure produced a dark oil. The crude material was purified on SiO₂ using dichloromethane with a methanol gradient to afford 2.7 grams (96% yield) of a yellow foam. ¹H NMR and mass spectrometry (MH⁺=550) were consistent with the desired compound (3).

### Part C. Preparation of 4-({4-[5-(3,3,4,4,4-pentafluorobutyl)-2-pyridyl]phenyl}sulfonyl)perhydro-2H-pyran-4-carboxylic acid (4)

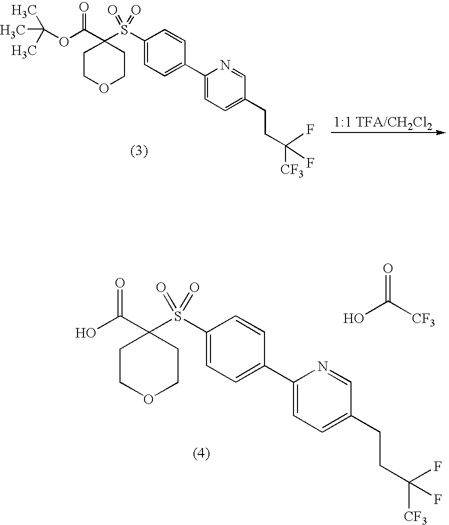

The tert-butyl 4-({4-[5-(3,3,4,4,4-pentafluorobutyl)-2-pyridyl]phenyl}sulfonyl)perhydro-2H-pyran-4-carboxylate product (3) from Part B (2.6 g, 4.7 mmol) was dissolved in 1:1 trifluoroacetic acid/dichloromethane (50 mL). The reaction was continued overnight at room temperature, after which time no starting material (3) was detected by HPLC. The mixture was concentrated under reduced pressure. Additional dichloromethane was then added, and the solvent was once again removed under reduced pressure to afford a tan solid (3.6 g, quantitative yield for the “di-TFA” salt). Mass spectrometry (MH⁺=494) was consistent with the desired product (4).

### Part D. Preparation of [4-({4-[5-(3,3,4,4,4-pentafluorobutyl)(2-pyridyl)]phenyl}sulfonyl)perhydro-2H-pyran-4-yl]-N-perhydro-2H-pyran-2-yloxycarboxamide (5)

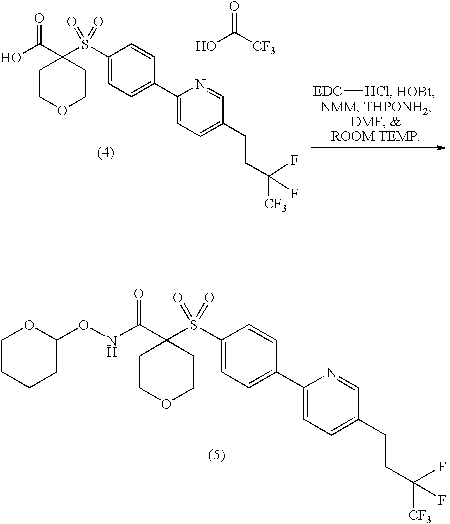

To a mixture of the 4-({4-[5-(3,3,4,4,4-pentafluorobutyl)-2-pyridyl]phenyl}sulfonyl)perhydro-2H-pyran-4-carboxylic acid product (4) from Part C (3.6 g, 5.0 mmol for “di-TFA”) in N,N-dimethylformamide (90 mL) were added N-hydroxybenzotriazole (“HOBt”) (0.94 g, 7.0 mmol), 4-methylmorpholine (“NMM”) (2.5 g, 2.7 mL, 25 mmol), 1-(3-dimethylaminopropyl)-3-ethylcarbodiimide hydrochloride (“EDC-HCl”) (3.4 g, 17.5 mmol), and O-(tetrahydro-2H-pyran-2-yl)hydroxylamine (“THPONH₂”) (2.0 g, 17.5 mmol). The reaction was continued overnight at room temperature under N₂, after which time no starting material (4) was detected by HPLC. The mixture was diluted with ethyl acetate, and the organic layer was extracted with water (3 times), extracted with saturated sodium bicarbonate (3 times), washed with saturated sodium chloride, and dried over anhydrous sodium sulfate. Filtration and evaporation of the solvent under reduced pressure afforded a dark oil. The crude material was purified by flash chromatography using dichloromethane with a methanol gradient (0-1%) to afford a white foam (1.1 g of pure material+another 1.7 g of slightly impure material). ¹H NMR and mass spectrometry (MH⁺+Na=615) were consistent with the desired product (5).

### Part E. Preparation of N-hydroxy-4-({4-[5-(3,3,4,4,4-pentafluorobutyl)pyridin-2-yl]phenyl}sulfonyl)tetrahydro-2H-pyran-4-carboxamide hydrochloride (6)

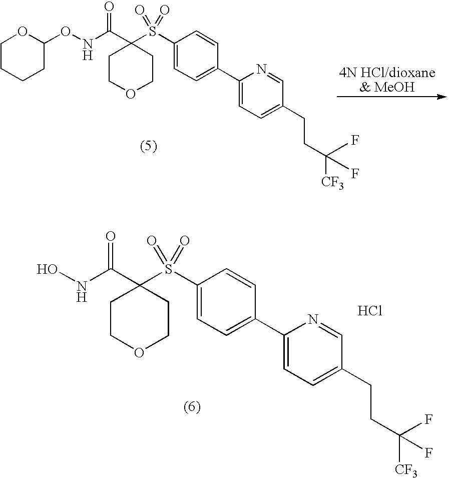

The [4-({4-[5-(3,3,4,4,4-pentafluorobutyl)(2-pyridyl)]phenyl }sulfonyl)perhydro-2H-pyran-4-yl]-N-perhydro-2H-pyran-2-yloxycarboxamide product (5) from Part D (1.1 g, 1.8 mmol) was dissolved in dioxane (8 mL), 4N HCl in dioxane (10 mL), and methanol (“MeOH”) (1 mL). The reaction was continued at ambient temperature for 2 hr. Afterward, HPLC indicated that the reaction was complete. The mixture was then concentrated under reduced pressure. The residue was triturated with diethyl ether, and the resulting white solid was collected by suction filtration (0.98 g, quantitative yield). ¹H NMR and mass spectrometry (MH⁺=508) were consistent with the desired product (6).

### Example 3

### Preparation of 4-({4-[4-(2-ethoxyethoxy)phenyl]phenyl}sulfonyl)perhydro-2H-pyran-4-carbohydroxamic acid

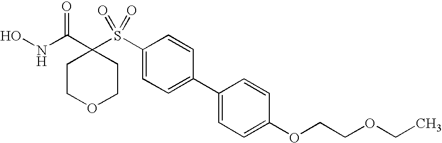

### Part A. Preparation of 1-bromo-4-(2-ethoxy-ethoxy)-benzene (2)

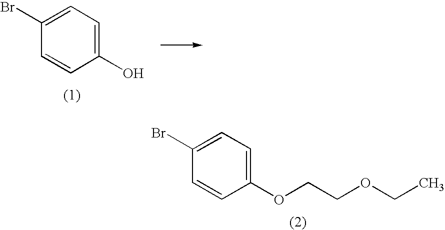

To a room temperature mixture of 4-bromophenol (1) (5.0 g, 28.9 mmol) in 15 mL DMF was added potassium carbonate (4.4 g, 31.8 mmol) and 2-bromoethyl ethyl ether (5.5 g, 36.4 mmol). The resulting mixture was stirred for 18 hr at room temperature. Subsequently, no starting material (1) was detectable by HPLC. The solvent was removed, and the resulting mixture was diluted in 100 mL ethyl acetate and filtered. The filtrate was concentrated to produce 6.2 g (86% yield) of the desired compound (2) in the form of a yellow oil.

### Part B. Preparation of 4′-(2-ethoxy-ethoxy)-4-methanesulfonyl-biphenyl (3)

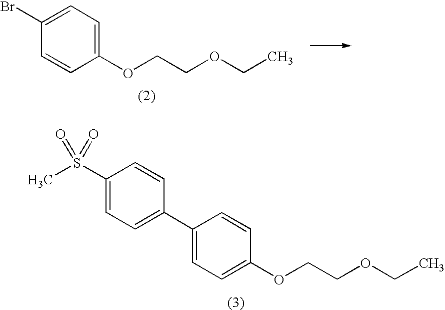

To a room temperature mixture of the 4′-(2-ethoxy-ethoxy)-4-methanesulfonyl-biphenyl product (2) from Part A (2.0 g, 8.10 mmol) in 18 mL DME (degassed) was added 4-(methanesulfonyl)phenyl boronic acid (3.3 g, 13.9 mmol), cesium carbonate (14 mL of 2M solution, 28.1 mmol), and tetrakistriphenylphosphine palladium (0.47 g, 0.41 mmol). The resulting mixture was heated at reflux for 18 hr. Subsequently, no starting material (2) was detectable by HPLC. The mixture was poured into 50 mL water and extracted with ethyl acetate (2×100 mL). The organic layers were combined, dried over MgSO₄, filtered, and concentrated to 10 g of black solid. The crude material was purified by flash column chromatography on silica eluting with 1:10 methanol:methylene chloride to produce 1.4 g (56% yield) of desired compound (3) in the form of a white solid. LCMS: [M+H]=321.1.

### Part C. Preparation of t-butyl-2-({4-[4-(2-ethoxyethoxy)phenyl]phenyl}sulfonyl) acetate (4)

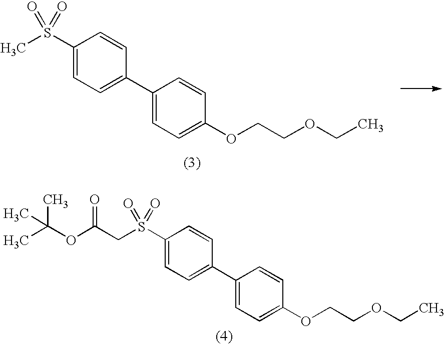

To a −78° C. mixture of the 4-bromo-1-(2-ethoxyethoxy)benzene product (3) from Part B (1.4 g, 4.4 mmol) and di-tert-butyldicarbonate (1.05 g, 4.8 mmol) in 15 mL anhydrous THF was added lithium hexamethyldisilazide (13 mL of 1.0M solution in THF, 13.1 mmol). The resulting mixture was warmed to 0° C. and stirred for 1 hr. Subsequently, no starting material (3) was detectable by HPLC. The reaction mixture was quenched with saturated NH₄Cl (30 mL), and warmed to room temperature. The organic layer was collected, and the aqueous layer was extracted with ethyl acetate (2×50 mL). The combined organic layers were washed with water (100 ML) and brine (100 mL), dried over MgSO₄, filtered, and concentrated to produce 1.7 g (92% yield) of a white solid. LCMS: [M+Na]=443.1.

### Part D. Preparation of t-butyl-4-({4-[4-(2-ethoxyethoxy)phenyl]phenyl}sulfonyl)perhydro-2H-pyran-4-carboxylate) (5)

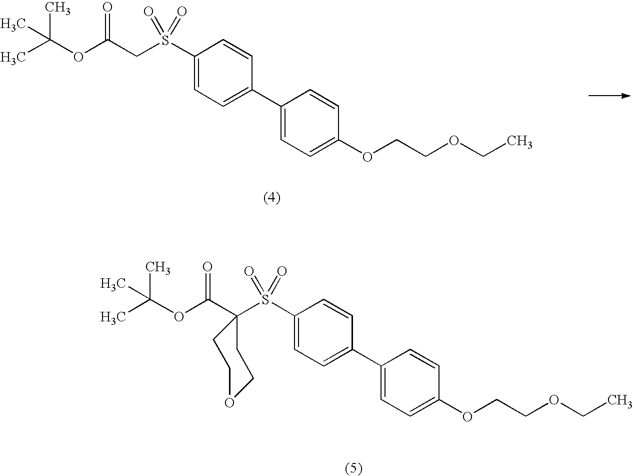

To a room temperature mixture of the t-butyl-2-({4-[4-(2-ethoxyethoxy)phenyl]phenyl}sulfonyl) acetate product (4) from Part C (800 mg, 1.9 mmol) in 8 mL DMF was added 18-crown-6 (150 mg, 0.6 mmol), potassium carbonate (1.3 g, 9.5 mmol), and bis(2-bromoethyl)ether (480 mg, 2.1 mmol). The mixture was stirred at room temperature for 18 hr. Subsequently, no starting material (4) was detectable by HPLC. The resulting mixture was concentrated, diluted in cold ether, and filtered to produce 800 mg (86% yield) of the desired compound (5). LCMS: [M+Na]=513.2.

### Part E. Preparation of 4-({4-[4-(2-ethoxyethoxy)phenyl]phenyl}sulfonyl)perhydro-2H-pyran-4-carboxylic acid (6)

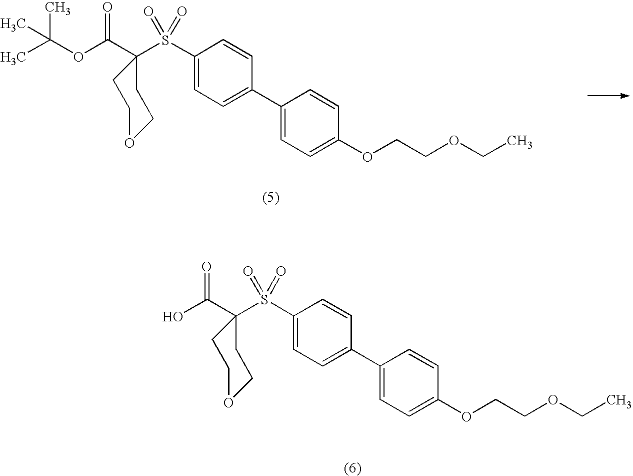

To a room temperature mixture of the t-butyl-4-({4-[4-(2-ethoxyethoxy)phenyl]phenyl}sulfonyl)perhydro-2H-pyran-4-carboxylate) product (5) from Part D (800 mg, 1.6 mmol) in 2.5 mL methylene chloride was added TFA (2.5 mL, 32.6 mmol). The reaction mixture was stirred for 18 hr at ambient temperature, after which no starting material was detected. The reaction mixture was concentrated, washed with ether, and filtered to produce 540 mg (78% yield) of the desired compound (6).

### Part F. Preparation of [4-({4-[4-(2-ethoxyethoxy)phenyl]phenyl}sulfonyl)perhydro-2H-pyran-4-yl]-N-perhydro-2H-pyran-2-yloxycarboxamide (7)

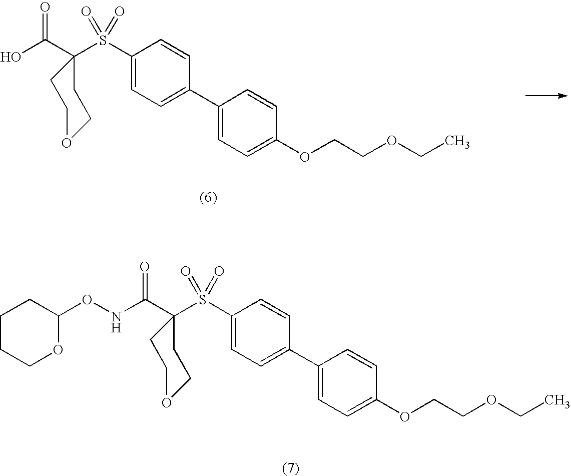

To a mixture of the 4-({4-[4-(2-ethoxyethoxy)phenyl]phenyl}sulfonyl)perhydro-2H-pyran-4-carboxylic acid product (6) from Part E (440 mg, 1.0 mmol) in 15 mL DMF was added triethylamine (310 uL, 2.2 mmol), 1-hydroxybenzatriazole (160 mg, 1.2 mmol), 2-(aminooxy)tetrahydro-2H-pyran (170 mg, 1.5 mmol), and 1-(3-dimethylaminopropyl)-3-ethylcarbodiimide hydrochloride (270 mg, 1.4 mmol). The reaction mixture was stirred for 18 hr at ambient temperature. Subsequently, no starting material (6) was detectable by HPLC. The reaction mixture was concentrated, and then partitioned in saturated NaHCO₃ and ethyl acetate. The organic layer was collected, and the aqueous layer extracted with ethyl acetate (2×25 mL). The combined organic layers were washed with water (50 mL) and brine (50 mL), dried over MgSO₄, filtered, and concentrated. The crude material (590 mg) was purified by flash column chromatography on silica eluting with 1:1 ethyl acetate: hexane to produce 350 mg (66% yield) of the desired compound (7) in the form of a white solid.

### Part G. Preparation of 4-({4-[4-(2-ethoxyethoxy)phenyl]phenyl}sulfonyl)perhydro-2H-pyran-4-carbohydroxamic acid (8)

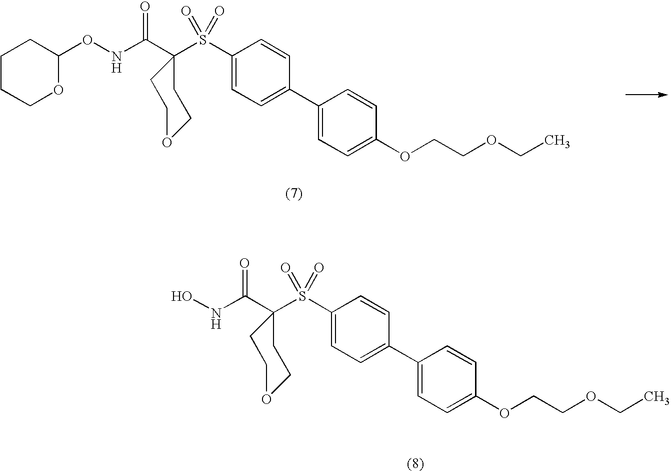

To a room temperature mixture of the [4-({4-[4-(2-ethoxyethoxy)phenyl]phenyl}sulfonyl)perhydro-2H-pyran-4-yl]-N-perhydro-2H-pyran-2-yloxycarboxamide product (7) from Part F (350 mg, 0.66 mmol) in 0.3 mL MeOH was added HCl (3.2 mL of 4.0M solution in dioxane, 13.1 mmol). The resulting mixture was stirred for 18 hr at ambient temperature. HPLC indicated presence of starting material (7). Additional HCl (4.0 mL of 4.0M solution in dioxane, 16.0 mmol) was added, and the reaction mixture was stirred for 18 hr at ambient temperature. Subsequently, no starting material (7) was detectable by HPLC. The mixture was added dropwise to a rapidly stirring solution of 75 mL ether. Afterward, 90% of the solvent removed in vacuo, and the mixture was triturated with ether. The solid was filtered to obtain 200 mg (68% yield) of the desired compound (8) in the form of an off-white amorphous solid. HRMS: [M+NH₄⁺](calc)=467.1847; [M+NH₄⁺](found)=467.1860.

### Example 4

### Preparation of 1-ethyl-4-[4′-(1,1,2,2-tetrafluoro-ethoxy)-biphenyl-4-sulfonyl]-piperidine-4-carboxylic acid hydroxyamide hydrochloride

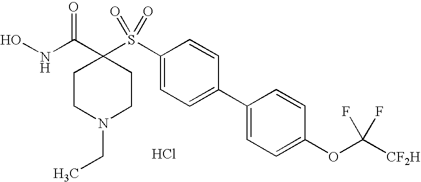

### Part A. Preparation of tert-butyl 1-benzyl-4-{[4′-(1,1,2,2-tetrafluoroethoxy)-1,1′-biphenyl-4-yl]sulfonyl}piperidine-4-carboxylate (3)

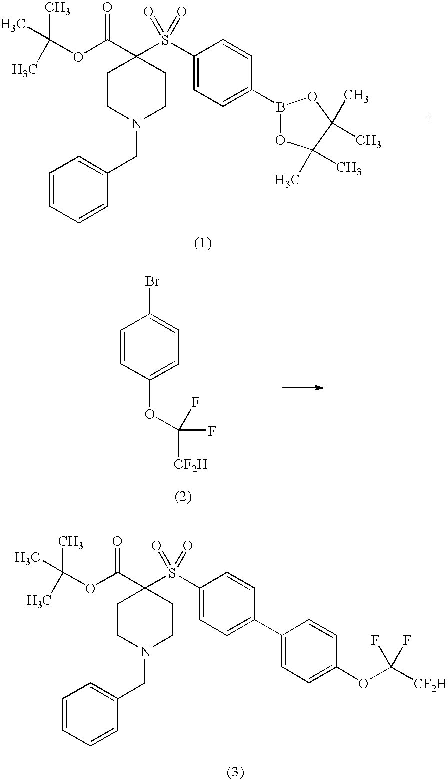

A stirred mixture of 1-benzyl-4-[4-(4,4,5,5-tetramethyl-[1,3,2]dioxaborolan-2-yl)-benzenesulfonyl]-piperidine-4-carboxylic acid tert-butyl ester (1) (6 g, 11 mmol), 1-bromo-4-(1,1,2,2-tetrafluoroethoxy)benzene (2) (3.7 g, 13.3 mmol), potassium carbonate (K₂CO₃, 4.7 g, 33.2 mmol), [1,1′-bis(diphenylphosphino)-ferrocene]dichloropalladium (II).CH₂Cl₂ (0.36 g, 0.44 mmol) in 1,2-dimethoxyethane (“DME”, 150 ml) was refluxed (approximately 90° C.) under N₂ for 5 hr. The resulting dark mixture was diluted with ethyl acetate, washed with brine, and dried over sodium sulfate. The solvent was then removed, and the residue was carefully chromatographed on silica gel (eluting with cyclohexane/ethyl acetate 5/1) to afford 5.1 g (76% yield) of the desired compound (3). ¹H NMR and mass spectrometry (MH⁺=608) were consistent with the desired compound (3).

### Part B. Preparation of 1 tert-butyl 4-{[4′-(1,1,2,2-tetrafluoroethoxy)-1,1′-biphenyl-4-yl]sulfonyl}piperidine-4-carboxylate (4)

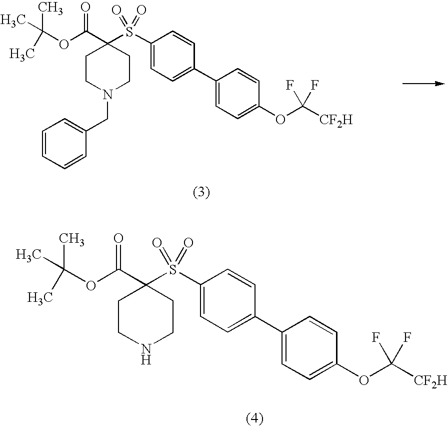

A mixture of the product (3) from Part A (5 g, 8,2 mmol), ammonium formate (1.6 g, HCOONH₄, 24.7 mmol), 10% Pd/C (0.5 g) in ethanol (EtOH) was refluxed under N₂ for 2 hr. After filtering over a pad of Celite, the solvent was removed, and the residue was chromatographed on silica gel (eluting with chloroform) to afford 3.5 g (83% yield) of the desired compound (4) as an oil that crystallized upon standing. ¹H NMR and mass spectrometry (MH⁺=518) were consistent with the desired compound (4)

### Part C. Preparation of tert-butyl 1-ethyl-4-{[4′-(1,1,2,2-tetrafluoroethoxy)-1,1′-biphenyl-4-yl]sulfonyl}piperidine-4-carboxylate (5)

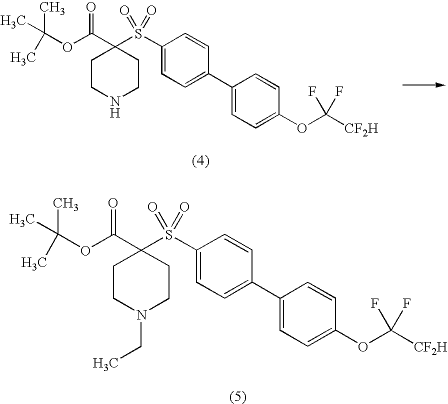

To a stirred mixture of the product (4) from Part B (1 g, 1.9 mmol), N-ethyl-N,N-diisopropylamine (“DIPEA”, 0.75 g, 5.8 mmol) in dimethylformamide (“DMF”, 10 ml) was added ethyl iodide (“EtI”, 0.33 g, 2.1 mmol) at room temperature. After stirring overnight at room temperature, the mixture was diluted with ethyl acetate, washed thoroughly with water, washed with brine, and dried over sodium sulfate. The solvent was then removed, and the residue was chromatographed on a small column of silica gel (eluting with cyclohexane/ethyl acetate 1/1) to afford 0.8 g (77% yield) of the desired compound (5). ¹H NMR and mass spectrometry (MH⁺=546) were consistent with the desired compound (5).

### Part D. Preparation of 1-ethyl-4-[4′-(1,1,2,2-tetrafluoro-ethoxy)-biphenyl-4-sulfonyl]-piperidine-4-carboxylic acid hydrochloride (6)

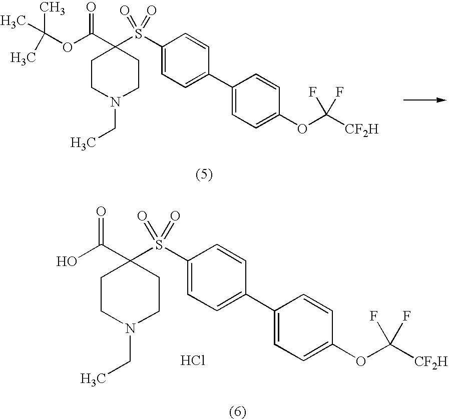

A mixture of the product (5) from Part C (0.8 g, 1.5 mmol) dissolved in 4 N HCl in dioxane (20 ml) was set aside overnight at room temperature. Subsequently, the solvent was removed. Toluene (25 ml) was then added and evaporated to afford 0.7 g (quantitative yield) of the desired compound (6) in the form of white crystals. ¹H NMR and mass spectrometry (MH⁺=490) were consistent with the desired compound (6).

### Part E. Preparation of 1-ethyl-4-{[4′-(1,1,2,2-tetrafluoroethoxy)-1,1′-biphenyl-4-yl]sulfonyl}-N-(tetrahydro-2H-pyran-2-yloxy)piperidine-4-carboxamide (7)

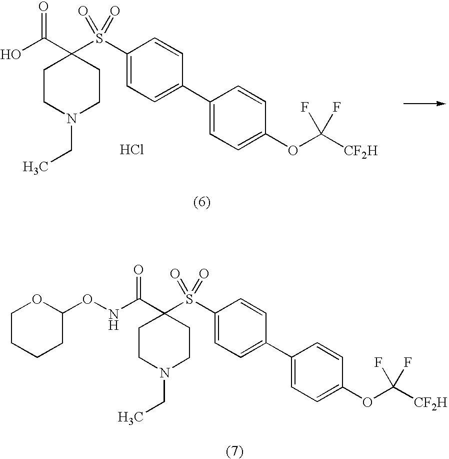

To a stirred mixture of the product (6) from Part D (0.7 g, 1.3 mmol) and triethylamine (“TEA”, 1.3 ml, 9.2 mmol) in DMF (10 ml) was added 2-(1H-benzotriazole-1-yl)-1,1,3,3-tetramethyluronium tetrafluoroborate (“TBTU”, 0.4 g, 1.7 mmol). The resulting suspension was stirred for 1 hr. Subsequently, O-tetrahydro-2H-pyran-2-ylhydroxylamine (“THP-ONH₂”, 1.1 g, 9.3 mmol) was added at room temperature. The resulting mixture was stirred overnight at room temperature. Subsequently, the mixture was taken up in ethyl acetate, washed twice with a saturated solution of sodium bicarbonate, washed with brine, and dried over sodium sulfate. The solvent was then evaporated off, and the residue was chromatographed on a small column of silica gel eluting with cyclohexane/ethyl acetate 4/1 to provide 0.5 g (68% yield) of the desired compound (7) in the form of a oil. ¹H NMR and mass spectrometry (MH⁺=565) were consistent with the desired compound (7).

### Part F. Preparation of 1-ethyl-4-[4′-(1,1,2,2-tetrafluoro-ethoxy)-biphenyl-4-sulfonyl]-piperidine-4-carboxylic acid hydroxyamide hydrochloride (8)

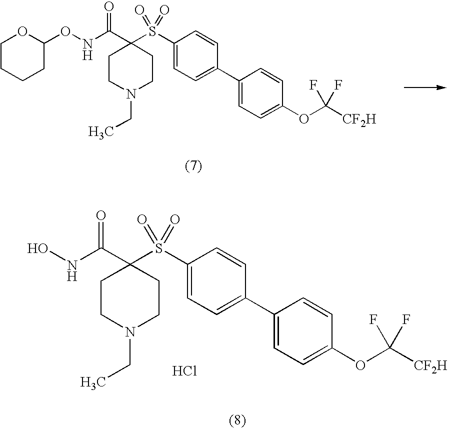

A mixture of the product (7) from Part E (0.6 g, 1.1 mmol) in 4 N HCl in dioxane (15 ml) and methanol (2 ml) was set-aside at room temperature for 2 hr. The solvent was then removed in vacuum, and the residue was crystallized from methanol. The crystals were collected, washed with a small volume of methanol, washed with diethyl ether, and dried in a vacuum at 45° C. for 7 hr to afford 0.4 g (67% yield) of the desired compound (8) in the form of white crystals. ¹H NMR and mass spectrometry (MH⁺=505) were consistent with the desired compound (8).

### Example 5

### Preparation of 1-(2-methoxy-ethyl)-4-[4′-(1,1,2,2-tetrafluoro-ethoxy)-biphenyl-4-sulfonyl]-piperidine-4-carboxylic acid hydroxyamide hydrochloride

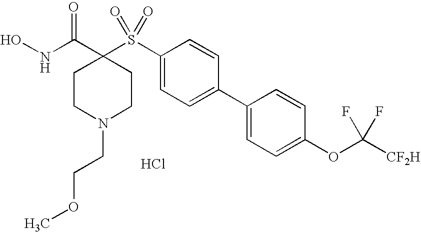

### Part A. Preparation of tert-butyl 1-(2-methoxyethyl)-4-{[4′-(1,1,2,2-tetrafluoroethoxy)-1,1′-biphenyl-4-yl]sulfonyl}piperidine-4-carboxylate (2)

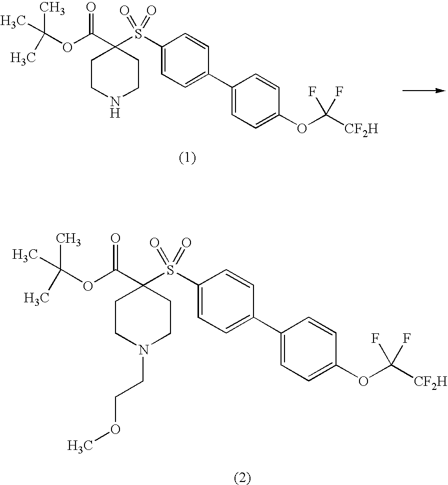

To a stirred mixture of 4-[4′-(1,1,2,2-tetrafluoro-ethoxy)-biphenyl-4-sulfonyl]-piperidine-4-carboxylic acid tert-butyl ester (1) (1 g, 1.9 mmol, prepared in accordance with Part B of Example 4), N-ethyl-N,N-diisopropylamine (“DIPEA”, 0.75 g, 5.8 mmol), and potassium iodide (KI, 0.16 g, 1 mmol) in dimethylformamide (“DMF”, 10 ml) was added 1-bromo-2-methoxyethane (0.3 g, 2.1 mmol). After stirring overnight at room temperature, the resulting mixture was diluted with ethyl acetate, washed thoroughly with water, washed with brine, and dried over sodium sulfate. The solvent was then removed, and the residue was chromatographed on a small column of silica gel (eluting with cyclohexane/ethyl acetate 7/3) to afford 0.7 g (63% yield) of the desired compound (2). ¹H NMR and mass spectrometry (MH⁺=576) were consistent with the desired compound (2).

### Part B. Preparation of 1-(2-methoxyethyl)-4-{[4′-(1,1,2,2-tetrafluoroethoxy)-1,1′-biphenyl-4-yl]sulfonyl}piperidine-4-carboxylic acid hydrochloride (3)

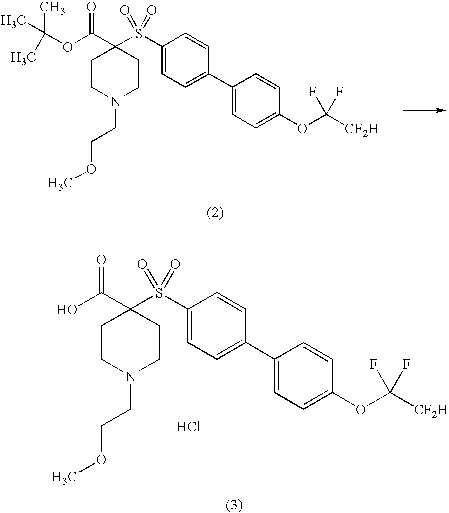

A mixture of the product (2) from Part A (0.7 g, 1.2 mmol) dissolved in 4 N HCl in dioxane (10 ml) was set aside overnight at room temperature. Subsequently, the solvent was removed. Toluene (25 ml) was then added and evaporated to afford 0.6 g (quantitative yield) of the desired compound (3) in the form of a white powder. ¹H NMR and mass spectrometry (MH⁺=520) were consistent with the desired compound (3).

### Part C. Preparation of 1-(2-methoxyethyl)-4-{[4′-(1,1,2,2-tetrafluoroethoxy)-1,1′-biphenyl-4-yl]sulfonyl}-N-(tetrahydro-2H-pyran-2-yloxy)piperidine-4-carboxamide (4)

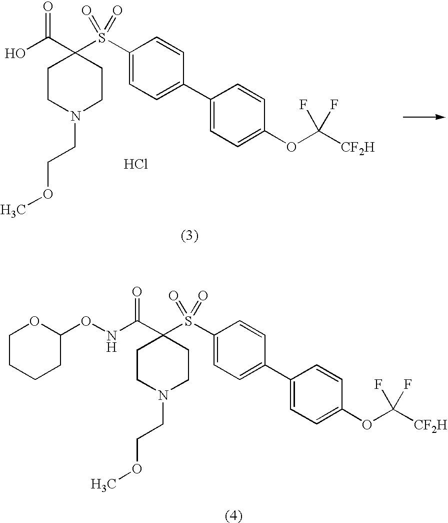

To a stirred mixture of the product (3) from Part B (0.6 g, 1.1 mmol) and triethylamine (“TEA”, 1.1 ml, 7.5 mmol) in dimethylformamide (“DMF”, 10 ml) was added 2-(1H-benzotriazole-1-yl)-1,1,3,3-tetramethyluronium tetrafluoroborate (“TBTU”, 0.32 g, 1.4 mmol). The resulting suspension was stirred at room temperature for 1 hr. Subsequently, O-tetrahydro-2H-pyran-2-ylhydroxylamine (“THP-ONH₂”, 0.6 g, 5.3 mmol) was added. The resulting mixture was stirred overnight at room temperature, and then taken up in ethyl acetate, washed twice with a saturated solution of sodium bicarbonate, washed with brine, and dried over sodium sulfate. The solvent was then evaporated off, and the residue was chromatographed on silica gel (eluting with cyclohexane/ethyl acetate 4/1) to afford 0.5 g (73% yield) of the desired compound (4) in the form of an oil. ¹H NMR and mass spectrometry (MH⁺=619) were consistent with the desired compound (4).

### Part D. Preparation of 1-(2-methoxy-ethyl)-4-[4′-(1,1,2,2-tetrafluoro-ethoxy)-biphenyl-4-sulfonyl]-piperidine-4-carboxylic acid hydroxyamide hydrochloride (5)

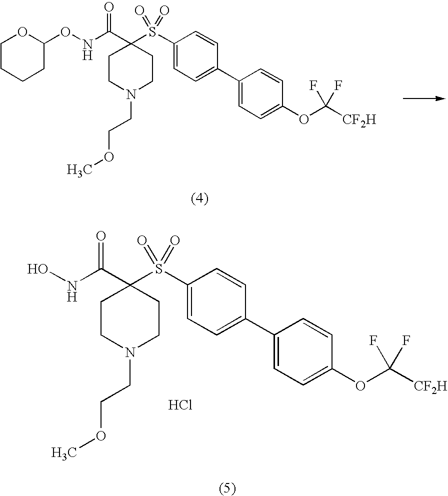

A mixture of the product (4) from Part C (0.5 g, 0.9 mmol) in 4 N HCl in dioxane (15 ml) and methanol (1 ml) was set-aside at room temperature for 2 hr. The solvent was then removed in vacuum, and the residue was crystallized from methanol. The crystals were collected, washed with a small volume of methanol, washed with diethyl ether, dried in vacuum at 45° C. for 5 hr to afford 0.3 g (58% yield) of the desired compound (5) in the form of white crystals. ¹H NMR and mass spectrometry (MH⁺=535) were consistent with the desired compound (5).

### Example 6

### Preparation of 1-cyclopropyl-4-[4′-(1,1,2,2-tetrafluoro-ethoxy)-biphenyl-4-sulfonyl]-piperidine-4-carboxylic acid hydroxyamide hydrochloride

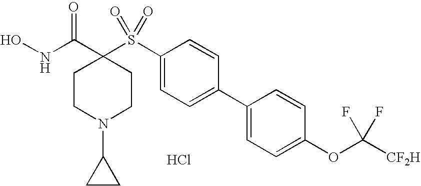

### Part A. Preparation of tert-butyl 1-cyclopropyl-4-{[4═-(1,1,2,2-tetrafluoroethoxy)-1,1′-biphenyl-4-yl]sulfonyl}piperidine-4-carboxylate (2)

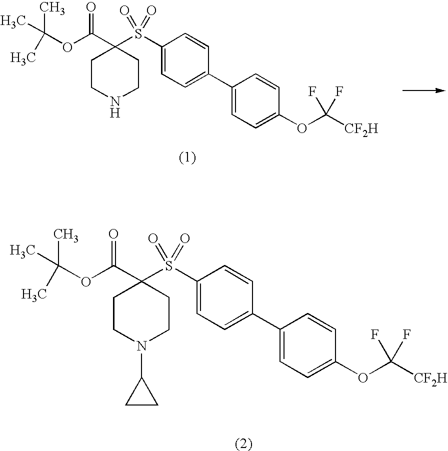

Sodiumcyanoborohydride (NaBH₃CN, 0.6 g, 9.6 mmol) was added portion wise to a stirred mixture of 4-[4′-(1,1,2,2-tetrafluoro-ethoxy)-biphenyl-4-sulfonyl]-piperidine-4-carboxylic acid tert-butyl ester (1) (1 g, 1.9 mmol, prepared in accordance with Part B of Example 4), acetic acid (CH₃COOH, 1.2 ml, 19.3 mmol), [(1-ethoxycyclopropyl)oxy](trimethyl)silane (2.35 ml, 11.6 mmol), and A4 molecular sieves (6 g) in methanol (CH₃OH, 50 ml) under N₂ at room temperature. After 10 min, the mixture was refluxed for 2 hr, and then filtered on a pad of Celite. The solvent was evaporated off, and the residue was dissolved in ethyl acetate, washed with 1 M sodium carbonate solution, washed with brine, and dried over sodium sulfate. The ethyl acetate was then removed, and the residue was filtered on a small column of silica gel (eluting with cyclohexane/ethyl acetate 8/3) to afford 0.7 g (66% yield) of the desired compound (2). ¹H NMR and mass spectrometry (MH⁺=558) were consistent with the desired compound (2).

### Part B. Preparation of 1-cyclopropyl-4-{[4′-(1,1,2,2-tetrafluoroethoxy)-1,1′-biphenyl-4-yl]sulfonyl}piperidine-4-carboxylic acid hydrochloride (3)

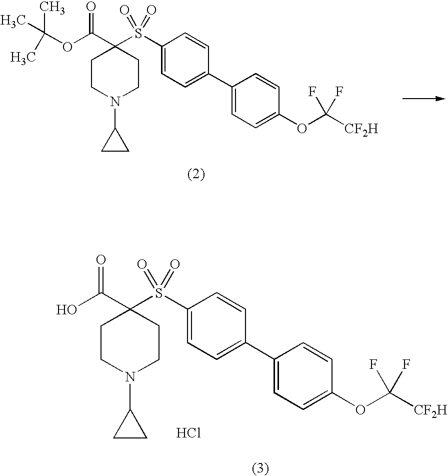

A mixture of the product (2) from Part A (0.6 g, 1,1 mmol) dissolved in 4 N HCl in dioxane (10 ml) was set aside overnight at room temperature. Subsequently, the solvent was removed. Toluene (5 ml) was then added and evaporated to afford 0.5 g (quantitative yield) of the desired compound (3) in the form of a white powder. ¹H NMR and mass spectrometry (MH⁺=502) were consistent with the desired compound (3).

### Part C. Preparation of 1-cyclopropyl-4-{[4′-(1,1,2,2-tetrafluoroethoxy)-1,1′-biphenyl-4-yl]sulfonyl}-N-(tetrahydro-2H-pyran-2-yloxy)piperidine-4-carboxamide (4)

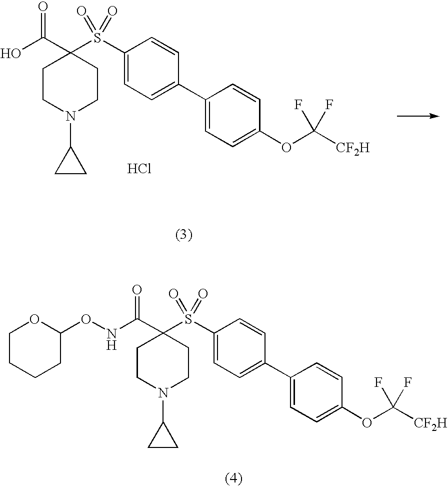

To a stirred mixture of the product (3) from Part B (0.5 g, 1 mmol) and triethylamine (“TEA”, 1 ml, 7 mmol) in dimethylformamide (“DMF”, 20 ml) was added 2-(1H-benzotriazole-1-yl)-1,1,3,3-tetramethyluronium tetrafluoroborate (“TBTU”, 0.3 g, 1.3 mmol). The resulting suspension was stirred at room temperature for 1 hr. Subsequently, O-tetrahydro-2H-pyran-2-ylhydroxylamine (“THP-ONH₂”, 0.8 g, 7 mmol) was added. The resulting mixture was stirred overnight at room temperature, and then taken up in ethyl acetate, washed twice with a saturated solution of NaHCO₃, washed with brine, and dried over sodium sulfate. Afterward, the solvent was evaporated off, and the residue was chromatographed on a small column of silica gel (eluting with cyclohexane/ethyl acetate 7/2) to afford 0.5 g (83% yield) of the desired compound (4) as oil. ¹H NMR and mass spectrometry (MH⁺=601) were consistent with the desired compound (4).

### Part D. Preparation of 1-cyclopropyl-4-[4′-(1,1,2,2-tetrafluoro-ethoxy)-biphenyl-4-sulfonyl]-piperidine-4-carboxylic acid hydroxyamide hydrochloride (5)

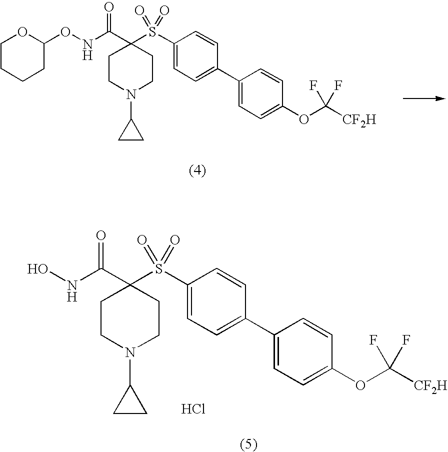

A mixture of the product (4) from Part D (0.4 g, 0.7 mmol) in 4 N HCl in dioxane (10 ml) and methanol (1 ml) was set-aside at room temperature for 2 hr. The solvent was then removed, and the residue was crystallized from methanol. The crystals were filtered off, washed with a small volume of ethanol, washed with diethyl ether, and dried in a vacuum at 45° C. for 10 hr to afford 0.3 g (78% yield) of the desired compound (5) as white crystals. ¹H NMR and mass spectrometry (MH⁺=517) were consistent with the desired compound (5).

### Example 7

### Preparation of N-hydroxy-4-{[4′-(3,3,4,4,4-pentafluorobutyl)-1,1′-biphenyl-4-yl]sulfonyl}tetrahydro-2H-pyran-4-carboxamide

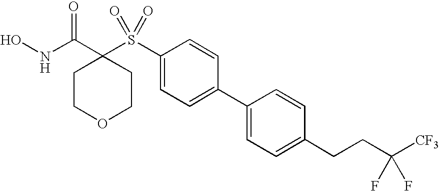

### Part A. Preparation of tert-butyl 4-{[4-(4-bromophenyl)phenyl]sulfonyl}perhydro-2H-pyran-4-carboxylate (3)

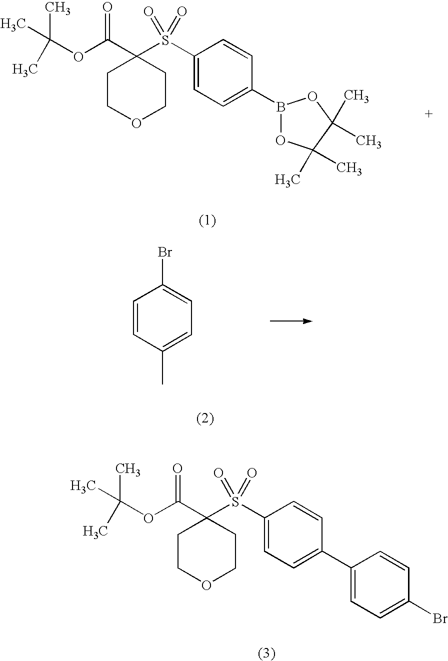

Into a 1L round-bottom flask (equipped with a stir bar, N₂ inlet, and water-cooled condenser) was placed 4-[4-(4,4,5,5-tetramethyl-[1,3,2]dioxaborolan-2-yl)-benzenesulfonyl]-tetrahydro-pyran-4-carboxylic acid tert-butyl ester (1) (56.5 g, 0.125 mol), 1-bromo-4-iodobenzene (2) (39.7 g, 0.14 mol), and [1,1′-bis(diphenylphosphino)ferrocene]dichloropalladium(II) (“Pd(dppf)Cl₂”, 5.1 g, 6.25 mmol). A solution of toluene (224 mL), 1M Na₂CO₃ (224 mL), and 56 mL ethanol (56 mL) was added. The resulting solution was refluxed for 1 hr, after which no starting material (1) was indicated by HPLC. The resulting mixture was cooled to room temperature, and then diluted with ethyl acetate water. The aqueous layer was removed and extracted with additional ethyl acetate (3×500 mL). The organic layers were combined, washed with brine, dried over magnesium sulfate, filtered, and concentrated. The crude product was purified by silica plug filtration (eluting with 1:1 ethyl acetate:hexane), concentrated, and triturated with cold ether affording 42.6 g (71% yield) of the desired compound (3) as a tan solid. Mass spectrometry (MNa⁺=505) was consistent with the desired compound (3).

### Part B. Preparation of tert-butyl 4-({4-[4-(3,3,4,4,4-pentafluorobutyl)phenyl]phenyl}sulfonyl)perhydro-2H-pyran-4-carboxylate (4)

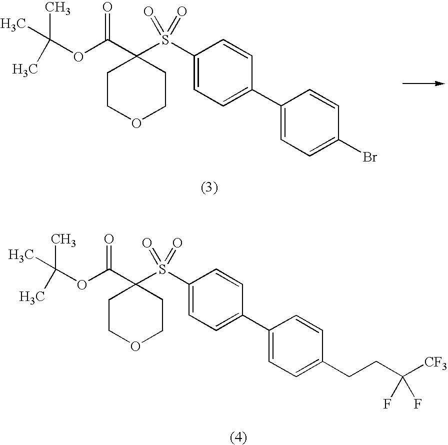

To a slurry of Zn dust (325 mesh, 12.13 mg, 0.1865 mol) and THF (500 mL) in a 500 mL 3-neck round-bottom flask (equipped with a stir bar, reflux condenser, temperature probe, and N₂ inlet) was added 1,4-dibromoethane (3.12 g, 16.6 mmol). The resulting mixture was stirred at 60° C. for 15 min. The mixture was then cooled to room temperature, and chlorotrimethylsilane (1.7 g, 15.7 mmol) was added via syringe. The resulting mixture was stirred 30 min at room temperature. Afterward, 1,1,1,2,2-pentafluoro-4-iodobutane (38 g, 0.14 mol) was added, and the mixture was heated at 45° C. under N₂ for 3 hr. A solution of the product (3) from Part A (40 g, 0.083 mol) in DMA (100 mL) was added, followed by palladium(II)(tri-o-tolylphosphine)dichloride (“pd(tri-O-tolyl)Cl₂”, 4.2 g, 5.4 mmol). The temperature was increased to 80° C., and the reaction was continued for 20 min, after which no starting material (3) was indicated by HPLC. The mixture was cooled to room temperature and diluted with ethyl acetate and saturated ammonium chloride. The layers of the filtrate were separated, and the organic layer was washed with saturated ammonium chloride (2 times), washed with saturated NaCl (1 time), and dried over anhydrous magnesium sulfate. Filtration and evaporation of the solvent under reduced pressure afforded a yellow oil. The crude material was dissolved in methylene chloride, and a mixture of ether/ethyl acetate was added, forming a white precipitate. The mixture was filtered, and the filtrate concentrated to afford the desired compound (4) as a yellow oil, which was carried to Part C without further purification. ¹H NMR and mass spectrometry (MNa⁺=571) were consistent with the desired compound (4).

### Part C. Preparation of 4-({4-[4-(3,3,4,4,4-pentafluorobutyl)phenyl]phenyl}sulfonyl)perhydro-2H-pyran-4-carboxylic acid (5)

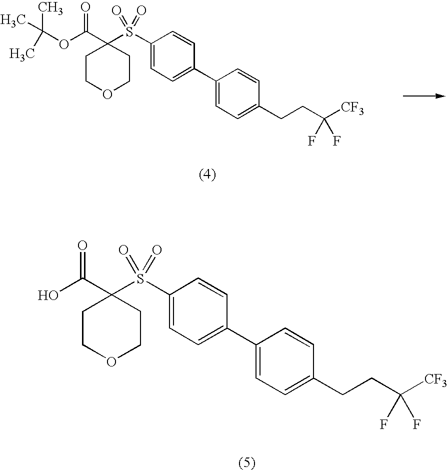

The compound (4) from Part B (0.083 mmol) was dissolved in 1:1 trifluoroacetic acid/dichloromethane (“TFA/CH₂Cl₂”, 150 mL). The reaction was continued overnight at room temperature, after which time no starting material (4) was indicated by HPLC. The mixture was concentrated under reduced pressure. Additional dichloromethane was added, and the solvent was once again removed under reduced pressure. Ether was added, and the product was collected by suction filtration to afford crude desired product (5) as a tan solid. Mass spectrometry (MNa⁺=515) was consistent with the desired product (5).

### Part D. Preparation of [4-({4-[4-(3,3,4,4,4-pentafluorobutyl)phenyl]phenyl}sulfonyl)perhydro-2H-pyran-4-yl]-N-perhydro-2H-pyran-2-yloxycarboxamide (6)

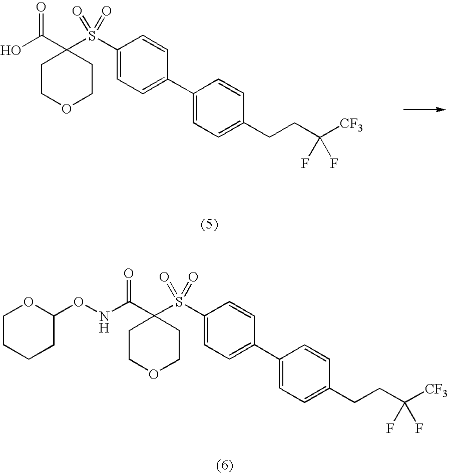

To a mixture of the compound (5) from Part C (37.16 g, 75.5 mmol ) in N,N-dimethylformamide (“DMF”, 300 mL) were added N-hydroxybenzotriazole (“HOBt”, 30.51 g, 0.226 mol), triethylamine (“TEA”, 31.5 mL, 0.226 mol), 1-(3-dimethylaminopropyl)-3-ethylcarbodiimide hydrochloride (“EDC.HCl”, 57.68 g, 0.302 mol), and O-(tetrahydro-2H-pyran-2-yl)hydroxylamine (“THPONH₂”, 30.51 g, 0.226 mol). The reaction was continued overnight at room temperature under N₂, after which no starting material (5) was detected by HPLC. The mixture was then diluted with ethyl acetate. The combined organic layer was extracted with water (3 times), extracted with saturated sodium bicarbonate (3 times), washed with saturated NaCl, and dried over anhydrous magnesium sulfate. Filtration and evaporation of the solvent under reduced pressure afforded 48.76 g of yellow oil. The crude material was purified by flash chromatography using an ethyl acetate gradient (40-100%) in hexane to afford 40 g of product containing impurities. This mixture was dissolved in diethyl ether, and then allowed to sit at room temperature overnight, at which time a white precipitate had formed. The slurry was filtered, and the resulting filter-cake was collected to afford 26.2 g (60% yield) of the desired compound (6) in the form of a white solid. ¹HNMR was consistent with the desired compound (6).

### Part E. Preparation of 4-({4-[4-(3,3,4,4,4-pentafluorobutyl)phenyl]phenyl}sulfonyl)perhydro-2H-pyran-4-carbohydroxamic acid (7)

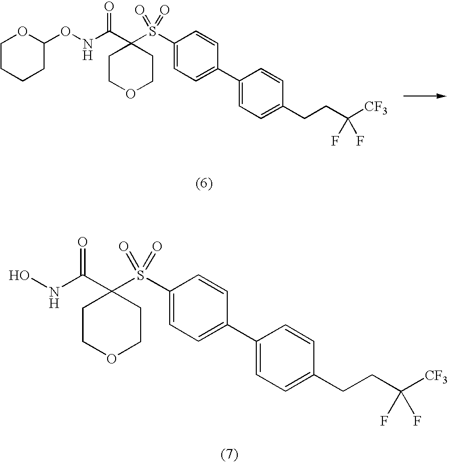

The compound (6) from Part E (26.2 g, 43.0 mmol) was dissolved in 4N HCl in dioxane (161 mL) and methanol (2 mL). The reaction was continued at ambient temperature for 18 hr, after which HPLC indicated that the reaction was complete. The solution was precipitated with diethyl ether/hexane, and the resulting white solid was collected by suction filtration affording 12.35 g (57% yield) of a white solid. ¹H NMR and mass spectrometry (MNa⁺=530) were consistent with the desired product (7). HRMS for C₂₃H₂₂N₂O₅S showed [M+NH₄]_{found}=525.1463 for [M+NH₄]_{calc}=525.1477.

### Example 8

### Preparation of N-hydroxy-4-{[4′-(4,4,4-trifluorobutyl)-1,1′-biphenyl-4-yl]sulfonyl}tetrahydro-2H-pyran-4-carboxamide

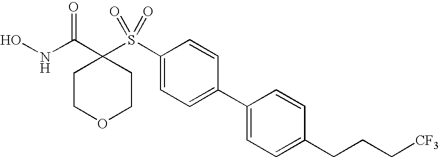

### Part A. Preparation of tert-butyl 4-{[4-(4-bromophenyl)phenyl]sulfonyl}perhydro-2H-pyran-4-carboxylate (3)

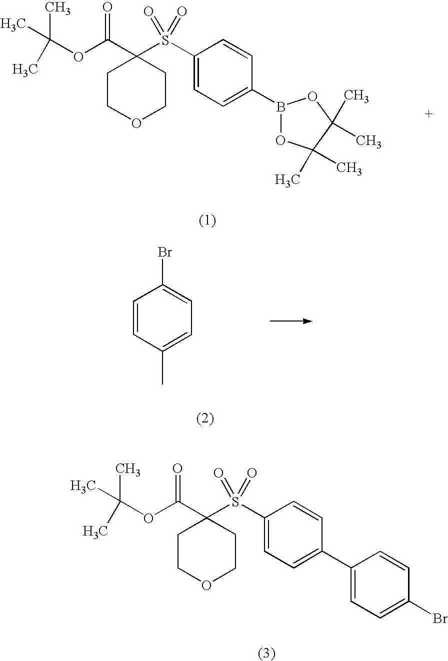

Into a 250 mL round-bottom flask (equipped with a stir bar, N₂ inlet, and water-cooled condenser) was placed 4-[4-(4,4,5,5-tetramethyl-[1,3,2]dioxaborolan-2-yl)-benzenesulfonyl]-tetrahydro-pyran-4-carboxylic acid tert-butyl ester (1) (12 g, 28.3 mmol), 1-bromo-4-iodobenzene (2) (10 g, 35.3 mmol) and [1,1′-bis(diphenylphosphino)ferrocene]dichloropalladium(II) (“Pd(dppf)Cl₂”, 1.15 g, 1.4 mmol). A solution of toluene (48 mL), 1M Na₂CO₃ (48 mL), and ethanol (12 mL) was then added via syringe. The resulting mixture was refluxed for 1 hr, after which no starting material (1) was indicated by HPLC. The mixture was cooled to room temperature and diluted with ethyl acetate water. The aqueous layer was removed and extracted with additional ethyl acetate (2×200 mL). The organic layers were combined, washed with brine, dried over magnesium sulfate, filtered, and concentrated. The crude product was purified by silica plug filtration (eluting with 1:1 ethyl acetate:hexane), concentrated, and triturated with cold ether affording 7.98 g (59% yield) of desired compound (3) as a tan solid. Mass spectrometry (MNa⁺=504) was consistent with the desired compound (3).

### Part B. Preparation of tert-butyl 4-({4-[4-(4,4,4-trifluorobutyl)phenyl]phenyl}sulfonyl)perhydro-2H-pyran-4-carboxylate (4)

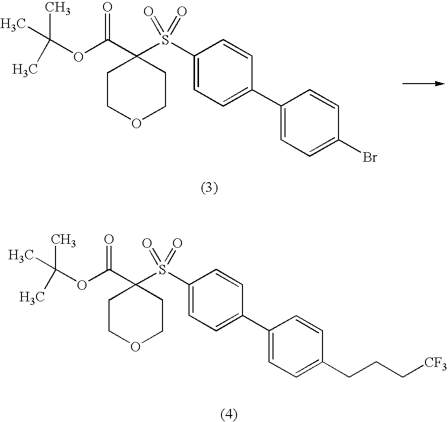

To a slurry of Zn dust (325 mesh, 608 mg, 9.36 mmol) and THF (5 mL) in a 50 mL round-bottom flask (equipped with a stir bar, reflux condenser, temperature probe, and N₂ inlet) was added 1,4-dibromoethane (156 mg, 0.83 mmol). The resulting mixture was stirred at 60° C. for 10 min. The mixture was then cooled to room temperature. Subsequently, chlorotrimethylsilane (100 uL, 0.78 mmol) was added via syringe. The resulting mixture was stirred for 30 min. Afterward, 1,1,1-trifluoro-4-iodobutane (1.67 g, 7.01 mmol) was added. The mixture was then heated at 45° C. under N₂ for 3 hr. A solution of the product (3) from Part A (2.0 g, 4.15 mmol) in THF (5 mL) was added, followed by palladium(II)(tri-o-tolylphosphine)dichloride (“Pd(tri-O-tolyl)Cl₂”, 0.21 g, 0.27 mmol). The temperature was increased to 80° C., and the reaction was continued overnight, after which only a small amount of starting material (3) was detected by HPLC. In a separate flask 1,1,1-trifluoro-4-iodobutane (990 mg, 4.16 mmol) and Rieke zinc (10.4 mL, 8.01 mmol) were combined. After 5 min, the resulting mixture was transferred via syringe to the initial reaction solution and warmed to 80° C. After 10 min, HPLC indicated that no starting material (3) remained. The mixture was cooled to room temperature and diluted with ethyl acetate and saturated ammonium chloride. The layers of the filtrate were separated, and the organic layer was washed with saturated ammonium chloride (2 times), washed with saturated NaCl (1 time), and dried over anhydrous magnesium sulfate. Filtration and evaporation of the solvent under reduced pressure afforded a yellow oil. The crude material was used in Part C without further purification. ¹H NMR and mass spectrometry (MNa⁺=535) were consistent with the desired compound (4).

### Part C. Preparation of 4-({4-[4-(4,4,4-trifluorobutyl)phenyl]phenyl}sulfonyl)perhydro-2H-pyran-4-carboxylic acid (5)

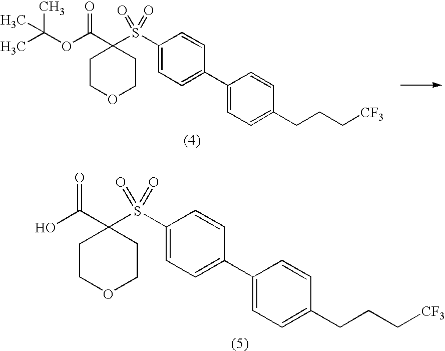

The product (4) from Part B (4.15 mmol) was dissolved in 1:1 trifluoroacetic acid/dichloromethane (“TFA/CH₂Cl₂”, 12 mL). The reaction was continued overnight at room temperature, after which no starting material (4) was detected by HPLC. The mixture was concentrated under reduced pressure. Additional dichloromethane was added, and the solvent was once again removed under reduced pressure. Ether was added, and the product was collected by suction filtration to afford the crude desired product (5) as a tan solid. Mass spectrometry (MNa⁺=479) was consistent with the desired product (5).

### Part D. Preparation of N-perhydro-2H-pyran-2-yloxy[4-({4-[4-(4,4,4-trifluorobutyl)phenyl]phenyl}sulfonyl)perhydro-2H-pyran-4-yl]carboxamide (6)

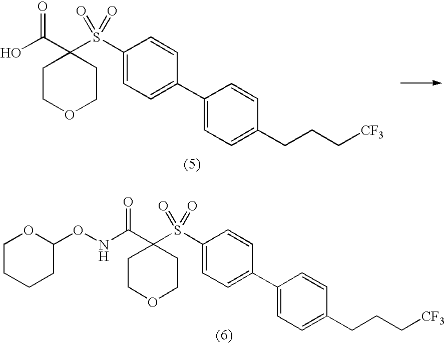

To a mixture of the product (5) of Part C (4.15 mmol ) in N,N-dimethylformamide (“DMF”, 20 mL) were added N-hydroxybenzotriazole (“HOBt”, 1.68 g, 12.45 mmol), triethylamine (“TEA”, 1.73 mL, 12.45 mmol), 1-(3-dimethylaminopropyl)-3-ethylcarbodiimide hydrochloride (“EDC.HCl”, 3.17 g, 16.6 mmol), and O-(tetrahydro-2H-pyran-2-yl)hydroxylamine (“THPONH₂”, 1.45 g, 12.45 mmol). The reaction was continued overnight at room temperature under N₂, after which no starting material (5) was detected by HPLC. The mixture was diluted with ethyl acetate. The combined organic layer was then extracted with water (3 times), extracted with saturated sodium bicarbonate (3 times), washed with saturated NaCl, and dried over anhydrous magnesium sulfate. Filtration and evaporation of the solvent under reduced pressure afforded 1.69 g of yellow oil. The crude material was purified by flash chromatography using an ethyl acetate gradient (10-40%) in hexane to afford 930 mg of the desired product (6) as a white solid. Mass spectrometry (MH⁺=556) was consistent with the desired compound (6).

### Part E. Preparation of 4-({4-[4-(4,4,4-trifluorobutyl)phenyl]phenyl}sulfonyl)perhydro-2H-pyran-4-carbohydroxamic acid (7)

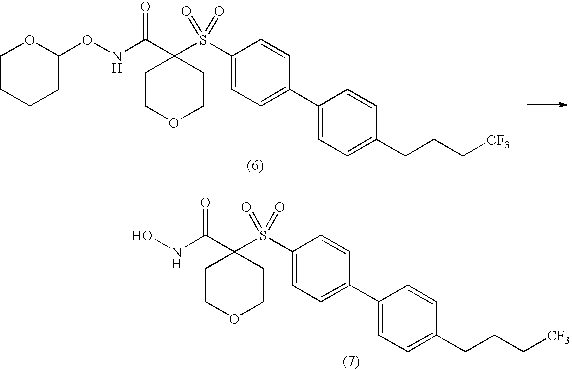

The compound (6) from Part D (0.93 g, 1.67 mmol) was dissolved in 4N HCl in dioxane (4 mL) and methanol (400 uL). The reaction was continued at ambient temperature for 18 hr, after which HPLC indicated that the reaction was complete. The solution was then precipitated with diethyl ether/hexane. The resulting white solid was collected by suction filtration to afford 320 mg of a white solid. The product was dissolved in CH₂Cl₂ and purified by flash chromatography using an acetonitrile gradient (5-10%) in ethyl acetate to afford 110 mg of the desired compound (7) as a white solid. ¹H NMR and mass spectrometry (MH⁺=472) were consistent with the desired product (7). HRMS for C₂₃H₂₂N₂O₅S showed [M−H]_{found}=470.1205 for [M−H]_{calc}=470.1244.

### Example 9

### Preparation of N-hydroxy-1-(2-methoxyethyl)-4-{[4′-(4,4,4-trifluorobutyl)-1,1′-biphenyl-4-yl]sulfonyl}piperidine-4-carboxamide hydrochloride

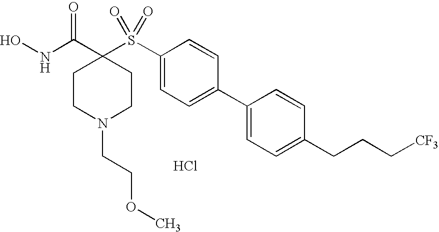

### Part A. Preparation of 4′-bromo-4-methanesulfonyl-biphenyl (3)

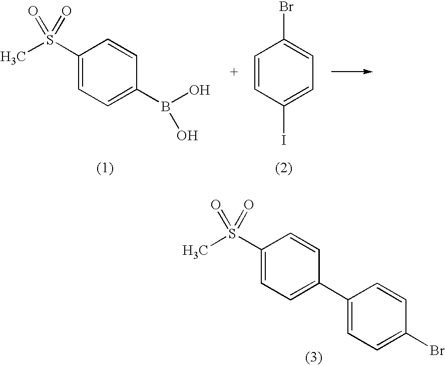

Into a 1 L round bottom flask (equipped with a stir bar, N₂ inlet, and water-cooled condenser) was placed 4-(methanesulfonyl)phenyl boronic acid (1) (10.0 g, 42.5 mmol), 1-bromo-4-iodobenzene (2) (15.0 g, 53.2 mmol), and [1,1′-bis(diphenylphosphino)ferrocene]dichloropalladium(II) (“Pd(dppf)Cl₂”, 1.7 g, 2.1 mmol). A mixture of toluene (40 mL), 2M Na₂CO₃ (40 mL), and ethanol (10 mL) was added. The resulting mixture was refluxed (at approximately 80° C.) for 1 hr, after which no starting material (1) was indicated by HPLC. The resulting mixture was cooled to room temperature and diluted with ethyl acetate. The aqueous layer was removed and extracted with additional ethyl acetate (3×100 mL). The organic layers were combined, washed with brine, dried over magnesium sulfate, filtered, and concentrated. The crude product was purified by silica plug filtration (eluting with 1:9 ethyl acetate:hexane), concentrated, and triturated with cold ether to afford 6 g (46% yield) of the desired product (3) as an off-white solid. Mass spectrometry (MNa⁺=344) was consistent with the desired product (3).

### Part B. Preparation of 4-methanesulfonyl-4′-(4,4,4-trifluoro-butyl)-biphenyl (4)

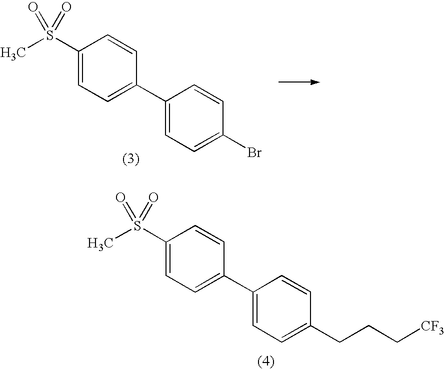

To a slurry of Zn/Cu couple (7.38 g, 0.11 mol) in a mixture of benzene (50 mL) and DMF (5 mL) in a 100 mL 3-neck round-bottom flask (equipped with a stir bar, reflux condenser, temperature probe, and N₂ inlet) was added 1,1,1-trifluorobutyliodide (18.0 g, 0.076 mol). The resulting mixture was stirred at 60° C. for 3 hr. A slurry of the product (3) from Part A (6 g, 0.025 mol) in benzene (10 mL) was added, followed by palladium(II)(tri-o-tolylphosphine)dichloride (“Pd(tri-o-tolylphosphine)Cl₂”, 0.99 g, 1.26 mmol). The temperature was then increased to 80° C., and then maintained at that temperature for 1 hr, after which no starting material (3) was detected by HPLC. The mixture was cooled to room temperature and diluted with ethyl acetate and saturated ammonium chloride. The layers of the filtrate were separated, and the organic layer was washed with saturated ammonium chloride (2 times), washed with saturated NaCl (1 time), and dried over anhydrous magnesium sulfate. Filtration and evaporation of the solvent under reduced pressure afforded a dark solid. The crude material was washed with ether and filtered to afford 5.65 g (65% yield) of desired product (4) in form of an orange solid, which was used in Part C without further purification. ¹H NMR and mass spectrometry (MNa⁺=365) were consistent with the desired compound (4).

### Part C. Preparation of [4′-(4,4,4-trifluoro-butyl)-biphenyl-4-sulfonyl]-acetic acid tert-butyl ester (6)

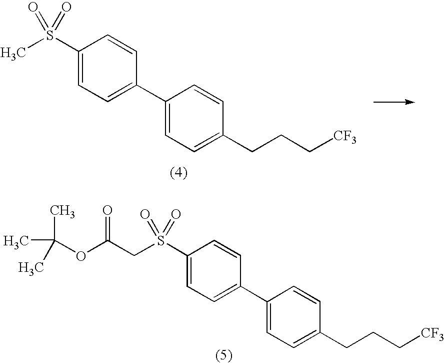

A mixture of the product (4) from Part B (5.6 g, 16.4 mmol) and di-tert-butyl dicarbonate (“(BOC)₂O”, 3.9 g, 18.0 mmol) was cooled to −78° C. in a 300 mL round-bottom flask (equipped with a stir bar, N₂ inlet, and addition funnel). A 1.0 M solution of lithium hexamethyldisilazide (“LiHMDS”, 49.0 mL, 49.2 mmol) was added slowly. The resulting mixture was stirred at −78° C. for 10 min, and then warmed to 0° C. After 5 min, no starting material (4) was detected by HPLC. The mixture was quenched with NH₄Cl and allowed to warm to ambient temperature. The aqueous layer was removed and extracted with ethyl acetate (3×100 mL). The organic layers were combined, washed with brine, dried over magnesium sulfate, filtered, and concentrated. The crude product was triturated with cold ether to afford 4 g (56% yield) of desired compound (5) in the form of an off-white solid. Mass spectrometry (MH⁺=443) was consistent with the desired product (5).

### Part D. Preparation of 1-(2-methoxy-ethyl)-4-[4′-(4,4,4-trifluoro-butyl)-biphenyl-4-sulfonyl]-piperidine-4-carboxylic acid tert-butyl ester (6)

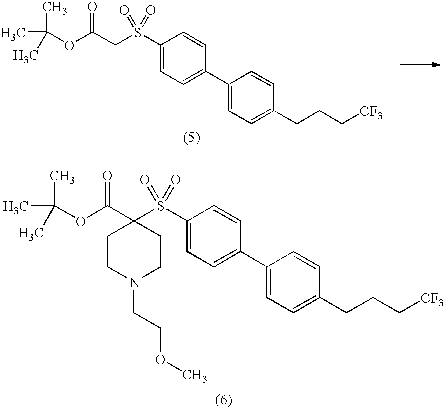

To a stirring mixture of the product (5) from Part C (2.0 g, 4.5 mmol) in DMF (20 mL) was added bis(2-chloroethyl)methoxy amine (1.2 g, 5.0 mmol), 18-crown-6 (“18-C-6”, 0.36 g, 1.35 mmol), and potassium carbonate (K₂CO₃, 3.1 g, 22.5 mmol). The resulting mixture was stirred for 18 hr at 60° C. under N₂. The reaction was then quenched with water (100 mL). The aqueous layer was removed and extracted with ethyl acetate (3×60 mL). The organic layers were combined, washed with brine, dried over magnesium sulfate, filtered, and concentrated to form an oil. The residue was dissolved in dichloromethane and purified on SiO₂ (using 10% acetonitrile/ethyl acetate) to afford 2.1 g of the desired compound (6) in form of a yellow oil (81 % yield). Mass spectrometry (MH⁺=570) was consistent with the desired compound (6).

### Part E. Preparation of trifluoroacetic acid salt of 1-(2-methoxy-ethyl)-4-[4′-(4,4,4-trifluoro-butyl)-biphenyl-4-sulfonyl]-piperidine-4-carboxylic acid (7)

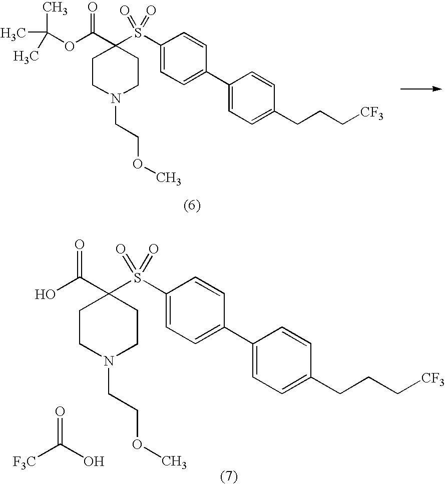

The product (6) from Part D (2.07 g, 3.5 mmol) was dissolved in 1:1 trifluoroacetic acid/dichloromethane (“TFA/CH₂Cl₂”, 30 mL). The reaction was continued overnight at room temperature, after which no starting material (6) was detected by HPLC. The mixture was concentrated under reduced pressure. Subsequently, the residue was stripped from diethyl ether several times under reduced pressure, and then dried under high vacuum. Mass spectrometry (MH⁺=514) was consistent with the desired product (7).

### Part F. Preparation of 1-(2-methoxy-ethyl)-4-[4′-(4,4,4-trifluoro-butyl)-biphenyl-4-sulfonyl]-piperidine-4-carboxylic acid (tetrahydro-pyran-2-yloxy)-amide (8)

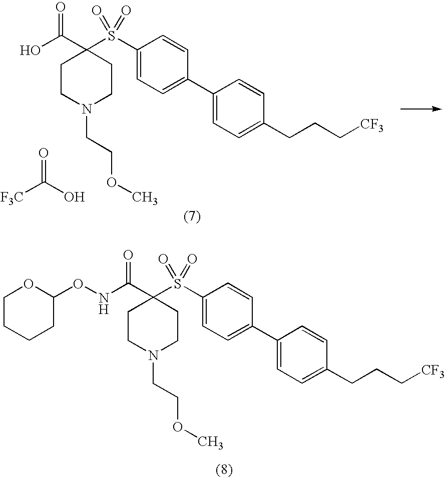

To a mixture of the product (7) from Part E (3.5 mmol) in N,N-dimethylformamide (“DMF”, 20 mL) was added N-hydroxybenzotriazole (“HOBt”, 1.42 g, 10.5 mmol), triethylamine (1.06 g, 1.5 mL, 10.5 mmol), 1-(3-dimethylaminopropyl)-3-ethylcarbodiimide hydrochloride (“EDC.HCl”, 2.64 g, 14.0 mmol), and O-(tetrahydro-2H-pyran-2-yl)hydroxylamine (“THPONH₂”, 1.23 g, 10.5 mmol). The mixture was stirred overnight at room temperature under N₂, after which no starting material (7) was detected by HPLC. The mixture was diluted with water (200 mL). Subsequently, the aqueous layer was removed and extracted with ethyl acetate (3×60 mL). The organic layers were combined, washed with brine, dried over magnesium sulfate, filtered, and concentrated to form an oil. The residue was dissolved in acetonitrile and purified on SiO₂ using 25% acetonitrile/ethyl acetate to afford 1.17 g of the desired compound (8) in the form of a yellow oil (55% yield). ¹H NMR and mass spectrometry (MH⁺=613) were consistent with the desired compound (8).

### Part G. Preparation of N-hydroxy-1-(2-methoxyethyl)-4-{[4′-(4,4,4-trifluorobutyl)-1,1′-biphenyl-4-yl]sulfonyl}piperidine-4-carboxamide hydrochloride (9)

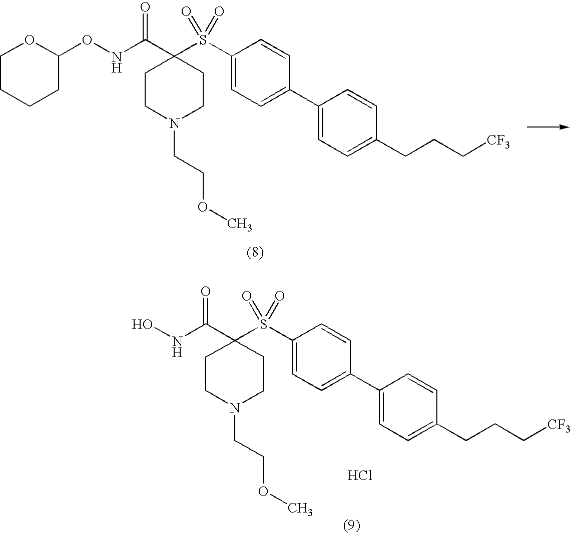

To a mixture of the product (8) from Part F (1.17 g, 2.0 mmol) in methanol (200 mL) was added 4N HCl in dioxane (5 mL). The mixture was stirred at ambient temperature for 2 hr, after which HPLC indicated that the reaction was complete. The mixture was then concentrated under reduced pressure. The resulting residue was triturated with diethyl ether to form an off-white solid, which, in turn, was collected by suction filtration and placed under vacuum. ¹H NMR and high resolution mass spectrometry (theoretical MH⁺=529.1979, actual MH⁺=529.2023) were consistent with the desired compound (9).

### Example 10

### Preparation of 1-cyclopropyl-N-hydroxy-4-{[4′-(4,4,4-trifluorobutyl)-1,1′-biphenyl-4-yl]sulfonyl}piperidine-4-carboxamide hydrochloride

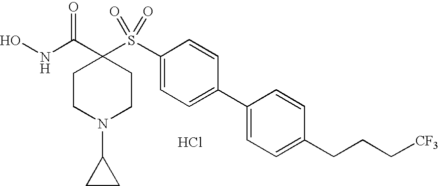

### Part A. Preparation of 4′-bromo-4-methanesulfonyl-biphenyl (3)

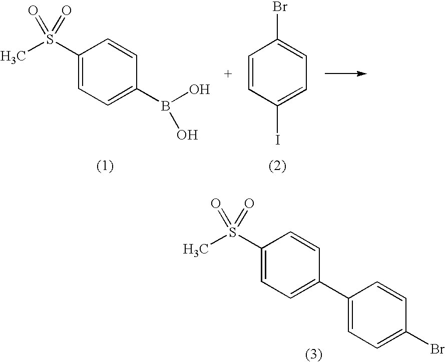

Into a 1 L round-bottom flask (equipped with a stir bar, N₂ inlet, and water-cooled condenser) was placed 4-(methanesulfonyl)phenyl boronic acid (1) (10.0 g, 42.5 mmol), 1-bromo-4-iodobenzene (2) (15.0 g, 53.2 mmol), and [1,1′-bis(diphenylphosphino)ferrocene]dichloropalladium(II) (1.7 g, 2.1 mmol). A solution of toluene (40 mL), 2M Na₂CO₃ (40 mL), and ethanol (10 mL) was then added. The resulting mixture was refluxed for 1 hr, after which no starting material (1) was indicated by HPLC. The mixture was cooled to room temperature and diluted with ethyl acetate water. The aqueous layer was removed and extracted with additional ethyl acetate (3×100 mL). The organic layers were combined, washed with brine, dried over magnesium sulfate, filtered, and concentrated. The crude product was purified by silica plug filtration (eluting with 1:9 ethyl acetate:hexane), concentrated, and triturated with cold ether to afford 6 g (46% yield) of the desired compound (3) as an off-white solid. Mass spectrometry (MNa⁺=344) was consistent with the desired compound (3).

### Part B. Preparation of 4-methanesulfonyl-4′-(4,4,4-trifluoro-butyl)-biphenyl (4)

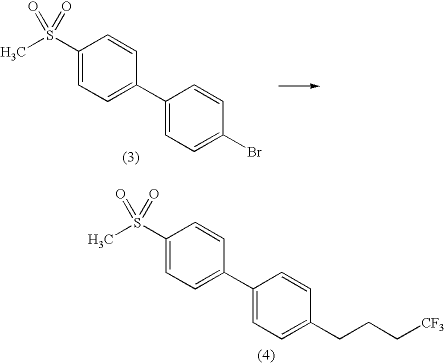

To a slurry of Zn/Cu couple (7.38 g, 0.11 mol) in a mixture of benzene (50 mL) and DMF (5 mL) in a 100 mL 3-neck round-bottom flask (equipped with a stir bar, reflux condenser, temperature probe, and N₂ inlet) was added 1,1,1-trifluorobutyliodide (18.0 g, 0.076 mol). The resulting mixture was stirred at 60° C. for 3 hr. A slurry of the product (3) from Part A (6 g, 0.025 mol) in benzene (10 mL) was added, followed by palladium(II)(tri-o-tolylphosphine)dichloride (0.99 g, 1.26 mmol). The temperature was increased to 80° C., and the reaction was continued for 1 hr, after which no starting material (3) remained by HPLC. The reaction was cooled to room temperature and diluted with ethyl acetate and saturated ammonium chloride. The layers of the filtrate were separated, and the organic layer was washed with saturated ammonium chloride (2 times), washed with saturated NaCl (1 time), and dried over anhydrous magnesium sulfate. Filtration and evaporation of the solvent under reduced pressure afforded a dark solid. The crude material was washed with ether and filtered to afford 5.65 g (65% yield) of the desired compound (4) as an orange solid which was carried on without further purification. ¹H NMR and mass spectrometry (MNa⁺=365) were consistent with the desired compound (4).

### Part C. Preparation of [4′-(4,4,4-trifluoro-butyl)-biphenyl-4-sulfonyl]-acetic acid tert-butyl ester (6)

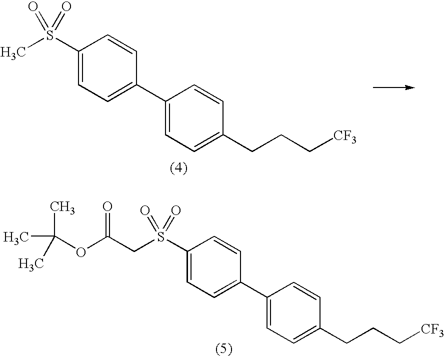

A solution of the product (4) from Part B (5.6 g, 16.4 mmol) and di-tert-butyl dicarbonate (“(BOC)₂O”, 3.9 g, 18.0 mmol) was cooled to −78 ° C. in a 300 mL round-bottom flask equipped with a stir bar, N₂ inlet, and an addition funnel. A 1.0 M solution of lithium hexamethyldisilazide (“LiHMDS”, 49.0 mL, 49.2 mmol) was added slowly. The resulting solution was stirred at −78° C. for 10 min, and then warmed to 0° C. After 5 min, no starting material (4) was indicated by HPLC. The mixture was quenched with NH₄Cl and allowed to warm to ambient temperature. The aqueous layer was removed and extracted with ethyl acetate (3×100 mL). The organic layers were combined, washed with brine, dried over magnesium sulfate, filtered, and concentrated. The crude product was triturated with cold ether to afford 4 g (56% yield) of the desired compound (5) as an off-white solid. Mass spectrometry (MH⁺=443) was consistent with the desired compound (5).

### Part D. Preparation of 1-cyclopropyl-4-[4′-(4,4,4-trifluoro-butyl)-biphenyl-4-sulfonyl]-piperidine-4-carboxylic acid tert-butyl ester (6)

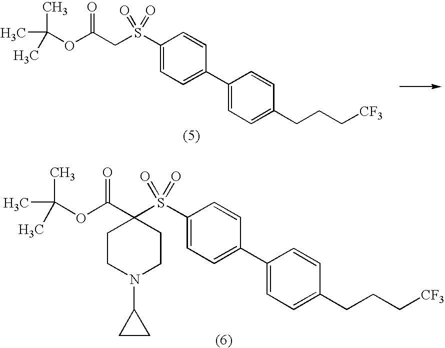

To a stirring solution of the product (5) from Part C (2.0 g, 4.5 mmol) in DMF (20 mL) was added N-cyclopropyl-bis(2-chloroehtyl) amine (1.1 g, 5.0 mmol), 18-crown-6 (0.36 g, 1.35 mmol), and potassium carbonate (3.1 g, 22.5 mmol). The resulting mixture was stirred for 3 days at 60° C. under N₂. The reaction was then quenched with water (100 mL). The aqueous layer was removed and extracted with ethyl acetate (2×100 mL). The organic layers were combined, washed with brine, dried over magnesium sulfate, filtered, and concentrated to an oil. The residue was dissolved in dichloromethane and purified on SiO₂ using 25% ethyl acetate/hexane to afford 1.9 g of the desired compound (6) in the form of a yellow oil (76% yield). Mass spectrometry (MH⁺=552) was consistent with the desired compound (6).

### Part E. Preparation of trifluoroacetic acid salt of 1-cyclopropyl-4-[4′-(4,4,4-trifluoro-butyl)-biphenyl-4-sulfonyl]-piperidine-4-carboxylic acid (7)

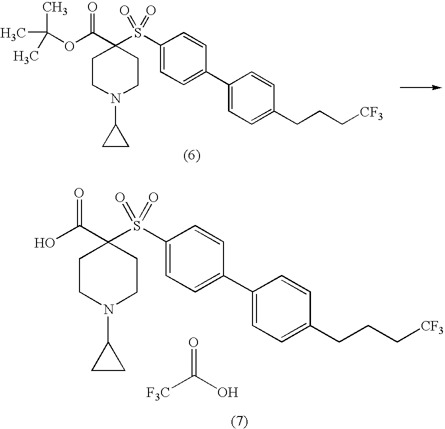

The product (6) from Part D (1.9 g, 3.5 mmol) was dissolved in 1:1 trifluoroacetic acid/dichloromethane (30 mL). The reaction was continued overnight at room temperature, after which no starting material (6) was detected by HPLC. The mixture was concentrated under reduced pressure. The residue was stripped from diethyl ether several times under reduced pressure before drying under high vacuum. Mass spectrometry (MH⁺=496) was consistent with the desired compound (7).

### Part F. Preparation of 1-cyclopropyl-4-[4′-(4,4,4-trifluoro-butyl)-biphenyl-4-sulfonyl]-piperidine-4-carboxylic acid (tetrahydro-pyran-2-yloxy)-amide (8)

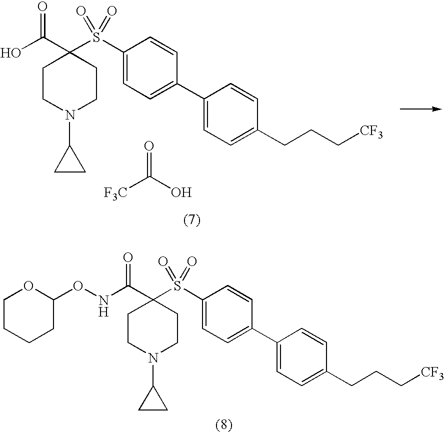

To a mixture of the product (7) from Part E (1.47 g, 2.96 mmol) in N,N-dimethylformamide (20 mL) was added N-hydroxybenzotriazole (1.2 g, 8.91 mmol), triethylamine (0.89 g, 1.2 mL, 8.91 mmol), 1-(3-dimethylaminopropyl)-3-ethylcarbodiimide hydrochloride (2.26 g, 11.8 mmol), and O-(tetrahydro-2H-pyran-2-yl)hydroxylamine (1.2 g, 8.91 mmol). The mixture was stirred overnight at room temperature under N₂, after which no starting material (7) was detected by HPLC. The mixture was diluted with water (200 mL). The aqueous layer was then removed and extracted with ethyl acetate (3×60 mL). The organic layers were combined, washed with brine, dried over magnesium sulfate, filtered, and concentrated to an oil. The residue was dissolved in acetonitrile and purified on SiO₂ using 25% acetonitrile/ethyl acetate to afford 1.5 g of the desired compound (8) in the form of a yellow oil (88% yield). ¹H NMR and mass spectrometry (MH⁺=595) were consistent with the desired compound (8).

### Part G. Preparation of 1-cyclopropyl-N-hydroxy-4-{[4′-(4,4,4-trifluorobutyl)-1,1′-biphenyl-4-yl]sulfonyl}piperidine-4-carboxamide hydrochloride (9)

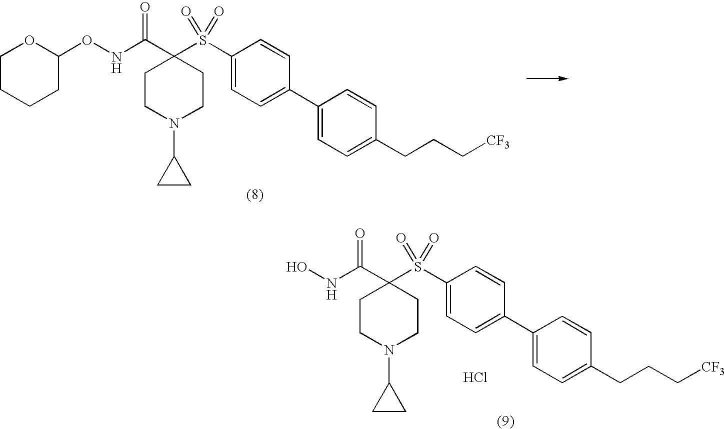

To a mixture of the product (8) from Part F (1.5 g, 2.5 mmol) in methanol (200 mL) was added 4N HCl in dioxane (5 mL). The mixture was stirred at ambient temperature for 2 hr, after which HPLC indicated that the reaction was complete. The mixture was then concentrated under reduced pressure. The resulting residue was triturated with diethyl ether to form an off-white solid, which, in turn, was collected by suction filtration and placed under vacuum to afford 1.23 g of product (9) (90% yield). ¹H NMR and high resolution mass spectrometry (theoretical MH⁺=511.1873, actual MH⁺=511.186) were consistent with the desired compound (9).

### Example 11

### Preparation of 1-cyclopropyl-N-hydroxy-4-{[4′-(3,3,4,4,4-pentafluorobutyl)-1,1′-biphenyl-4-yl]sulfonyl}piperidine-4-carboxamide hydrochloride

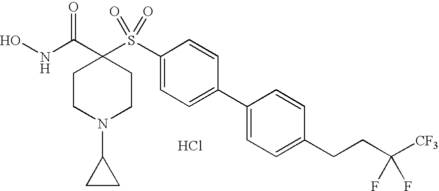

### Part A. Preparation of [4-(4,4,5,5-tetramethyl-[1,3,2]dioxaborolan-2-yl)-benzenesulfonyl]-acetic acid tert-butyl ester (2)

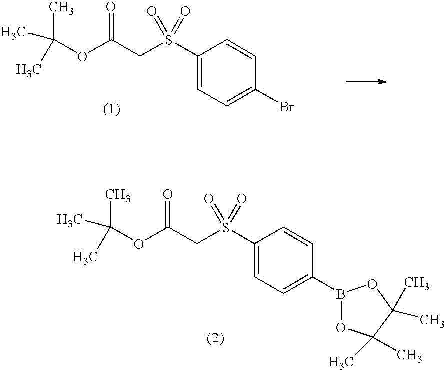

Into a 500 L round-bottom flask (equipped with a stir bar, N₂ inlet, and air-cooled condenser) was placed (4-bromo-benzenesulfonyl)-acetic acid tert-butyl ester (1) (37 g, 0.11 mol), bispinacolediborane (31 g, 0.12 mol), potassium acetate (“KOAc”, 36 g, 0.37 mol), and [1,1′-bis(diphenylphosphino)ferrocene]dichloropalladium(II) (“Pd(dppf)Cl₂”, 3.0 g, 3.6 mmol) in DMF (200 mL). The resulting mixture was heated at 80° C. for 18 hr, after which no starting material (1) was indicated by HPLC. The mixture was cooled to room temperature and partitioned in 1:1 water:ethyl acetate. The aqueous layer was removed and extracted with additional ethyl acetate (3×100 mL). The organic layers were combined, washed with NaHCO₃, washed with brine, dried over magnesium sulfate, filtered, and concentrated. The crude product was used in Part B without further purification. ¹H NMR was consistent with the desired compound (2).

### Part B. Preparation of (4′-bromo-biphenyl-4-sulfonyl)-acetic acid tert-butyl ester (4)

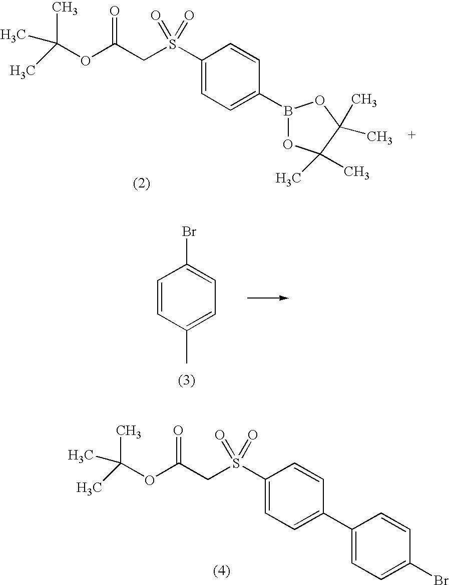

Into a 1L round bottom (equipped with a stir bar, N₂ inlet, and water-cooled condenser) was placed the product (2) from Part A (0.11 mol), 1-bromo-4-iodobenzene (3) (34.2 g, 0.12 mol), and [1,1′-bis(diphenylphosphino)ferrocene]dichloropalladium(II) (“Pd(dppf)Cl₂”, 4.5 g, 5.5 mmol). A mixture of toluene (40 mL), 2M Na₂CO₃ (40 mL), and ethanol (10 mL) was then added. The resulting mixture was refluxed (at approximately 80° C.) for 1 hr, after which no starting material (2) was indicated by HPLC. The resulting mixture was cooled to room temperature and diluted with ethyl acetate. The aqueous layer was removed and extracted with additional ethyl acetate (3×100 mL). The organic layers were combined, washed with brine, dried over magnesium sulfate, filtered, and concentrated. The crude product was triturated with cold ether to afford 30.4 g (57% yield) of the desired compound (4) in the form of a tan solid. ¹H NMR was consistent with the desired compound (4).

### Part C. Preparation of [4′-(3,3,4,4,4-pentafluoro-butyl)-biphenyl-4-sulfonyl]-acetic acid tert-butyl ester (5)

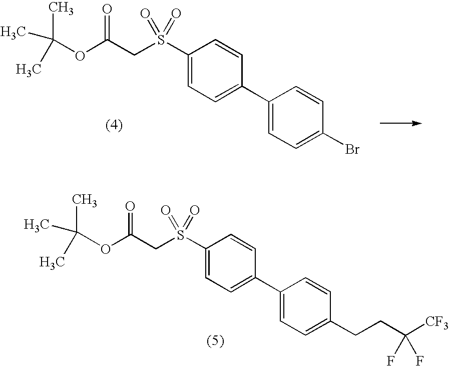

To a slurry of Zn dust (325 mesh, 4.0 mg, 0.062 mmol) and THF (30 mL) in a 250 mL 3-neck round-bottom flask (equipped with a stir bar, reflux condenser, temperature probe, and N₂ inlet) was added 1,4-dibromoethane (1.4 g, 7.0 mmol). The resulting mixture was stirred at 60° C. for 15 min. The mixture was then cooled to 0° C. Afterward, chlorotrimethylsilane (0.93 g, 7.0 mmol) was added via syringe. The resulting mixture was stirred for 30 min at room temperature. Subsequently, 1,1,1,2,2-pentafluoro-4-iodobutane (11.2 g, 0.041 mol) was added slowly. The mixture was then stirred at room temperature under N₂ for 1 hr. Afterward, a mixture of the product (4) from Part B (10 g, 0.021 mol) in DMA (50 mL) was added, followed by palladium(II)(tri-o-tolylphosphine)dichloride (1.0 g, 1.3 mmol). The resulting mixture was heated to 90° C. and stirred 18 hr, after which no starting material (4) was detected by HPLC. The mixture was cooled to room temperature and quenched with saturated ammonium chloride. The aqueous layer was then removed and extracted with additional ethyl acetate (3×100 mL). The organic layers were combined, washed with brine, dried over magnesium sulfate, filtered, and concentrated. The crude material was purified on SiO₂ using 1:1 ethyl acetate:hexane, and concentrated. The desired product (5) was obtained through ether trituration as 5.7 g off-white solid (57% yield). ¹H NMR was consistent with the desired compound (5).

### Part D. Preparation of 1-cyclopropyl-4-[4′-(3,3,4,4,4-pentafluoro-butyl)-biphenyl-4-sulfonyl]-piperidine-4-carboxylic acid tert-butyl ester (6)

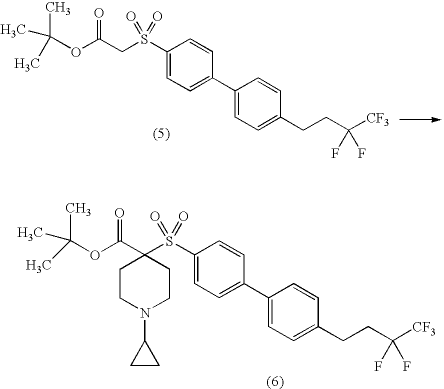

To a stirring mixture of the product (5) from Part C (1.5 g, 3.0 mmol) in DMF (20 mL) was added N-cyclopropyl-bis(2-chloroehtyl)amine (0.75 g, 3.4 mmol), 18-crown-6 (“18-C-6”, 0.24 g, 0.9 mmol), and potassium carbonate (K₂CO₃, 2.07 g, 15.0 mmol). The resulting solution was stirred for 2 days at 80° C. under N₂. Subsequently, the reaction was quenched with water (100 mL). The aqueous layer was removed and extracted with ethyl acetate (2×100 mL). The organic layers were combined, washed with brine, dried over magnesium sulfate, filtered, and concentrated to form an oil. The crude product was used in Part E without further purification. Mass spectrometry (MH⁺=588) was consistent with the desired compound (6).

### Part E. Preparation of the trifluoroacetic acid salt of 1-cyclopropyl-4-[4′-(3,3,4,4,4-pentafluoro-butyl)-biphenyl-4-sulfonyl]-piperidine-4-carboxylic acid (7)

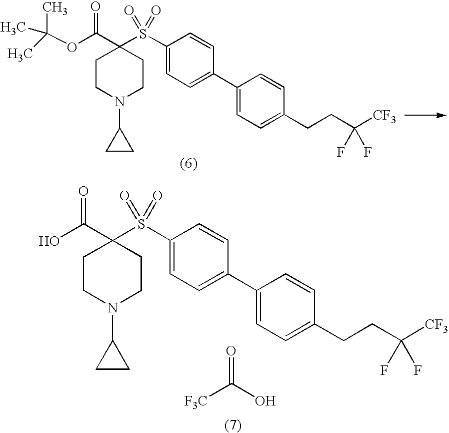

The product (6) from Part D (3.5 mmol) was dissolved in 1:1 trifluoroacetic acid/dichloromethane (“TFA/CH₂Cl₂”, 10 mL). The reaction was continued overnight at room temperature, after which no starting material (6) was detected by HPLC. The mixture was concentrated under reduced pressure. The residue was then stripped from diethyl ether several times under reduced pressure, and then dried under high vacuum. Mass spectrometry (MH⁺=532) was consistent with the desired product (7).

### Part F. Preparation of 1-cyclopropyl-4-[4′-(3,3,4,4,4-pentafluoro-butyl)-biphenyl-4-sulfonyl]-piperidine-4-carboxylic acid (tetrahydro-pyran-2-yloxy)-amide (8)

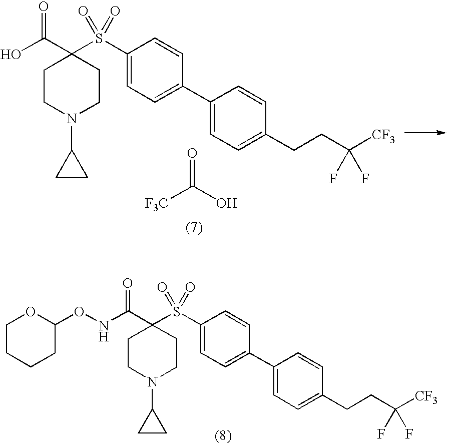

To a mixture of the product (7) from Part E (3.0 mmol) in N,N-dimethylformamide (“DMF”, 10 mL) was added N-hydroxybenzotriazole (“HOBt”, 1.2 g, 9.0 mmol), triethylamine (“TEA”, 0.91 g, 1.2 mL, 9.0 mmol), 1-(3-dimethylaminopropyl)-3-ethylcarbodiimide hydrochloride (“EDC.HCl”, 2.3 g, 12.0 mmol), and O-(tetrahydro-2H-pyran-2-yl)hydroxylamine (“THPONH₂”, 1.0 g, 9.0 mmol). The resulting mixture was stirred overnight at room temperature under N₂, after which no starting material (7) was detected by HPLC. The mixture was diluted with water (200 mL). The aqueous layer was removed and extracted with ethyl acetate (3×60 mL). The organic layers were combined, washed with brine, dried over magnesium sulfate, filtered, and concentrated to form an oil. The residue was dissolved in ethyl acetate and purified on SiO₂ using 50% ethyl acetate/hexane to afford 0.9 g of the desired compound (8) in the form of a yellow oil (50% yield). ¹H NMR was consistent with the desired product (8).

### Part G. Preparation of 1-cyclopropyl-N-hydroxy-4-{[4′-(3,3,4,4,4-pentafluorobutyl)-1,1′-biphenyl-4-yl]sulfonyl}piperidine-4-carboxamide hydrochloride (9)

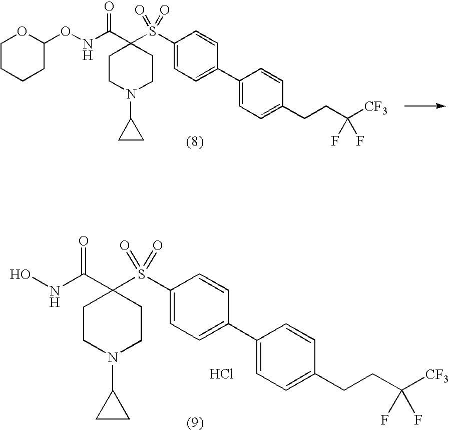

To a mixture of the product (8) from Part F (0.9 g, 1.4 mmol) in ethyl acetate (“EtOAc”, 10 mL) and ethanol (2 mL) was added 4N HCl in dioxane (5 mL). The mixture was then stirred at ambient temperature for 18 hr, after which HPLC indicated that the reaction was complete. The mixture was then concentrated under reduced pressure. The resulting residue was triturated with diethyl ether and hexane to form a white solid, which, in turn, was collected by suction filtration and placed under vacuum to afford 0.58 g of product (9) (72% yield). ¹H NMR and high resolution mass spectrometry (theoretical MH⁺=584.0279, actual MH⁺=584.-311) were consistent with the desired product (9).

### Example 12

### Preparation of N-hydroxy-1-(2-methoxyethyl)-4-{[4′-(3,3,4,4,4-pentafluorobutyl)-1,1′-biphenyl-4-yl]sulfonyl}piperidine-4-carboxamide hydrochloride

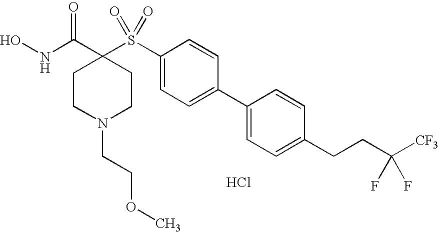

### Part A. Preparation of [4-(4,4,5,5-tetramethyl-[1,3,2]dioxaborolan-2-yl)-benzenesulfonyl]-acetic acid tert-butyl ester (2)

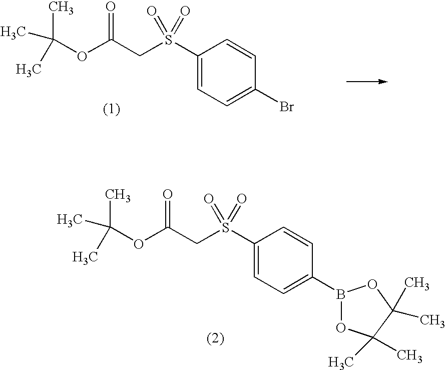

Into a 500 L round-bottom flask (equipped with a stir bar, N₂ inlet, and air-cooled condenser) was placed (4-bromo-benzenesulfonyl)-acetic acid tert-butyl ester (1) (37 g, 0.11 mol), bispinacolediborane (31 g, 0.12 mol), potassium acetate (“KOAc”, 36 g, 0.37 mol), and [1,1′-bis(diphenylphosphino)ferrocene]dichloropalladium(II) (“Pd(dppf)Cl₂”, 3.0 g, 3.6 mmol) in DMF (200 mL). The resulting solution was heated at 80° C. for 18 hr, after which no starting material (1) was indicated by HPLC. The mixture was cooled to room temperature and partitioned in 1:1 water:ethyl acetate. The aqueous layer was then removed and extracted with additional ethyl acetate (3×100 mL). The organic layers were combined, washed with NaHCO₃, brine, dried over magnesium sulfate, filtered, and concentrated. The crude product was used in Part B without further purification. ¹H NMR was consistent with the desired compound (2).

### Part B. Preparation of (4′-bromo-biphenyl-4-sulfonyl)-acetic acid tert-butyl ester (4)

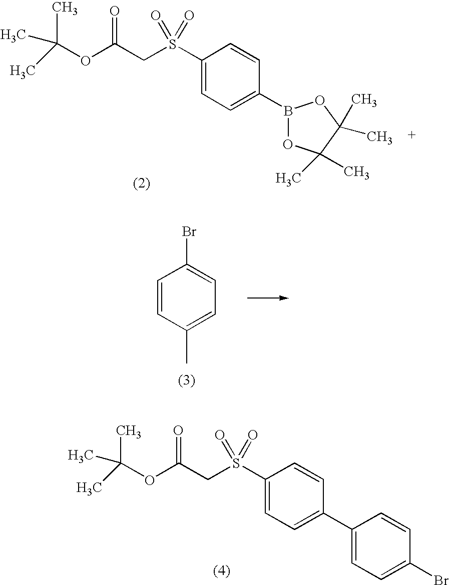

Into a 1 L round-bottom flask (equipped with a stir bar, N₂ inlet, and water-cooled condenser) was placed the product (2) from Part A (0.11 mol), 1-bromo-4-iodobenzene (3) (34.2 g, 0.12 mol), and [1,1′-bis(diphenylphosphino)ferrocene]dichloropalladium(II) ((“Pd(dppf)Cl₂”, 4.5 g, 5.5 mmol). Afterward, a solution of toluene (40 mL), 2M Na₂CO₃ (40 mL), and ethanol (10 mL) was added. The resulting solution was refluxed (at approximately 80° C.) for 1 hr, after which no starting material (2) was indicated by HPLC. The resulting mixture was cooled to room temperature and diluted with ethyl acetate. The aqueous layer was removed and extracted with additional ethyl acetate (3×100 mL). The organic layers were combined, washed with brine, dried over magnesium sulfate, filtered, and concentrated. The crude product was triturated with cold ether affording 30.4 g (57% yield) of the desired compound (4) as a tan solid. ¹H NMR was consistent with the desired compound (4).

Part C. Preparation of [4′-(3,3,4,4,4-pentafluoro-butyl)-biphenyl-4-sulfonyl]-acetic acid tert-butyl ester (5)

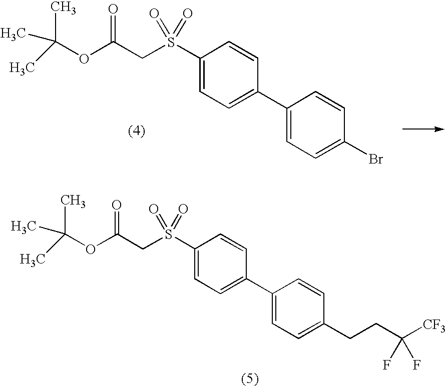

To a slurry of Zn dust (325 mesh, 4.0 mg, 0.062 mmol) and THF (30 mL) in a 250 mL 3-neck round-bottom flask (equipped with a stir bar, reflux condenser, temperature probe, and N₂ inlet) was added 1,4-dibromoethane (1.4 g, 7.0 mmol). The resulting mixture was stirred at 60° C. for 15 min. Afterward, the mixture was cooled to 0° C., and chlorotrimethylsilane (0.93 g, 7.0 mmol) was added via syringe. The resulting mixture was stirred for 30 min. at room temperature. Subsequently, 1,1,1,2,2-pentafluoro-4-iodobutane (11.2 g, 0.041 mol) was added slowly, and the mixture was stirred at room temperature under N₂ for 1 hr. A solution of the product (4) from Part B (10 g, 0.021 mol) in DMA (50 mL) was added, followed by palladium(II)(tri-o-tolylphosphine)dichloride (1.0 g, 1.3 mmol). The mixture was then heated to 90° C. and then stirred 18 hr, after which no starting material (4) was detected by HPLC. The mixture was cooled to room temperature and quenched with saturated ammonium chloride. The aqueous layer was removed and extracted with additional ethyl acetate (3×100 mL). The organic layers were combined, washed with brine, dried over magnesium sulfate, filtered, and concentrated. The crude material was purified on SiO₂ using 1:1 ethyl acetate:hexane and then concentrated. The desired compound (5) was obtained through ether trituration as 5.7 g of an off-white solid (57% yield). ¹H NMR was consistent with the desired compound (5).

### Part D. Preparation of 1-(2-methoxy-ethyl)-4-[4′-(3,3,4,4,4-pentafluoro-butyl)-biphenyl-4-sulfonyl]-piperidine-4-carboxylic acid tert-butyl ester (6)

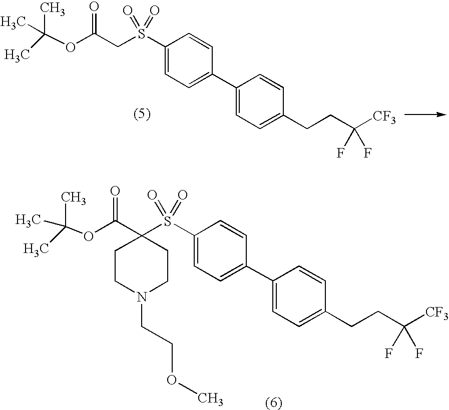

To a stirring solution of the product (5) from Part C (1.3 g, 2.71 mmol) in DMF (20 mL) was added ) was added N-methoxyethyl bis(2-chloroethyl)amine (0.60 g, 3.0 mmol), 18-crown-6 (“18-C-6”, 0.22 g, 1.0 mmol), and potassium carbonate (K₂CO₃, 1.86 g, 13.5 mmol). The resulting mixture was stirred for 18 hr at 80° C. under N₂. The reaction was then quenched with water (100 mL). Afterward, the aqueous layer was removed and extracted with ethyl acetate (2×100 mL). The organic layers were combined, washed with brine, dried over magnesium sulfate, filtered, and concentrated to form an oil. The crude product was used in Part E without further purification. Mass spectrometry (MH⁺=606) was consistent with the desired compound (6).

Part E. Preparation of the trifluoroacetic acid salt of 1-(2-methoxy-ethyl)-4-[4′-(3,3,4,4,4-pentafluoro-butyl)-biphenyl-4-sulfonyl]-piperidine-4-carboxylic acid (7): 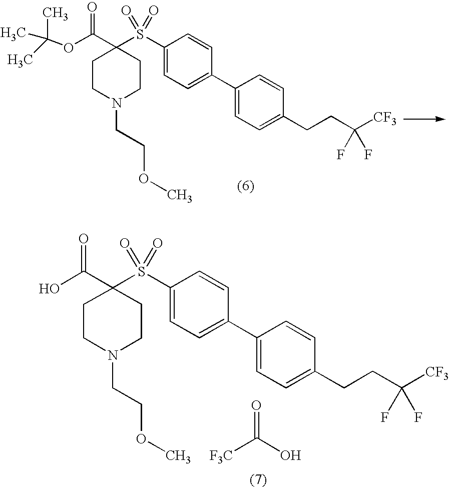
The product (6) from Part D (2.7 mmol) was dissolved in 1:1 trifluoroacetic acid/dichloromethane (“TFA/CH₂Cl₂”, 10 mL). The reaction was continued overnight at room temperature, after which no starting material (6) remained by HPLC. The mixture was concentrated under reduced pressure. The resulting residue was stripped from diethyl ether several times under reduced pressure, and then dried under high vacuum. Mass spectrometry (MH⁺=532) was consistent with the desired product (7).

Part F. Preparation of 1-(2-methoxy-ethyl)-4-[4′-(3,3,4,4,4-pentafluoro-butyl)-biphenyl-4-sulfonyl]-piperidine-4-carboxylic acid (tetrahydro-pyran-2-yloxy)-amide (8): 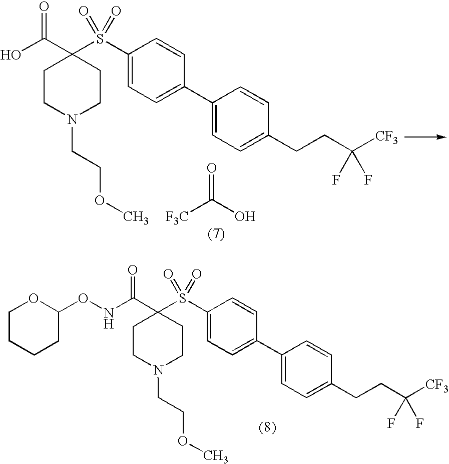
To a mixture of the product (7) from Part E (2.7 mmol) in N,N-dimethylformamide (“DMF”, 10 mL) was added N-hydroxybenzotriazole (“HOBt”, 1.1 g, 8.1 mmol), triethylamine (“TEA”, 1.4 g, 1.8 mL, 13.5 mmol), 1-(3-dimethylaminopropyl)-3-ethylcarbodiimide hydrochloride (“EDC.HCl”, 2.1 g, 10.8 mmol), and O-(tetrahydro-2H-pyran-2-yl)hydroxylamine (“THPONH₂”, 1.0 g, 8.1 mmol). The mixture was stirred overnight at room temperature under N₂, after which no starting material (7) was detected by HPLC. The mixture was diluted with water (200 mL), and then the aqueous layer was removed and extracted with ethyl acetate (3×60 mL). The organic layers were combined, washed with brine, dried over magnesium sulfate, filtered, and concentrated to afford 1.16 g of an orange oil (66% yield). Mass spectrometry (MH⁺=649) was consistent with the desired product (8).

Part G. Preparation of N-hydroxy-1-(2-methoxyethyl)-4-{[4′-(3,3,4,4,4-pentafluorobutyl)-1,1′-biphenyl-4-yl]sulfonyl}piperidine-4-carboxamide hydrochloride: 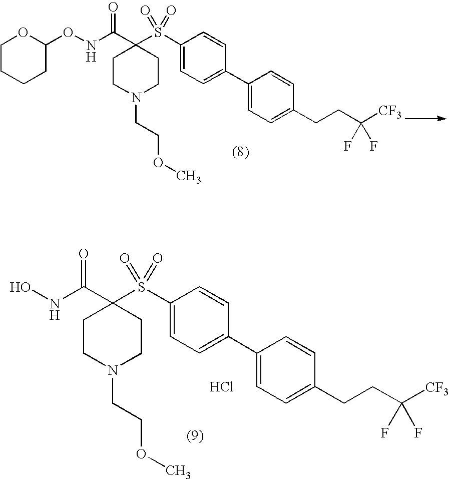
To a mixture of the product (8) from Part G (1.2 g, 1.8 mmol) in ethyl acetate (“EtOAc”, 10 mL) and ethanol (1 mL) was added 4N HCl in dioxane (5 mL). The resulting mixture was stirred at ambient temperature for 18 hr, after which HPLC indicated that the reaction was complete. The mixture was then concentrated under reduced pressure. The resulting residue was triturated with diethyl ether and hexane to form a white solid, which, in turn, was collected by suction filtration and placed under vacuum to afford 0.13 g of product (9) (13% yield). ¹H NMR and high resolution mass spectrometry (theoretical MH⁺=565.2441, actual MH⁺=565.2451) were consistent with the desired product (9).

### Example 13

### Preparation of N-hydroxy-4-methoxy-2-{[4′-(4,4,4-trifluorobutyl)-1,1′-biphenyl-4-yl]sulfonyl}butanamide

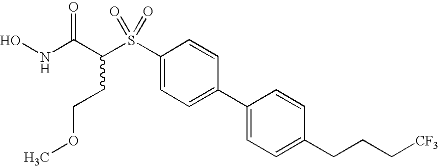

Part A. Preparation of 4-[4-(4,4,5,5-tetramethyl-[1,3,2]dioxaborolan-2-yl)-benzenesulfonyl]-tetrahydro-pyran-4-carboxylic acid tert-butyl ester (2): 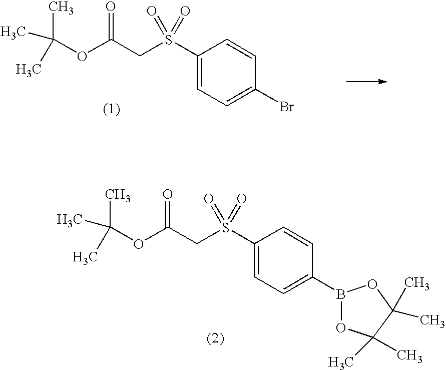
Into a 500 L round-bottom flask (equipped with a stir bar, N₂ inlet, and air-cooled condenser) was placed (4-bromo-benzenesulfonyl)-acetic acid tert-butyl ester (1) ( 37 g, 0.11 mol), bispinacolediborane (31 g, 0.12 mol), potassium acetate (“KOAc”, 36 g, 0.37 mol), and [1,1′-bis(diphenylphosphino)ferrocene]dichloropalladium(II) (“Pd(dppf)Cl₂”, 3.0 g, 3.6 mmol) in DMF (200 mL). The resulting solution was heated at 80° C. for 18 hr, after which no starting material (1) was indicated by HPLC. The mixture was cooled to room temperature and partitioned in 1:1 water:ethyl acetate. The aqueous layer was removed and extracted with additional ethyl acetate (3×100 mL). The organic layers were combined, washed with NaHCO₃, brine, dried over magnesium sulfate, filtered, and concentrated. The crude product was used in Part B without further purification. ¹H NMR was consistent with the desired compound (2).

Part B. Preparation of (4′-bromo-biphenyl-4-sulfonyl)-acetic acid tert-butyl ester (4): 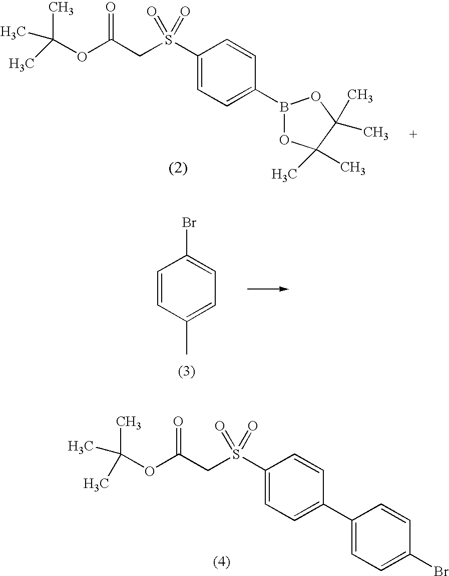
Into a 1 L round-bottom flask (equipped with a stir bar, N₂ inlet, and water-cooled condenser) was placed the product (2) from Part A (0.11 mol), 1 -bromo-4-iodobenzene (3) (34.2 g, 0.12 mol), and [1,1′-bis(diphenylphosphino)ferrocene]dichloropalladium(II) ((“Pd(dppf)Cl₂”, 4.5 g, 5.5 mmol). Subsequently, a mixture of toluene (40 mL), 2M Na₂CO₃ (40 mL), and ethanol (10 mL) was added. The resulting mixture was refluxed (at approximately 80° C.) for 1 hr, after which no starting material (2) was indicated by HPLC. The resulting mixture was cooled to room temperature and diluted with ethyl acetate. The aqueous layer was removed and extracted with additional ethyl acetate (3×100 mL). The organic layers were combined, washed with brine, dried over magnesium sulfate, filtered, and concentrated. The crude product was triturated with cold ether to afford 30.4 g (57% yield) of desired product (4) as a tan solid. ¹H NMR was consistent with the desired compound (4).

Part C. Preparation of [4′-(4,4,4-trifluoro-butyl)-biphenyl-4-sulfonyl]-acetic acid tert-butyl ester (5): 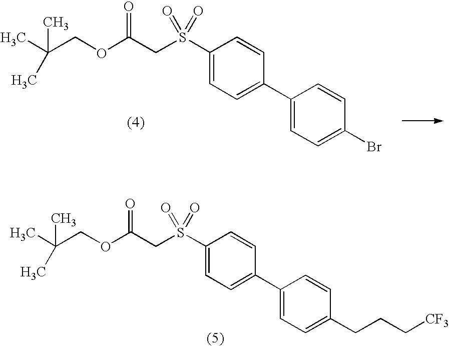
To a slurry of Zn dust (325 mesh, 4.0 mg, 0.062 mmol) and THF (30 mL) in a 250 mL 3-neck round-bottom flask (equipped with a stir bar, reflux condenser, temperature probe, and N₂ inlet) was added 1,4-dibromoethane (1.4 g, 7.0 mmol). The resulting mixture was stirred at 60° C. for 15 min. The mixture was then cooled to 0° C., and chlorotrimethylsilane (0.93 g, 7.0 mmol) was added via syringe. The resulting mixture was stirred for 30 min at room temperature. Subsequently, 1,1,1-trifluoro-4-iodobutane (11.2 g, 0.041 mol) was added slowly, and the mixture was stirred at room temperature under N₂ for 1 hr. A mixture of the product (4) from Part B (10 g, 0.021 mol) in DMA (50 mL) was added, followed by palladium(II)(tri-o-tolylphosphine)dichloride (1.0 g, 1.3 mmol). The resulting mixture was heated to 90° C., and stirred 18 hr, after which no starting material (4) was detected by HPLC. The mixture was cooled to room temperature and quenched with saturated ammonium chloride. The aqueous layer was removed and extracted with additional ethyl acetate (3×100 mL). The organic layers were combined, washed with brine, dried over magnesium sulfate, filtered, and concentrated. The crude material was purified on SiO₂ (using 1:1 ethyl acetate:hexane) and concentrated. The desired product (5) was obtained through ether trituration as 5.7 g of an off-white solid (57% yield). ¹H NMR was consistent with the desired compound (5).

Part D. Preparation of 4-methoxy-2-[4′-(4,4,4-trifluoro-butyl)-biphenyl-4-sulfonyl]-butyric acid tert-butyl ester (6): 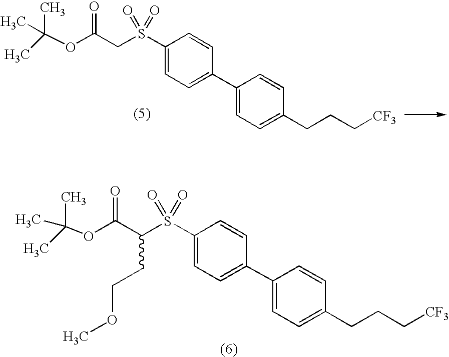
To a stirring mixture of the product (5) from Part C (1.0 g, 2.3 mmol) in DMF (20 mL) was added) 2-bromoethyl methyl ether (0.35 g, 2.5 mmol), 18-crown-6 (“18-C-6”, 0.18 g, 0.68 mmol), and potassium carbonate (K₂CO₃, 1.5 g, 11.3 mmol). The resulting mixture was stirred for 18 hr at 60° C. under N₂. The reaction was then quenched with water (100 mL). The aqueous layer was removed and extracted with ethyl acetate (2×100 mL). The organic layers were combined, washed with brine, dried over magnesium sulfate, filtered, and concentrated to form an oil. The crude product was used in Part E without further purification. ¹H NMR was consistent with the desired compound (6).

Part E. Preparation of 4-methoxy-2-[4′-(4,4,4-trifluoro-butyl)-biphenyl-4-sulfonyl]-butyric acid (7): 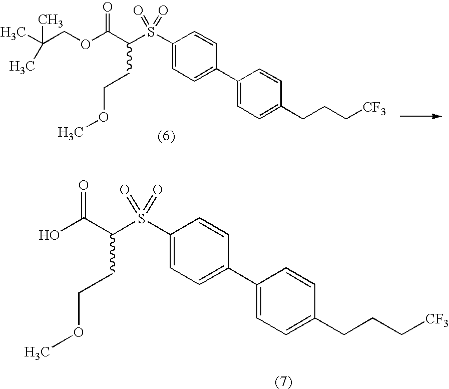
The product (6) from Part D (2.3 mmol) was dissolved in 1:1 trifluoroacetic acid/dichloromethane (“TFA/CH₂Cl₂”, 5 mL). The mixture was then stirred for 2 hr at room temperature, after which no starting material was detected by HPLC. The mixture was concentrated under reduced pressure. The residue was stripped from diethyl ether several times under reduced pressure and dried under high vacuum. The resulting crude product was used in Step F without further purification.

Part F. Preparation of 4-methoxy-N-(tetrahydro-pyran-2-yloxy)-2-[4′-(4,4,4-trifluoro-butyl)-biphenyl-4-sulfonyl]-butyramide (8): 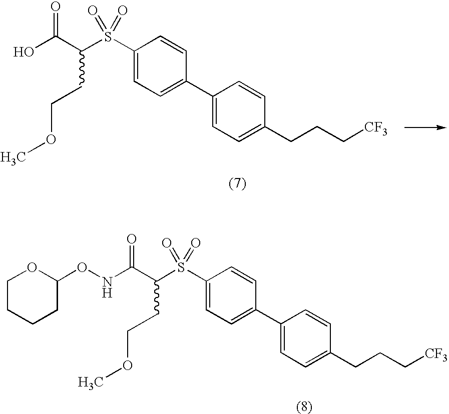
To a mixture of the product (7) from Part E (2.3 mmol) in N,N-dimethylformamide (“DMF”, 20 mL) was added N-hydroxybenzotriazole (“HOBt”, 0.92 g, 6.78 mmol), triethylamine (“TEA”, 1.14 g, 1.6 mL, 11.3 mmol), 1-(3-dimethylaminopropyl)-3-ethylcarbodiimide hydrochloride (“EDC.HCl”, 1.3 g, 6.78 mmol), and O-(tetrahydro-2H-pyran-2-yl)hydroxylamine (“THPONH₂”, 0.61 g, 5.2 mmol). The mixture was stirred overnight at room temperature under N₂, after which no starting material was detected by HPLC. The mixture was diluted with water (200 mL). The aqueous layer was removed and extracted with ethyl acetate (3×60 mL). The organic layers were combined, washed with brine, dried over magnesium sulfate, filtered, and concentrated to form a dark oil. The residue was dissolved in dichloromethane and purified on SiO₂ using 50-70% ethyl acetate/hexane to afford 530 mg of a yellow oil (44% yield).

Part G. Preparation of N-hydroxy-4-methoxy-2-{[4′-(4,4,4-trifluorobutyl)-1,1′-biphenyl-4-yl]sulfonyl}butanamide: 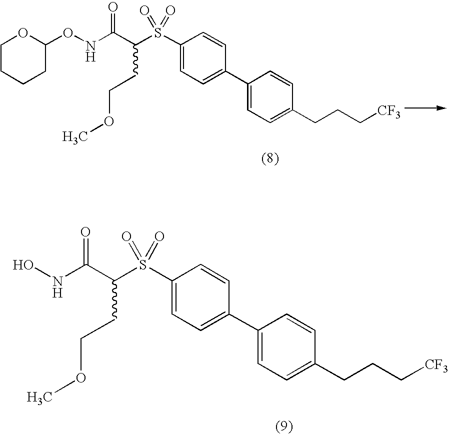
To a mixture of the product (8) from Part F (0.5 mg, 0.9 mmol) in ethyl acetate (“EtOAc”, 5 mL) and ethanol (0.2 mL) was added 4N HCl in dioxane (1 mL). The mixture was stirred at ambient temperature for 18 hr, after which HPLC indicated that the reaction was complete. The mixture was concentrated under reduced pressure. The residue was triturated with diethyl ether and hexane to form a white solid, which, in turn, was collected by suction filtration and placed under vacuum to afford 0.25 g of product (9) (56% yield). ¹H NMR and high resolution mass spectrometry (theoretical MH⁺=459.8772, actual MH⁺=459.8783) were consistent with the desired product (9).

### Example 14

### Preparation of 1-tert-butyl-N-hydroxy-4-{[4′-(4,4,4-trifluorobutyl)-1,1′-biphenyl-4-yl]sulfonyl}piperidine-4-carboxamide hydrochloride

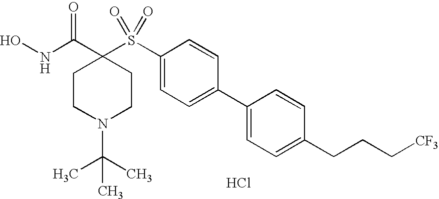

Part A. Preparation of [4-(4,4,5,5-tetramethyl-[1,3,2]dioxaborolan-2-yl)-benzenesulfonyl]-acetic acid tert-butyl ester (2): 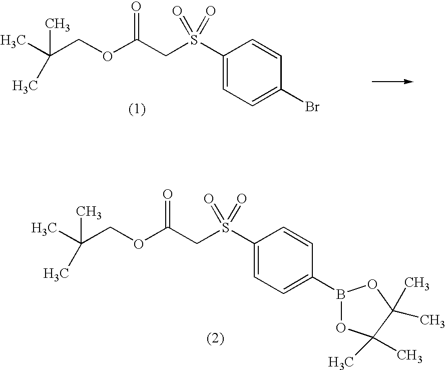
Into a 500 L round-bottom flask (equipped with a stir bar, N₂ inlet, and air-cooled condenser) was placed (4-bromo-benzenesulfonyl)-acetic acid tert-butyl ester (1) (37 g, 0.11 mol), bispinacolediborane (31 g, 0.12 mol), potassium acetate (“KOAc”, 36 g, 0.37 mol), and [1,1′-bis(diphenylphosphino)ferrocene]dichloropalladium(II) (“Pd(dppf)Cl₂”, 3.0 g, 3.6 mmol) in DMF (200 mL). The resulting mixture was heated at 80° C. for 18 hr, after which no starting material (1) was indicated by HPLC. The mixture was cooled to room temperature and partitioned in 1:1 water:ethyl acetate. The aqueous layer was removed and extracted with additional ethyl acetate (3×100 mL). The organic layers were combined, washed with NaHCO₃, brine, dried over magnesium sulfate, filtered, and concentrated. The crude product was used in Part B without further purification. ¹H NMR was consistent with the desired compound (2).

Part B. Preparation of (4′-bromo-biphenyl-4-sulfonyl)-acetic acid tert-butyl ester (4): 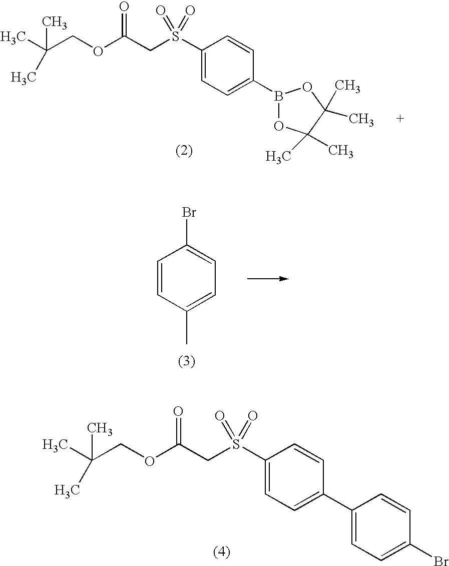
Into a 1 L round-bottom flask (equipped with a stir bar, N₂ inlet, and water-cooled condenser) was placed the product (2) from Part A (0.11 mol), 1-bromo-4-iodobenzene (3) (34.2 g, 0.12 mol), and [1,1′-bis(diphenylphosphino)ferrocene]dichloropalladium(II) (4.5 g, 5.5 mmol). A mixture of toluene (40 mL), 2M Na₂CO₃ (40 mL) and ethanol (10 mL) was added. The resulting mixture was refluxed (at approximately 80° C.) for 1 hr, after which no starting material was indicated by HPLC. The resulting mixture was cooled to room temperature and diluted with ethyl acetate. The aqueous layer was removed and extracted with additional ethyl acetate (3×100 mL). The organic layers were combined, washed with brine, dried over magnesium sulfate, filtered, and concentrated. The crude product was triturated with cold ether to afford 30.4 g (57% yield) of the desired compound (4) as a tan solid. ¹H NMR was consistent with the desired compound (4).

Part C. Preparation of [4′-(4,4,4-trifluoro-butyl)-biphenyl-4-sulfonyl]-acetic acid tert-butyl ester (5): 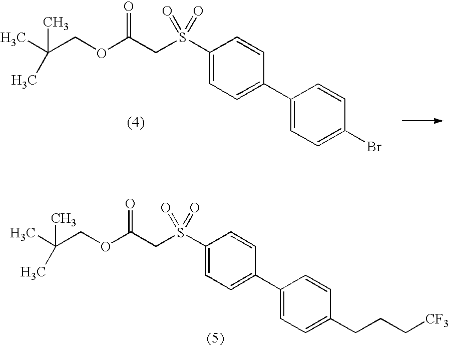
To a slurry of Zn dust (325 mesh, 4.0 mg, 0.062 mmol) and THF (30 mL) in a 250 mL 3-neck round-bottom flask (equipped with a stir bar, reflux condenser, temperature probe, and N₂ inlet) was added 1,4-dibromoethane (1.4 g, 7.0 mmol). The resulting mixture was stirred at 60° C. for 15 min. The mixture was then cooled to 0° C., and chlorotrimethylsilane (0.93 g, 7.0 mmol) was added via syringe. The resulting mixture was stirred for 30 min at room temperature. Subsequently, 1,1,1-trifluoro-4-iodobutane (11.2 g, 0.041 mol) was added slowly, and the mixture was stirred at room temperature under N₂ for 1 hr. A mixture of the product (4) from Part B (10 g, 0.021 mol) in DMA (50 mL) was added, followed by palladium(II)(tri-o-tolylphosphine)dichloride (1.0 g, 1.3 mmol). The resulting mixture was heated to 90° C., and stirred for 18 hr, after which no starting material (4) was detected by HPLC. The mixture was cooled to room temperature and quenched with saturated ammonium chloride. The aqueous layer was removed and extracted with additional ethyl acetate (3×100 mL). The organic layers were combined, washed with brine, dried over magnesium sulfate, filtered, and concentrated. The crude material was purified on SiO₂ using 1:1 ethyl acetate:hexane and concentrated. The desired compound (5) was obtained through ether trituration as 5.7 g of an off-white solid (57% yield). ¹H NMR was consistent with the desired compound (5).

Part D. Preparation of 1-tert-butyl-4-[4′-(4,4,4-trifluoro-butyl)-biphenyl-4-sulfonyl]-piperidine-4-carboxylic acid tert-butyl ester (6): 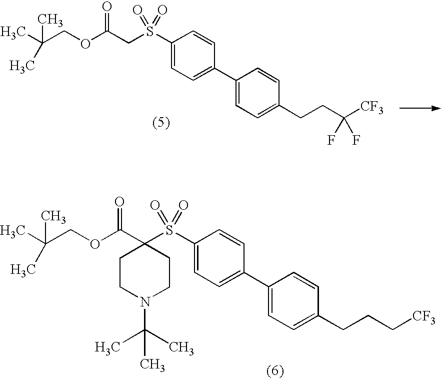
To a stirring mixture of the product (5) from Part C (2.0 g, 4.5 mmol) in DMF (20 mL) was added bis(2-chloroethyl)amine (1.0 g, 5.0 mmol), 18-crown-6 (“18-C-6”, 0.36 g, 1.4 mmol), and potassium carbonate (K₂CO₃, 3.1 g, 22.5 mmol). The resulting mixture was stirred for 18 hr at 80° C. under N₂. The reaction was then quenched with water (100 mL). The aqueous layer was removed and extracted with ethyl acetate (2×100 mL). The organic layers were combined, washed with brine, dried over magnesium sulfate, filtered, and concentrated to form an oil. The crude product used in Part E without further purification. Mass spectrometry (MNa⁺=585) was consistent with the desired product (6).

Part E. Preparation of the trifluoroacetic acid salt of 1-tert-butyl-4-[4′-(4,4,4-trifluoro-butyl)-biphenyl-4-sulfonyl]-piperidine-4-carboxylic acid (7): 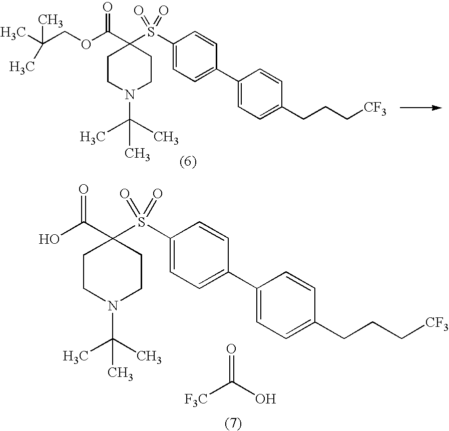
The product (6) from Part D (2.7 mmol) was dissolved in 1:1 trifluoroacetic acid/dichloromethane (“TFA/CH₂Cl₂”, 10 mL). The reaction was continued overnight at room temperature, after which no starting material (6) remained by HPLC. The mixture was concentrated under reduced pressure. The residue was stripped from diethyl ether several times under reduced pressure and then dried under high vacuum. The product was then used in Step F without further purification.

Part F. Preparation of 1-tert-butyl-4-[4′-(4,4,4-trifluoro-butyl)-biphenyl-4-sulfonyl]-piperidine-4-carboxylic acid (tetrahydro-pyran-2-yloxy)-amide (8): 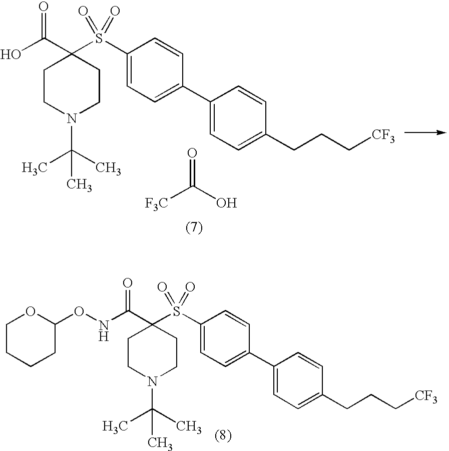
To a mixture of the product (7) from Part E (2.7 mmol) in N,N-dimethylformamide (“DMF”, 20 mL) was added 2-(1H-benzotriazole-1-yl)-1,1,3,3-tetramethyluronium tetrafluoroborate (“TBTU”, 1.6 g, 5.0 mmol), diisopropylethylamine (2.9 g, 4.0 mL, 22.5 mmol), and O-(tetrahydro-2H-pyran-2-yl)hydroxylamine (“THPONH₂”, 0.53 g, 4.5 mmol). The mixture was stirred overnight at room temperature under N₂, after which time no starting material (7) was detected by HPLC. The mixture was diluted with water (200 mL). The aqueous layer was removed-and extracted with ethyl acetate (3×60 mL). The organic layers were combined, washed with brine, dried over magnesium sulfate, filtered, and concentrated to form a dark oil. This oil was used in Step G without further purification.

Part G. Preparation of 1-tert-butyl-N-hydroxy-4-{[4′-(4,4,4-trifluorobutyl)-1,1′-biphenyl-4-yl]sulfonyl}piperidine-4-carboxamide hydrochloride: 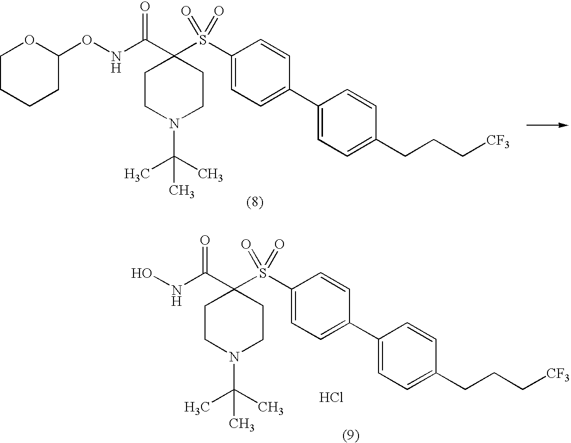
To a mixture of the product (8) from Part F (4.5 mmol) in ethyl acetate (10 mL) and ethanol (1 mL) was added 4N HCl in dioxane (5 mL). The resulting mixture was stirred at ambient temperature for 18 hr, after which HPLC indicated that the reaction was complete. The mixture was concentrated under reduced pressure. The resulting residue was triturated with diethyl ether and hexane to form a white solid, which, in turn, was collected by suction filtration and placed under vacuum to afford 0.78 g of product (9) (32% yield). ¹H NMR and high resolution mass spectrometry (theoretical MH⁺=527.1403, actual MH⁺=527.1378) were consistent with the desired product (9).

### Example 15

### Preparation of 4-({4-[5-(2-cyclopropylethyl)pyrazin-2-yl]phenyl}sulfonyl)-N-hydroxytetrahydro-2H-pyran-4-carboxamide hydrochloride

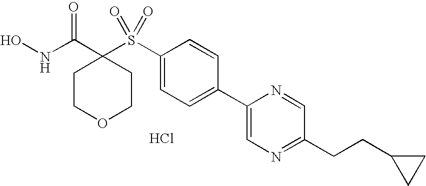

Part A. 2-Cyclopropylethanol, (21.35 g, 248 mmol, Lancaster), imidazole (25.32 g, 372.4 mmol, Aldrich), and triphenylphosphine (84.64 g, 323 mmol, Aldrich) were dissolved into methylene chloride (300 mL). The resulting mixture was cooled to 0° C. in an ice bath. Afterward, iodine (75.37 g, 298 mmol, Aldrich) was added portion-wise such that the temperature remained at less than 30° C. After this addition was complete, the mixture was allowed to warm to ambient temperature and mix under N₂ overnight. The mixture was then diluted with deionized water (250 mL). Subsequently, the layers were separated. The methylene chloride layer was washed with 200 mL each of 10% HCl(aq) (200 mL), saturated NaHCO3(aq) (200 mL), and 10 g Na₂S₂O₃ in deionized water (200 mL). The methylene chloride layer was dried over MgSO₄, filtered, and concentrated in vacuo with a rotovap having a bath temperature of less than 25° C. to form solids. Hexanes (150 mL) were added to the solids, and the mixture was slurried for approximately 1 hr. The solids were then filtered and washed with hexanes (150 mL). The filtrate was passed through a pad of silica (pre-washed with hexanes), with the silica being washed with hexanes to elute the product through the silica. Five bulk fractions of 350 mL each were taken. Product was detected in the first 3 fractions, and had little triphenylphosphine contamination. Those fractions were combined and concentrated in vacuo with a rototrap having a bath temperature of less than 25° C. to form 30.08 g of an oil (62% yield). ¹H NMR was consistent with the desired cyclopropyl ethyl iodide intermediate product.

Part B. Zinc dust (325 mesh, 19.9 g, 306 mmol, Aldrich) and THF (65 mL) were combined and stirred under N₂ at ambient temperature for 10 min. 1,2-Dibromoethane (2.11 mL, 24.5 mmol, Aldrich) was then added, and the resulting mixture was brought to reflux 3 times under N₂, cooling to ambient temperature in a water bath after each reflux. The mixture was then cooled to 0° C. in an ice bath, and chlorotrimethylsilane (3.42 mL, 26.9 mmol, Aldrich) was added over a few min under N₂. The mixture was then stirred at 0° C. for 5 min and allowed to warm to ambient temperature over 20 min with stirring under N₂. The cyclopropyl ethyl iodide prepared in Part A (30.04 g, 153 mmol) was added to the mixture. The mixture was then mixed at 40° C. under N₂ for 2 hr. Subsequently, tert-butyl 4-{[4-(5-bromopyrazin-2-yl)phenyl]sulfonyl}tetrahydro-2H-pyran-4-carboxylate (33 g, 68.12 mmol) and N,N-dimethylacetamide (260 mL) were combined in a separate flask. To this mixture was added the organozinc iodide prepared above (liquid was decanted into the flask containing DMA and the bromide after letting the solid Zn settle). Bis(benzonitrile)dichloropalladium(II) (1.67 g, 4.36 mmol, Aldrich) and 2-(dicyclohexylphosphino)-2′-methylbiphenyl (2.66 g, 7.3 mmol, Strem Chemicals) were then added. The resulting mixture was stirred at 55° C. under N₂ for 3 hr, and then cooled to ambient temperature overnight with mixing under N₂. The reaction was quenched by slow addition of deionized water (50 mL), and then letting the resulting mixture stir for approximately 20 min. The mixture was then diluted with ethyl acetate (500 mL) and deionized water (200 mL), and filtered through a bed of Celite. The filter cake was rinsed with ethyl acetate (100 mL), and the resulting filtrate layers were separated. The aqueous layer was back-extracted with ethyl acetate (200 mL). The combined ethyl acetate layers were then washed with a 1:1 mixture of deionized water/saturated NaCl(aq) (300 mL), washed with brine (300 mL), dried over MgSO₄, filtered, and concentrated. The resulting residue (48 g) was used in Step C without further purification.

Part C. The product from Part B was dissolved in CH₂Cl₂ (150 mL). To this mixture was added trifluoroacetic acid (150 mL). The resulting mixture was allowed to mix at ambient temperature in a vessel stoppered with a syringe needle vent. Afterward, the mixture was concentrated in vacuo to form a residue, which, in turn, was triturated with methanol (200 mL). The solids were filtered, washed with methanol, and dried in vacuo at 50° C. to a constant weight of 21.93 g (77% yield). ¹H NMR was consistent with the desired intermediate product.

Part D. The solids from Part C (21.93 g, 52.65 mmol), 1-hydroxybenzotriazole (14.22 g, 105 mmol, Aldrich), and 1-[3-dimethylamino)propyl]-3-ethylcarbodiimide hydrochloride (20.19 g, 105 mmol, Aldrich) were dissolved in N,N-dimethylformamide (200mL). The resulting mixture was allowed to mix in a stoppered vessel at ambient temperature for 10 min. Subsequently, 4-methylmorpholine (23.1 mL, 211 mmol) and O-(tetrahydropyranyl) hydroxylamine (12.32 g, 105 mmol, Carbogen) were added. The mixture was mixed in a covered vessel at ambient temperature overnight. The mixture was then poured into ethyl acetate (300 mL) and deionized water (200 mL). The layers were separated, and the aqueous layer was back-extracted with ethyl acetate (200 mL). The combined ethyl acetate layers were washed with a 1:1 mixture of deionized water/saturated NaCl(aq) (250 mL), washed with saturated NaCl(aq) (250 mL), dried over MgSO₄, and concentrated in vacuo to form an oil. The oil was purified by silica chromatography (hexanes/ethyl acetate (with 20% methanol)). The good column fractions were concentrated in vacuo to afford 18.6 g (68.6% yield) of solids. ¹H NMR was consistent with the desired intermediate product.

Part E. To the solids (18.6 g, 36.07 mmol) from Part D was added 1.25 N HCl/methanol (200 mL, Fluka). The mixture was allowed to stir in a covered vessel at ambient temperature over a weekend. Afterward, the mixture was concentrated in vacuo to solids. The solids were evaporated with a fresh portion of 1.25 N HCl/methanol (100 mL). Subsequently, solids were precipitated from 1.25 N HCl/methanol and deionized water. This mixture was stirred at ambient temperature for 2 hr. The solids were then filtered, washed with deionized water, and dried to a constant weight in vacuo at 50° C. to afford 14.6 g (94% yield) of the 4-({4-[5-(2-cyclopropylethyl)pyrazin-2-yl]phenyl}sulfonyl)-N-hydroxytetrahydro-2H-pyran-4-carboxamide hydrochloride product. HR-MS: M+H calculated for C₁₉H₂₃F₅N₄O₅S: 432.1588, found: 432.1590.

### Example 16

### Preparation of 1-cyclopropyl-4-({4-[5-(3,3,4,4,4-pentafluorobutyl)(2-pyridyl)]phenyl}sulfonyl)piperidine-4-carbohydroxamic acid, dihydrochloride

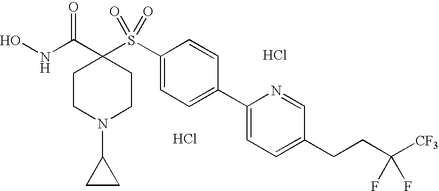

Part A. Preparation of tert-butyl 4-{[4-(5-bromo(2-pyridyl))phenyl]sulfonyl}-1-benzylpiperidine-4-carboxylate (3): 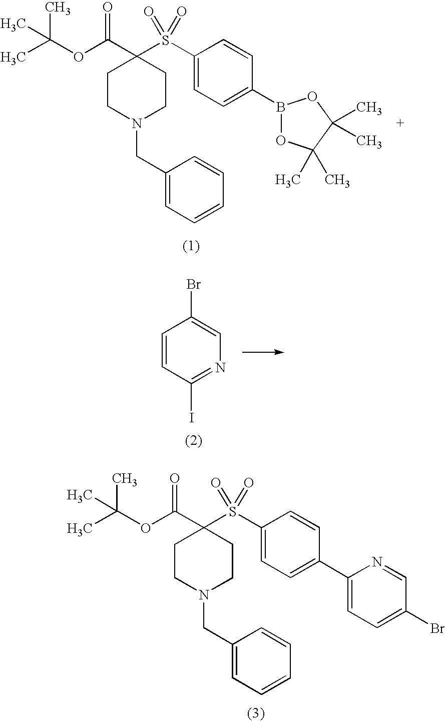
To a mixture of 1-benzyl-4-[4-(4,4,5,5-tetramethyl-[1,3,2]dioxaborolan-2-yl)-benzenesulfonyl]-piperidine-4-carboxylic acid tert-butyl ester (1) (1.5 g, 2.8 mmol) in toluene (8 mL), ethanol (2 mL), and 1M sodium carbonate (Na₂CO₃, 8 mL) under N₂ were added 2-iodo-5-bromopyridine (2) (0.87 g, 3.1 mmol) and [1,1′-bis(diphenylphosphino)ferrocene]dichloropalladium(II) (“Pd(dppf)C₁₂”, 0.11 g, 0.14 mmol). The mixture was then heated at 80° C. under N₂ for 6 hr, after which no starting material (1) was detected by LC/MS. The mixture was cooled to room temperature and diluted with ethyl acetate and water. The mixture was then filtered through a pad of Celite. The layers of the filtrate were separated, and the organic layer was washed with water (2 times), washed with saturated NaCl (1 time), and dried over anhydrous sodium sulfate. Filtration and evaporation of the solvent under reduced pressure formed a dark oil. The residue was dissolved in dichloromethane and purified on SiO₂ (using 30% ethyl acetate/hexane followed by 40% ethyl acetate/hexane) to afford 1.1 g of light yellow solid (67% yield). ¹H NMR and mass spectrometry (MH⁺=571.1) were consistent with the desired compound (3).

Part B. Preparation of tert-butyl 4-({4-[5-(3,3,4,4,4-pentafluorobutyl)(2-pyridyl]phenyl}sulfonyl)-1-benzylpiperidine-4-carboxylate (4): 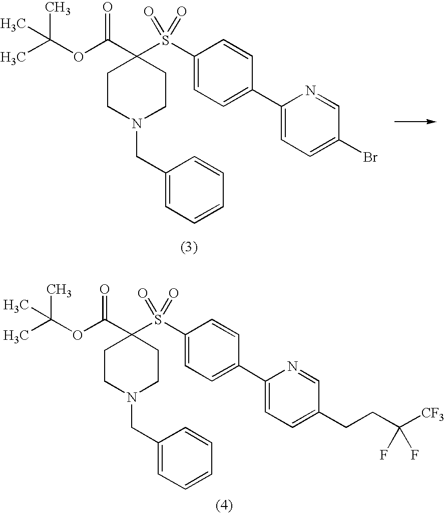
To a slurry of ZnCu couple (0.52 g, 8.1 mmol) in benzene (11 mL) and DNV (0.6 mL) was added 1,1,1,2,2-pentafluoro-4-iodobutane (1.5 g, 5.3 mmol). The resulting mixture was heated at 65° C. under N₂ for 3 hr. Subsequently, a mixture of the product (3) from Part A (1.0 g, 1.8 mmol) in benzene (3 mL) and DUT (1 mL) was added, followed by [1,1′-bis(diphenylphosphino)ferrocene]dichloropalladium(II) (“Pd(dppf)Cl₂”, 0.071 g, 0.087 mmol). The temperature was increased to 75° C., and the reaction was continued overnight, after which no starting material (3) remained by BPLC. The mixture was cooled to room temperature and diluted with ethyl acetate and water. The mixture was then filtered through a pad of Celite. The layers of the filtrate were separated, and the organic layer was washed with water (2 times), washed with saturated NaCl (1 time), and dried over anhydrous sodium sulfate. Filtration and evaporation of the solvent under reduced pressure formed a dark oil. The crude material was purified on SiO₂ using dichloromethane with a methanol gradient to afford 0.95 grams (83% yield) of an orange foam. ¹H NMR and mass spectrometry (MH⁺=639.1) were consistent with the desired compound (4).

Part C. Preparation of tert-butyl 4-({4-[5-(3,3,4,4,4-pentafluorobutyl)-2-pyridyl]phenyl}sulfonyl)piperidine-4-carboxylate (5): 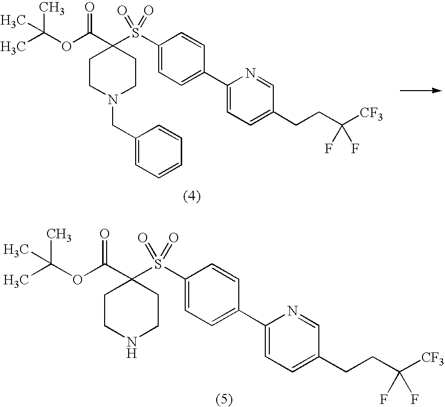
Cyclohexene (6.4 mL) and 10% Pd/C (0.94 g) were added to a methanol solution (16 mL) of the product (4) from Part B (0.94 g, 1.5 mmol). The resulting mixture was refluxed for 7 hr, after which HPLC indicated that the reaction was complete. The mixture was cooled to room temperature and filtered through Celite. The filtrate was concentrated under reduced pressure to form a yellow oil, which solidified upon standing (0.71 g, 86% yield). ¹H NMR and mass spectrometry (MH⁺=549.1) were consistent with the desired compound (5). This material was used in Step D without further purification.

Part D. Preparation of tert-butyl 1-cyclopropyl-4-({4-[5-(3,3,4,4,4-pentafluorobutyl)(2-pyridyl]phenyl}sulfonyl)piperidine-4-carboxylate (6): 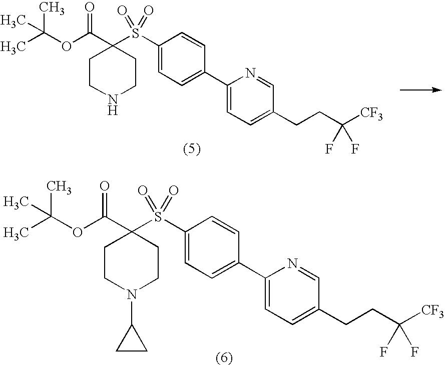
To a mixture of methanol (5 mL) and the product (5) from Part C (0.70 g, 1.3 mmol) were added [(1-ethoxycyclopropyl)oxy]trimethylsilane (0.33 g, 1.9 mmol), sodium cyanoborohydride (0.12 g, 2.0 mmol), acetic acid (0.77 g, 13 mmol, 0.73 mL), and 3 angstrom molecular sieves. The resulting mixture was stirred at 65° C. for 5 hr, after which LC/MS indicated that the reaction was complete. The mixture was then diluted with ethyl acetate and saturated sodium bicarbonate, and filtered through Celite. The filtrate was transferred to a separatory funnel, and the layers were separated. The organic layer was washed with saturated sodium bicarbonate and saturated NaCl, and then dried over anhydrous sodium sulfate. Filtration and evaporation of the solvent under reduced pressure afforded 0.73 g of a white solid (97% yield). ¹H NMR and mass spectrometry (MH⁺=589.1) were consistent with the desired compound (6). This material was used in Step E without further purification.

Part E. Preparation of the trifluoroacetic acid salt of 1-cyclopropyl-4-({4-[5-(3,3,4,4,4-pentafluorobutyl)(2-pyridyl]phenyl}sulfonyl)piperidine-4-carboxylic acid (7): 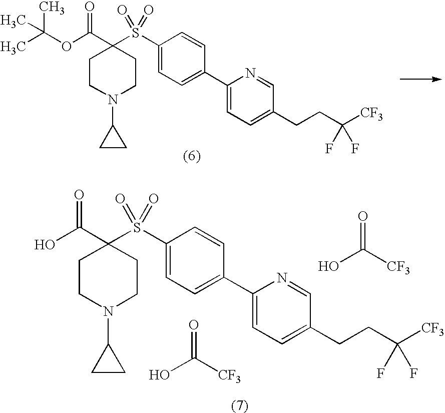
The product (6) of Part D (0.73 g, 1.2 mmol) was dissolved in 1:1 trifluoroacetic acid/dichloromethane (“TFA/CH₂Cl₂”, 20 mL). The reaction was continued overnight at room temperature, after which time no starting material (6) was detected by HPLC. The mixture was concentrated under reduced pressure. The residue was stripped from diethyl ether several times under reduced pressure, and then precipitated a final time and collected by suction filtration to afford 0.50 g of a solid (55% yield for the di-TFA salt). Mass spectrometry (MH⁺=533) was consistent with the desired product (7).

Part F. Preparation of [1-cyclopropyl-4-({4-[5-(3,3,4,4,4-pentafluorobutyl)(2-pyridyl)]phenyl}sulfonyl)(4-piperidyl)]N-perhydro-2H-pyran-2-yloxycarboxaminde (8): 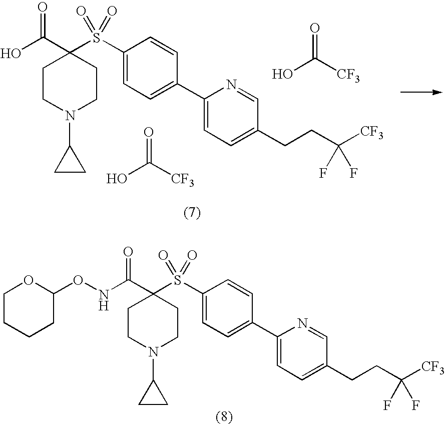
To a mixture of the product (7) from Part E (0.49 g, 0.64 mmol for di-TFA) in N,N-dimethylformamide (“DMF”, 16 mL) were added N-hydroxybenzotriazole (“HOBt”, 0.12 g, 0.90 mmol), 4-methylmorpholine (“NMM”, 0.32 g, 0.35 mL, 3.2 mmol), 1-(3-dimethylaminopropyl)-3-ethylcarbodiimide hydrochloride (“EDC.HCl”, 0.43 g, 2.2 mmol), and O-(tetrahydro-2H-pyran-2-yl)hydroxylamine (“THPONH₂”, 0.26 g, 2.2 mmol). The reaction was continued overnight at room temperature under N₂, after which time no starting material (7) was detected by HPLC. The mixture was diluted with ethyl acetate. The organic layer was extracted with water (3 times) and saturated sodium bicarbonate (3 times), washed with saturated NaCl, and dried over anhydrous sodium sulfate. Filtration and evaporation of the solvent under reduced pressure formed an oil. The crude material was purified by flash chromatography (using dichloromethane with a methanol gradient (0-2%)) to yield a white foam (0.26 g of pure material (64% yield), plus another 0.14 g of slightly impure material). ¹H NMR and mass spectrometry (MH⁺=632) were consistent with the desired product (8).

Part G. Preparation of 1-cyclopropyl-4-({4-[5-(3,3,4,4,4-pentafluorobutyl)(2-pyridyl)]phenyl}sulfonyl)piperidine-4-carbohydroxamic acid, dihydrochloride (9): 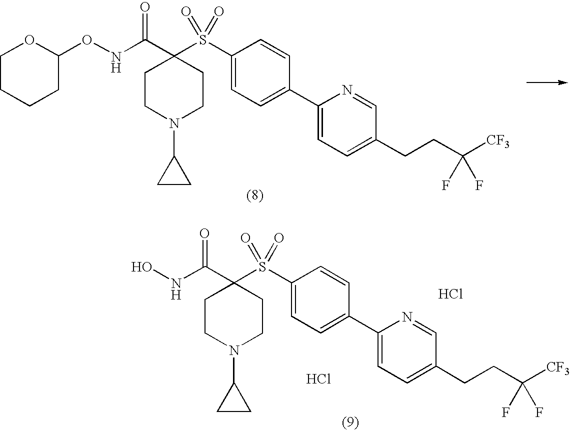
The product (8) from Part F (0.26 g, 0.41 mmol) was dissolved in dioxane (2 mL), 4N HCl in dioxane (2.5 mL), and methanol (0.25 mL). The reaction was continued at ambient temperature overnight, after which HPLC indicated that the reaction was complete. The mixture was concentrated under reduced pressure. The resulting residue was triturated with diethyl ether to form a white solid, which, in turn, was collected by suction filtration (0.25 g, quantitative yield). ¹H NMR and high resolution mass spectrometry (theoretical MH⁺=548.1637, observed MH⁺=548.1644) were consistent with the desired product (9).

### Example 17

### Preparation of 1-cyclopropyl-4-({4-[5-(4,4,4-trifluorobutyl)(2-pyridyl)]phenyl}sulfonyl)piperidine-4-carbohydroxamic acid, dihydrochloride

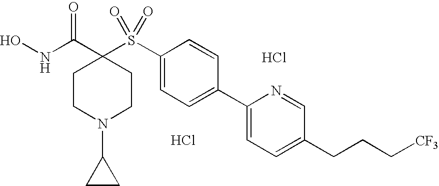

Part A. Preparation of tert-butyl 4-{[4-(5-bromo(2-pyridyl))phenyl]sulfonyl}-1-benzylpiperidine-4-carboxylate (3): 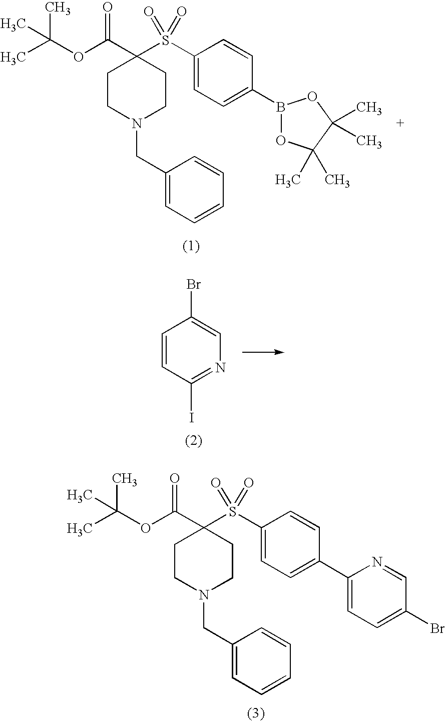
To a mixture of 1-benzyl-4-[4-(4,4,5,5-tetramethyl-[1,3,2]dioxaborolan-2-yl)-benzenesulfonyl]-piperidine-4-carboxylic acid tert-butyl ester (1) (5.0 g, 9.2 mmol) in toluene (28 mL), ethanol (7 mL), and 1M sodium carbonate (Na₂CO₃, 28 mL) under N₂ were added 2-iodo-5-bromopyridine (2) (2.9 g, 10.2 mmol) and [1,1′-bis(diphenylphosphino)ferrocene]dichloropalladium(II) (“Pd(dppf)Cl₂”, 0.38 g, 0.46 mmol). The resulting mixture was heated at 80° C. under N₂ for 6 hr, after which no starting material (2) was detected By LC/MS. The mixture was cooled to room temperature, diluted with ethyl acetate and water, and filtered through a pad of Celite. The layers of the filtrate were separated, and the organic layer was washed with water (2 times), washed with saturated NaCl (1 time), and dried over anhydrous sodium sulfate. Filtration and evaporation of the solvent under reduced pressure formed a dark oil. The residue was dissolved in dichloromethane and purified on SiO₂ (using 30% ethyl acetate/hexane) to afford 3.6 g of light yellow solid (69% yield). ¹H NMR and mass spectrometry (MH⁺=571.1) were consistent with the desired compound (3).

Part B. Preparation of tert-butyl 4-({4-[5-(4,4,4-trifluorobutyl)(2-pyridyl]phenyl}sulfonyl)-1-benzylpiperidine-4-carboxylate (4): 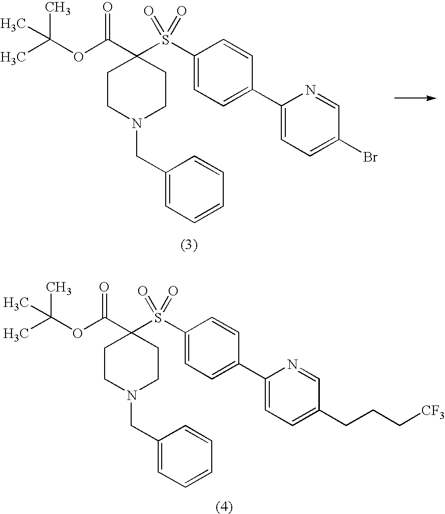
To a slurry of ZnCu couple (1.3 g, 19.3 mmol) in benzene (28 mL) and DMF (1.5 mL) was added 1,1,1-trifluoro-4-iodobutane (3.0 g, 12.6 mmol). The resulting mixture was heated at 65° C. under N₂ for 3 hr. A solution of the product (3) from Part A (2.4 g, 4.2 mmol) in benzene (7.2 mL) and DMF (2.5 mL) was subsequently added, followed by [1,1′-bis(diphenylphosphino)ferrocene]dichloropalladium(II) (“Pd(dppf)Cl₂”, 0.071 g, 0.087 mmol). The temperature was then increased to 75° C., and the reaction was continued overnight, after which no starting material (3) was detected by HPLC. The mixture was cooled to room temperature and diluted with ethyl acetate and water, and filtered through a pad of Celite. The layers of the filtrate were separated, and the organic layer was washed with water (2 times), washed with saturated NaCl (1 time), and dried over anhydrous sodium sulfate. Filtration and evaporation of the solvent under reduced pressure formed a reddish foam. The crude material was purified on SiO₂ (using dichloromethane with a methanol gradient (0-2%)) to afford 1.5 grams (60% yield) of a foam. ¹H NMR and mass spectrometry (MH⁺=603.1) were consistent with the desired compound (4).

Part C. Preparation of tert-butyl 4-({4-[5-(4,4,4-trifluorobutyl)-2-pyridyl]phenyl}sulfonyl)piperidine-4-carboxylate (5): 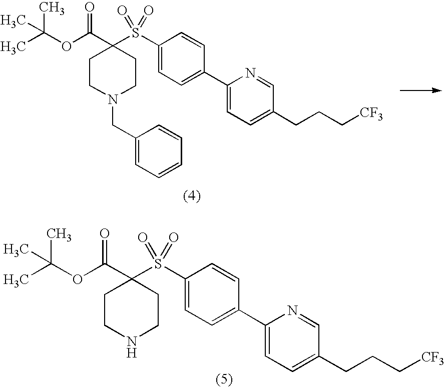
The product (4) from Part B (1.9 g, 3.2 mmol) was dissolved in 4:1 ethanol/formic acid (20 and 5 mL, respectively), and then 10% Pd/C (1.0 g) was added. The mixture was heated at 55° C. for 1 hr, and then cooled to room temperature. Subsequently, the mixture was filtered through Celite to remove the catalyst. The filtrate was then concentrated under reduced pressure. The residue was re-dissolved in water, and the aqueous mixture was made basic with 2.5 N NaOH. The product was then extracted into ethyl acetate. The organic layer was washed with water (3×), washed with saturated NaCl (1×), and dried over anhydrous sodium sulfate. Filtration and evaporation of the solvent under reduced pressure formed a foam. The crude material was purified on SiO₂ (using dichloromethane with a methanol gradient (0-10%)) to afford 0.64 grams of product (5) (40% yield). ¹H NMR and mass spectrometry (MH⁺=513) were consistent with the desired compound (5).

Part D. Preparation of tert-butyl 1-cyclopropyl-4-({4-[5-(4,4,4-trifluorobutyl)(2-pyridyl]phenyl}sulfonyl)piperidine-4-carboxylate (6): 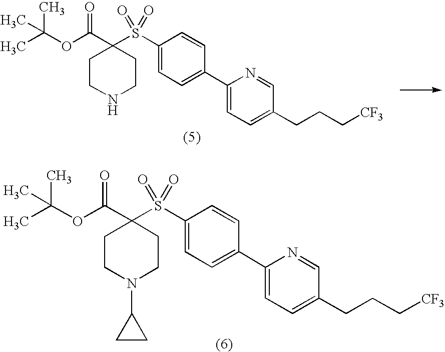
To a mixture of methanol (4 mL) and the product (5) from Part C (0.42 g, 0.82 mmol) were added [(1-ethoxycyclopropyl)oxy]trimethylsilane (0.21 g, 1.2 mmol), sodium cyanoborohydride (0.079 g, 1.3 mmol), acetic acid (0.49 g, 8.2 mmol, 0.47 mL), and 3 angstrom molecular sieves. The resulting mixture was stirred at 65° C. for 5 hr, after which LC/MS indicated that the reaction was complete. The mixture was then diluted with ethyl acetate and saturated sodium bicarbonate, and filtered through Celite. The filtrate was transferred to a separatory funnel, and the layers were separated. The organic layer was washed with saturated sodium bicarbonate and saturated NaCl, and then dried over anhydrous sodium sulfate. Filtration and evaporation of the solvent under reduced pressure afforded 0.38 g of a white solid (84% yield). ¹H NMR and mass spectrometry (MH⁺=553) were consistent with the desired compound (6). This material was used in Step E without further purification.

Part E. Preparation of the trifluoroacetic acid salt of 1-cyclopropyl-4-({4-[5-(4,4,4-trifluorobutyl)(2-pyridyl]phenyl}sulfonyl)piperidine-4-carboxylic acid (7): 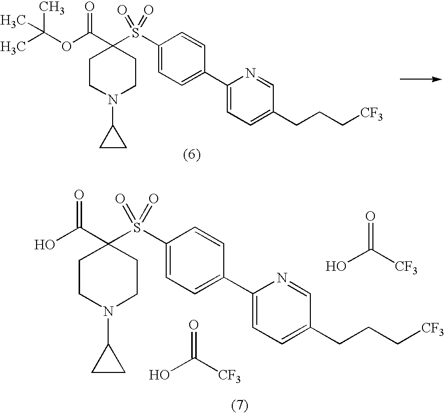
The product (6) from Part D (0.37 g, 0.67 mmol) was dissolved into 1:1 trifluoroacetic acid/dichloromethane (“TFA/CH₂Cl₂”, 10 mL). The reaction was continued overnight at room temperature, after which time no starting material (6) was detected by HPLC. The mixture was concentrated under reduced pressure. The residue was stripped from diethyl ether several times under reduced pressure, and then dried under high vacuum to afford the desired compound (7) (0.58 g, quantitative yield for the di-TFA salt+1 extra mol of TFA). Mass spectrometry (MH⁺=497) was consistent with the desired compound (7).

Part F. Preparation of [1-cyclopropyl-4-({4-[5-(4,4,4-trifluorobutyl)(2-pyridyl)]phenyl}sulfonyl)(4-piperidyl)]-N-perhydro-2H-pyran-2-yloxycarboxamide (8): 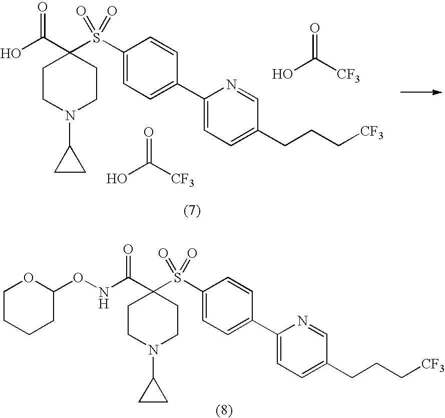
To a mixture of the product (7) from Part E (0.58 g, 0.69 mmol for “tri-TFA”) in N,N-dimethylformamide (“DMF”, 18 mL) were added N-hydroxybenzotriazole (“HOBt”, 0.13 g, 0.97 mmol), 4-methylmorpholine (“NMM”, 0.42 g, 0.46 mL, 4.1 mmol), 1-(3-dimethylaminopropyl)-3-ethylcarbodiimide hydrochloride (“EDC.HCl”, 0.60 g, 3.1 mmol), and O-(tetrahydro-2H-pyran-2-yl)hydroxylamine (“THPOHN₂”, 0.36 g, 3.1 mmol). The reaction was continued overnight at room temperature under N₂, after which no starting material (7) was detected by HPLC. The mixture was diluted with ethyl acetate. The organic layer was then extracted with water (3 times) and saturated sodium bicarbonate (3 times), washed with saturated NaCl, and dried over anhydrous sodium sulfate. Filtration and evaporation of the solvent under reduced pressure formed an oil. The crude material was purified by flash chromatography (using dichloromethane with a methanol gradient (0-2%)) to afford a white foam (0.30 g, 77% yield). ¹H NMR and mass spectrometry (MH⁺=596) were consistent with the desired product (8).

Part G. Preparation of 1-cyclopropyl-4-({4-[5-(4,4,4-trifluorobutyl)(2-pyridyl)]phenyl}sulfonyl)piperidine-4-carbohydroxamic acid, dihydrochloride (9): 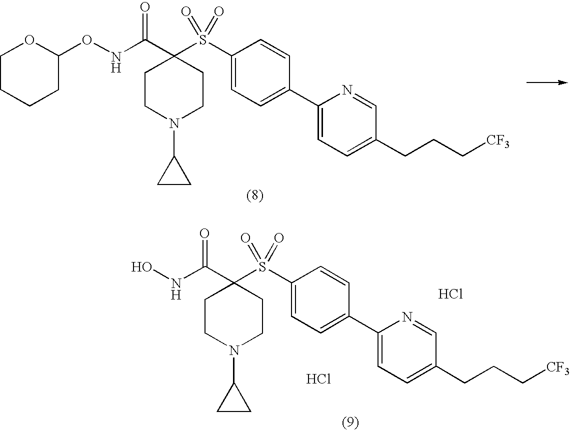
The product (8) from Part F (0.29 g, 0.49 mmol) was dissolved into dioxane (2 ML), 4N HCl in dioxane (2.5 mL), and methanol (0.25 mL). The reaction was continued at ambient temperature for 4 hr, after which HPLC indicated that the reaction was complete. The mixture was then concentrated under reduced pressure. The residue was triturated with diethyl ether to form a white solid, which, in turn, was collected by suction filtration to afford 0.25 g of product (quantitative yield). ¹H NMR and high resolution mass spectrometry (theoretical MH⁺=512.1825, actual MH⁺=512.1846) were consistent with the desired compound (9).

### Example 18

### Preparation of 1-ethyl-4-({4-[5-(3,3,4,4,4-pentafluorobutyl)(2-pyridyl)]phenylasulfonyl)piperidine-4-carbohydroxanic acid, dihydrochloride

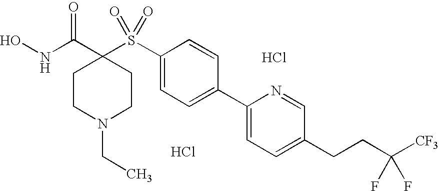

Part A. Preparation of tert-butyl 4-{[4-(5-bromo(2-pyridyl))phenyl]sulfonyl}-1-benzylpiperidine-4-carboxylate (3): 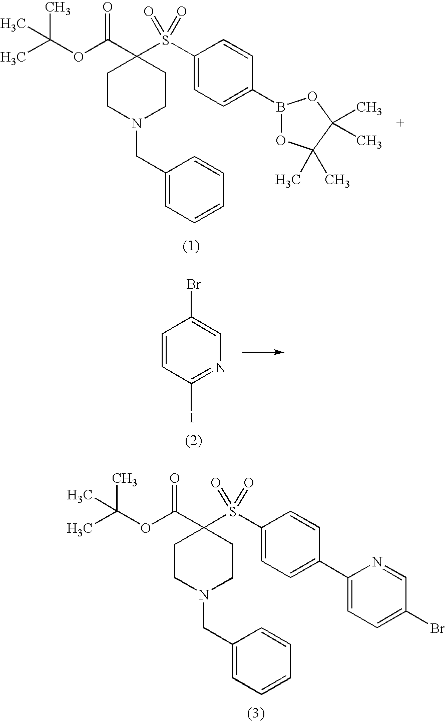
To a mixture of 1-benzyl-4-[4-(4,4,5,5-tetramethyl-[1,3,2]dioxaborolan-2-yl)-benzenesulfonyl]-piperidine-4-carboxylic acid tert-butyl ester (1) (1.5 g, 2.8 mmol) in toluene (8 mL), ethanol (2 mL), and 1M sodium carbonate (Na₂CO₃, 8 mL) under N₂ were added 2-iodo-5-bromopyridine (2) (0.87 g, 3.1 mmol) and [1,1′-bis(diphenylphosphino)ferrocene]dichloropalladium(II) (“Pd(dppf)Cl₂”, 0.11 g, 0.14 mmol). The resulting mixture was heated at 80° C. under N₂ for 6 hr, after which no starting material (1) was detected by LC/MS. The mixture was cooled to room temperature, diluted with ethyl acetate and water, and filtered through a pad of Celite. The layers of the filtrate were separated, and the organic layer was washed with water (2 times), washed with saturated NaCl (1 time), and dried over anhydrous sodium sulfate. Filtration and evaporation of the solvent under reduced pressure formed a dark oil. The residue was dissolved in dichloromethane and purified on SiO₂ (using 30% ethyl acetate/hexane, followed by 40% ethyl acetate/hexane) to afford 1.1 g of light yellow solid (67% yield). ¹H NMR and mass spectrometry (MH⁺=571.1) were consistent with the desired compound (3).

Part B. Preparation of tert-butyl 4-({4-[5-(3,3,4,4,4-pentafluorobutyl)(2-pyridyl]phenyl}sulfonyl)-1-benzylpiperidine-4-carboxylate (4): 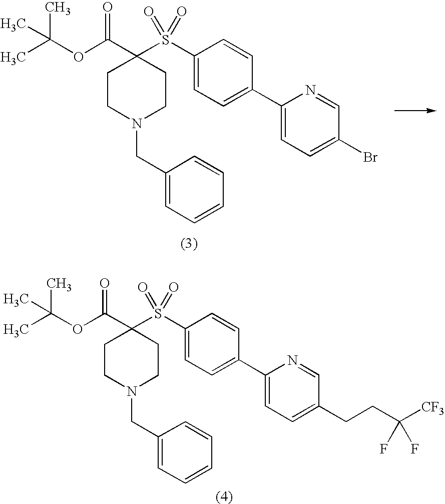
To a slurry of ZnCu couple (0.52 g, 8.1 mmol) in benzene (11 mL) and DMF (0.6 mL) was added 1,1,1,2,2-pentafluoro-4-iodobutane (1.5 g, 5.3 mmol). The resulting mixture was heated at 65° C. under N₂ for 3 hr. A mixture of the product (3) from Part A (1.0 g, 1.8 mmol) in benzene (3 mL) and DMF (1 mL) was subsequently added, followed by [1,1′-bis(diphenylphosphino)ferrocene]dichloropalladium(II) (“Pd(dppf)Cl₂”, 0.071 g, 0.087 mmol). The temperature was then increased to 75° C., and the reaction was continued overnight, after which no starting material (3) was detected by HPLC. The mixture was cooled to room temperature, diluted with ethyl acetate and water, and filtered through a pad of Celite. The layers of the filtrate were separated, and the organic layer was washed with water (2 times), washed with saturated NaCl (1 time), and dried over anhydrous sodium sulfate. Filtration and evaporation of the solvent under reduced pressure formed a dark oil. The crude material was purified on SiO₂ (using dichloromethane with a methanol gradient) to afford 0.95 grams (83% yield) of an orange foam. ¹H NMR and mass spectrometry (MH⁺=639.1) were consistent with the desired compound (4).

Part C. Preparation of tert-butyl 1-ethyl-4-({4-[5-(3,3,4,4,4-pentafluorobutyl)(2-pyridyl]phenyl}sulfonyl)piperidine-4-carboxylate (5): 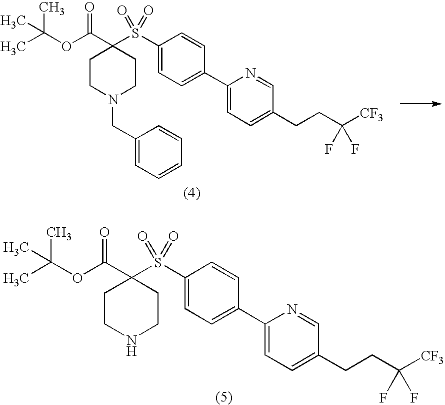
Cyclohexene (6.4 mL) and 10% Pd/C (0.94 g) were added to a mixture of methanol (16 mL) and the product (4) from Part B (0.94 g, 1.5 mmol). The mixture was refluxed for 7 hr, after which HPLC indicated that reaction was complete. The mixture was cooled to room temperature and filtered through Celite. The filtrate was concentrated under reduced pressure to form a yellow oil, which, in turn, solidified upon standing to form 0.71 g of product (86% yield). ¹H NMR and mass spectrometry (MH⁺=549.1) were consistent with the desired compound (5). This material was used in Part D without further purification.

Part D. Preparation of tert-butyl 1-ethyl-4-({4-[5-(3,3,4,4,4-pentafluorobutyl)(2-pyridyl]phenyl}sulfonyl)piperidine-4-carboxylate (6): 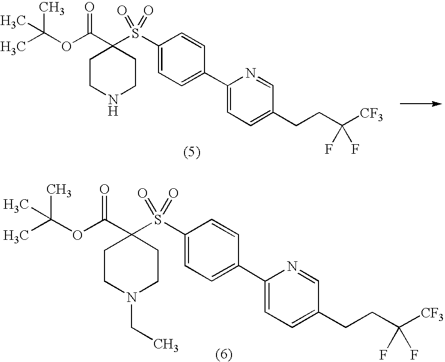
The product (5) from Part C (1.5 g, 2.7 mmol), iodoethane (0.45 g, 2.9 mmol), and N,N-diisopropylethylamine (0.37 g, 2.9 mmol) were dissolved in DMF (45 mL). Subsequently, the reaction was continued overnight at room temperature. Because there was still some starting material remaining afterward, additional iodoethane and DIEA (0.6 mmol each) were added. The reaction was once again continued overnight, after which LC/MS indicated that the reaction was complete. The mixture was then diluted with ethyl acetate. The organic layer was washed with water (3×), washed with saturated NaCl (1×), and dried over anhydrous sodium sulfate. Filtration and evaporation of the solvent under reduced pressure afforded 1.3 g of a solid (84% yield). ¹H NMR and mass spectrometry (MH⁺=577) were consistent with the desired compound (6). This material was used in Step E without further purification.

Part E. Preparation of the trifluoroacetic acid salt of 1-ethyl-4-({4-[5-(3,3,4,4,4-pentafluorobutyl)(2-pyridyl]phenyl}sulfonyl)piperidine-4-carboxylic acid (7): 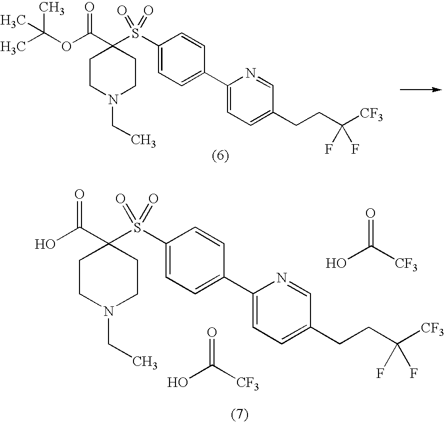
The product (6) from Part D (1.3 g, 2.3 mmol) was dissolved in 1:1 trifluoroacetic acid/dichloromethane (40 mL). The reaction was continued overnight at room temperature, after which no starting material (6) was detected by HPLC. The mixture was concentrated under reduced pressure. The resulting residue was stripped from diethyl ether several times under reduced pressure, and then precipitated a final time and collected by suction filtration to afford 1.14 g of a solid (66% yield for the di-TFA salt). Mass spectrometry (MH⁺=521) was consistent with the desired compound (7).

Part F. Preparation of [1-ethyl-4-({4-[5-(3,3,4,4,4-pentafluorobutyl)(2-pyridyl)]phenyl}sulfonyl)(4-piperidyl)]-N-perhydro-2H-pyran-2-yloxycarboxamide (8): 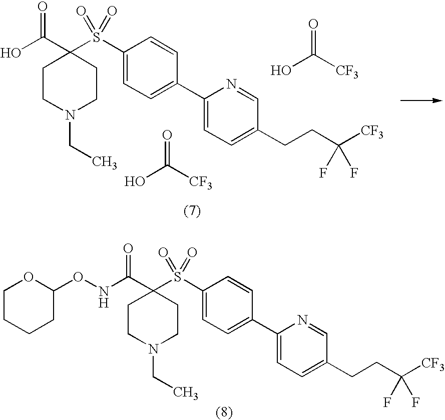
To a solution of the product (7) from Part E (1.1 g, 1.5 mmol for the di-TFA salt) in N,N-dimethylformamide (“DMF”, 40 mL) were added N-hydroxybenzotriazole (“HOBt”, 0.29 g, 2.1 mmol), 4-methylmorpholine (“NMM”, 0.77 g, 0.84 mL, 7.6 mmol), 1-(3-dimethylaminopropyl)-3-ethylcarbodiimide hydrochloride (“EDC.HCl”, 1.0 g, 5.3 mmol), and O-(tetrahydro-2H-pyran-2-yl)hydroxylamine (THPONH₂”, 0.62 g, 5.3 mmol). The resulting mixture was heated for 2 hr at 55° C., and then cooled to room temperature. Stirring was continued at room temperature under N₂ over a weekend, after which no starting material (7) was detected by HPLC. The mixture was diluted with ethyl acetate. The organic layer was extracted with water (3 times) and saturated sodium bicarbonate (3 times), washed with saturated NaCl, and then dried over anhydrous sodium sulfate. Filtration and evaporation of the solvent under reduced pressure formed an oil. The crude material was purified by flash chromatography (using dichloromethane with a methanol gradient (0-3%)) to afford 0.73 g of an off-white foam (79% yield). ¹H NMR and mass spectrometry (MH⁺=620) were consistent with the desired product (8).

Part G. Preparation of 1-ethyl-4-({4-[5-(3,3,4,4,4-pentafluorobutyl)(2-pyridyl)]phenyl}sulfonyl)piperidine-4-carbohydroxamic acid, dihydrochloride (9): 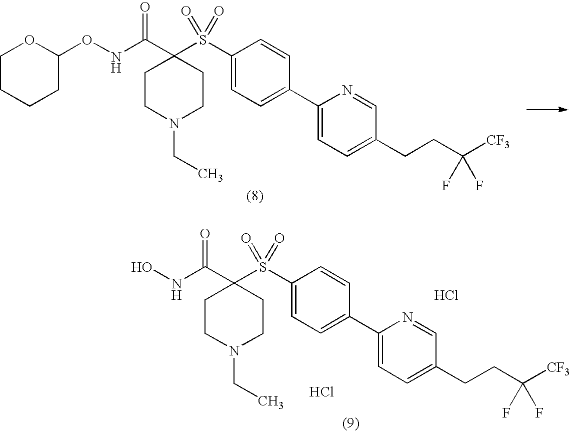
The product (8) from Part F (0.72 g, 1.1 mmol) was dissolved in dioxane (8 mL), 4N HCl in dioxane (10 mL), and methanol (1 mL). The reaction was continued at ambient temperature overnight, after which HPLC indicated that the reaction was complete. The mixture was concentrated under reduced pressure. The resulting residue was triturated with diethyl ether to form a white solid, which, in turn, was collected by suction filtration to form 0.66 g of product (quantitative yield). ¹H NMR and high resolution mass spectrometry (theoretical MH⁺=536.1637, actual MH⁺=536.1606) were consistent with the desired compound (9).

### Example 19

### Preparation of 1-ethyl-4-({4-[5-(4,4,4-trifluorobutyl)(2-pyridyl)]phenyl}sulfonyl)piperidine-4-carbohydroxamic acid, dihydrochloride

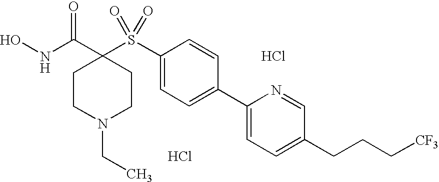

Part A. Preparation of tert-butyl 4-{[4-(5-bromo(2-pyridyl))phenyl]sulfonyl}-1-benzylpiperidine-4-carboxylate (3): 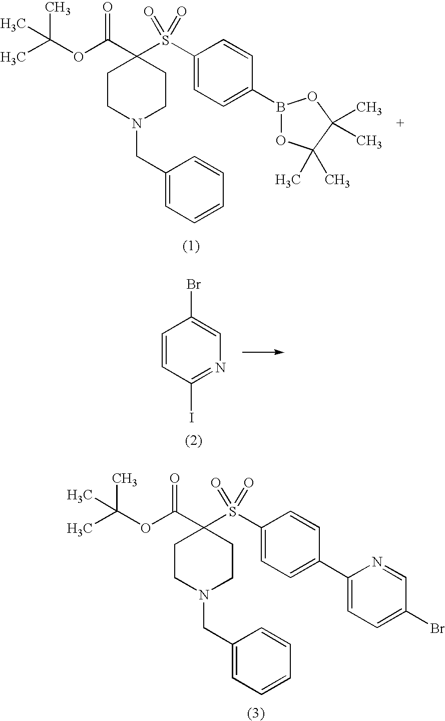
To a mixture of 1-benzyl-4-[4-(4,4,5,5-tetramethyl-[1,3,2]dioxaborolan-2-yl)-benzenesulfonyl]-piperidine-4-carboxylic acid tert-butyl ester (1) (5.0 g, 9.2 mmol) in toluene (28 mL), ethanol (7 mL), and 1 M sodium carbonate (Na₂CO₃, 28 mL) under N₂ were added 2-iodo-5-bromopyridine (2) (2.9 g, 10.2 mmol) and [1,1′-bis(diphenylphosphino)ferrocene]dichloropalladium(II) (“Pd(dppf)Cl₂”, 0.38 g, 0.46 mmol). The resulting mixture was heated at 80° C. under N₂ for 6 hr, after which LC/MS detected no starting material (1). The mixture was cooled to room temperature, diluted with ethyl acetate and water, and filtered through a pad of Celite. The layers of the filtrate were separated, and the organic layer was washed with water (2 times), washed with saturated NaCl (1 time), and dried over anhydrous sodium sulfate. Filtration and evaporation of the solvent under reduced pressure formed a dark oil. The residue was dissolved in dichloromethane and purified on SiO₂ (using 30% ethyl acetate/hexane) to afford 3.6 g of a light yellow solid (69% yield). ¹H NMR and mass spectrometry (MH⁺=571.1) were consistent with the desired compound (3).

Part B. Preparation of tert-butyl 4-({4-[5-(4,4,4-trifluorobutyl)(2-pyridyl]phenyl}sulfonyl)-1-benzylpiperidine-4-carboxylate (4): 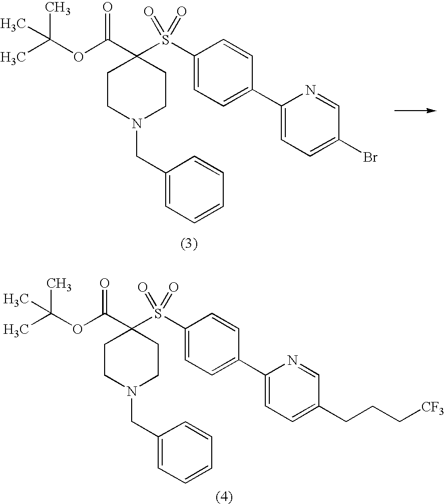
To a slurry of ZnCu couple (1.3 g, 19.3 mmol) in benzene (28 mL) and DMF (1.5 mL) was added 1,1,1-trifluoro-4-iodobutane (3.0 g, 12.6 mmol). The resulting mixture was heated at 65° C. under N₂ for 3 hr. A mixture of the product (3) from Part A (2.4 g, 4.2 mmol) in benzene (7.2 mL) and DMF (2.5 mL) was subsequently added, followed by [1,1′-bis(diphenylphosphino)ferrocene]dichloropalladium(II) (0.071 g, 0.087 mmol). The temperature was increased to 75° C., and the reaction was continued overnight, after which no starting material was detected by HPLC. The mixture was cooled to room temperature and diluted with ethyl acetate and water, and filtered through a pad of Celite. The layers of the filtrate were separated, and the organic layer was washed with water (2 times), washed with saturated NaCl (1 time), and dried over anhydrous sodium sulfate. Filtration and evaporation of the solvent under reduced pressure formed a reddish foam. The crude material was purified on SiO₂ (using dichloromethane with a methanol gradient (0-2%)) to afford 1.5 g (60% yield) of a foam. ¹H NMR and mass spectrometry (MH⁺=603.1) were consistent with the desired compound (4).

Part C. Preparation of tert-butyl 4-({4-[5-(4,4,4-trifluorobutyl)-2-pyridyl]phenyl}sulfonyl)piperidine-4-carboxylate (5): 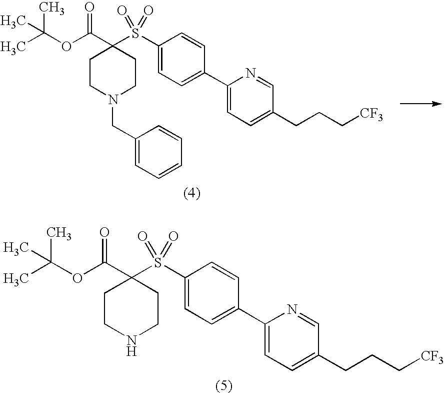
The product (4) from Part B (1.9 g, 3.2 mmol) was dissolved in 4:1 ethanol/formic acid (20 and 5 mL, respectively). Subsequently, 10% Pd/C (1.0 g) was added. The resulting mixture was heated at 55° C. for 1 hr, and then cooled to room temperature and filtered through Celite to remove the catalyst. The filtrate was concentrated under reduced pressure, and the residue was re-dissolved in water. The resulting aqueous mixture was made basic with 2.5 N NaOH. The product was then extracted into ethyl acetate. The organic layer was washed with water (3×), washed with saturated NaCl (1×), and dried over anhydrous-sodium sulfate. Filtration and evaporation of the solvent under reduced pressure formed a foam. The crude material was purified on SiO₂ (using dichloromethane with a methanol gradient (0-10%)) to afford 0.64 grams of product (5) (40% yield). ¹H NMR and mass spectrometry (MH⁺=513) were consistent with the desired compound (5).

Part D. Preparation of tert-butyl 1-ethyl-4-({4-[5-(4,4,4-trifluorobutyl)(2-pyridyl]phenyl}sulfonyl)piperidine-4-carboxylate (6): 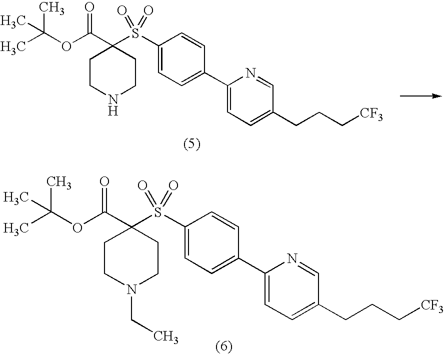
The product (5) from Part C (0.64 g, 1.3 mmol), iodoethane (0.23 g, 1.5 mmol), and N,N-diisopropylethylamine (0.23 g, 1.5 mmol) were dissolved in DMF (21 mL). The reaction was then continued overnight at room temperature, after which LC/MS indicated that the reaction was complete. The mixture was then diluted with ethyl acetate, and the organic layer was washed with water (3×), washed with saturated NaCl (1×), and dried over anhydrous sodium sulfate. Filtration and evaporation of the solvent under reduced pressure afforded 0.61 g of an oil (90% yield). ¹H NMR and mass spectrometry (MH⁺=541.2) were consistent with the desired compound (6). This material was used in Step E without further purification.

Part E. Preparation of the trifluoroacetic acid salt of 1-ethyl-4-({4-[5-(4,4,4-trifluorobutyl)(2-pyridyl]phenyl}sulfonyl)piperidine-4-carboxylic acid (7): 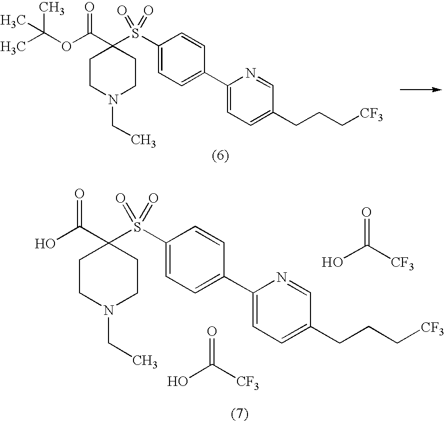
The product (6) from Part D (0.60 g, 1.1 mmol) was dissolved in 1:1 trifluoroacetic acid/dichloromethane (“TFA/CH₂Cl₂”, 30 mL). The reaction was then continued overnight at room temperature, after which HPLC detected no starting material (6). The mixture was concentrated under reduced pressure. The residue was then stripped from diethyl ether several times under reduced pressure, and then dried under high vacuum to afford 0.92 g of product (quantitative yield for the di-TFA salt+1 extra TFA). Mass spectrometry (MH⁺=485.1) was consistent with the desired product (7).

Part F. Preparation of [1-ethyl-4-({4-[5-(4,4,4-trifluorobutyl)(2-pyridyl)]phenyl}sulfonyl)(4-piperidyl)]-N-perhydro-2H-pyran-2-yloxycarboxamide (8): 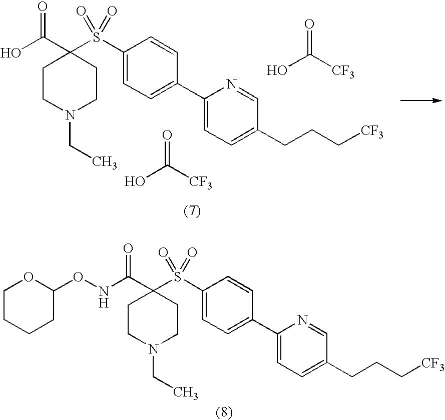
To a mixture of the product (7) from Part E (0.92 g, 1.1 mmol for “tri-TFA”) in N,N-dimethylformamide (“DMF”, 33 mL) were added N-hydroxybenzotriazole (“HOBt”, 0.21 g, 1.6 mmol), 4-methylmorpholine (“NMM”, 0.56 g, 0.60 mL, 5.5 mmol), 1-(3-dimethylaminopropyl)-3-ethylcarbodiimide hydrochloride (“EDC.HCl”, 0.74 g, 3.9 mmol), and O-(tetrahydro-2H-pyran-2-yl)hydroxylamine (“THPONH₂”, 0.46 g, 3.9 mmol). The resulting mixture was heated at 50° C. for 1 hr, and then held at room temperature under N₂ overnight. Afterward, LC/MS detected a small amount of starting material (7), so additional NMM, EDC, and THPONH₂ (one equivalent each) were added. After stirring for one more night at room temperature, no starting material (7) was detected by HPLC. The reaction mixture was then diluted with ethyl acetate. The organic layer was extracted with water (3 times) and saturated sodium bicarbonate (3 times), washed with saturated NaCl, and dried over anhydrous sodium sulfate. Filtration and evaporation of the solvent under reduced pressure formed an oil. The crude material was purified by flash chromatography (using dichloromethane with a methanol gradient (0-5%)) to afford the product(0.10 g of pure material+260 mg of mixed fractions). ¹H NMR and mass spectrometry (MH⁺=584.2) were consistent with the desired compound (8).

Part G. Preparation of 1-ethyl-4-({4-[5-(4,4,4-trifluorobutyl)(2-pyridyl)]phenyl}sulfonyl)piperidine-4-carbohydroxamic acid, dihydrochloride (9): 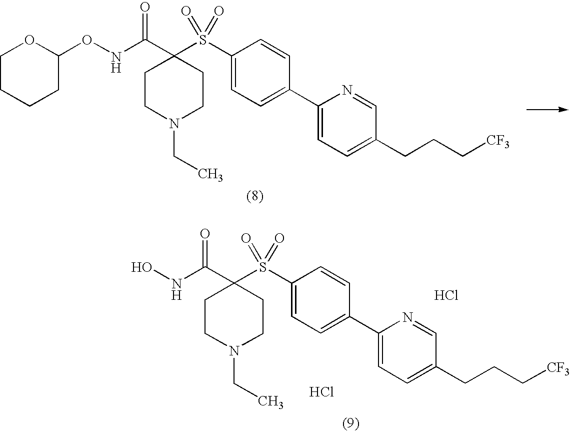
The product (8) from Part F (0.10 g, 0.49 mmol) was dissolved in dioxane (2 mL), 4N HCl in dioxane (2.5 mL), and methanol (0.25 mL). The reaction was continued at ambient temperature overnight, after which BPLC indicated that the reaction was complete. The mixture was concentrated under reduced pressure. The resulting residue was triturated with diethyl ether to form a white solid, which, in turn, was collected by suction filtration to form 0.078 g of product (32% yield). ¹H NMR and high resolution mass spectrometry (theoretical MH⁺=500.1825, actual MH⁺=500.1809) were consistent with the desired compound (9).

### Example 20

### Preparation of 1-(2-methoxyethyl)-4-({4-[5-(4,4,4-trifluorobutyl)(2-pyridyl)]phenyl}sulfonyl)piperidine-4-carbohydroxamic acid, dihydrochloride

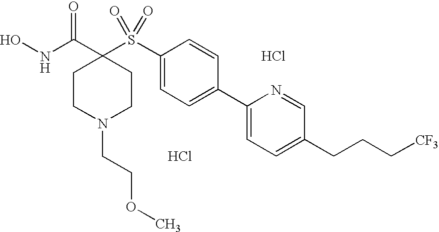

Part A. Preparation of tert-butyl 4-{[4-(5-bromo(2-pyridyl))phenyl]sulfonyl}-1-benzylpiperidine-4-carboxylate (3): 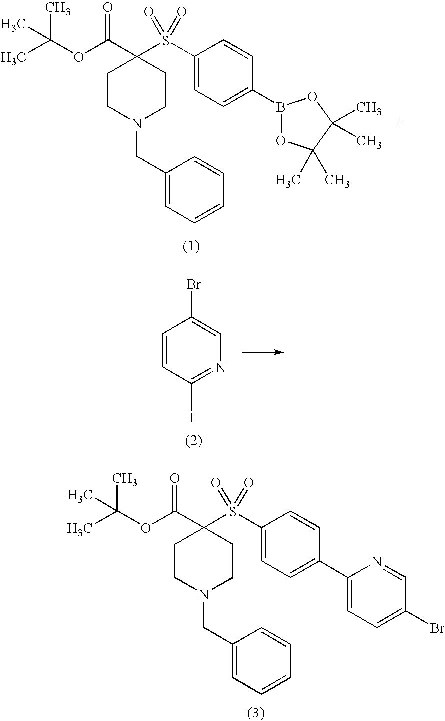
To a mixture of 1-benzyl-4-[4-(4,4,5,5-tetramethyl-[1,3,2]dioxaborolan-2-yl)-benzenesulfonyl]-piperidine-4-carboxylic acid tert-butyl ester (1) (5.0 g, 9.2 mmol) in toluene (28 mL), ethanol (7 mL), and 1 M sodium carbonate (Na₂CO₃, 28 mL) under N₂ were added 2-iodo-5-bromopyridine (2) (2.9 g, 10.2 mmol) and [1,1′-bis(diphenylphosphino)ferrocene]dichloropalladium(II) (“Pd(dppf)Cl₂”, 0.38 g, 0.46 mmol). The resulting mixture was heated at 80° C. under N₂ for 6 hr, after which LC/MS detected no starting material (1). The mixture was then cooled to room temperature, diluted with ethyl acetate and water, and filtered through a pad of Celite. The layers of the filtrate were separated, and the organic layer was washed with water (2 times), washed with saturated NaCl (1 time), and dried over anhydrous sodium sulfate. Filtration and evaporation of the solvent under reduced pressure formed a dark oil. The residue was dissolved in dichloromethane and purified on SiO₂ (using 30% ethyl acetate/hexane) to afford 3.6 g of light yellow solid (69% yield). ¹H NMR and mass spectrometry (MH⁺=571.1) were consistent with the desired compound (3).

Part B. Preparation of tert-butyl 4-({4-[5-(4,4,4-trifluorobutyl)(2-pyridyl]phenyl}sulfonyl)-1-benzylpiperidine-4-carboxylate (4): 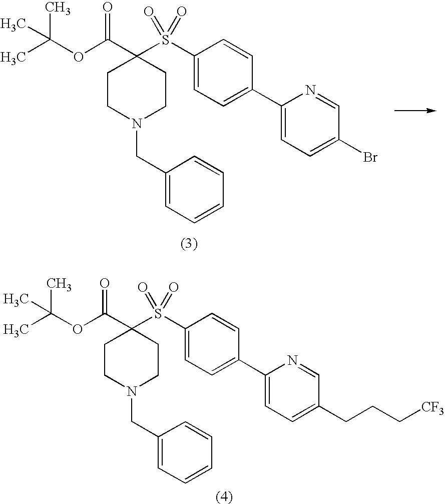
To a slurry of ZnCu couple (1.3 g, 19.3 mmol) in benzene (28 mL) and DMF (1.5 mL) was added 1,1,1-trifluoro-4-iodobutane (3.0 g, 12.6 mmol). The resulting mixture was heated at 65° C. under N₂ for 3 hr. A mixture of the product (3) from Part A (2.4 g, 4.2 mmol) in benzene (7.2 mL) and DMF (2.5 mL) was subsequently added, followed by [1,1′-bis(diphenylphosphino)ferrocene]dichloropalladium(II) (“Pd(dppf)Cl₂”, 0.071 g, 0.087 mmol). The temperature was increased to 75° C., and the reaction was continued overnight, after which no starting material was detected by HPLC. The mixture was cooled to room temperature, diluted with ethyl acetate and water, and filtered through a pad of Celite. The layers of the filtrate were separated, and the organic layer was washed with water (2 times), washed with saturated NaCl (1 time), and dried over anhydrous sodium sulfate. Filtration and evaporation of the solvent under reduced pressure formed a reddish foam. The crude material was purified on SiO₂ (using dichloromethane with a methanol gradient (0-2%)) to afford 1.5 grams of a form (60% yield). ¹H NMR and mass spectrometry (MH⁺=603.1) were consistent with the desired compound (4).

Part C. Preparation of tert-butyl 4-({4-[5-(4,4,4-trifluorobutyl)-2-pyridyl]phenyl}sulfonyl)piperidine-4-carboxylate (5): 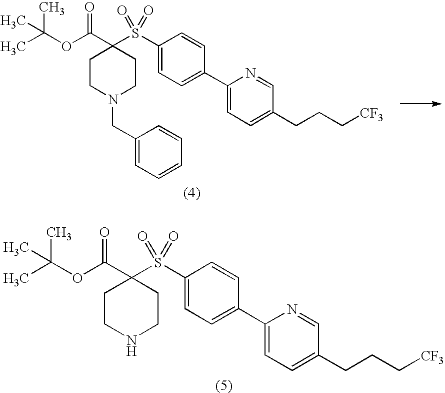
The product (4) from Part B (1.9 g, 3.2 mmol) was dissolved in 4:1 ethanol/formic acid (20 and 5 mL, respectively). Subsequently, 10% Pd/C (1.0 g) was added. The resulting mixture was heated at 55° C. for 1 hr, and then cooled to room temperature. Afterward, the mixture was filtered through Celite to remove the catalyst, and the filtrate was concentrated under reduced pressure. The residue was re-dissolved in water, and the aqueous mixture was made basic with 2.5 N NaOH. The product was then extracted into ethyl acetate. The organic layer was washed with water (3×), washed with and saturated NaCl (1×), and dried over anhydrous sodium sulfate. Filtration and evaporation of the solvent under reduced pressure formed a foam. The crude material was purified on SiO₂ (using dichloromethane with a methanol gradient (0-10%)) to afford 0.64 grams of product (40% yield). ¹H NMR and mass spectrometry (MH⁺=513) were consistent with the desired compound (5).

Part D. Preparation of tert-butyl 1-(2-methoxyethyl)-4-({4-[5-(4,4,4-trifluorobutyl)(2-pyridyl)]phenyl}sulfonyl)piperidine-4-carboxylate (6): 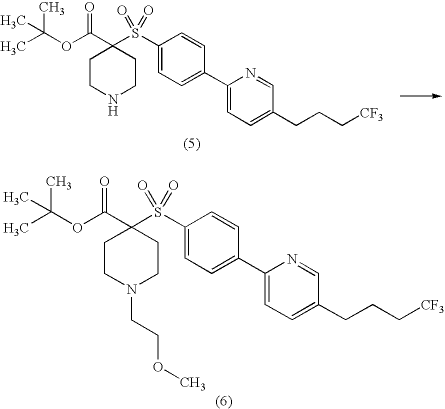
The product (5) from Part C (0.75 g, 1.5 mmol), 2-bromoethyl methyl ether (0.24 g, 1.8 mmol), and diisopropylethylamine (“DIEA”, 0.23 g, 1.8 mmol) were dissolved in DMF (25 mL). The reaction was then continued overnight at room temperature. Afterward, starting material (5) was still present. Additional bromide and diisopropylethylamine were therefore added, and the mixture was stirred at 45° C. overnight, after which LC/MS indicated that the reaction was complete. The mixture was then diluted with ethyl acetate, and the organic layer was washed with water (3×), washed with saturated NaCl (1×), and dried over anhydrous sodium sulfate. Filtration and evaporation of the solvent under reduced pressure formed an oil (0.81 g, 95% yield). ¹H NMR and mass spectrometry (MH⁺=571.2) were consistent with the desired compound (6). This material was used in Step E without further purification.

Part E. Preparation of the trifluoroacetic acid salt of 1-(2-methoxyethyl)-4-({4-[5-(4,4,4-trifluorobutyl)(2-pyridyl)]phenyl}sulfonyl)piperidine-4-carboxylic acid (7): 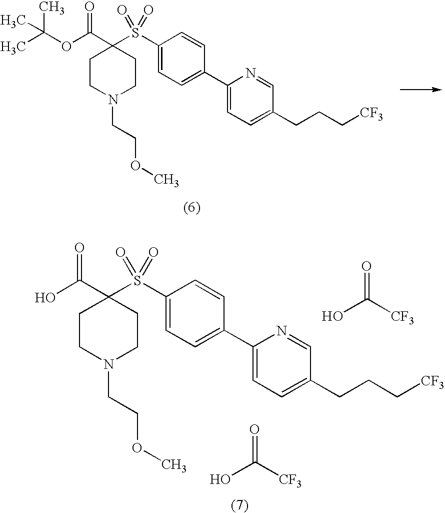
The product (6) from Part D (0.80 g, 1.2 mmol) was dissolved in 1:1 trifluoroacetic acid/dichloromethane (“TFA/CH₂Cl₂”, 30 mL). The reaction was then continued overnight at room temperature, after which no starting material (6) was detected by HPLC. The mixture was concentrated under reduced pressure. The resulting residue was stripped from diethyl ether several times under reduced pressure, and then dried under high vacuum to afford 1.3 g of product (quantitative yield for the di-TFA salt+1 extra mol of TFA). Mass spectrometry (MH⁺=515.1) was consistent with the desired product (7).

Part F. Preparation of [1-2-methoxyethyl)-4-({4-[5-(4,4,4-trifluorobutyl)(2-pyridyl)]phenyl}sulfonyl)(4-piperidyl)]-N-perhydro-2H-pyran-2-yloxycarboxamide (8): 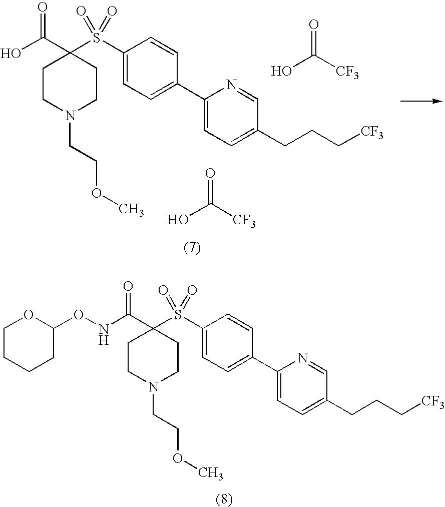
To a mixture of the product (7) from Part E (1.3 g, 1.5 mmol for “tri-TFA”) in N,N-dimethylformamide (“DMF”, 40 mL) were added N-hydroxybenzotriazole (“HOBt”, 0.29 g, 2.1 mmol), 4-methylmorpholine (“NMM”, 0.76 g, 0.83 mL, 7.5 mmol), 1-(3-dimethylaminopropyl)-3-ethylcarbodiimide hydrochloride (“EDC.HCl”, 1.0 g, 5.3 mmol), and O-(tetrahydro-2H-pyran-2-yl)hydroxylamine (“THPONH₂”, 0.62 g, 5.3 mmol). The reaction was then continued overnight at room temperature under N₂, after which no starting material (7) was detected by HPLC. The mixture was diluted with ethyl acetate. The organic layer was extracted with water (3 times) and saturated sodium bicarbonate (3 times), washed with saturated NaCl, and dried over anhydrous sodium sulfate. Filtration and evaporation of the solvent under reduced pressure formed an oil. The crude material was purified by flash chromatography (using dichloromethane with a methanol gradient (0-3%)) to afford 0.46 g of a white foam (50% yield). ¹H NMR and mass spectrometry (MH⁺=614.2) were consistent with the desired product (8).

Part G. Preparation of 1-(2-methoxyethyl)-4-({4-[5-(4,4,4-trifluorobutyl)(2-pyridyl)]phenyl}sulfonyl)piperidine-4-carbohydroxamic acid, dihydrochloride (9): 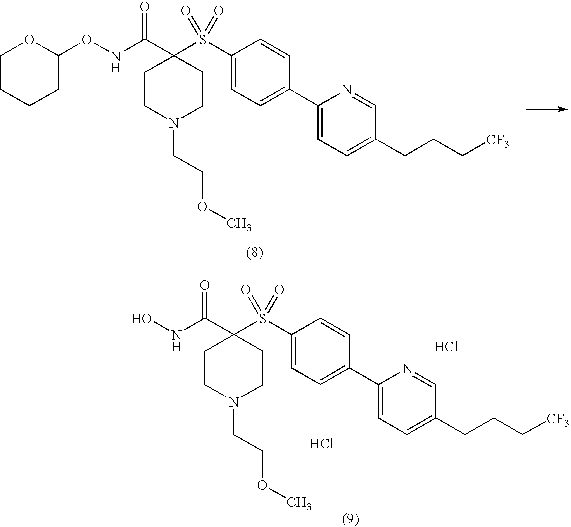
The product (8) from Part F (0.46 g, 0.75 mmol) was dissolved in dioxane (4 mL), 4N HCl in dioxane (5 mL), and methanol (0.5 mL). The reaction was then continued at ambient temperature for 2 hr, after which HPLC indicated that the reaction was complete. The mixture was concentrated under reduced pressure. The resulting residue was triturated with diethyl ether to form a white solid, which, in turn, was collected by suction filtration to form 0.40 g of product (quantitative yield). ¹H NMR and high resolution mass spectrometry (theoretical MH⁺=530.1931, actual MH⁺=530.1921) were consistent with the desired compound (9).

### Example 21

### Preparation of N-hydroxy-4-({4-[5-(3,3,4,4,4-pentafluorobutyl)pyrazin-2-yl]phenyl}sulfonyl)tetrahydro-2H-pyran-4-carboxamide hydrochloride

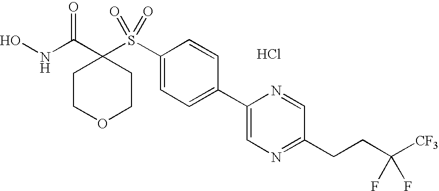

Part A. Preparation of 5-bromo-pyrazin-2-ylanine (2): 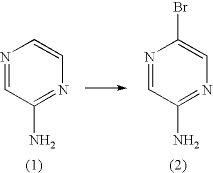
To a CH₂Cl₂ (150 mL) solution of aminopyrazine (1) (5.90 g, 62 mmol) in an ice bath was added N-bromosuccinimide (“NBS”, 11.1 g, 62 mmol) as a solid. The resulting mixture was stirred for 1 hr to form a brown slurry. The slurry was poured into 2 N Na₂CO₃ (150 mL), and extracted with CH₂Cl₂ (3×100 mL). The combined organic extracts were washed with brine, dried over MgSO₄, and evaporated to afford a brown solid. The crude material was purified on silica gel (eluting with 20-40% ethyl acetate in hexane) to afford 5.80 g (54% yield) of the desired compound (2) as an off-white solid. LCMS: m/z=174.0, 176.0 (M+H).

Part B. Preparation of 2-bromo-5-iodo-pyrazine (3): 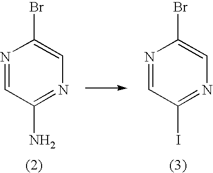
To a mixture of DME (30 mL) and the product (2) from Part A (1.25 g, 7.2 mmol) was added CsI (1.86 g, 7.2 mmol), iodine (0.92 g, 3.6 mmol), CuI (0.42 g, 2.2 mmol), and isoamyl nitrite (5.8 mL, 43.2 mmol). The dark mixture was heated to 60° C., causing gas evolution. After heating for 35 min, and the mixture was cooled to room temperature, partitioned between saturated aqueous NH₄Cl (100 mL) and EtOAc (100 mL), and filtered through celite. The organic layer was separated, washed with 5% Na₂S₂O₃, dried over MgSO₄, and evaporated to afford 1.50 g (74% yield) of the desired product (3) as an yellow solid. GCMS: m/z=284, 286 (M⁺).

Part C. Preparation of 4-[4-(5-bromo-pyrazin-2-yl)-benzenesulfonyl]-tetrahydro-pyran-4-carboxylic acid tert-butyl ester (5): 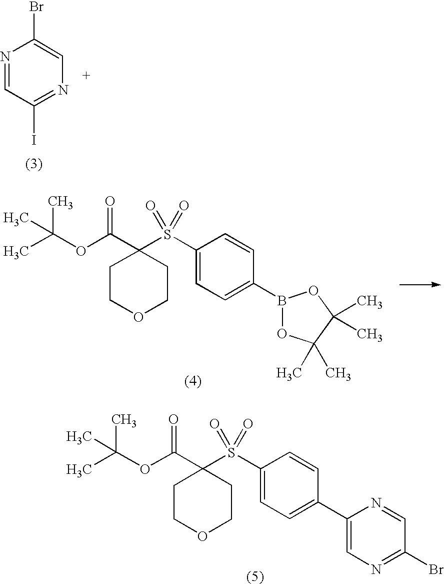
To a slurry of 4-[4-(4,4,5,5-tetramethyl-[1,3,2]dioxaborolan-2-yl)-benzenesulfonyl]-tetrahydro-pyran-4-carboxylic acid tert-butyl ester (4) (1.22 g, 2.7 mmol) in toluene (8 mL) was added solid product (3) from Part B (0.78 g, 2.7 mmol), 2 N Na₂CO₃ (5 mL), ethanol (2.5 mL), and Pd(dppf)Cl₂ (0.11 g, 0.13 mmol). The resulting mixture was heated to 75° C. for 3 hr. The mixture was then partitioned between EtOAc and water, and filtered to remove insolubles. The organic layer was separated, washed with brine, dried over MgSO₄, and evaporated to a form brown solid. The crude material was purified by flash column chromatography on silica gel (eluting with 10-50% ethyl acetate in hexane) to afford 0.70 g (54% yield) of the desired product (5) as an off-white solid. LCMS: m/z=505, 507.

Part D. Preparation of 4-{4-[5-(3,3,4,4,4-pentafluoro-butyl)-pyrazin-2-yl]-benzenesulfonyl}-tetrahydro-pyran-4-carboxylic acid tert-butyl ester (6): 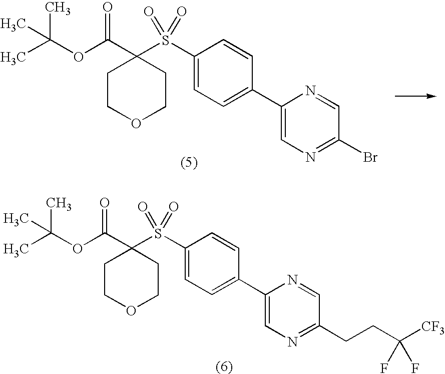
To a slurry of Zn dust (12.1 g, 186 mmol) in THF (30 mL) was added dibromoethane (1.90 mL, 22 mmol). The resulting slurry was heated to reflux briefly and cooled three times. Neat chlorotrimethylsilane (2.8 mL, 22 mmol) was then slowly added, and the mixture was stirred for 15 min. Neat 4-iodo-1,1,1,2,2-pentafluorobutane (32.5 g, 125 mmol) was added slowly, causing an exothermic reaction. The zinc mixture was stirred for 1 hr at room temperature. Subsequently, the supernatant was transferred by cannula into a DMA (100 mL) mixture of the product (5) from Part C (36.6 g, 76 mmol) and Pd(P(o-tolyl)₃)₂Cl₂ (3.16 g, 4.0 mmol). After heating for 30 min at 90° C., the mixture was quenched with saturated NH₄Cl (50 mL), and partitioned between EtOAc (800 mL) and water (500 mL). After filtering through Celite, the organic layer was separated, washed with brine, dried over MgSO₄, and evaporated to form a brown solid. The crude material was purified through a plug of silica gel (150 g) (eluting with 5% ethyl acetate in hexane) to afford 24.0 g (70% yield) of the desired product (6) as an off-white solid. LCMS: m/z=551.2 (M+H).

Part E. Preparation of 4-{4-[5-(3,3,4,4,4-pentafluoro-butyl)-pyrazin-2-yl]-benzenesulfonyl}-tetrahydro-pyran-4-carboxylic acid (tetrahydro-pyran-2-yloxy)-amide (7): 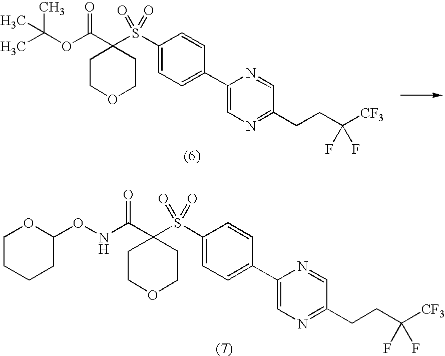
To a mixture of the product (6) from Part D (0.39 g, 0.82 mmol) in CH₂Cl₂ (2 mL) was added trifluoroacetic acid (“TFA”, 4 mL). This mixture was stirred for 3 hr at room temperature. Afterward, the mixture was stripped in vacuo to form a curde carboxylic acid product. To a mixture of the crude carboxylic acid in DMF (5 mL) was added O-(tetrahydro-2H-pyran-2-yl)hydroxylamine (“TBPONH₂”, 0.25 g, 2.1 mmol), 1-(3-dimethylaminopropyl)-3-ethylcarbodiimide hydrochloride (“EDC.HCl”, 0.41 g, 2.1 mmol), 1-hydroxybenzotriazole hydrate (“HOBt”, 0.28 g, 2.1 mmol), and triethylamine (“Et₃N”, 0.39 mL, 2.8 mmol). The resulting mixture was stirred for 16 hr at room temperature. The solvent was then stripped in vacuo, and the residue partitioned between ethyl acetate and water. The organic layer was separated, washed with saturated NaHCO₃ and brine, dried over MgSO₄, and evaporated to an oil. The crude material was purified by flash column chromatography on silica gel (eluting with 20-40% ethyl acetate (containing 10% methanol) in hexane) to afford 0.31 g (74% yield) of the desired THP protected hydroxamic acid (7) as a pale yellow foam. LCMS: m/z=616.2

Part F. Preparation of N-hydroxy-4-({4-[5-(3,3,4,4,4-pentafluorobutyl)pyrazin-2-yl]phenyl}sulfonyl)tetrahydro-2H-pyran-4-carboxamide hydrochloride (8): 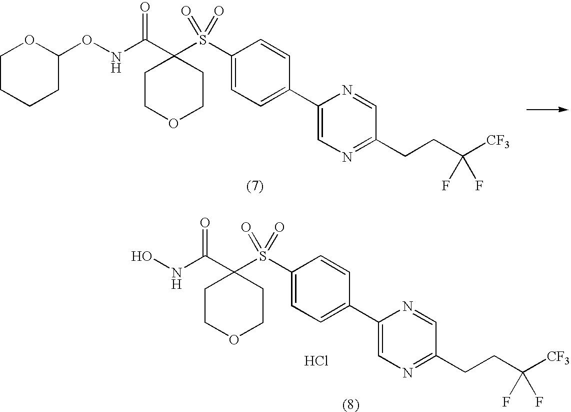
To solid product (7) from Part E (0.30 g, 0.51 mmol) was added methanol (0.3 mL) and 4 N HCl in dioxane (3.0 mL). The resulting colorless mixture was stirred for 1.5 hr. The mixture was then diluted with diethyl ether (25 mL). The resulting cloudy mixture was stirred for 3 hr. Hexane was then added (25 mL), and the slurry was concentrated under N₂ by half. Afterward, the slurry was filtered, and the solid was washed with hexane (2×20 mL). The precipitate was dried in vacuo for 16 hr to afford 0.23 g (82% yield) of the desired product as a hydrochloride salt (8). LCMS: m/z=510.1 (M+H). HRMS calcd. for C₂₀H₂₁F₅N₃O₅S: m/z=510.1117 [M+H]⁺, found: 510.1117.

### Example 22

### Preparation of 4-{[4′-(3,3-difluorobutyl)-1,1′-biphenyl-4-yl]sulfonyl}-N-hydroxytetrahydro-2H-pyran-4-carboxamide:

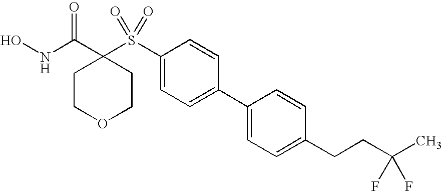

Part A. Preparation of trifluoro-methanesulfonic acid 4-(3-oxo-butyl)-phenyl ester (2): 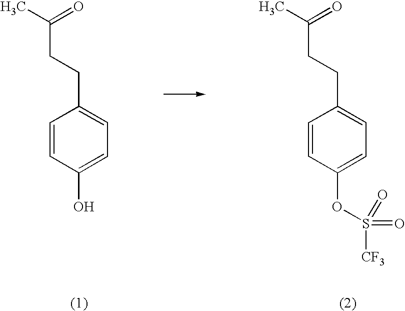
To a mixture of CH₂Cl₂ (30 mL) and 4-(4-hydroxyphenyl)-2-butanone (1.64 g, 10.0 mmol) were added 2-[N,N-bis(trifluoromethylsulfonyl)amino]-5-chloropyridine (3.93 g, 10.0 mmol) and triethylamine (1.40 mL, 10 mmol). The resulting mixture was stirred for 2.5 hr at room temperature, and then diluted with ethyl acetate (100 mL). The organic layer was washed with brine, dried over MgSO₄, and evaporated to form a brown oil. The crude material was purified on silica gel (eluting with 20% ethyl acetate in hexane) to afford 2.80 g (95% yield) of the desired product (2) as an oil. LCMS: m/z=297.0 (M+H).

Part B. Preparation of trifluoro-methanesulfonic acid 4-(3,3-difluoro-butyl)-phenyl ester (3): 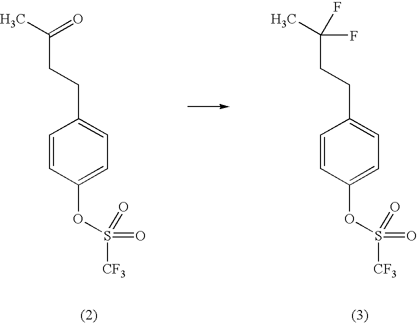
To a mixture of CH₂Cl₂ (3 mL) and the product (2) from Part A (2.80 g, 9.5 mmol) in a 30 mL Teflon bottle was added [bis(2-methoxyethyl)amino]sulfur trifluoride (3.76 g, 17 mmol). Ethanol (0.116 mL, 2.0 mmol) was then added, and the resulting mixture was stirred for 16 hr at room temperature under N₂. The mixture was then slowly added to saturated NaHCO₃ (50 mL). After gas evolution stopped, the mixture was extracted with CH₂Cl₂ (3×25 mL). The combined organic layers were dried over MgSO₄ and evaporated to form a yellow oil. The crude material was purified on silica gel (eluting with 10-20% diethyl ether in hexane) to afford 2.34 g (78% yield) of the desired product (3) as a clear colorless liquid. GCMS: m/z=318 (M⁺).

Part C. Preparation of 4-[4′-(3,3-difluoro-butyl)-biphenyl-4-sulfonyl]-tetrahydro-pyran-4-carboxylic acid tert-butyl ester (5): 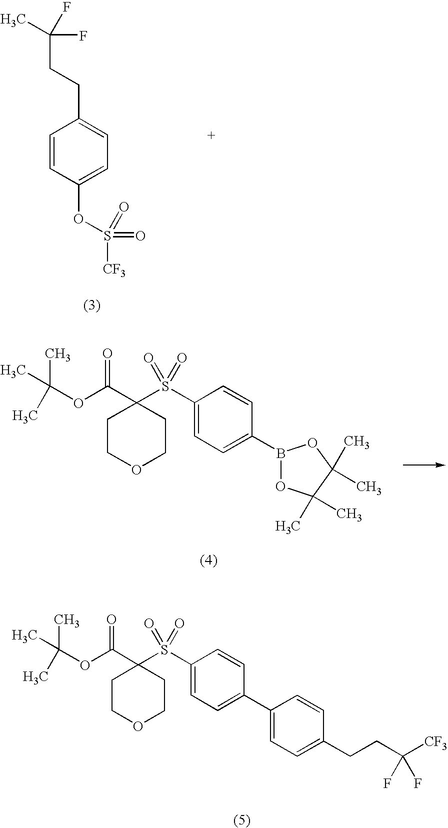
To a slurry of 4-[4-(4,4,5,5-tetramethyl-[1,3,2]dioxaborolan-2-yl)-benzenesulfonyl]-tetrahydro-pyran-4-carboxylic acid tert-butyl ester (4) (1.4 g, 3.0 mmol) in toluene (8 mL) was added solid product (3) from Part B (1.0 g, 3.1 mmol), 2 N Na₂CO₃ (5 mL), ethanol (2.5 mL), and Pd(dppf)Cl₂ (0.15 g, 0.20 mmol). The resulting mixture was refluxed 16 hr. A second portion of Pd(dppf)Cl₂ (0.15 g, 0.20 mmol) was then added, and the mixture was again refluxed for an additional 24 hr. The mixture was then partitioned between EtOAc and water. Afterward, the mixture was filtered to remove insolubles. The organic layer was separated, washed with brine, dried over MgSO₄, and evaporated to a brown solid. The crude material was purified on silica gel (eluting with 10-40% ethyl acetate in hexane) to form an off-white solid. The product was further purified by triturating with diethyl ether:hexane (1:1). The resulting precipitate was filtered and washed with hexane to afford 0.66 g (44% yield) of the desired product (5) as a white solid. LCMS: m/z=517.2 (M+H).

Part D. Preparation of 4-[4′-(3,3-difluoro-butyl)-biphenyl-4-sulfonyl]-tetrahydro-pyran-4-carboxylic acid (tetrahydro-pyran-2-yloxy)-amide (6): 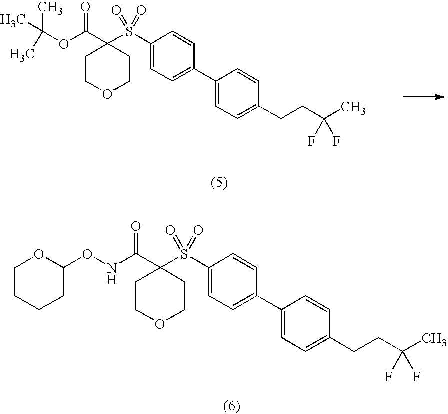
To a mixture of the product (5) from Part C (0.51 g, 1.0 mmol) in CH₂Cl₂ (2 mL) was added trifluoroacetic acid (“TFA”, 4 mL) and the solution was stirred 3 hr at room temperature. The solution was stripped in vacuo to form a crude carboxylic acid product. To a mixture of the crude carboxylic acid in DMF (5 mL) was added O-(tetrahydro-2H-pyran-2-yl)hydroxylamine (“THPONH₂”, 0.35 g, 3.0 mmol), 1-(3-dimethylaminopropyl)-3-ethylcarbodiimide hydrochloride (“EDC.HCl”, 0.58 g, 3.0 mmol), 1-hydroxybenzotriazole hydrate (“HOBt”, 0.41 g, 3.0 mmol), and triethylamine (“Et₃N”, 0.56 mL, 4.0 mmol). The reaction mixture was stirred 16 hr at room temperature, the solvent was stripped in vacuo, and the residue partitioned between ethyl acetate and water. The organic layer was separated, washed with saturated NaHCO₃ and brine, dried over MgSO₄, and evaporated to an oil. The crude material was purified by flash column chromatography on silica gel (eluting with 10-60% ethyl acetate in hexane) to afford 0.30 g (56% yield) of the desired THP protected hydroxamic acid (7) as a white solid. LCMS: m/z=560.2 (M+H).

Part E. Preparation of 4-{[4′-(3,3-difluorobutyl)-1,1′-biphenyl-4-yl]sulfonyl}-N-hydroxytetrahydro-2H-pyran-4-carboxamide (7): 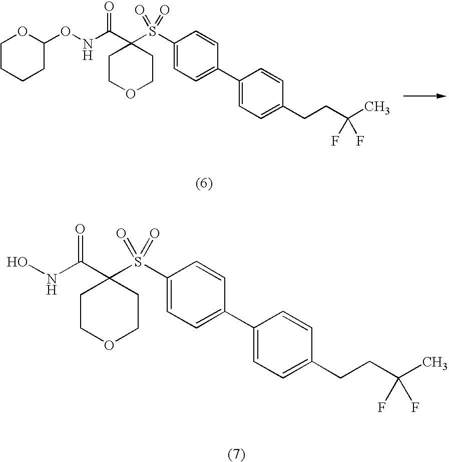
To solid product (6) from Part D (0.30 g, 0.60 mmol) was added MeOH (0.3 mL) and 4 N HCl in dioxane (3.0 mL). The resulting mixture was stirred for 2.0 hr at room temperature. Afterward, the mixture was added to 30 mL of 1:1 diethyl ether:hexane, and the resulting cloudy solution was concentrated by half. After stirring for 2 hr, the slurry was filtered, and the solid washed with hexane (2×20 mL). The precipitate was dried in vacuo for 16 hr to afford 0.20 g (79% yield) of the desired hydroxamic acid product (7) as a white solid. LCMS: m/z=454.0 (M+H). HRMS calcd. for C₂₂H₂₆F₂NO₅S: m/z=454.1494 [M+H]⁺, found: 454.1445.

### Example 23

### Preparation of 4-{[4′-(4,4-difluorobutyl)-1,1′-biphenyl-4-yl]sulfonyl}-N-hydroxytetrahydro-2H-pyran-4-carboxamide

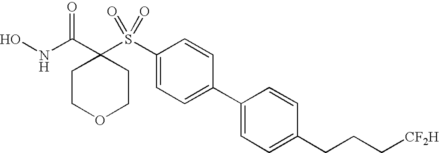

Part A. Preparation of 4-(4-iodo-phenyl)-butyraldehyde (2): 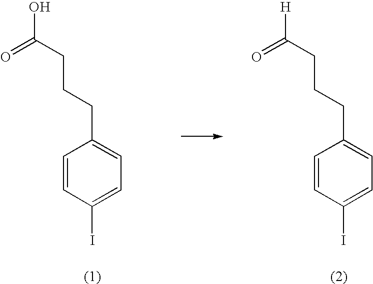
To an ice-cooled THF (20 mL) mixture of 4-(4-iodophenyl)butanoic acid (1) (2.90 g, 10.0 mmol) was added borane-tetrahydrofuran complex (20 mL, 1.0 M, 20 mmol) dropwise over 30 min. The resulting mixture was stirred for 1.5 hr at room temperature, and then quenched with a 1:1 HOAc/MeOH (1 mL). The solvent was stripped, and the residue was then partitioned between EtOAc and water. The organic layer was separated, washed with saturated NaHCO₃ and brine, dried over MgSO₄, and evaporated to produce a quantitative yield of the crude alcohol as an oil. To a CH₂Cl₂ (12 mL) mixture of the crude alcohol (1.75 g, 6.3 mmol) was added 4-methylmorpholine N-oxide (1.11 g, 9.5), 4 Å powdered molecular sieves (3 g), and tetrapropylammonium perruthenate (“TRAP”, 0.11 g, 0.3 mmol). The resulting slurry was stirred for 1 hr at room temperature. The crude material was purified on silica gel (eluting with 5-50% ethyl acetate in hexane) to afford 1.11 g (64% yield) of the desired aldehyde product (2) as an oil. LCMS: m/z=257.0 (M+H—H₂O).

Part B. Preparation of 1-(4,4-difluoro-butyl)-4-iodo-benzene (3): 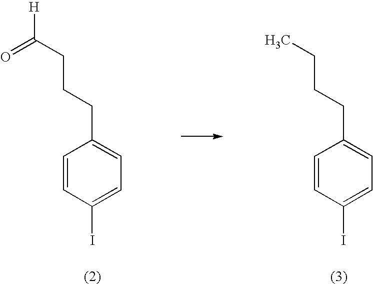
To a CH₂Cl₂ (4 mL) mixture of the product (2) from Part A (1.1 g, 4.1 mmol) in a 30 mL Teflon bottle was added [bis(2-methoxyethyl)amino]sulfur trifluoride (1.27 g, 6.9 mmol). Ethanol (0.023 mL, 0.41 mmol) was added, and the mixture was then stirred for 16 hr at room temperature under N₂. The mixture was slowly added to saturated NaHCO₃ (50 mL), and after gas evolution stopped, it was extracted with CH₂Cl₂ (3×25 mL). The combined organic layers were dried over MgSO₄, and evaporated to a yellow oil. The crude material was purified on silica gel (eluting with 10-20% diethyl ether in hexane) to afford 0.93 g (77% yield) of the desired product (3) as a clear colorless liquid. GCMS: m/z=296 (M⁺).

Part C. Preparation of 4-[4′-(4,4-difluoro-butyl)-biphenyl-4-sulfonyl]-tetrahydro-pyran-4-carboxylic acid tert-butyl ester (5): 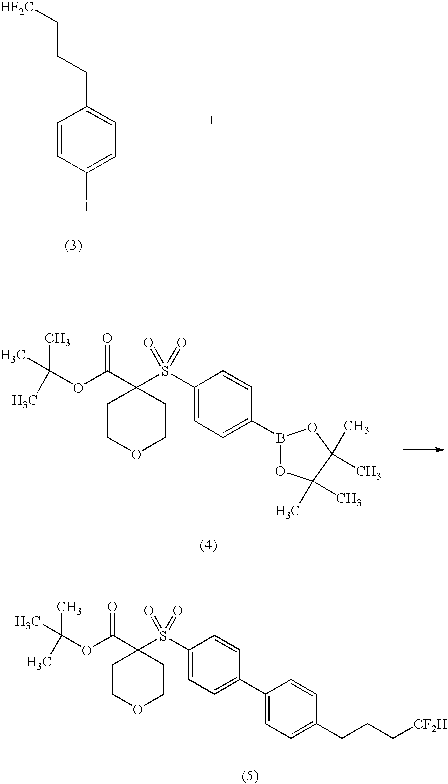
To a slurry of 4-[4-(4,4,5,5-tetramethyl-[1,3,2]dioxaborolan-2-yl)-benzenesulfonyl]-tetrahydro-pyran-4-carboxylic acid tert-butyl ester (4) (1.4 g, 3.0 mmol) in toluene (8 mL) was added solid the product (3) from Part B (0.86 g, 2.9 mmol), 2 N Na₂CO₃ (5 mL), ethanol (2.5 mL), and Pd(dppf)Cl₂ (0.15 g, 0.20 mmol). The resulting mixture was refluxed for 16 hr. A second portion of Pd(dppf)Cl₂ (0.15 g, 0.20 mmol) was added, and the mixture was again refluxed for an additional 24 hr. Afterward, the mixture was partitioned between EtOAc and water, and then filtered to remove insolubles. The organic layer was separated, washed with brine, dried over MgSO₄, and evaporated to form a brown solid. The crude material was purified on silica gel (eluting with 10-40% ethyl acetate in hexane) to afford 0.88 g (62% yield) of the desired product (5) as a white solid. LCMS: m/z=517.2 (M+H).

Part D. Preparation of 4-[4′-(4,4-difluoro-butyl)-biphenyl-4-sulfonyl]-tetrahydro-pyran-4-carboxylic acid (tetrahydro-pyran-2-yloxy)-amide (6): 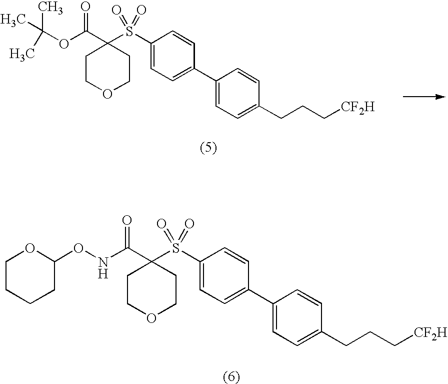
To a mixture of the product (5) from Part C (0.88 g, 1.8 mmol) in CH₂Cl₂ (2 mL) was added trifluoroacetic acid (“TFA”, 4 mL). The resulting mixture was stirred for 3 hr at room temperature, and then stripped in vacuo to form a curde carboxylic acid. To a mixture of the crude acid in DMF (8 mL) was added O-(tetrahydro-2H-pyran-2-yl)hydroxylamine (“THPONH₂”, 0.62 g, 5.3 mmol), 1-(3-dimethylaminopropyl)-3-ethylcarbodiimide hydrochloride (“EDC.HCl”, 1.03 g, 5.3 mmol), 1-hydroxybenzotriazole hydrate (“HOBT”, 0.72 g, 5.3 mmol), and triethylamine (“Et₃N”, 1.0 mL, 7.2 mmol). The resulting mixture was stirred for 16 hr at room temperature. Afterward, the solvent was stripped in vacuo, and the residue was partitioned between ethyl acetate and water. The organic layer was separated, washed with saturated NaHCO₃ and brine, dried over MgSO₄, and evaporated to form an oil. The crude material was purified by flash column chromatography on silica gel (eluting with 20-80% ethyl acetate in hexane) to afford 0.76 g (79% yield) of the desired THP protected hydroxamic acid (6) as a white solid. LCMS: m/z=560.2 (M+H).

Part E. Preparation of 4-{[4′-(4,4-difluorobutyl)-1,1′-biphenyl-4-yl]sulfonyl}-N-hydroxytetrahydro-2H-pyran-4-carboxamide (7): 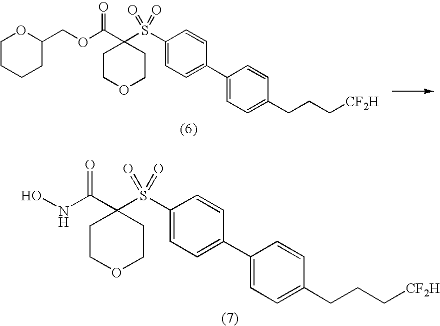
To solid product (6) from Part D (0.75 g, 1.4 mmol) was added methanol (0.4 mL) and 4 N HCl in dioxane (4.0 mL). The resulting mixture was stirred for 1.5 hr at room temperature. The mixture was then added to 30 mL of 1:1 diethyl ether:hexane. The resulting cloudy mixture was concentrated by half to form an oily precipitate. The oil was triturated with Et₂O to form a solid white precipitate. The slurry was filtered. The resulting solid was washed with Et₂O (20 mL). The precipitate was then dried in vacuo for 16 hr to afford 0.41 g (65% yield) of the desired hydroxamic acid product (8) as a white solid. LCMS: m/z=454.1 (M+H). HRMS calcd. for C₂₂H₂₆F₂NO₅S: m/z=454.1494 [M+H]⁺, found: 454.1468.

### Example 24

### Preparation of N-hydroxy-4-({4-[5-(4,4,4-trifluorobutyl)pyrazin-2-yl]phenyl}sulfonyl)tetrahydro-2H-pyran-4-carboxamide hydrochloride:

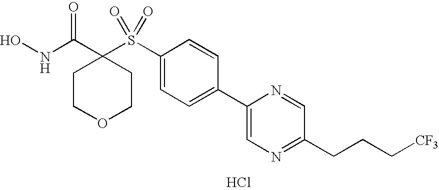

Part A. Preparation of 4-{4-[5-(4,4,4-trifluoro-butyl)-pyrazin-2-yl]-benzenesulfonyl}-tetrahydro-pyran-4-carboxylic acid tert-butyl ester (2): 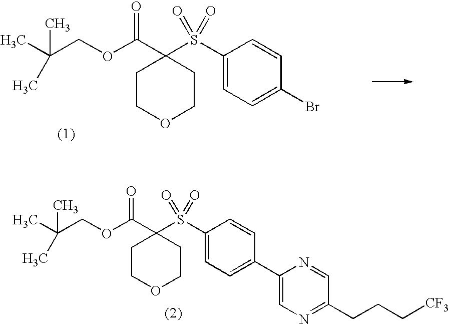
To a slurry of Zn dust (1.24 g, 18.6 mmol) in THF (3 mL) was added dibromoethane (0.19 mL, 2.2 mmol). The resulting slurry was heated to reflux briefly, and cooled 3 times. Neat chlorotrimethylsilane (0.28 mL, 2.2 mmol) was slowly added, and then the mixture was stirred for 15 min. Neat 4-iodo-1,1,1-trifluorobutane (3.6 g, 13.9 mmol) was added, which slowly caused an exothermic reaction. The zinc mixture was stirred 1 hr at room temperature and 1 hr at 60° C. The supernatant of the resulting mixture was transferred by canulla into mixture of DMA (20 mL), 4-[4-(5-bromo-pyrazin-2-yl)-benzenesulfonyl]-tetrahydro-pyran-4-carboxylic acid tert-butyl ester (1) (4.0 g, 8.3 mmol, made in accordance with from Part C of Example 21), and Pd(P(o-tolyl)₃)₂Cl₂ (0.33 g, 0.4 mmol). After heating 1 hr at 60° C., the reaction mixture was quenched with saturated NH4Cl (5 mL), and partitioned between EtOAc (100 mL) and water (50 mL). After filtering through Celite, the organic layer was separated, washed with brine, dried over MgSO₄, and evaporated to form a brown solid. The crude material was purified by flash column chromatography on silica gel (eluting with 5-50% ethyl acetate in CH₂Cl₂) to afford 2.64 g (62% yield) of the desired product (2) as a white solid. LCMS: m/z=515.2 (M+H).

Part B. Preparation of 4-{4-[5-(4,4,4-trifluoro-butyl)-pyrazin-2-yl]-benzenesulfonyl}-tetrahydro-pyran-4-carboxylic acid (tetrahydro-pyran-2-yloxy)-amide (3): 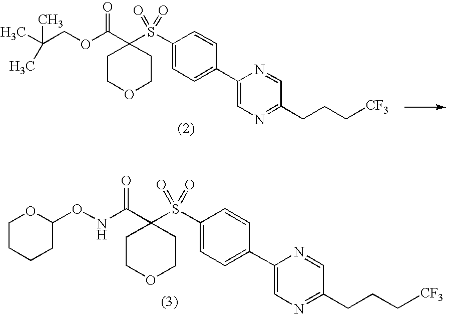
To a mixture of the product (2) from Part A (2.58 g, 5.0 mmol) in CH₂Cl₂ (3 mL) was added trifluoroacetic acid (“TFA”, 6 mL) and the solution was stirred 3 hr at room temperature. The solution was stripped in vacuo to form a crude carboxylic acid product. To a mixture of the crude carboxylic acid product in DMF (20 mL) was added O-(tetrahydro-2H-pyran-2-yl)hydroxylamine (“THPONH₂”, 1.61 g, 13.8 mmol), 1-(3-dimethylaminopropyl)-3-ethylcarbodiimide hydrochloride (“EDC.HCl”, 2.65 g, 13.8 mmol), 1-hydroxybenzotriazole hydrate (“HOBT”, 1.86 g, 13.8 mmol), and triethylamine (“Et₃N”, 2.6 mL, 19 mmol). The reaction mixture was stirred 16 hr at room temperature. The solvent was stripped in vacuo, and the residue partitioned between ethyl acetate and water. The organic layer was separated, washed with saturated NaHCO₃ and brine, dried over MgSO₄, and evaporated to an oil. The crude material was purified by flash column chromatography on silica gel (eluting with 20-80% ethyl acetate (containing 20% CH₃CN) in hexane) to afford 2.49 g (97% yield) of the desired THP protected hydroxamic acid (4) as a pale yellow foam. LCMS: m/z=580.2 (M+Na)

Part C. Preparation of N-hydroxy-4-({4-[5-(4,4,4-trifluorobutyl)pyrazin-2-yl]phenyl}sulfonyl)tetrahydro-2H-pyran-4-carboxamide hydrochloride (4): 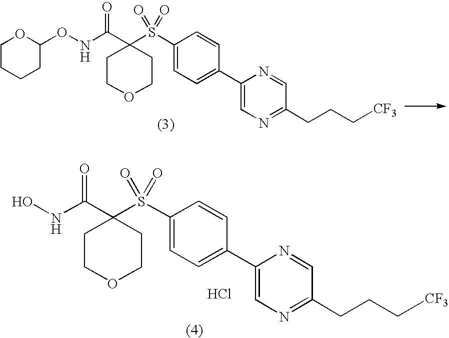
To a mixture of EtOAc (40 mL) and the product (3) from Part B (2.37 g, 4.25 mmol) was added 1.25 N HCl in ethanol (5 mL). The resulting colorless mixture was stirred for 2 hr to form a white precipitate. The slurry was diluted with hexanes (20 mL) and stirred for 1 hr. Subsequently, the slurry was filtered, and the resulting solid was washed with hexane (2×15 mL). The precipitate was then dried in vacuo for 16 hr to afford 1.82 g (84% yield) of the desired product as a hydrochloride salt (4). LCMS: m/z=474.2 (M+H). HRMS calcd. for C₂₀H₂₁F₃N₃O₅S: m/z=472.1149 [M−H]⁻, found: 472.1157.

### Example 25

### Preparation of 1-cyclopropyl-4-{[4′-(3,3-difluorobutyl)-1,1′-biphenyl-4-yl]sulfonyl}-N-hydroxypiperidine-4-carboxamide hydrochloride

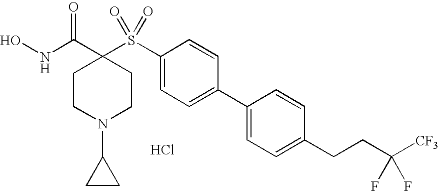

Part A. Preparation of 1-benzyl-4-[4′-(3,3-difluoro-butyl)-biphenyl-4-sulfonyl]-piperidine-4-carboxylic acid tert-butyl ester (3): 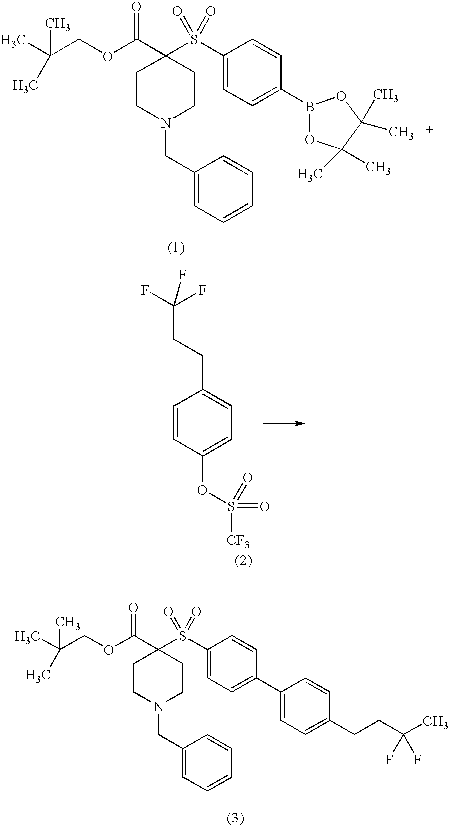
To a slurry of 1-benzyl-4-[4-(4,4,5,5-tetramethyl-[1,3,2]dioxaborolan-2-yl)-benzenesulfonyl]-piperidine-4-carboxylic acid tert-butyl ester (1) (2.2 g, 4.1 mmol) in toluene (10 mL) was added solid trifluoro-methanesulfonic acid 4-(3,3-difluoro-butyl)-phenyl ester (2) (1.3 g, 4.1 mmol, made in accordance with Part B of Example 22), 2 N Na₂CO₃ (6 mL), ethanol (3 mL), and Pd(dppf)Cl₂ (0.15 g, 0.20 mmol). The resulting mixture was refluxed for 16 hr. Subsequently, the mixture was partitioned between EtOAc and water, and then filtered to remove insolubles. The organic layer was separated, washed with brine, dried over MgSO₄, and evaporated to form a brown solid. The crude material was purified on silica gel (eluting with 20-50% ethyl acetate (containing 20% CH₃CN) in hexane) to afford 1.92 g (80% yield) of the desired product (3) as an oil. LCMS: m/z=584.2 (M+H).

Part B. Preparation of 4-[4′-(3,3-difluoro-butyl)-biphenyl-4-sulfonyl]-piperidine-4-carboxylic acid tert-butyl ester (4): 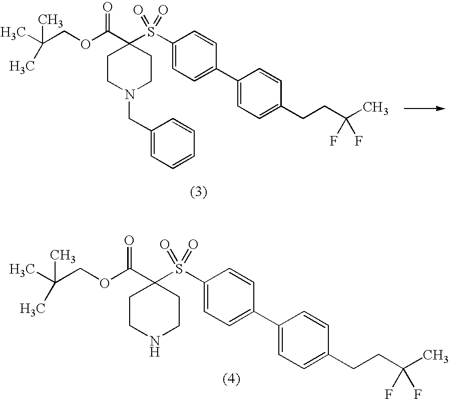
To a MeOH (30 mL) solution of the product (3) from Part A (1.7 g, 2.9 mmol) was added cyclohexene (3mL) and 10% Pd/C (0.30 g). The slurry was refluxed for 6 hr, and then cooled to room temperature. Afterward, the catalyst was removed by filtration through Celite. The filtrate was stripped to afford 1.35 g (94% yield) of the desired product (4) as an off-white foam. LCMS: m/z=494.2 (M+H).

Part C. Preparation of 1-cyclopropyl-4-[4′-(3,3-difluoro-butyl)-biphenyl-4-sulfonyl]-piperidine-4-carboxylic acid tert-butyl ester (5): 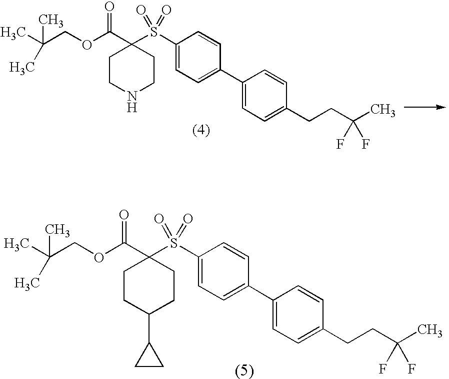
To a mixture of methanol (5 mL) and the product (4) from Part B (0.73 g, 1.5 mmol) was added [(1-ethoxycyclopropyl)oxy]trimethylsilane (0.38 g, 2.2 mmol), sodium cyanoborohydride (NaBH₃CN, 0.14 g, 2.3 mmol), and HOAc (0.86 mL, 15 mmol). Molecular sieves (3 Å) were added, and the mixture was resulting refluxed for 4 hr. The solvent was stripped, and the residue was partitioned between EtOAc and water. The organic layer was separated, washed with saturated NaHCO₃ and brine, dried over MgSO₄, and evaporated to form an oil. The crude material was purified on silica gel (eluting with 10-40% ethyl acetate (containing 10% CH₃CN) in hexane) to afford 0.35 g (44% yield) of the desired product (5) as a white solid.

Part D. Preparation of 1-cyclopropyl-4-[4′-(3,3-difluoro-butyl)-biphenyl-4-sulfonyl]-piperidine-4-carboxylic acid (tetrahydro-pyran-2-yloxy)-amide (6): 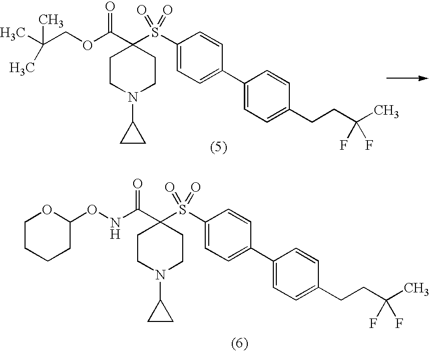
To a mixture of the product (5) from Part C (0.34 g, 0.64 mmol) in CH₂Cl₂ (2 mL) was added trifluoroacetic acid (“TFA”, 3 mL). The resulting mixture was stirred for 3 hr at room temperature. The mixture was then stripped in vacuo to form a carboxylic acid. To a mixture of the crude acid in DMF (5 mL) was added O-(tetrahydro-2H-pyran-2-yl)hydroxylamine (“THPONH₂”, 0.22 g, 1.9 mmol), 1-(3-dimethylaminopropyl)-3-ethylcarbodiimide hydrochloride (“EDC.HCl”, 0.37 g, 1.9 mmol), 1-hydroxybenzotriazole hydrate (“HOBT”, 0.26 g, 1.9 mmol), and triethylamine (“Et₃N”, 0.36 mL, 2.6 mmol). The resulting mixture was stirred for 16 hr at room temperature. Afterward, an additional 0.9 mmol of each of O-(tetrahydro-2H-pyran-2-yl)hydroxylamine, 1-(3-dimethylaminopropyl)-3-ethylcarbodiimide hydrochloride, and 1-hydroxybenzotriazole hydrate was added. After 16 additional hours at room temperature, the solvent was stripped in vacuo, and the resulting residue was partitioned between ethyl acetate and water. The organic layer was separated, washed with saturated NaHCO₃ and brine, dried over MgSO₄, and evaporated to an oil. The crude material was purified by flash column chromatography on silica gel (eluting with 20-80% ethyl acetate (containing 20% CH₃CN) in hexane) to afford 0.24 g (65% yield) of the desired THP protected hydroxamic acid (6) as a white solid. LCMS: m/z=577.3 (M+H).

Part E. Preparation of 1-cyclopropyl-4-[4′-(3,3-difluoro-butyl)-biphenyl-4-sulfonyl]-piperidine-4-carboxylic acid hydroxyamide hydrochloride (7): 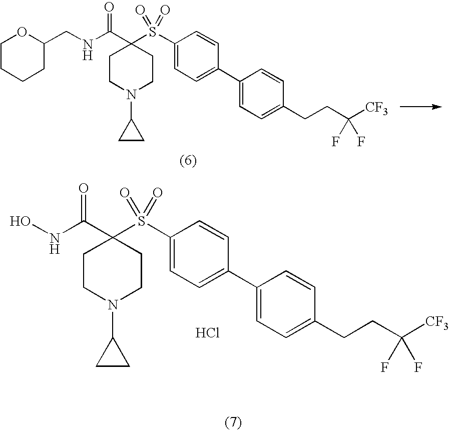
To solid product (6) from Part D (0.24 g, 0.60 mmol) was added methanol (0.2 mL) and 4 N HCl in dioxane (2.0 mL). The resulting mixture was stirred for 1 hr at room temperature. The mixture was then added to 30 mL of Et₂O, and the resulting slurry was stirred for 2 hr. The slurry was filtered, and the solid washed with Et₂O (2×10 mL). The precipitate was dried in vacuo for 16 hr to afford 0.20 g (91% yield) of the desired hydroxamic acid product (7) as a white solid. LCMS: m/z=454.0 (M+H). HRMS calcd. for C₂₅H₃₂F₂N₂O₄S: m/z=493.1967 [M+H]⁺, found: 493.1960.

### Example 26

### Preparation of 4-({4-[5-(3,3-difluorobutyl)pyrazin-2-yl]phenyl}sulfonyl)-N-hydroxytetrahydro-2H-pyran-4-carboxamide hydrochloride

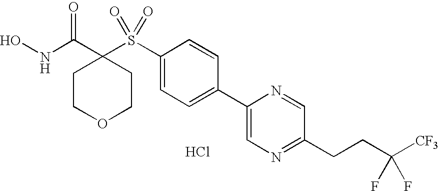

Part A. Preparation of 3,3-difluoro-1-iodo-butane (2): 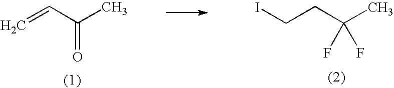
A mixture of dichloromethane (CH₂Cl₂, 50 mL) and methyl vinyl ketone (1) (7.0 g, 100 mmol) was stirred vigorously with aqueous hydroiodic acid (HI, 55-58%, 45 g, 200 mmol) at room temperature. After 2 hr, the organic layer was separated, and washed with saturated NaHCO₃, saturated Na₂S₂O₃, and brine to form a yellow mixture containing crude 4-iodo-2-butanone. The mixture was dried over MgSO₄, filtered, and transferred to a plastic (HDPE) bottle. Neat bis(2-methoxyethyl)aminosulfur trifluoride (37 g, 170 mmol) was slowly added to the mixture. Ethanol (1 mL) was then added dropwise. The resulting dark mixture was stirred 16 hr at room temperature. Subsequently, the mixture was poured into saturated NaHCO₃. The organic layer was separated and washed with brine. The mixture was then distilled at ambient pressure. The fraction boiling at 100-110° C. was collected to afford 6.0 g (27% yield) of the desired product as a colorless oil. ¹H (CDCl₃): δ1.61 (t, 3H), 2.25 (m, 2H), 3.20 (dd, 2H).

Part B. Preparation of 4-{4-[5-(3,3-difluoro-butyl)-pyrazin-2-yl]-benzenesulfonyl}-tetrahydro-pyran-4-carboxylic acid tert-butyl ester (4): 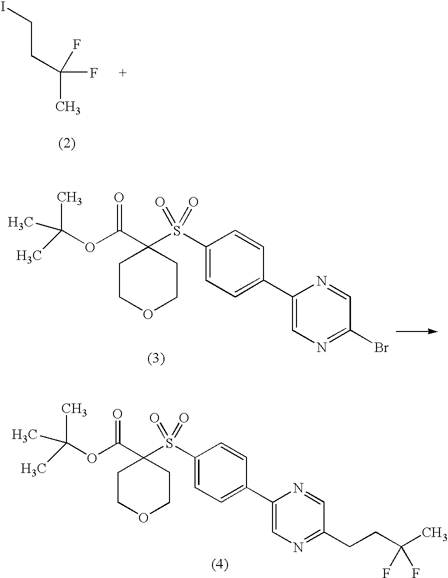
To a slurry Zn dust (3.6 g, 55 mmol) in THF (8 mL) was added dibromoethane (0.42 mL, 4.8 mmol). The slurry was heated to reflux briefly and cooled 3 times. Neat product (2) from Part A (6.0 g, 27 mmol) was added slowly, causing an exothermic reaction. The zinc mixture was subsequently stirred for 2 hr at 60° C. The supernatant of the resulting mixture was transferred by canulla into a mixture of DMA (50 mL), 4-[4-(5-bromo-pyrazin-2-yl)-benzenesulfonyl]-tetrahydro-pyran-4-carboxylic acid tert-butyl ester (3) (8.9 g, 18.5 mmol, prepared in accordance with Example 21, Part C), and Pd(P(o-tolyl)₃)₂Cl₂ (0.72 g, 0.92 mmol). After stirring for 16 hr at room temperature, the reaction mixture was quenched with saturated NH₄Cl, and partitioned between EtOAc and water. After filtering through Celite, the organic layer was separated, washed with brine, dried over MgSO₄, and evaporated to a brown solid. The crude material was purified by flash column chromatography on silica gel (eluting with 20-60% ethyl acetate in hexane). The product was further purified by recrystallization from diethyl ether:hexane. The resulting precipitate was then filtered and washed with hexane to afford 3.42 g (38% yield) of the desired product (4) as a white solid. LCMS: m/z=497.1 (M+H).

Part C. Preparation of 4-{4-[5-(3,3-difluoro-butyl)-pyrazin-2-yl]-benzenesulfonyl}-tetrahydro-pyran-4-carboxylic acid (tetrahydro-pyran-2-yloxy)-amide (5): 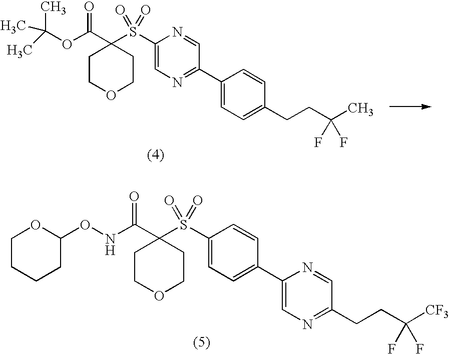
To a mixture of the product (4) from Part B (3.4 g, 6.8 mmol) in CH₂Cl₂ (5 mL) was added trifluoroacetic acid (“TFA”, 10 mL). The resulting mixture was then stirred for 3 hr at room temperature. The solution was stripped in vacuo to form a crude carboxylic acid. To a mixture of the crude carboxylic acid in DMF (25 mL) was added O-(tetrahydro-2H-pyran-2-yl)hydroxylamine (“THPONH₂”, 2.4 g, 20 mmol), 1-(3-dimethylaminopropyl)-3-ethylcarbodiimide hydrochloride (“EDC.HCl”, 3.9 g, 20 mmol), 1-hydroxybenzotriazole hydrate (“HOBT”, 2.8 g, 20 mmol), and triethylamine (“Et₃N”, 3.8 mL, 27 mmol). The mixture was subsequently stirred for 16 hr at room temperature. The solvent was then stripped in vacuo, and the resulting residue partitioned between ethyl acetate and water. The organic layer was separated, washed with saturated NaHCO₃ and brine, dried over MgSO₄, and evaporated to form an oil. The crude material was purified by flash column chromatography on silica gel (eluting with 10-70% ethyl acetate (containing 20% CH₃CN) in hexane) to afford 3.62 g (99% yield) of the desired THP protected hydroxamic acid (5) as a pale yellow foam. LCMS: m/z=544.2 (M+H).

Part D. Preparation of 4-({4-[5-(3,3-difluorobutyl)pyrazin-2-yl]phenyl}sulfonyl)-N-hydroxytetrahydro-2H-pyran-4-carboxamide hydrochloride (6): 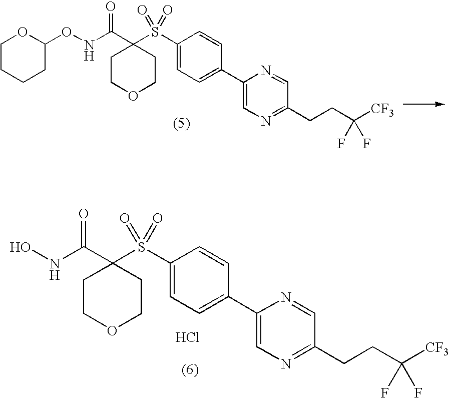
To a mixture of EtOAc (70 mL) and the product (5) from Part C (4.86 g, 9.1 mmol) was added 1.25 N HCl in ethanol (10 mL). The resulting colorless mixture was stirred for 2 hr, forming a white precipitate. The slurry was diluted with diethyl ether (100 mL), and then stirred 1 hr. Afterward, the slurry was filtered. The solid was then washed with Et₂O (2×20 mL). The precipitate was then dried in vacuo for 16 hr to afford 3.88 g (87% yield) of the desired product as a hydrochloride salt (6). LCMS: m/z=456.1 (M+H). HRMS calcd. for C₂₀H₂₄F₂N₃O₅S: m/z=456.1399 [M+H]⁺, found: 456.1396.

### Example 27

### Preparation of 4-{[4′-(3,3-difluorobutyl)-1,1′-biphenyl-4-yl]sulfonyl}-1-ethyl-N-hydroxypiperidine-4-carboxamide hydrochloride

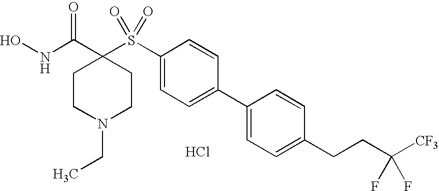

Part A. Preparation of 4-[4′-(3,3-difluoro-butyl)-biphenyl-4-sulfonyl]-1-ethyl-piperidine-4-carboxylic acid tert-butyl ester (2): 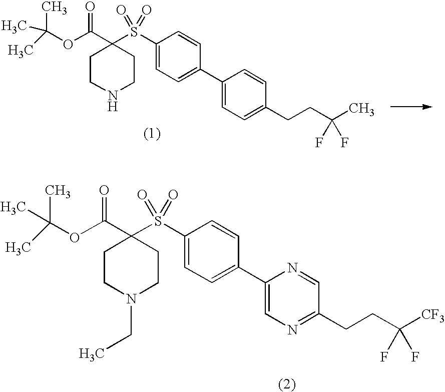
To a mixture of DMF (10 mL) and 4-[4′-(3,3-difluoro-butyl)-biphenyl-4-sulfonyl]-piperidine-4-carboxylic acid tert-butyl ester (1) (1.75 g, 3.55 mmol made in accordance with Example 25, Part B) was added ethyliodide (“EtI”, 0.31 mL, 3.9 mmol) and diisopropylethyl amine (“DIEA”, 0.93 mL, 5.3 mmol). The resulting mixture was stirred at room temperature for 4 hr. The solvent was then stripped, and the resulting residue was partitioned between EtOAc and water. The organic layer was separated, washed with brine, dried over MgSO₄, and evaporated to form an oil. The crude material was purified on silica gel (eluting with 10-80% ethyl acetate (containing 10% MeOH) in hexane) to form an oil. The product was further purified by recrystallization from diethyl ether:hexane to afford 1.0 g (54% yield) of the desired product (2) as a white solid. LCMS: m/z=522.2 (M+H).

Part B. Preparation of 4′-(3,3-difluoro-butyl)-biphenyl-4-sulfonyl]-1-ethyl-piperidine-4-carboxylic acid (tetrahydro-pyran-2-yloxy)-amide (3): 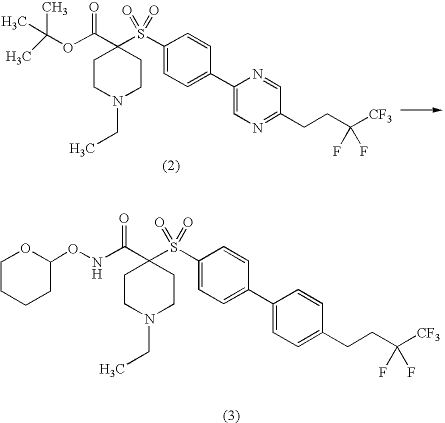
To a mixture of EtOAc (5 mL) and the product (2) from Part A (0.97 g, 0.1.86 mmol) was added 4 N HCl in dioxane (10 mL). The resulting mixture was stirred for 16 hr at room temperature. Additional 4 N HCl in dioxane (10 mL) was added, and the mixture was heated to 90° C. for 4 hr. The mixture was then stripped in vacuo to form a crude carboxylic acid product. To a mixture of the crude carboxylic acid in DMF (12 mL) was added O-(tetrahydro-2H-pyran-2-yl)hydroxylamine (“THPONH₂”, 0.70 g, 6.0 mmol), 1-(3-dimethylaminopropyl)-3-ethylcarbodiimide hydrochloride (“EDC.HCl”, 1.15 g, 6.0 mmol), 1-hydroxybenzotriazole hydrate (“HOBT”, 0.81 g, 6.0 mmol), and triethylamine (“Et₃N”, 1.3 mL, 9.3 mmol). The resulting mixture was stirred for 16 hr at room temperature. An additional 6.0 mmol of each O-(tetrahydro-2H-pyran-2-yl)hydroxylamine, 1-(3-dimethylaminopropyl)-3-ethylcarbodiimide hydrochloride and 1-hydroxybenzotriazole hydrate were then added. After an additional 16 hours at room temperature, the solvent was stripped in vacuo, and the resulting residue partitioned between ethyl acetate and water. The organic layer was separated, washed with saturated NaHCO₃ and brine, dried over MgSO₄, and evaporated to form an oil. The crude material was purified by flash column chromatography on silica gel (eluting with 1 to 10% MeOH in CH₂Cl₂) to afford 0.57 g (54% yield) of the desired THP protected hydroxamic acid (3) as a white solid. LCMS: m/z=565.3 (M+H).

Part C. Preparation of 4-{[4′-(3,3-difluorobutyl)-1,1′-biphenyl-4-yl]sulfonyl}-1-ethyl-N-hydroxypiperidine-4-carboxamide hydrochloride (4): 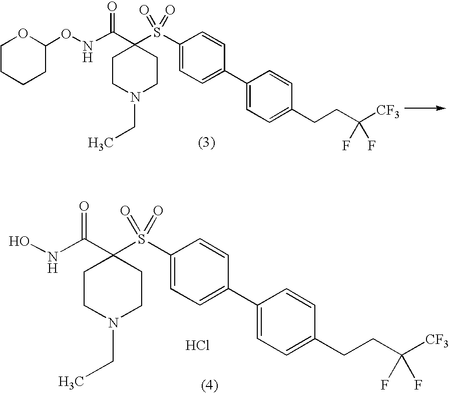
To a mixture of EtOAc (5 mL) and the product (3) of Part B (0.57 g, 1.0 mmol) was added 1.25 N HCl in ethanol (1.2 mL). The resulting colorless mixture was stirred for 5 hr, forming a white precipitate. Afterward, the slurry was filtered, and the resulting solid was washed with EtOAc (2×5 mL) and Et₂O (2×5 mL). The precipitate was dried in vacuo for 16 hr to afford 0.36 g (69% yield) of the desired product as a hydrochloride salt (4). LCMS: m/z=481.4 (M+H). HRMS calcd. for C₂₄H₃₁F₂N₂O₄S: m/z=481.1967 [M+H]⁺, found: 481.1936.

### Example 28

### Preparation of N-hydroxy-4-({4-[5-(3,3,3-trifluoropropyl)pyrazin-2-yl]phenyl}sulfonyl)tetrahydro-2H-pyran-4-carboxamide hydrochloride

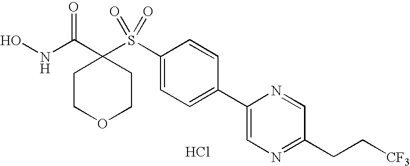

Part A. Preparation of 4-{4-[5-(3,3,3-trifluoro-propyl)-pyrazin-2-yl]-benzenesulfonyl}-tetrahydro-pyran-4-carboxylic acid tert-butyl ester (2): 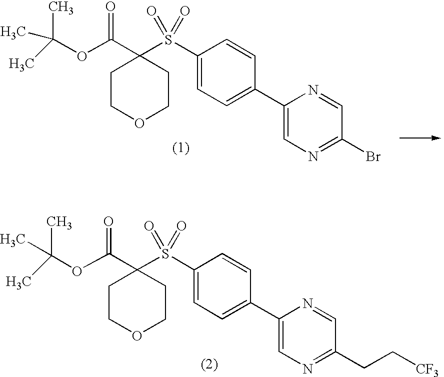
To a slurry of Zn dust (1.21 g, 18.6 mmol) in THF (3 mL) was added dibromoethane (0.19 mL, 2.2 mmol). The resulting slurry was heated to reflux briefly and cooled 3 times. Neat chlorotrimethylsilane (0.28 mL, 2.2 mmol) was then slowly added, and the mixture was stirred for 15 min. Neat 1,1,1-trifluoro-3-iodopropane (2.85 g, 12.7 mmol) was added, which slowly caused an exothermic reaction. The zinc mixture was stirred for 1 hr at room temperature. The supernatant of the resulting mixture was transferred by canulla into a mixture of DMA (15 mL) and 4-[4-(5-bromo-pyrazin-2-yl)-benzenesulfonyl]-tetrahydro-pyran-4-carboxylic acid tert-butyl ester (1) (3.5 g, 7.3 mmol, prepared in accordance with Example 21, Part C) and Pd(P(o-tolyl)₃)₂Cl₂ (0.29 g, 0.4 mmol). After stirring for 16 hr at room temperature, the reaction mixture was quenched with saturated NH₄Cl (5 mL), and then partitioned between EtOAc (100 mL) and water (50 mL). After filtering the mixture through Celite, the organic layer was separated, washed with brine, dried over MgSO₄, and evaporated to form a brown solid. The crude material was purified by flash column chromatography on silica gel (eluting with 20-100% ethyl acetate in CH₂Cl₂) to afford 1.40 g (38% yield) of the desired product (2) as a white solid. LCMS: m/z=501.4 (M+H).

Part B. Preparation of 4-{4-[5-(3,3,3-trifluoro-propyl)-pyrazin-2-yl]-benzenesulfonyl}-tetrahydro-pyran-4-carboxylic acid (tetrahydro-pyran-2-yloxy)-amide (3): 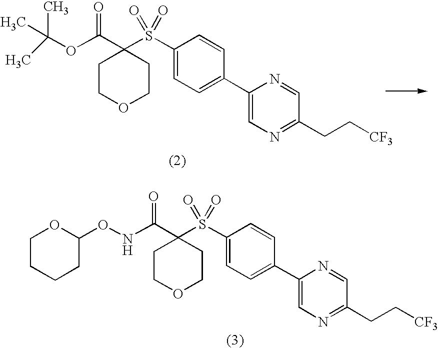
To a mixture of the product (2) from Part A (1.30 g, 2.6 mmol) in CH₂Cl₂ (2 mL) was added trifluoroacetic acid (“TFA”, 4 mL). The resulting mixture was stirred 3 hr at room temperature. The mixture was then stripped in vacuo to form a crude carboxylic acid product. To a mixture of the crude acid product in DMF (25 mL) was added O-(tetrahydro-2H-pyran-2-yl)hydroxylamine (“THPONH₂”, 0.91 g, 7.8 mmol), 1-(3-dimethylaminopropyl)-3-ethylcarbodiimide hydrochloride (“EDC.HCl”, 1.50 g, 7.8 mmol), 1-hydroxybenzotriazole hydrate (1.05 g, 7.8 mmol), and triethylamine (“Et₃N”, 1.45 mL, 10.4 mmol). The resulting mixture was stirred for 16 hr at room temperature. The solvent was then stripped in vacuo, and the resulting residue was partitioned between ethyl acetate and water. The organic layer was separated, washed with saturated NaHCO₃ and brine, dried over MgSO₄, and evaporated to form an oil. The crude material was purified by flash column chromatography on silica gel (eluting with 0-25% ethyl acetate in CH₂Cl₂) to afford 1.26 g (89% yield) of the desired THP protected hydroxamic acid (3) as a pale yellow foam. LCMS: m/z=544.2

Part C. Preparation of N-hydroxy-4-({4-[5-(3,3,3-trifluoropropyl)pyrazin-2-yl]phenyl}sulfonyl)tetrahydro-2H-pyran-4-carboxamide hydrochloride (3): 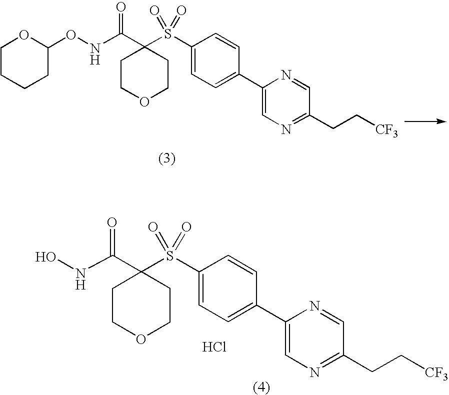
To a mixture of EtOAc (10 mL) and the product (3) from Part C (1.2 g, 2.2 mmol) was added 1.25 N HCl in ethanol (2.5 mL). The resulting colorless mixture was stirred for 2 hr, causing a white precipitate to form. The slurry was diluted with diethyl ether (20 mL) and hexane (5 mL), and then stirred for 1 hr. Afterward, the slurry was filtered, and the solid was washed with Et₂O (2×5 mL). The precipitate was then dried in vacuo for 16 hr to afford 0.89 g (85% yield) of the desired product as a hydrochloride salt (4). LCMS: m/z=460.1 (M+H). HRMS calcd. for C₁₉H₂₁F₃N₃O₅S: m/z=460.1149 [M+H]⁺, found: 460.1163.

### Example 29

### Preparation of 4-{[4′-(3,3-difluorobutyl)-1,1′-biphenyl-4-yl]sulfonyl}-N-hydroxy-1-(2-methoxyethyl)piperidine-4-carboxamide hydrochloride

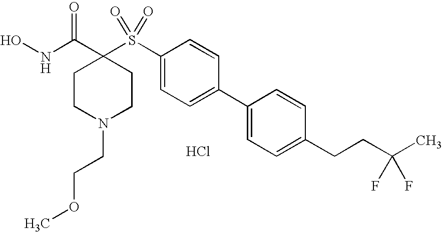

Part A. Preparation of 4-[4′-(3,3-difluoro-butyl)-biphenyl-4-sulfonyl]-1-(2-methoxy-ethyl)-piperidine-4-carboxylic acid tert-butyl ester (2): 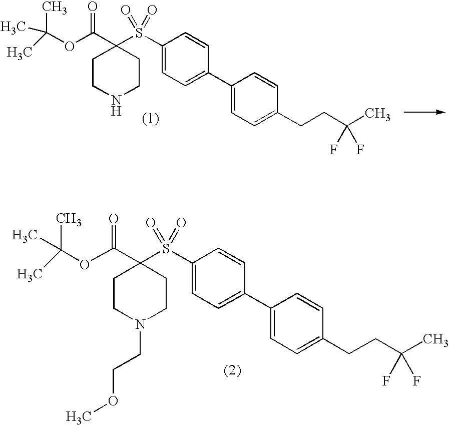
To a mixture of DMF (15 mL) and 4-[4′-(3,3-difluoro-butyl)-biphenyl-4-sulfonyl]-piperidine-4-carboxylic acid tert-butyl ester (1) (2.90 g, 5.9 mmol, prepared in accordance with Example 25, Part B) was added 2-bromoethyl methyl ether (0.61 mL, 6.5 mmol) and diisopropylethyl amine (“DIEA”, 1.55 mL, 8.9 mmol). The resulting mixture was stirred at room temperature for 16 hr, and then heated at 60° C. for 16 hr. Afterward, the solvent was stripped, and the residue was partitioned between EtOAc and water. The organic layer was separated, washed with brine, dried over MgSO₄, and evaporated to form an oil . The crude material was purified on silica gel (eluting with 10-80% ethyl acetate (containing 20% CH₃CN) in hexane) to form an oil. The product was triturated with hexane to form an off-white solid, which, in turn, was isolated by filtration and washed with hexane to afford 1.48 g (45% yield) of the desired product (2). LCMS: m/z=552.2 (M+H).

Part B. Preparation of 4-[4′-(3,3-difluoro-butyl)-biphenyl-4-sulfonyl]-1-(2-methoxy-ethyl)-piperidine-4-carboxylic acid (tetrahydro-pyran-2-yloxy)-amide (3): 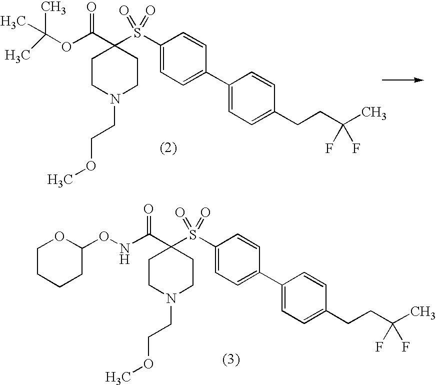
Solid product (2) from Part A (1.48 g, 2.7 mmol) was dissolved in trifluoroacetic acid (5 mL. The resulting mixture was stirred 4 hr at room temperature. The mixture was then stripped in vacuo to form a crude carboxylic acid product. To a mixture of the crude acid product in DMF (30 mL) was added O-(tetrahydro-2H-pyran-2-yl)hydroxylamine (“THPONH₂”, 0.91 g, 7.8 mmol), 1-(3-dimethylaminopropyl)-3-ethylcarbodiimide hydrochloride (“EDC.HCl”, 1.5 g, 7.8 mmol), 1-hydroxybenzotriazole hydrate (“HOBT”, 1.05 g, 7.8 mmol), and triethylamine (“Et₃N”, 1.5 mL, 10.8 mmol). After 16 hr at room temperature, the solvent was stripped in vacuo, and the resulting residue was partitioned between ethyl acetate and water. The organic layer was separated, washed with saturated NaHCO₃ and brine, dried over MgSO₄, and evaporated to form an oil. The crude material was purified by flash column chromatography on silica gel (eluting with 5-50% CH₃CN (containing 1% NH₄OH) in EtOAc) to afford 1.16 g (97% yield) of the desired THP protected hydroxamic acid (3) as a white solid. LCMS: m/z=595.5 (M+H).

Part C. Preparation of 4-{[4′-(3,3-difluorobutyl)-1,1′-biphenyl-4-yl]sulfonyl}-N-hydroxy-1-(2-methoxyethyl)piperidine-4-carboxamide hydrochloride (4): 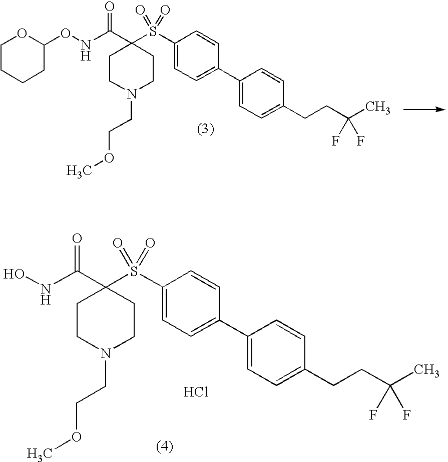
To a mixture of EtOAc (15 mL) and the product (3) from Part B (1.10 g, 1.85 mmol) was added 1.25 N HCl in ethanol (2.0 mL). The resulting colorless mixture was stirred for 5 hr, causing a white precipitate to form. The slurry was diluted with diethyl ether (25 mL) and stirred 1 hr. The slurry was then filtered, and the solid was washed with Et₂O (2×20 mL). The precipitate was then dried in vacuo for 16 hr to afford 0.87 g (87% yield) of the desired product as a hydrochloride salt (4). LCMS: m/z=511.4 (M+H). HRMS calcd. for C₂₅H₃₃F₂N₂O₅S: m/z=511.2073 [M+H]⁺, found: 511.2059.

### Example 30

### Preparation of 1-ethyl-N-hydroxy-4-({4-[5-(3,3,4,4,4-pentafluorobutyl)pyrazin-2-yl]phenyl}sulfonyl)piperidine-4-carboxamide dihydrochloride

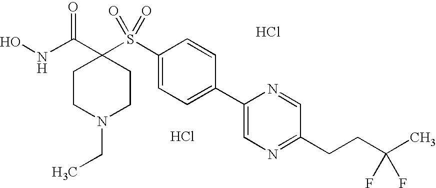

Part A. Preparation of 1-benzyl-4-[4-(5-bromo-pyrazin-2-yl)-benzenesulfonyl]-piperidine-4-carboxylic acid tert-butyl ester (3): 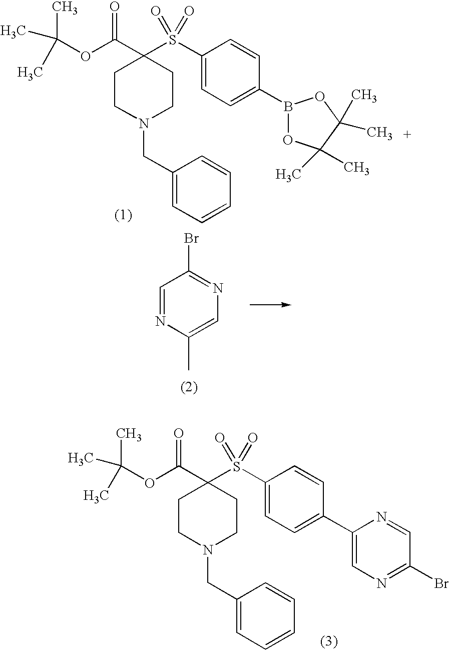
To a slurry of 1-benzyl-4-[4-(4,4,5,5-tetramethyl-[1,3,2]dioxaborolan-2-yl)-benzenesulfonyl]-piperidine-4-carboxylic acid tert-butyl ester (1) (12.0 g, 22.2 mmol) in toluene (40 mL) was added solid 2-bromo-5-iodo-pyrazine (2) (6.98 g, 24.5 mmol, prepared in accordance with Example 21, Part B), 2 N Na₂CO₃ (25 mL), ethanol (2.5 mL), and Pd(dppf)Cl₂ (0.90 g, 1.1 mmol). The resulting mixture was heated at 60° C. for 16 hr. Afterward, the mixture was partitioned between EtOAc and water, and then filtered to remove insolubles. The organic layer was separated, washed with brine, dried over MgSO₄, and evaporated to a brown solid. The crude material was purified by flash column chromatography on silica gel (eluting with 5-80% ethyl acetate in hexane) to afford 6.60 g (52% yield) of the desired product (3) as an off-white solid. LCMS: m/z=572.3, 574.3.

Part B. Preparation of 1-benzyl-4-{4-[5-(3,3,4,4,4-pentafluoro-butyl)-pyrazin-2-yl]-benzenesulfonyl}-piperidine-4-carboxylic acid tert-butyl ester (4): 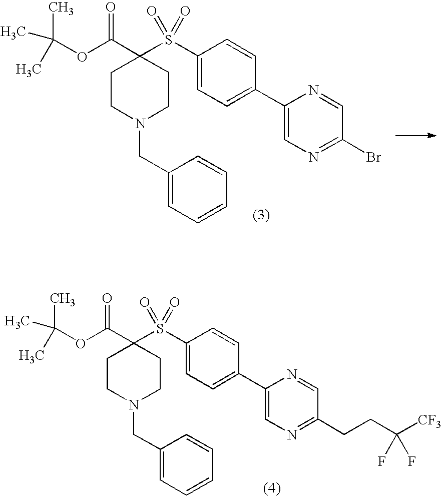
To a slurry of Zn dust (1.95 g, 30 mmol) in THE (5 mL) was added dibromoethane (0.30 mL, 3.4 mmol). The resulting slurry was heated to reflux briefly and cooled 3 times. Neat chlorotrimethylsilane (0.43 mL, 3.4 mmol) was slowly added, and the mixture was stirred for 15 min. Neat 4-iodo-1,1,1,2,2-pentafluorobutane (5.2 g, 20 mmol) was then added slowly, causing an exothermic reaction. The zinc mixture was stirred for 1 hr at room temperature. Afterward, the supernatant was transferred by cannula into a DMA (25 mL) mixture of the product (3) from Part A (6.3 g, 11.0 mmol) and Pd(P(o-tolyl)₃)₂Cl₂ (0.43 g, 0.55 mmol). After heating for 30 min at 90° C., the reaction mixture was quenched with saturated NH₄Cl (25 mL), and then partitioned between EtOAc and water. The mixture was filtered through Celite, and then the organic layer was separated, washed with brine, dried over MgSO₄, and evaporated to a brown oil. The crude material was purified by flash column chromatography on silica gel (eluting with 10-50% ethyl acetate in hexane to afford 5.00 g (71% yield) of the desired product (4) as an off-white solid. LCMS: m/z=640.5

Part C. Preparation of 4-{4-[5-(3,3,4,4,4-pentafluoro-butyl)-pyrazin-2-yl]-benzenesulfonyl}-piperidine-4-carboxylic acid tert-butyl ester (5): 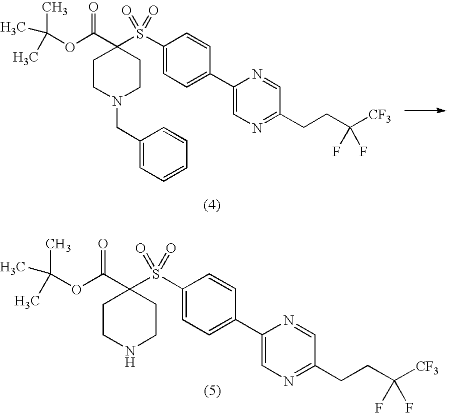
To a mixture of methanol (70 mL) and the product (4) from Part B (4.69 g, 2.9 mmol) was added cyclohexene (7.5 mL) and 10% Pd/C (wet, Degussa type E101, 1.5 g). The slurry was refluxed for 6 hr and then cooled to room temperature. Afterward, the catalyst was removed by filtration through Celite. The filtrate was stripped to afford 3.63 g (91% yield) of the desired product (5) as an off-white foam. LCMS: m/z=550.5 (M+H).

Part D. Preparation of 1-ethyl-4-{4-[5-(3,3,4,4,4-pentafluoro-butyl)-pyrazin-2-yl]-benzenesulfonyl}-piperidine-4-carboxylic acid tert-butyl ester (6): 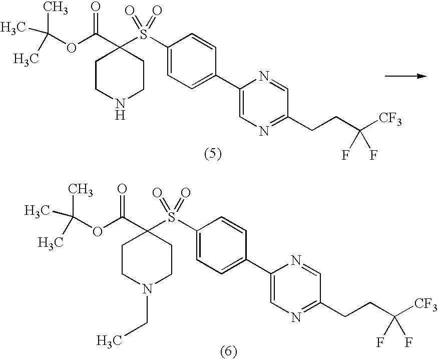
To a mixture of DMF (7 mL) and the product (5) from Part C (1.1 g, 2.0 mmol) was added ethyliodide (“EtI”, 0.18 mL, 2.2 mmol) and diisopropylethyl amine (“DIEA”, 0.52 mL, 3.0 mmol). The mixture was stirred at room temperature for 16 hr. The solvent was then stripped, and the residue was partitioned between EtOAc and water. The organic layer was separated, washed brine, dried over MgSO₄, and evaporated to afford 1.15 g (100% yield) of the desired product (6) as an off-white solid. LCMS: m/z=578.5 (M+H).

Part E. Preparation of 1-ethyl-4-{4-[5-(3,3,4,4,4-pentafluoro-butyl)-pyrazin-2-yl]-benzenesulfonyl}-piperidine-4-carboxylic acid (tetrahydro-pyran-2-yloxy)-amide (7): 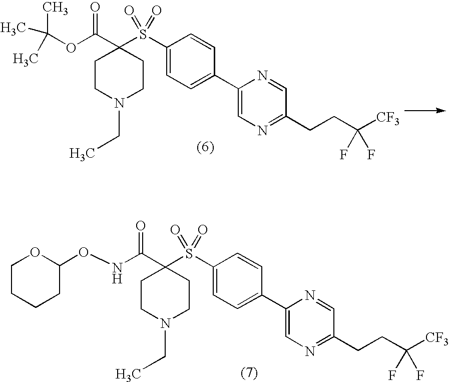
Solid product (6) from Part D (1.13 g, 1.96 mmol) was dissolved in trifluoroacetic acid (“TFA”, 5 mL). The resulting mixture was stirred for 4 hr at room temperature. The mixture was then stripped in vacuo to form a crude carboxylic acid product. To a mixture of the crude acid in DMF (20 mL) was added 1-(3-dimethylaminopropyl)-3-ethylcarbodiimide hydrochloride (“EDC.HCl”, 1.15 g, 6.0 mmol), 1-hydroxybenzotriazole hydrate (“HOBT”, 0.81 g, 6.0 mmol), and 4-methylmorpholine (1.5 mL, 10.8 mmol). The slurry was heated to 60° C. for 30 min, and then O-(tetrahydro-2H-pyran-2-yl)hydroxylamine (“THPONH₂”, 0.70 g, 6.0 mmol) was added. Heat was continued for 2 hr. After 16 hr at room temperature, the solvent was stripped in vacuo, and the residue was partitioned between ethyl acetate and water. The organic layer was separated, washed with saturated NaHCO₃ and brine, dried over MgSO₄, and evaporated to an oil. The crude material was purified by flash column chromatography on silica gel (eluting with 1-10% MeOH (containing 1% NH₄OH) in CH₂Cl₂) to afford 0.68 g (56% yield) of the desired THP protected hydroxamic acid (7) as a white solid. LCMS: m/z=621.3 (M+H).

Part F. Preparation of 1-ethyl-N-hydroxy-4-({4-[5-(3,3,4,4,4-pentafluorobutyl)pyrazin-2-yl]phenyl}sulfonyl)piperidine-4-carboxamide dihydrochloride (8): 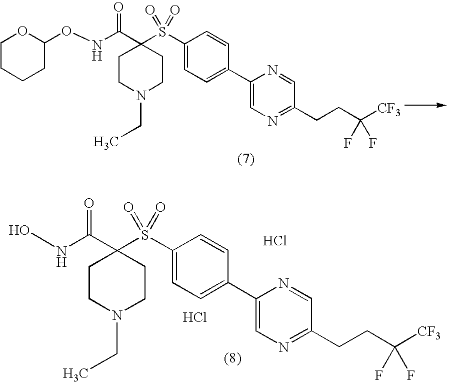
To a mixture of EtOAc (6 mL) and the product (7) from Part E (0.62 g, 1.0 mmol) was added 1.25 N HCl in ethanol (1.2 mL). After 30 min, the resulting viscous slurry was diluted with EtOAc (5 mL). Additional 1.25 N HCl in ethanol (1.2 mL) was added, and the slurry was stirred for 2 hr, diluted with hexane (20 mL), and stirred for an additional hour. Subsequently, the slurry was filtered, and the solid was washed with hexane (2×5 mL) and Et₂O (2×5 mL). The precipitate was dried in vacuo for 16 hr to afford 0.36 g (54% yield) of the desired product as a hydrochloride salt (8). LCMS: m/z=537.3 (M+H). HRMS calcd. for C₂₂H₂₆F₅N₄O₄S: m/z=537.1589 [M+H]⁺, found: 537.1584.

### Example 31

### Preparation of N-hydroxy-1-(2-methoxyethyl)-4-({4-[5-(3,3,4,4,4-pentafluorobutyl)pyrazin-2-yl]phenyl}sulfonyl)piperidine-4-carboxamide dihydrochloride

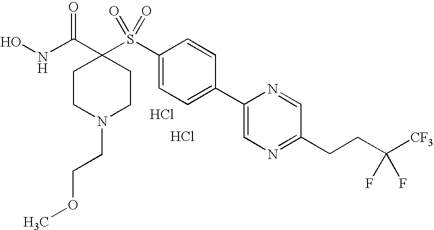

Part A. Preparation of 1-(2-methoxy-ethyl)-4-{4-[5-(3,3,4,4,4-pentafluoro-butyl)-pyrazin-2-yl]-benzenesulfonyl}-piperidine-4-carboxylic acid tert-butyl ester (2): 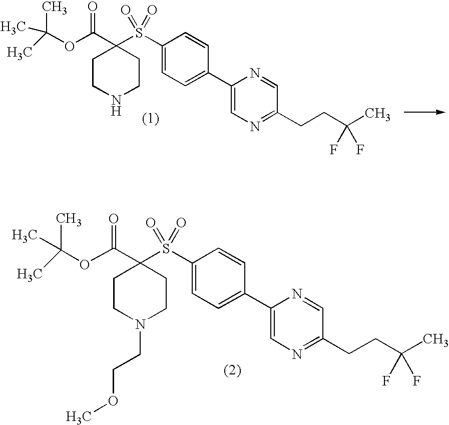
To mixture of DMF (7 mL) and 4-{4-[5-(3,3,4,4,4-pentafluoro-butyl)-pyrazin-2-yl]-benzenesulfonyl}-piperidine-4-carboxylic acid tert-butyl ester (1) (1.1 g, 2.0 mmol, prepared in accordance with Example 30, Part C) was added 2-bromoethyl methyl ether (0.52 mL, 2.2 mmol), diisopropylethyl amine (“DIEA”, 0.52 mL, 3.0 mmol), and potassium iodide (0.03 g, 0.2 mmol). The mixture was stirred at room temperature for 16 hr. Additional potassium iodide (0.03 g, 0.3 mmol) was then added, and the mixture was stirred for 16 hr at 50° C. Afterward, the solvent was stripped, and the residue was partitioned between EtOAc and water. The organic layer was separated, washed with brine, dried over MgSO₄, and evaporated to form an oil. The crude material was purified on silica gel (eluting with 5-50% CH₃CN (containing 1% NH₄OH) in EtOAc) to afford the 0.89 g (74% yield) of the desired product (2) as an oil, which solidified upon standing. LCMS: m/z=608.5 (M+H).

Part B. Preparation of 1-(2-methoxy-ethyl)-4-{4-[5-(3,3,4,4,4-pentafluoro-butyl)-pyrazin-2-yl]-benzenesulfonyl}-piperidine-4-carboxylic acid (tetrahydro-pyran-2-yloxy)-amide (3): 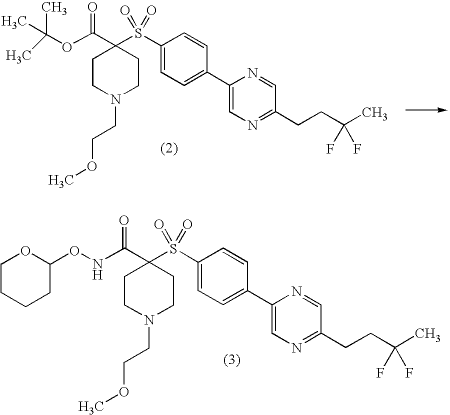
To a mixture of the product (2) from Part A (0.81 g, 1.3 mmol) in CH₂Cl₂ (3 mL) was added trifluoroacetic acid (“TFA”, 6 mL). The resulting mixture was stirred 3 hr at room temperature. Afterward, the mixture was stripped in vacuo to form a crude carboxylic acid product. To a mixture of the crude carboxylic acid product in DMF (12 mL) was added O-(tetrahydro-2H-pyran-2-yl)hydroxylamine (“THPONH₂”, 0.47 g, 4.0 mmol), 1-(3-dimethylaminopropyl)-3-ethylcarbodiimide hydrochloride (“EDC.HCl”, 0.77 g, 4.0 mmol), 1-hydroxybenzotriazole hydrate (“HOBT”, 0.54 g, 4.0 mmol), and triethylamine (“Et₃N”, 0.74 mL, 5.3 mmol). The reaction mixture was stirred 16 hr at room temperature, the solvent was stripped in vacuo, and the residue partitioned between ethyl acetate and water. The organic layer was separated, washed with saturated NaHCO₃ and brine, dried over MgSO₄, and evaporated to an oil. The crude material was purified by flash column chromatography on silica gel (eluting with 5-50% CH₃CN (containing 1% NH₄OH) in EtOAc) to afford 0.60 g (70% yield) of the desired THP protected hydroxamic acid (3) as a white solid. LCMS: m/z=651.5 (M+H).

Part C. Preparation of N-hydroxy-1-(2-methoxyethyl)-4-({4-[5-(3,3,4,4,4-pentafluorobutyl)pyrazin-2-yl]phenyl}sulfonyl)piperidine-4-carboxamide dihydrochloride (4): 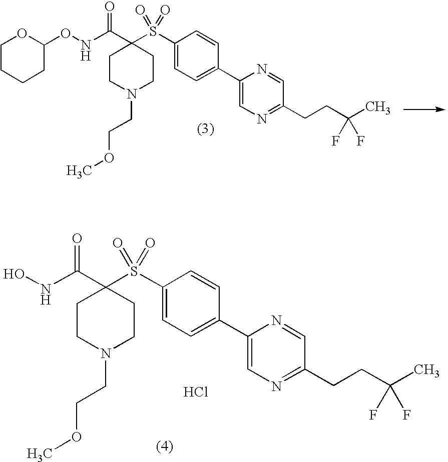
To a mixture of EtOAc (10 mL) and the product (3) of Part B (0.55 g, 0.85 mmol) was added 1.25 N HCl in ethanol (1.0 mL). The resulting colorless mixture was stirred for 3 hr. Afterward, the mixture was diluted with hexane (10 mL) and stirred for 1 hr. The resulting slurry was filtered, and the resulting solid was washed with hexane (2×20 mL). The precipitate was dried in vacuo for 16 hr to afford 0.87 g (87% yield) of the desired product as a hydrochloride salt (4). LCMS: m/z=567.3 (M+H). HRMS calcd. for C₂₃H₂₈F₅N₄O₅S: m/z=567.1695 [M+H]⁺, found: 567.1695.

### Example 32

### Preparation of 1-cyclopropyl-N-hydroxy-4-({4-[5-(3,3,4,4,4-pentafluorobutyl)pyrazin-2-yl]phenyl}sulfonyl)piperidine-4-carboxamide dihydrochloride

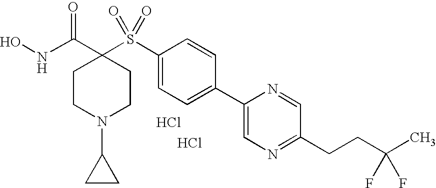

Part A. Preparation of 1-cyclopropyl-4-{4-[5-(3,3,4,4,4-pentafluoro-butyl)-pyrazin-2-yl]-benzenesulfonyl}-piperidine-4-carboxylic acid tert-butyl ester (2): 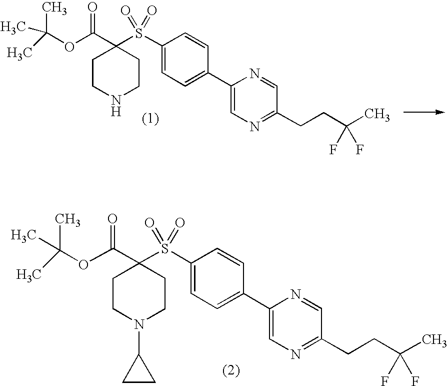
To a mixture of methanol (10 mL) and 4-{4-[5-(3,3,4,4,4-pentafluoro-butyl)-pyrazin-2-yl]-benzenesulfonyl}-piperidine-4-carboxylic acid tert-butyl ester (1) (1.4 g, 2.6 mmol, prepared in accordance with Example 30, Part C) was added [(1-ethoxycyclopropyl)oxy]trimethylsilane (0.78 g, 3.9 mmol), sodium cyanoborohydride (NaBH₃CN, 0.25 g, 4.0 mmol), and HOAc (1.5 mL, 26 mmol). The resulting mixture was refluxed for 6 hr, and then stirred at room temperature 16 hr. Afterward, the solvent was stripped, and the residue partitioned between EtOAc and water. The organic layer was separated, washed with saturated NaHCO₃ and brine, dried over MgSO₄, and evaporated to form an oil. The crude material was purified on silica gel (eluting with 10-50% ethyl acetate (containing 10% CH₃CN) in hexane) to afford 0.89 g (58% yield) of the desired product (2) as a white solid.

Part B. Preparation of 1-cyclopropyl-4-{4-[5-(3,3,4,4,4-pentafluoro-butyl)-pyrazin-2-yl]-benzenesulfonyl}-piperidine-4-carboxylic acid (tetrahydro-pyran-2-yloxy)-amide (3): 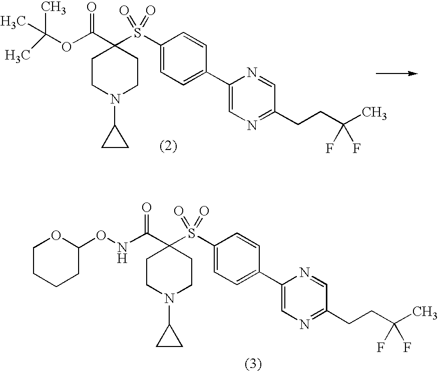
To a mixture of the product (2) from Part A (0.84 g, 1.4 mmol) in CH₂Cl₂ (3 mL) was added trifluoroacetic acid (“TFA”, 6 mL). The resulting mixture was stirred 3 hr at room temperature, and then stripped in vacuo to form a crude carboxylic acid product. To a mixture of the crude carboxylic acid product in DMF (10 mL) was added O-(tetrahydro-2H-pyran-2-yl)hydroxylamine (“THPONH₂”, 0.50 g, 4.3 mmol), 1-(3-dimethylaminopropyl)-3-ethylcarbodiimide hydrochloride (“EDC.HCl”, 0.82 g, 4.3 mmol), 1-hydroxybenzotriazole hydrate (“HOBT”, 0.58 g, 4.3 mmol), and triethylamine (“Et₃N”, 1.0 mL, 7.2 mmol). The resulting mixture was stirred for 16 hr at room. Subsequently, the solvent was stripped in vacuo, and the residue was partitioned between ethyl acetate and water. The organic layer was separated, washed with saturated NaHCO₃ and brine, dried over MgSO₄, and evaporated to form an oil. The crude material was purified by flash column chromatography on silica gel (eluting with 20-100% CH₃CN (containing 1% NH₄OH) in EtOAc) to afford 0.60 g (70% yield) of the desired THP protected hydroxarnic acid (3) as a white solid. LCMS: m/z=633.5 (M+H).

Part C. Preparation of 1-cyclopropyl-N-hydroxy-4-({4-[5-(3,3,4,4,4-pentafluorobutyl)pyrazin-2-yl]phenyl}sulfonyl)piperidine-4-carboxamide dihydrochloride (4): 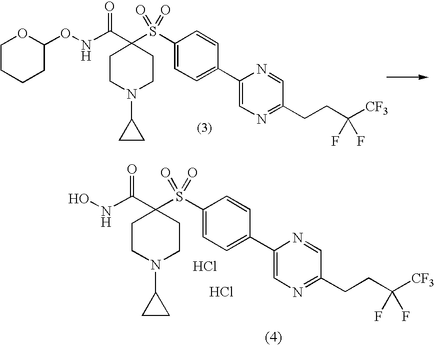
To a mixture of EtOAc (10 mL) and the product (3) from Part B (0.53 g, 0.84 mmol) was added 1.25 N HCl in ethanol (1.0 mL). The resulting colorless mixture was stirred for 3 hr. The resulting slurry was diluted with hexane (20 mL) and then stirred 1 hr. Afterward, the slurry was filtered, and the solid was washed with hexane (2×20 mL). The precipitate was dried in vacuo for 16 hr to afford 0.41 g (79% yield) of the desired product as a hydrochloride salt (4). LCMS: m/z=549.5 (M+H). HRMS calcd. for C₂₃H₂₆F₅N₄O₄S: m/z=549.1589 [M+H]⁺, found: 549.1596.

### Example 33

### Preparation of 1-(2-methoxyethyl)-4-({4-[5-(3,3,4,4,4-pentafluorobutyl)(2-pyridyl)]phenylasulfonyl)piperidine-4-carbohydroxanic acid, dihydrochloride:

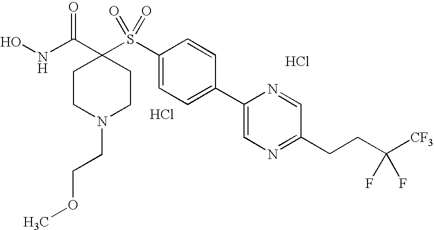

Part A. Preparation of 1-benzyl-4-[4-(5-bromo-pyridin-2-yl)-benzenesulfonyl]-piperidine-4-carboxylic acid tert-butyl ester (3): 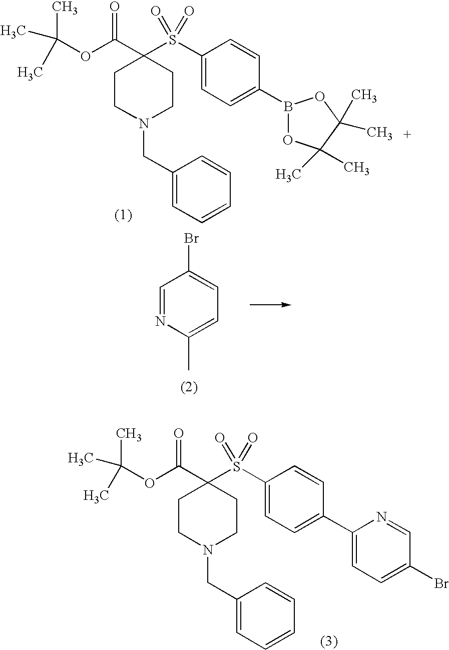
To a mixture of 1-benzyl-4-[4-(4,4,5,5-tetramethyl-[1,3,2]dioxaborolan-2-yl)-benzenesulfonyl]-piperidine-4-carboxylic acid tert-butyl ester (1) (5.0 g, 9.2 mmol) in toluene (28 mL), ethanol (7 mL), and 1 M sodium carbonate (Na₂CO₃, 28 mL) under N₂ were added 2-iodo-5-bromopyridine (2) (2.9 g, 10.2 mmol) and [1,1′-bis(diphenylphosphino)ferrocene]dichloropalladium(II) (“Pd(dppf)Cl₂”, 0.38 g, 0.46 mmol). The resulting mixture was heated at 80° C. under N₂ for 6 hr, after which LC/MS detected no starting material (1). The mixture was cooled to room temperature and diluted with ethyl acetate and water. The mixture was then filtered through a pad of Celite. The layers of the filtrate were separated, and the organic layer was washed with water (2 times), washed with saturated NaCl (1 time), and dried over anhydrous sodium sulfate. Filtration and evaporation of the solvent under reduced pressure formed a dark oil. The residue was dissolved in dichloromethane and purified on SiO₂ (using 30% ethyl acetate/hexane, followed by 40% ethyl acetate/hexane) to afford 3.6 g of light yellow solid (69% yield). ¹H NMR and mass spectrometry (MH⁺=571.1) were consistent with the desired compound (3).

Part B. Preparation of tert-butyl 4-({4-[5-(3,3,4,4,4-pentafluorobutyl)(2-pyridyl]phenyl}sulfonyl)-1-benzylpiperidine-4-carboxylate (4): 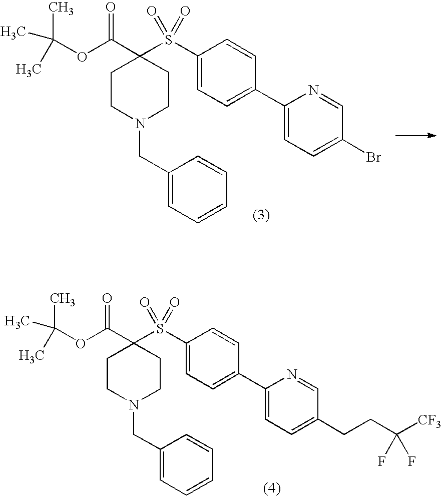
To a slurry of the ZnCu couple (3.1 g, 48.3 mmol) in benzene (65 mL) and DMF (3.5 mL) was added 1,1,1,2,2-pentafluoro-4-iodobutane (8.7 g, 31.6 mmol). The resulting mixture was heated at 65° C. under N₂ for 3 hr. A mixture of the product (3) from Part A (6.0 g, 10.5 mmol) in benzene (15 mL) and DMF (5 mL) was subsequently added, followed by [1,1′-bis(diphenylphosphino)ferrocene]dichloropalladium(II) (“Pd(dppf)Cl₂”, 0.43 g, 0.53 mmol). The temperature was then increased to 75° C., and the reaction was continued overnight, after which no starting material was detected by HPLC. The mixture was cooled to room temperature and diluted with ethyl acetate and water. The mixture was then filtered through a pad of Celite. The layers of the filtrate were separated, and the organic layer was washed with water (2 times), washed with saturated NaCl (1 time), and dried over anhydrous sodium sulfate. Filtration and evaporation of the solvent under reduced pressure formed a dark oil. The crude material was purified on SiO₂ (using dichloromethane with a methanol gradient) to afford 5.3 grams (79% yield) of an orange foam. ¹H NMR and mass spectrometry (MH⁺=639.1) were consistent with the desired compound (4).

Part C. Preparation of tert-butyl 4-({4-[5-(3,3,4,4,4-pentafluorobutyl)-2-pyridyl]phenyl}sulfonyl)piperidine-4-carboxylate (5): 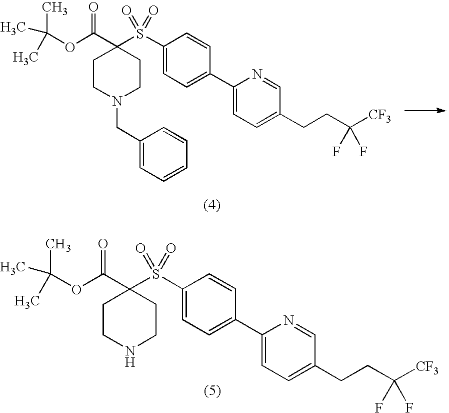
Cyclohexene (27 mL) and 10% Pd/C (2.7 g) were added to a mixture of methanol (80 mL) and the product (4) from Part B (5.3 g, 8.3 mmol). After refluxing for 7 hr, HPLC indicated that the reaction was complete. The mixture was cooled to room temperature and filtered through Celite. The filtrate was concentrated under reduced pressure to form a yellow oil, which solidified upon standing (4.1 g, 91% yield). ¹H NMR and mass spectrometry (MH⁺=549.1) were consistent with the desired compound (5). This material was used in Part D without further purification.

Part D. Preparation of tert-butyl 1-(2-methoxyethyl)-4-({4-[5-(3,3,4,4,4-pentafluorobutyl)(2-pyridyl]phenyl}sulfonyl)piperidine-4-carboxylate (6): 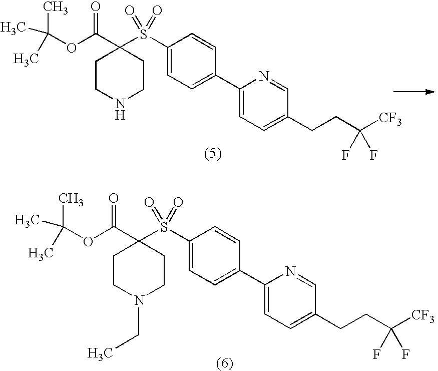
The product (5) from Part C (1.3 g, 2.3 mmol), bromoethyl methyl ether (0.34 g, 2.4 mmol), and N,N-diisopropylethylamine (“DIEA”, 0.31 g, 2.4 mmol) were dissolved in DMF (40 mL). The reaction was then allowed to continue overnight at room temperature. Because some starting material (5) continued to be present, additional bromoethyl methyl ether and DIEA (0.5 eq. each) were added. The mixture was heated at 45° C. over the weekend, after which LC/MS indicated that the reaction was complete. The mixture was then diluted with ethyl acetate, and the organic layer was washed with water (3×), washed with saturated NaCl (1×), and dried over anhydrous sodium sulfate. Filtration and evaporation of the solvent under reduced pressure afforded 1.3 g of a solid product (93% yield). ¹H NMR and mass spectrometry (MH⁺=607) were consistent with the desired compound (6). This material was used in Part E without further purification.

Part E. Preparation of the trifluoroacetic acid salt of 1-(2-methoxyethyl)-4-({4-[5-(3,3,4,4,4-pentafluorobutyl)(2-pyridyl]phenyl}sulfonyl)piperidine-4-carboxylic acid (7): 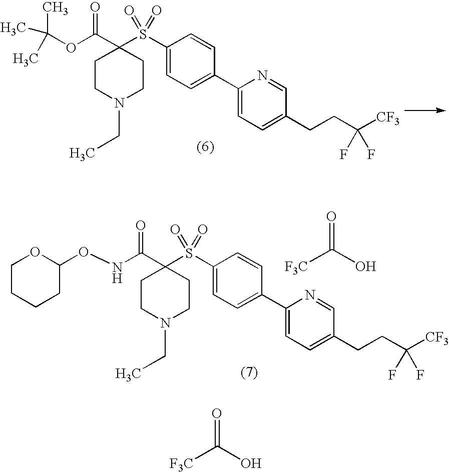
The product (6) from Part D (1.3 g, 2.1 mmol) was dissolved in 1:1 trifluoroacetic acid/dichloromethane (“TFA/CH₂Cl₂”, 40 mL). The reaction was then continued overnight at room temperature, after which no starting material (6) was detected by HPLC. The mixture was then concentrated under reduced pressure, and the resulting residue was stripped from diethyl ether several times under reduced pressure. After precipitating a final time, the precipitate was collected by suction filtration to afford 1.2 g of a solid product (75% yield for the di-TFA salt). Mass spectrometry (MH⁺=551) was consistent with the desired product (7).

Part F. Preparation of [1-(2-methoxyethyl)-4-({4-[5-(3,3,4,4,4-pentafluorobutyl)(2-pyridyl)]phenyl}sulfonyl)(4-piperidyl)]-N-perhydro-2H-pyran-2-yloxycarboxamide (8): 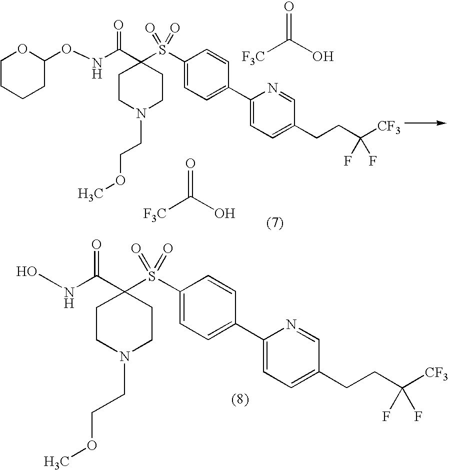
To a mixture of the product (7) from Part E (1.2 g, 1.5 mmol for di-TFA) in N,N-dimethylformamide (“DMF”, 46 mL) were added N-hydroxybenzotriazole (“HOBt”, 0.20 g, 2.1 mmol), 4-methylmorpholine (“NMM”, 0.77 g, 0.84 mL, 7.6 mmol), 1-(3-dimethylaminopropyl)-3-ethylcarbodiimide hydrochloride (“EDC.HCL”, 1.0 g, 5.3 mmol), and O-(tetrahydro-2H-pyran-2-yl)hydroxylamine (“THPONH₂”, 0.62 g, 5.3 mmol). The resulting mixture was heated for 2 hr at 45° C., cooled to room temperature, and then stirred over the weekend under N₂. Afterward, HPLC detected no starting material (7). The mixture was diluted with ethyl acetate. The organic layer was then extracted with water (3 times), extracted with saturated sodium bicarbonate (3 times), washed with saturated NaCl, and dried over anhydrous sodium sulfate. Filtration and evaporation of the solvent under reduced pressure formed a foam. The crude material was purified by flash chromatography (using dichloromethane with a methanol gradient (0-2%)) to afford 0.80 g of a white foam (82% yield). ¹H NMR and mass spectrometry (MH⁺=620) were consistent with the desired product (8).

Part G. Preparation of 1-(2-methoxyethyl)-4-({4-[5-(3,3,4,4,4-pentafluorobutyl)(2-pyridyl)]phenyl}sulfonyl)piperidine-4-carbohydroxamic acid, dihydrochloride (9): 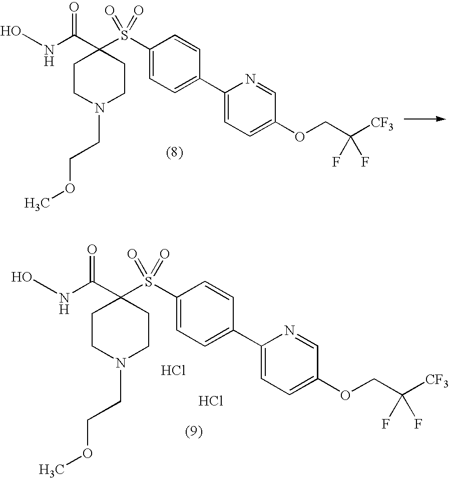
The product (8) from Part F (0.78 g, 1.2 mmol) was dissolved in dioxane (8 mL), 4N HCl in dioxane (10 mL), and methanol (1 mL). The reaction was then continued at ambient temperature overnight, after which HPLC indicated that the reaction was complete. The mixture was then concentrated under reduced pressure. The resulting residue was triturated with diethyl ether to form a white solid, which, in turn, was collected by suction filtration to afford 0.76 g of product (quantitative yield). ¹H NMR and high resolution mass spectrometry (theoretical MH⁺=566.1743, actual MH⁺=566.1716) were consistent with the desired product (9).

### Example 34

### Preparation of 4-({4-[4-(1,1,2,2-tetrafluoroethoxy)phenyl]phenyl}sulfonyl)perhydro-2H-pyran-4-carbohydroxamic acid

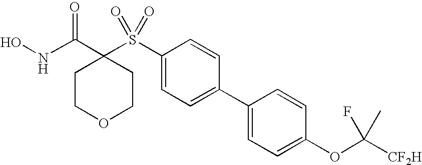

Part A. Preparation of tert-butyl 4-({4-[4-(1,1,2,2-tetrafluoroethoxy)phenyl]phenyl}sulfonyl)perhydro-2H-pyran-4-carboxylate (3): 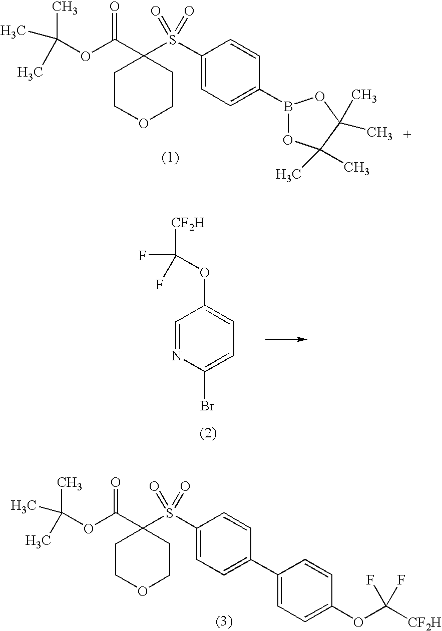
Into a 1 L round-bottom flask (equipped with a stir bar, N₂ inlet, and water-cooled condenser) was placed 4-[4-(4,4,5,5-tetramethyl-[1,3,2]dioxaborolan-2-yl)-benzenesulfonyl]-tetrahydro-pyran-4-carboxylic acid tert-butyl ester (1) (60 g, 0.133 mol), 1-bromo-4-(1,1,2,2-tetrafluoroethoxy)benzene (2) (45 g, 0.166 mol), and [1,1′-bis(diphenylphosphino)ferrocene]dichloropalladium(II) (“Pd(dppf)Cl₂”, 5.4 g, 6.6 mmol). A mixture of toluene (240 mL), 1M Na₂CO₃ (240 mL), and ethanol (60 mL) was then added. The resulting mixture was refluxed for 1 hr, after which no starting material (1) was indicated by HPLC. The mixture was then cooled to room temperature and diluted with ethyl acetate and water. The aqueous layer was removed and extracted with additional ethyl acetate (3×300 mL). The organic layers were combined, washed with brine, dried over magnesium sulfate, filtered, and concentrated. The crude product was purified by silica plug filtration (eluting with 1:1 ethyl acetate:hexane), concentrated, and triturated with cold ether to afford 49 g (71% yield) of desired product (3) as a tan solid. Mass spectrometry (MN⁺=504) was consistent with the desired product (3).

Part B. Preparation of 4-({4-[4-(1,1,2,2-tetrafluoroethoxy)phenyl]phenyl}sulfonyl)perhydro-2H-pyran-4-carboxylic acid (4): 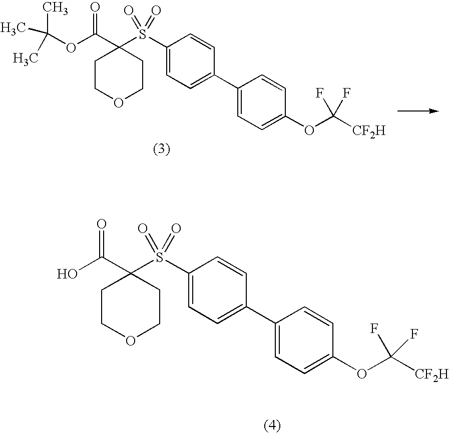
The product (3) from Part A (56 g, 0.108 mmol) was dissolved in 1:1 trifluoroacetic acid/dichloromethane (“TFA/CH₂Cl₂”, 180 mL). The reaction was then continued overnight at room temperature, after which no starting material (3) was detected by HPLC. The mixture was concentrated under reduced pressure. Additional dichloromethane was added, and the solvent was once again removed under reduced pressure. Ether was added and the precipitate was collected by suction filtration to afford the crude product (4) as a tan solid. Mass spectrometry (MN⁺=479) was consistent with the desired compound (4).

Part C. Preparation of N-perhydro-2H-pyran-2-yloxy[4-({4-[4-(1,1,2,2-tetrafluoroethoxy)phenyl]phenyl}sulfonyl)perhydro-2H-pyran-4-yl]carboxamide (5): 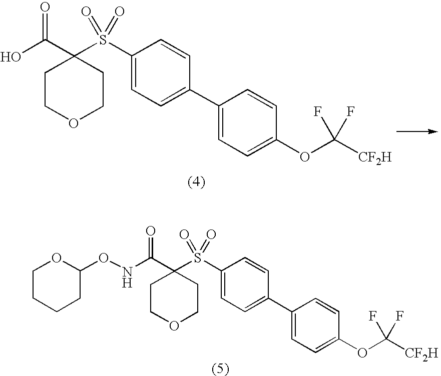
To a mixture of the product (4) from Part B (48 g, 0.104 mol) in N,N-dimethylformamide (“DMF”, 300 mL) were added N-hydroxybenzotriazole (“HOBt”, 42 g, 0.311 mol), triethylamine (“TEA”, 43 mL, 0.311 mol), 1-(3-dimethylaminopropyl)-3-ethylcarbodiimide hydrochloride (“EDC.HCl”, 79 g, 0.416 mol), and O-(tetrahydro-2H-pyran-2-yl)hydroxylamine (“THPONH₂”, 36 g, 0.311 mol). The reaction was then continued overnight at room temperature under N₂, after which no starting material (4) was detected by HPLC. The mixture was then diluted with ethyl acetate. The combined organic layers were extracted with water (3 times), extracted with saturated sodium bicarbonate (3 times), washed with saturated NaCl, and dried over anhydrous magnesium sulfate. Filtration and evaporation of the solvent under reduced pressure formed a yellow oil. This crude material was purified by plug filtration (using ethyl acetate (25%, followed by 50%) in hexane) to afford the desired product (5).¹HNMR was consistent with the desired compound (5).

Part D. Preparation of 4-({4-[4-(1,1,2,2-tetrafluoroethoxy)phenyl]phenyl}sulfonyl)perhydro-2H-pyran-4-carbohydroxamic acid (6): 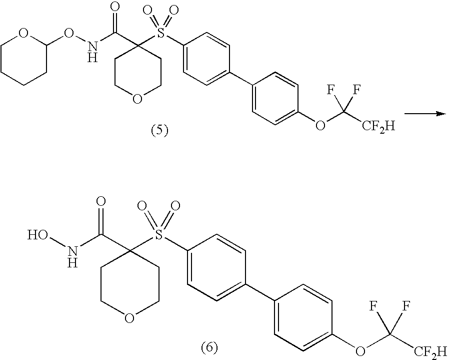
The product (5) from Part C (0.104 mol) was dissolved in 4N HCl in dioxane (390 mL), and methanol (10 mL). The reaction was then continued at ambient temperature for 18 hr, after which HPLC indicated that the reaction was complete. The mixture was then precipitated with diethyl ether/hexane, and the resulting white solid was collected by suction filtration. The product was dissolved in acetonitrile with heating, cooled, and added to stirring solution of deionized water. The desired product (6) precipitated as 31.4 g (63% yield) of a white solid free of impurities. ¹H NMR was consistent with the desired product (6). HRMS for C₂₀H₁₉NO₆SF₄ showed [M−H]_{found}=476.0742 for [M−H]_{calc}=476.0785.

### Example 35

### Preparation of N-hydroxy-4-({4-[5-(4,4,4-trifluorobutyl)pyridin-2-yl]phenyl}sulfonyl)tetrahydro-2H-pyran-4-carboxamide hydrochloride

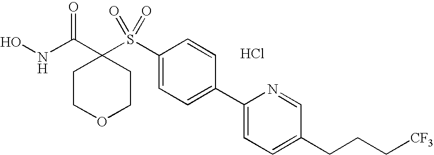

Part A. Preparation of tert-butyl 4-{[4-(5-bromo-2-pyridyl)phenyl]sulfonyl}perhydro-2H-pyran-4-carboxylate (3): 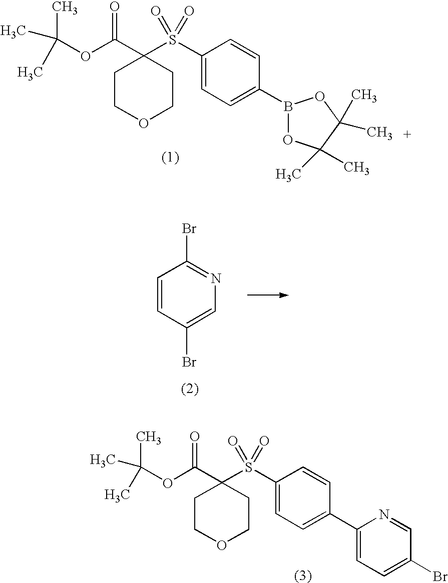
To a mixture of 4-[4-(4,4,5,5-tetramethyl-[1,3,2]dioxaborolan-2-yl)-benzenesulfonyl]-tetrahydro-pyran-4-carboxylic acid tert-butyl ester (1) (10.0 g, 22.2 mmol) in toluene (40 mL), ethanol (10 mL), and 1 M sodium carbonate (Na₂CO₃, 40 mL) under N₂ were added 2,5-dibromopyridine (2) (6.54 g, 27.6 mmol) and [1,1′-bis(diphenylphosphino)ferrocene]dichloropalladium(II) (“PD(dppf)Cl₂”, 0.90 g, 1.12 mmol). The resulting mixture was heated at 80° C. under N₂ overnight. Afterward, the mixture was cooled to room temperature and diluted with ethyl acetate and water. The mixture was then filtered through a pad of Celite. The layers of the filtrate were separated, and the organic layer was washed with water (2 times), washed with saturated NaCl (1 time), and dried over anhydrous sodium sulfate. Filtration and evaporation of the solvent under reduced pressure formed a dark oil. The residue was dissolved in dichloromethane and purified on SiO₂ (using 25% ethyl acetate/hexane). Clean, product-containing fractions were combined to afford 2.6 g of white solid (25% yield). ¹H NMR and mass spectrometry (MH⁺=482) were consistent with the desired compound (3).

Part B. Preparation of tert-butyl 4-({4-[5-(4,4,4-trifluorobutyl)pyridin-2-yl]phenyl}sulfonyl)tetrahydro-2H-pyran-4-carboxylate (4): 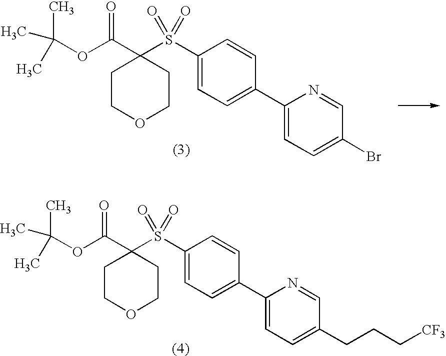
To a slurry of the ZnCu couple (2.23 g, 34.3 mmol) in benzene (57 mL) and DMF (3 mL) was added 1,1,1-trifluoro-4-iodobutane (5.33 g, 22.4 mmol). The resulting mixture was heated at 60° C. under N₂ for 3 hr. A mixture of the product (3) from Part A (3.59 g, 7.46 mmol) in benzene (14 mL) and DMF (3.5 mL) was subsequently added, followed by [1,1′-bis(diphenylphosphino)ferrocene]dichloropalladium(II) (“PD(dppf)Cl₂”, 0.30 g, 0.37 mmol). The temperature was then increased to 75° C., and the reaction was continued overnight, after which no starting material (3) was detected by HPLC. The mixture was then cooled to room temperature and diluted with ethyl acetate and water, and filtered through a pad of Celite. The layers of the filtrate were separated, and the organic layer was washed with water (2 times), washed with saturated NaCl (1 time), and dried over anhydrous sodium sulfate. Filtration and evaporation of the solvent under reduced pressure formed a dark oil. The crude material was purified on SiO₂ (using dichloromethane with a methanol gradient (0-0.5% methanol)) to afford 2.2 grams (57% yield) of a yellow foam. ¹H NMR and mass spectrometry (MH⁺=514.2) were consistent with the desired compound (4).

Part C. Preparation of the trifluoroacetic acid salt of 4-({4-[5-(4,4,4-trifluorobutyl)pyridin-2-yl]phenyl}sulfonyl)tetrahydro-2H-pyran-4-carboxylic acid (5): 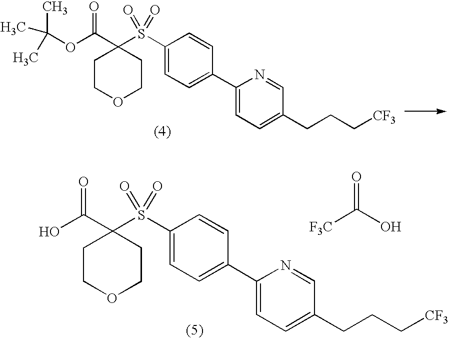
The product (4) from Part B (2.1 g, 4.1 mmol) was dissolved in 1:1 trifluoroacetic acid/dichloromethane (“TFA/CH₂Cl₂”, 50 mL). The reaction was then continued overnight at room temperature, after which no starting material (4) was detected by HPLC. The mixture was concentrated under reduced pressure. Diethyl ether was added, and the solvent was once again removed under reduced pressure. Diethyl ether was added a final time, and 1.8 g of white solid was collected by suction filtration (77% yield for the TFA salt). ¹H NMR and mass spectrometry (MH⁺=458.1) were consistent with the desired compound (5).

Part D. Preparation of N-(tetrahydro-2H-pyran-2-yloxy)-4-({4-[5-(4,4,4-trifluorobutyl)pyridin-2-yl]phenyl}sulfonyl)tetrahydro-2H-pyran-4-carboxamide (6): 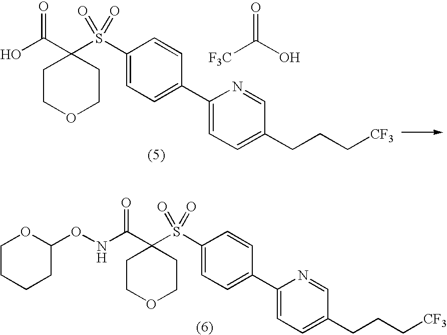
To a mixture of the product (5) from Part C (1.77 g, 3.10 mmol for the TFA salt) in N,N-dimethylformamide (“DMF”, 57 mL) were added N-hydroxybenzotriazole (“HOBt”, 0.59 g, 4.34 mmol), 4-methylmorpholine (“NNM”, 1.25 g, 1.36 mL, 12.4 mmol), 1-(3-dimethylaminopropyl)-3-ethylcarbodiimide hydrochloride (“EDC.HCl”, 1.49 g, 7.75 mmol), and O-(tetrahydro-2H-pyran-2-yl)hydroxylamine (“THPONH₂”, 0.91 g, 7.75 mmol). The reaction was then continued overnight at room temperature under N₂, after which no starting material (5) was detected by HPLC. The mixture was then diluted with ethyl acetate. The organic layer was extracted with water (3 times), saturated sodium bicarbonate (3 times), washed with saturated NaCl, and dried over anhydrous sodium sulfate. Filtration and evaporation of the solvent under reduced pressure formed a white foam. The crude material was purified by flash chromatography (using dichloromethane with a methanol gradient (0-1%) to afford 1.3 g of a white foam (76% yield). ¹H NMR and mass spectrometry (MH⁺=557.2) were consistent with the desired compound (6).

Part E. Preparation of N-hydroxy-4-({4-[5-(4,4,4-trifluorobutyl)pyridin-2-yl]phenyl}sulfonyl)tetrahydro-2H-pyran-4-carboxamide hydrochloride (7): 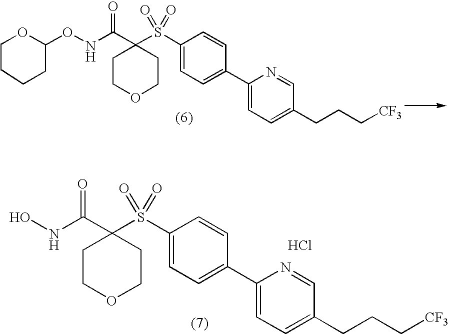
The product (6) from Part D (1.3 g, 2.3 mmol) was dissolved in dioxane (8 mL), 4N HCl in dioxane (10 mL), and methanol (1 mL). The reaction was then continued at ambient temperature overnight. Afterward, HPLC indicated that a small amount of starting material (6) was still present. The mixture was concentrated under reduced pressure, and the residue was resubmitted to the reaction conditions described above. After 1 hr, HPLC indicated that the reaction was complete. The solvent was then removed under reduced pressure, and the resulting residue was triturated with diethyl ether to form a white solid, which, in turn, was collected by suction filtration to afford 1.1 g of product (92% yield). ¹H NMR and high resolution mass spectrometry (theoretical MH⁺=473.1353, actual MH⁺=473.1356) were consistent with the desired product (7).

### Examples 36-69

### In Vitro MMP Inhibition Analysis

Several compounds and salts were analyzed in an in vitro assay to determine their ability to inhibit the MMP cleavage of peptide substrates. Inhibition constant (Kᵢ) were calculated from the assayed compound-MMP interactions.

Human recombinant MMP-1, MMP-2, MMP-9, MMP-13, and MMP-14 were used in this assay. All enzymes were prepared in Assignee's laboratories following usual laboratory procedures. Protocols for the preparation and use of these enzymes are available in the scientific literature. See, e.g., Enzyme Nomenclature (Academic Press, San Diego, Calif., 1992) (and the citations therein). See also, Frije et al., J. Biol. Chem., 26(24), 16766-73 (1994).

The MMP-1 proenzyme was purified from the spent media of MMP-1-transfected HT-1080 cells provided by Dr. Harold Welgus of Washington University (St. Louis, Mo.). The protein was purified on a zinc chelating column.

The MMP-2 proenzyme was purified by gelatin Sepharose chromatography from MMP-2-transfected p2AHT2 cells provided by Dr. Gregory Goldberg of Washington University (St. Louis, Mo.).

The MMP-9 proenzyme was purified by gelatin Sepharose chromatography from spent media of MMP-9-transfected HT1080 cells provided by Dr. Howard Welgus of Washington University (St. Louis, Mo.).

The MMP-13 was obtained as a proenzyme from a full-length cDNA clone using baculovirus, as described by V. A. Luckow, “Insect Cell Expression Technology,” Protein Engineering: Principles and Practice, pp. 183-218 (edited by J. L. Cleland et al., Wiley-Liss, Inc., 1996). The expressed proenzyme was first purified over a heparin agarose column, and then over a chelating zinc chloride column. The proenzyme was then activated by APMA for use in the assay. Further details on baculovirus expression systems may be found in, for example, Luckow et al., J. Virol., 67, 4566-79 (1993). See also, O'Reilly et al, Baculovirus Expression Vectors: A Laboratory Manual (W.H. Freeman and Co., New York, N.Y., 1992). See also, King et al., The Baculovirus Expression System: A Laboratory Guide (Chapman & Hall, London, England, 1992).

The MMP-14 full length cDNA was provided by Dr. Gregory Goldberg of Washington University (St. Louis, Mo.). The catalytic domain enzyme was expressed in E. coli inclusion bodies, solubilized in urea, purified on a preparative C-14 reverse phase HPLC column, and then refolded in the presence of zinc acetate and purified for use.

All MMPs were activated using 4-aminophenylmercuric acetate (“APMA”, Sigma Chemical, St. Louis, Mo.) or trypsin. MMP-9 also was activated using human recombinant MMP-3 (purified in Assignee's laboratory following standard cloning and purification techniques).

The following fluorogenic, methoxycoumarin-containing polypeptide substrate (I) was used in the MMP inhibition assays:
MCA-ArgProLeuGlyLeuDpaAlaArgGluArgNH₂ (I)
“MCA” is 7-methoxycoumarin-4-yl acetyl. Substrate (I) was prepared Assignee's laboratory. In the absence of MMP inhibitory activity, the substrate is cleaved at the Gly-Leu peptide bond. This cleavage separates the highly fluorogenic peptide from the 2,4-dinitrophenyl quencher, thus resulting in increase of fluorescent intensity.

The stock solutions of the assayed compounds and salts were prepared in 1% dimethyl sulfoxide (DMSO). These stock solutions were diluted in Buffer A (100 mM Tris-HCl, 100 mM NaCl, 10 mM CaCl₂, 0.05% polyoxyethylene 23 lauryl ether, pH 7.5) to obtain solutions with different compound concentrations, i.e., assay solutions with different concentrations of the assayed M inhibitory compound. The experiment controls contained the same amount of Buffer A/DMSO as the assayed sample, but contained none of the tested compound or salt.

The assays from which the Kᵢ determinations were made were performed as follows. The assayed compound samples were incubated in separate wells of untreated white polystyrene plates (Nunc Nalgene International, Rochester, N.Y.), and analyzed on a Tecan SpectraFlour Plus plate reader. The excitation wavelength was 330 nm, and the emission wavelength—420 nm. All samples (assayed compounds and controls) were incubated in separate plate wells at room temperature for 1 hr in the presence of 4 μM of MMP substrate (I). In the absence of MMP inhibitory activity, substrate (I) was cleaved at the Gly-Leu bond resulting in an increase of relative fluorescence. Inhibition was observed as a reduced rate of this increase in relative fluorescence. The various compounds were analyzed using a single low enzyme concentration with a single substrate concentration fixed at or below the Kₘ. This protocol is a modification of method by Knight et al., FEBS Lett., 296(3), 263-266 (1992). Apparent inhibitory constants were determined by non-linear regression of reaction velocity as a function of inhibitor and enzyme concentration using Morrison's equation, as described by Kuzmic, Anal. Biochem. 286, 45-50 (2000). Modifications were made in the non-linear regression method to allow a common control reaction rate and effective enzyme concentration to be shared between all dose-response relationships on a given assay plate. Since the substrate concentration was chosen to be at or below the Kₘ, the apparent Kᵢ's from this analysis were reported as Kᵢ's without correction for the influence of substrate.

The above protocols were used to determine Kᵢ constants for the compounds in Examples 2-35 above. The results are shown in Table 1. All Kᵢ values in Table 1 are given in nM units.

TABLE 1

| Ex. No. | Structure | MMP-1 | MMP-2 | MMP-9 | MMP-13 | MMP-14 |
|---|---|---|---|---|---|---|
| 36 | 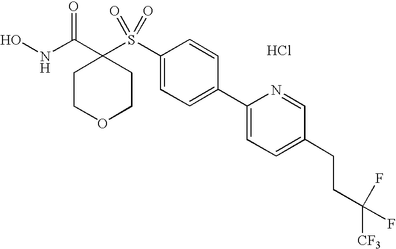 | 4723 | 0.0708 | 0.258 | 0.0403 | 523 |
| | Prepared in Example 2 Above | | | | | |
| 37 | 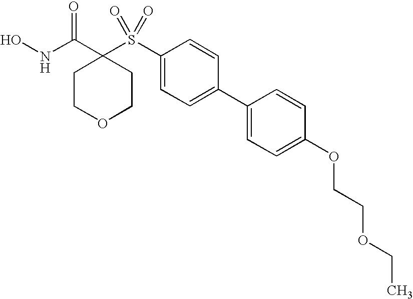 | 1450 | 0.089 | 3.51 | 0.028 | 632 |
| | Prepared in Example 3 Above | | | | | |
| 38 | 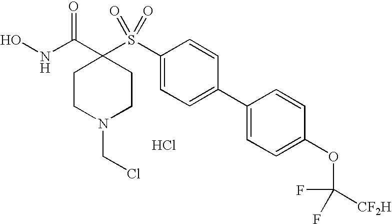 | 650 | 0.037 | 0.126 | 0.02 | 446 |
| | Prepared in Example 4 Above | | | | | |
| 39 | 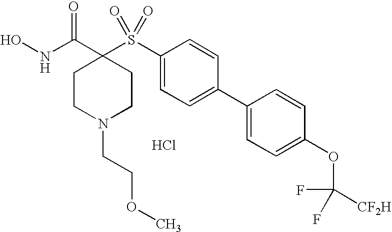 | 335 | 0.009 | 0.045 | 0.011 | 185 |
| | Prepared in Example 5 Above | | | | | |
| 40 | 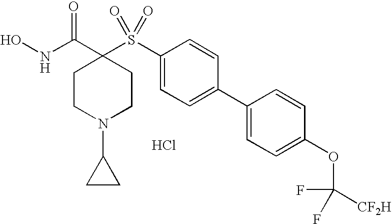 | 508 | 0.074 | 0.102 | 0.066 | 292 |
| | Prepared in Example 6 Above | | | | | |
| 41 | 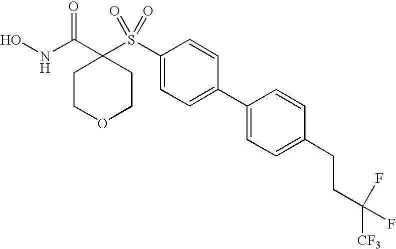 | 5115 | 0.375 | 0.937 | 0.224 | 1140 |
| | Prepared in Example 7 Above | | | | | |
| 42 | 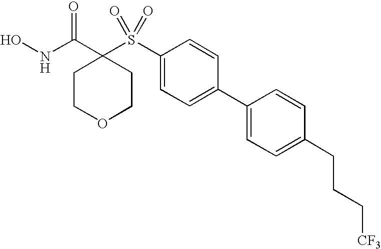 | 2710 | 0.339 | 0.642 | 0.072 | 800 |
| | Prepared in Example 8 Above | | | | | |
| 43 | 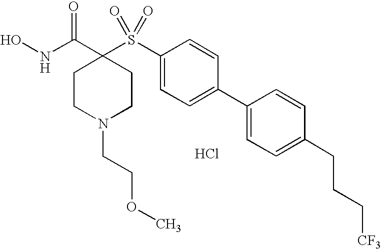 | 6870 | 0.29 | 0.43 | 0.06 | 1590 |
| | Prepared in Example 9 Above | | | | | |
| 44 | 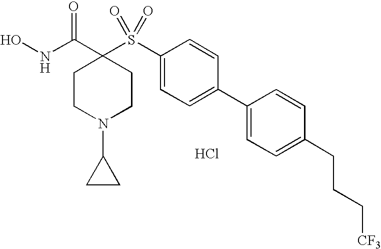 | 8690 | 0.48 | 0.68 | 0.13 | 2160 |
| | Prepared in Example 10 Above | | | | | |
| 45 | 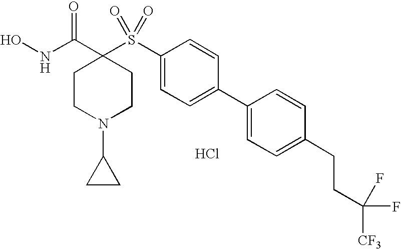 | >10000 | 0.55 | 1.16 | 0.46 | 1730 |
| | Prepared in Example 11 Above | | | | | |
| 46 | 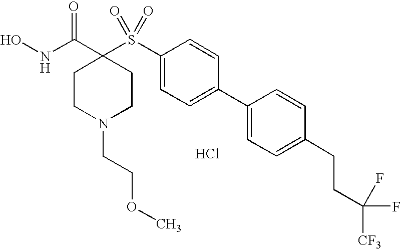 | >10000 | 0.21 | 0.38 | 0.11 | 1860 |
| | Prepared in Example 12 Above | | | | | |
| 47 | 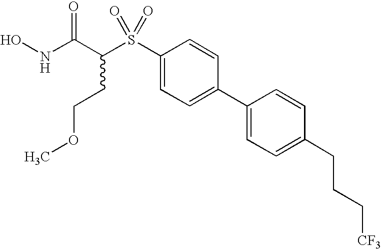 | >10000 | 1.17 | 2.8 | 0.40 | 5260 |
| | Prepared in Example 13 Above | | | | | |
| 48 | 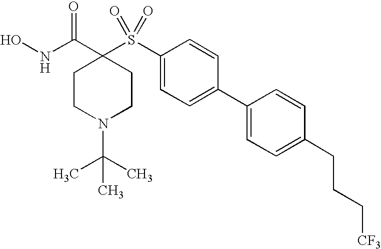 | >10000 | 1.84 | 3.54 | 0.221 | 8450 |
| | Prepared in Example 14 Above | | | | | |
| 49 | 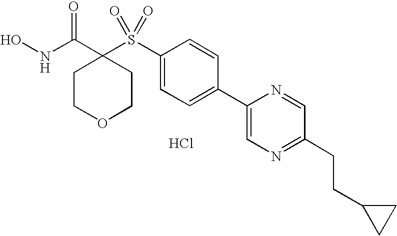 | >10000 | 2.36 | 0.88 | 0.71 | 3620 |
| | Prepared in Example 15 Above | | | | | |
| 50 | 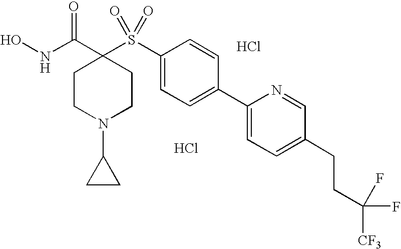 | 7880 | 0.19 | 0.22 | 0.12 | 1190 |
| | Prepared in Example 16 Above | | | | | |
| 51 | 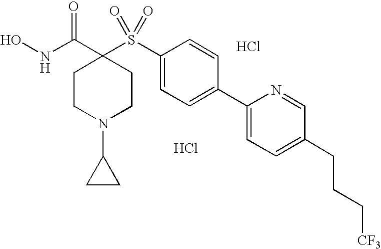 | 9600 | 0.16 | 0.22 | 0.05 | 1890 |
| | Prepared in Example 17 Above | | | | | |
| 52 | 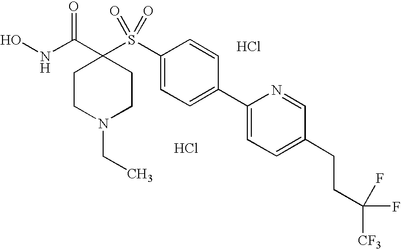 | >10000 | 0.21 | 0.58 | 0.07 | 2580 |
| | Prepared in Example 18 Above | | | | | |
| 53 | 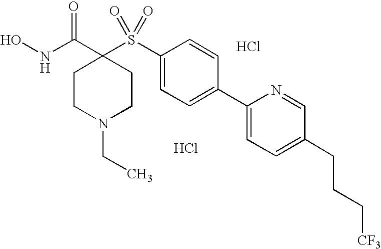 | >10000 | 0.46 | 1 | 0.06 | 3960 |
| | Prepared in Example 19 Above | | | | | |
| 54 | 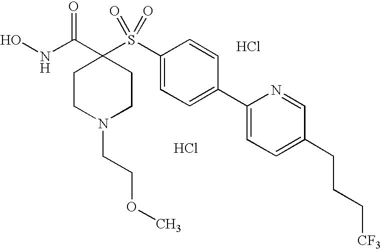 | 6370 | 0.12 | 0.25 | 0.3 | 1110 |
| | Prepared in Example 20 Above | | | | | |
| 55 | 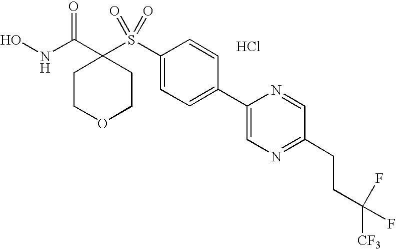 | >10000 | 0.586 | 0.347 | 0.222 | 2560 |
| | Prepared in Example 21 Above | | | | | |
| 56 | 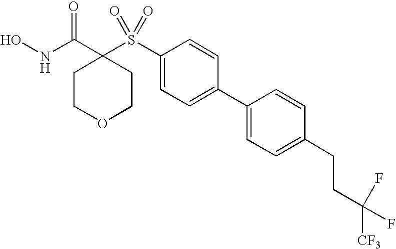 | 1120 | 0.046 | 0.157 | 0.022 | 230 |
| | Prepared in Example 22 Above | | | | | |
| 57 | 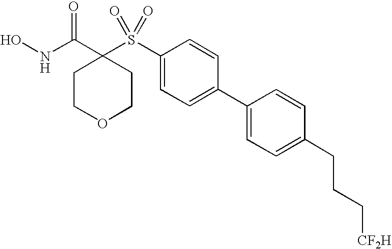 | 2120 | 0.15 | 0.38 | 0.04 | 453 |
| | Prepared in Example 23 Above | | | | | |
| 58 | 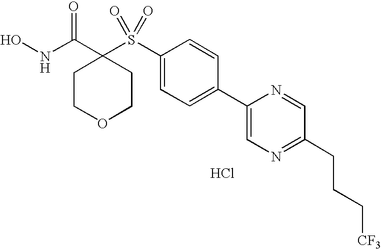 | >10,000 | 2.02 | 1.67 | 0.45 | >10,000 |
| | Prepared in Example 24 Above | | | | | |
| 59 | 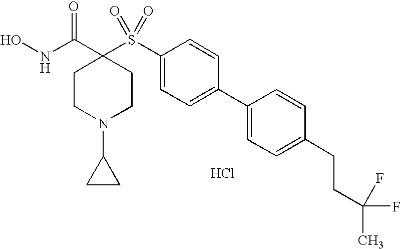 | 2170 | 0.11 | 0.16 | 0.05 | 390 |
| | Prepared in Example 25 Above | | | | | |
| 60 | 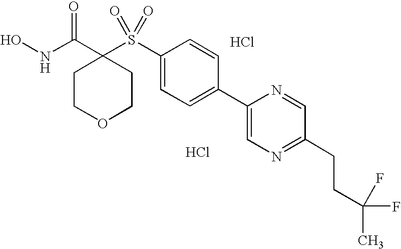 | >10000 | 0.463 | 0.623 | 0.212 | 1880 |
| | Prepared in Example 26 Above | | | | | |
| 61 | 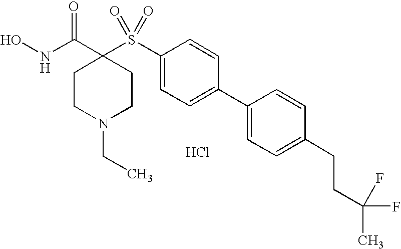 | 3040 | 0.11 | 0.26 | 0.02 | 570 |
| | Prepared in Example 27 Above | | | | | |
| 62 | 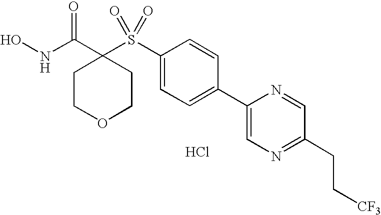 | >10000 | 0.88 | 1.83 | 0.45 | 2930 |
| | Prepared in Example 28 Above | | | | | |
| 63 | 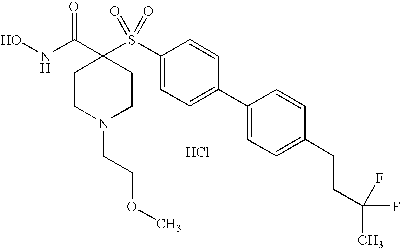 | 1750 | 0.04 | 0.08 | 0.01 | 237 |
| | Prepared in Example 29 Above | | | | | |
| 64 | 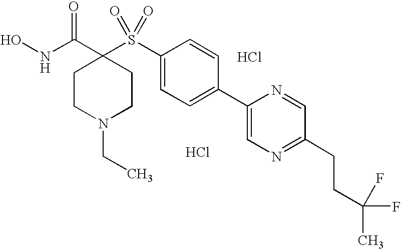 | >10000 | 1.65 | 0.677 | 0.296 | >10000 |
| | Prepared in Example 30 Above | | | | | |
| 65 | 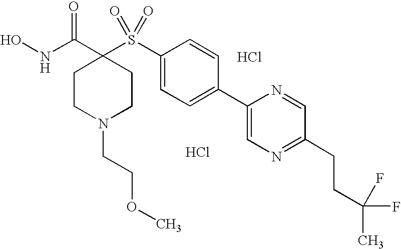 | >10000 | 0.952 | 0.375 | 0.27 | 5970 |
| | Prepared in Example 31 Above | | | | | |
| 66 | 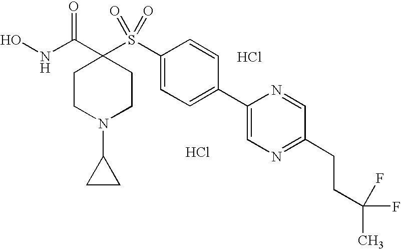 | >10000 | 0.79 | 0.31 | 0.23 | 4260 |
| | Prepared in Example 32 Above | | | | | |
| 67 | 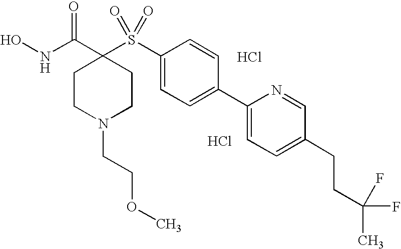 | 8470 | 0.14 | 0.19 | 0.07 | 831 |
| | Prepared in Example 33 Above | | | | | |
| 68 | 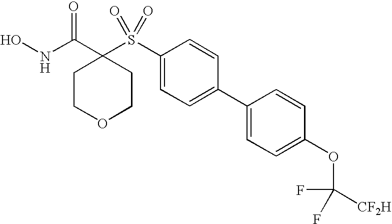 | 231 | 0.0611 | 0.142 | 0.0509 | 235 |
| | Prepared in Example 34 Above | | | | | |
| 69 | 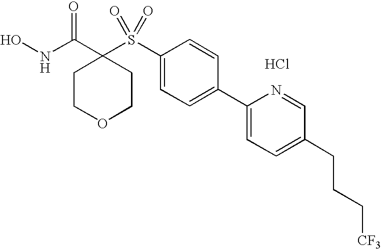 | 3530 | 0.322 | 0.719 | 0.082 | 708 |
| | Prepared in Example 35 Above | | | | | |

### Examples 70-223

Additional compounds and salts can be prepared by one skilled in the art using methods similar to those described in Examples 1-35 alone or in combination with techniques well known in the art. Such compounds and salts include, for example, the compounds summarized in the following Table 2. Table 2 also summarizes in vitro MMP inhibition results obtained by Applicants with the listed compounds and salts. All Kᵢ results in Table 2 are given in nM units.

TABLE 2

| | | | | | | | Calc. | Observed |
|---|---|---|---|---|---|---|---|---|
| Ex. No. | Structure | MMP-1 | MMP-2 | MMP-9 | MMP-13 | MMP-14 | Mass | Mass |
| 70 | 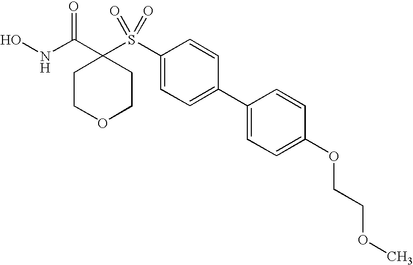 | 969 | 0.177 | 1.11 | 0.03 | 299 | 436.6782 | 436.6219 |
| 71 | 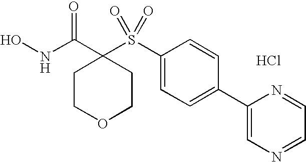 | 2960 | 5.96 | 42.8 | 1.73 | 224 | 364.0962 | 364.0985 |
| 72 | 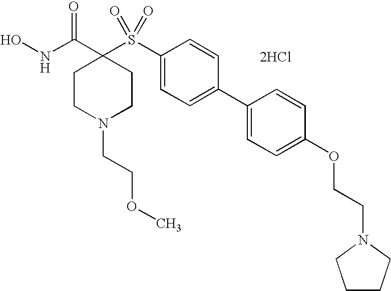 | >10000 | 5.81 | 12.9 | 2.25 | 2276 532.2476 | 532.2443 | |
| 73 | 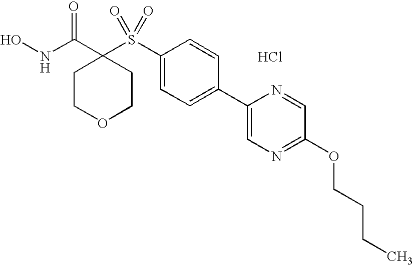 | 1740 | 0.0835 | 0.112 | 0.028 | 159 | 436.1537 | 436.1565 |
| 74 | 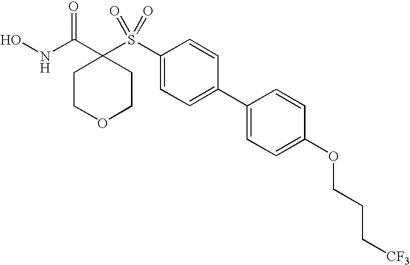 | 1630 | 0.304 | 3.40 | 0.102 | 585 | 505.1615 | 505.1623 |
| 75 | 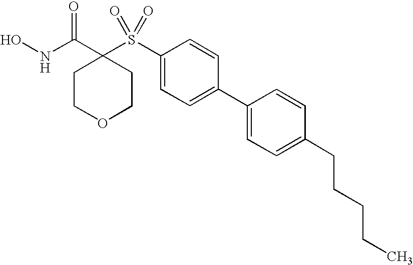 | 3970 | 0.674 | 1.01 | 0.299 | 621 | 432.1789 | 432.1802 |
| 76 | 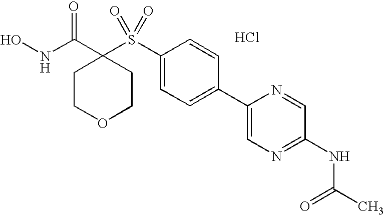 | 9630 | 10.3 | 114 | 31.7 | 3480 | 421.1176 | 421.1165 |
| 77 | 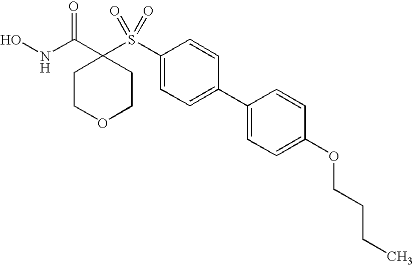 | 1290 | 0.180 | 0.398 | 0.0485 | 174 | 434.1632 | 434.1657 |
| 78 | 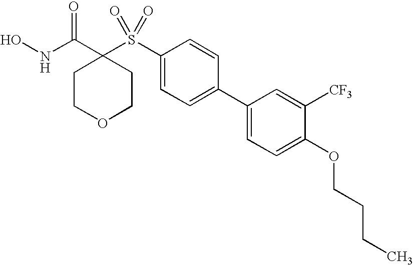 | >10000 | 55.5 | 132 | 16.6 | 9570 | 519.1771 | 519.1778 |
| 79 | 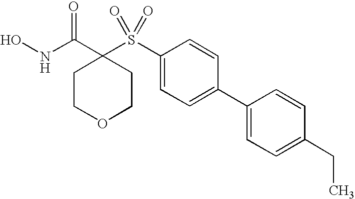 | 471 | 0.184 | 0.635 | 0.091 | 27.4 | 390.137 | 390.1374 |
| 80 | 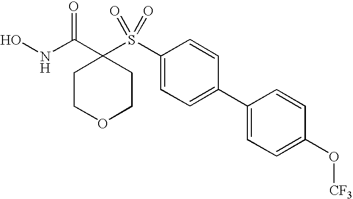 | 577 | 0.098 | 0.622 | 0.068 | 89.1 | 463.1145 | 463.1141 |
| 81 | 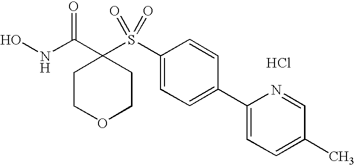 | 354 | 0.106 | 0.993 | 0.118 | 22.6 | 377.1166 | 377.12 |
| 82 | 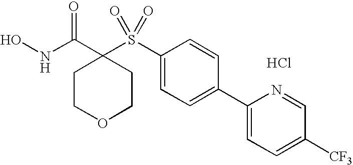 | 256 | 0.200 | 1.99 | 0.13 | 65.4 | 431.0083 | 431.0897 |
| 83 | 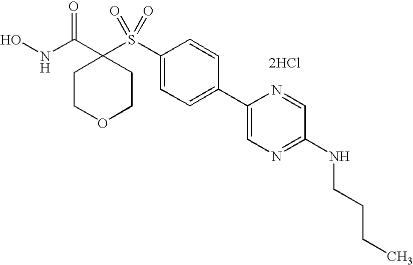 | >10000 | 7.44 | 2.75 | 1.31 | 6360 | 435.1697 | 435.1686 |
| 84 | 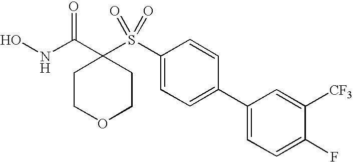 | >10000 | 10.1 | 97.1 | 1.85 | 989 | 465.1102 | 465.1079 |
| 85 | 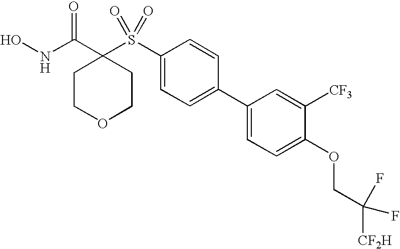 | >10000 | 33.4 | 248 | 14.7 | 2800 | 560.0972 | 560.0963 |
| 86 | 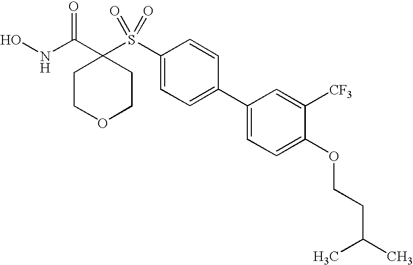 | >10000 | 85.7 | 414 | 36.2 | >10000 | 516.1662 | 516.1677 |
| 87 | 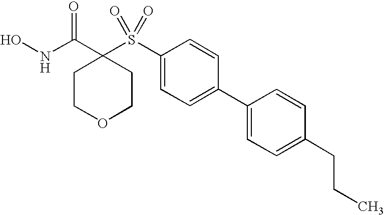 | 1110 | 0.158 | 0.176 | 0.138 | 62.3 | 404.1526 | 404.1518 |
| 88 | 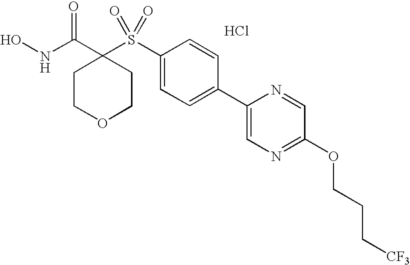 | 3523 | 0.15 | 2.31 | 0.0368 | 539 | 490.1254 | 490.1258 |
| 89 | 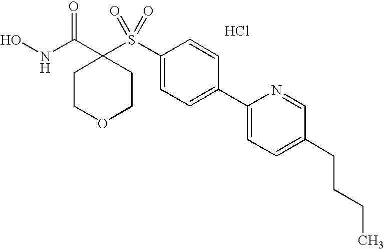 | 2263 | 0.0488 | 0.0528 | 0.0332 | 139 | 419.1635 | 419.1637 |
| 90 | 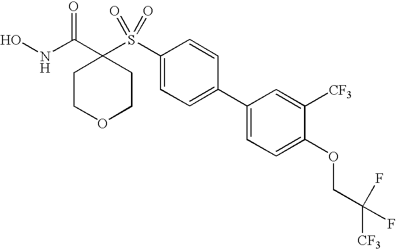 | >10000 | 28.3 | 216 | 19.7 | 2100 | 595.1144 | 595.1112 |
| 91 | 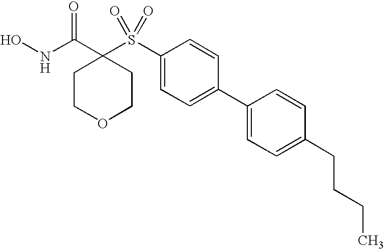 | 3900 | 0.374 | 0.266 | 0.217 | 321 | 418.1683 | 418.1672 |
| 92 | 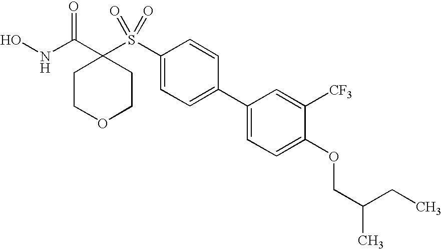 | >10000 | 58.1 | 106 | 29.7 | 7410 | 516.1662 | 516.1621 |
| 93 | 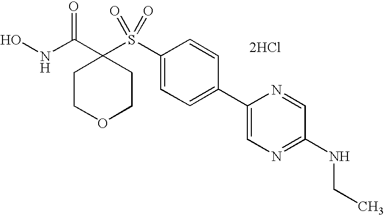 | >10000 | 4.16 | 4.86 | 4.45 | 4450 | 407.1384 | 407.1377 |
| 94 | 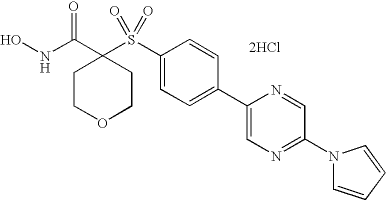 | >10000 | 1.09 | 3.4 | 0.428 | 2940 | 429.1227 | 429.1206 |
| 95 | 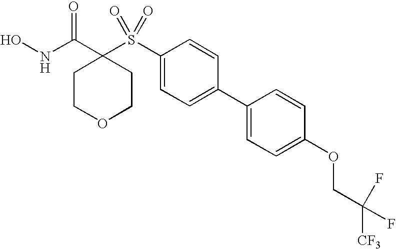 | 1020 | 0.170 | 0.752 | 0.155 | 463 | 510.1004 | 510.0996 |
| 96 | 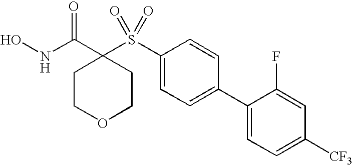 | 602 | 0.668 | 2.14 | 0.416 | 224 | 448.0808 | 448.0836 |
| 97 | 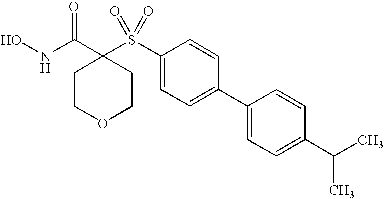 | 1040 | 1.14 | 5.40 | 0.521 | 109 | 404.1526 | 404.1538 |
| 98 | 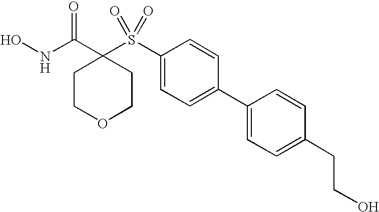 | 990 | 0.158 | 3.1 | 0.147 | 52.6 | 406.1319 | 406.1327 |
| 99 | 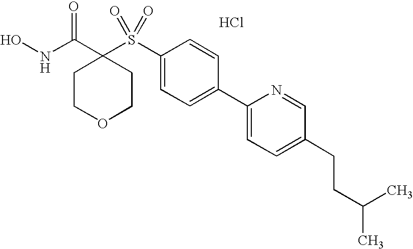 | 3080 | 0.112 | 0.211 | 0.052 | 421 | 433.1768 | 433.1792 |
| 100 | 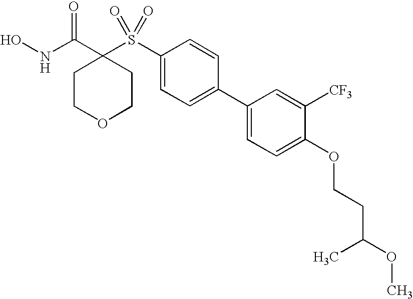 | >10000 | 32.5 | 849 | 9.4 | >10000 | 532.1611 | 532.1624 |
| 101 | 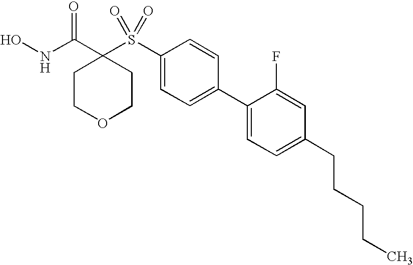 | 9030 | 0.878 | 3.05 | 0.4 | 929 | 450.1745 | 450.1729 |
| 102 | 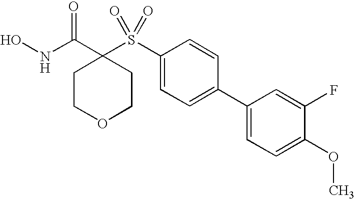 | 669 | 0.078 | 1.01 | 0.075 | 11.1 | 410.1068 | 410.1088 |
| 103 | 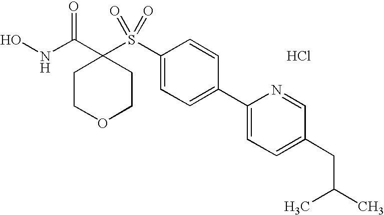 | 2320 | 0.278 | 1.38 | 0.117 | 342 | 419.1635 | 419.162 |
| 104 | 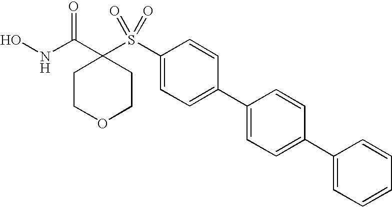 | 6910 | 0.087 | 0.074 | 0.032 | 247 | 438.137 | 438.1357 |
| 105 | 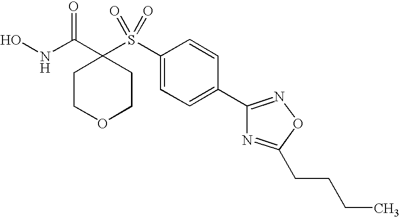 | >10000 | 5.79 | 2.15 | 3.67 | 1730 | 410.138 | 410.1402 |
| 106 | 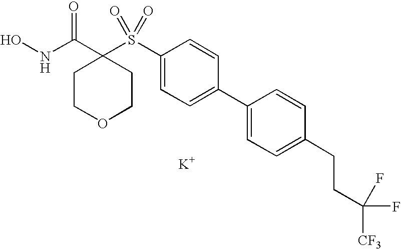 | | | | | | | |
| 107 | 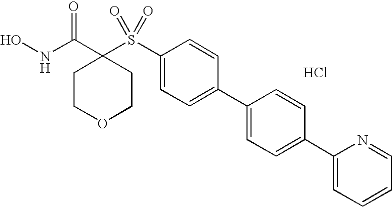 | 2255 | 0.777 | 0.601 | 0.23 | 3405 | 525.1477 | 525.1463 |
| 108 | 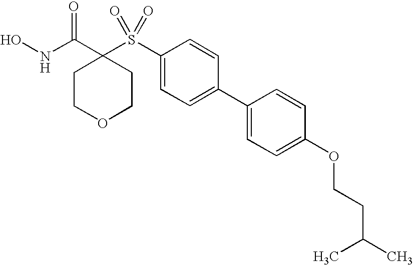 | 2120 | 0.497 | 2.29 | 0.199 | 744 | 448.1788 | 448.182 |
| 109 | 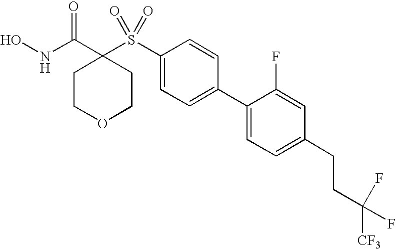 | >10000 | 0.541 | 4.33 | 0.52 | 1640 | 439.1322 | 439.1329 |
| 110 | 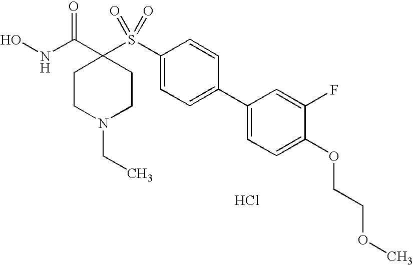 | >10000 | 0.539 | 11.2 | 0.144 | 1850 | 481.1809 | 481.1823 |
| 111 | 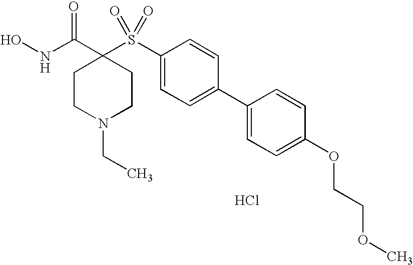 | 7360 | 0.645 | 3.3 | 0.066 | 1790 | 463.1963 | 463.1918 |
| 112 | 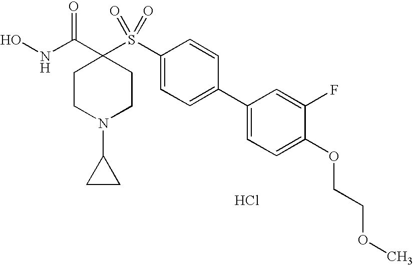 | 8580 | 0.168 | 2.21 | 0.058 | 681 | 493.1809 | 493.1817 |
| 113 | 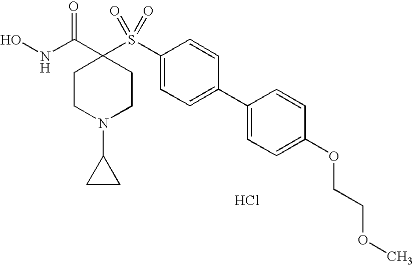 | 2910 | 0.170 | 0.732 | 0.031 | 499 | 475.1903 | 475.1909 |
| 114 | 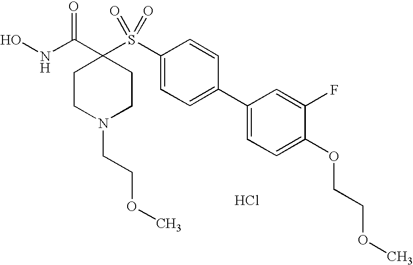 | 6550 | 0.217 | 2.77 | 0.042 | 585 | 511.1914 | 511.1914 |
| 115 | 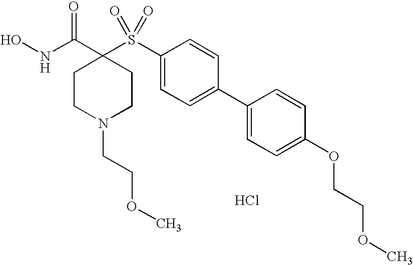 | 3370 | 0.276 | 1.22 | 0.025 | 530 | 493.2009 | 493.2008 |
| 116 | 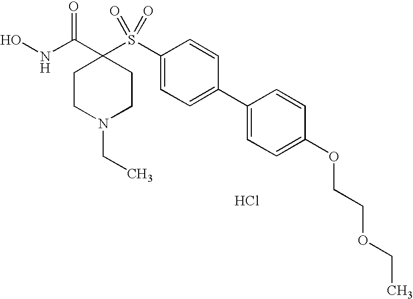 | 8940 | 0.514 | 9.39 | 0.05 | 2930 | 477.2059 | 477.206 |
| 117 | 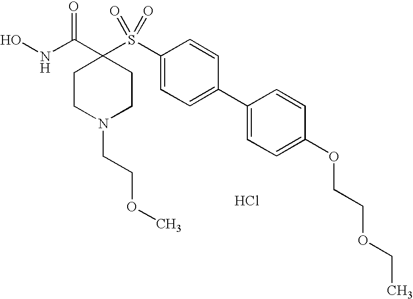 | 4900 | 0.208 | 3.93 | 0.037 | 1110 | 507.2165 | 507.2158 |
| 118 | 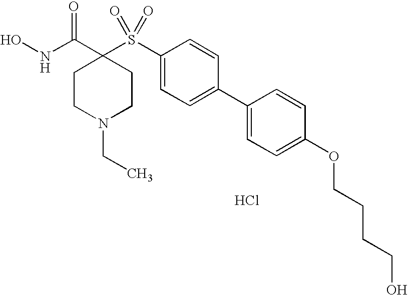 | 2170 | 0.146 | 0.867 | 0.012 | 741 | 477.2059 | 477.2068 |
| 119 | 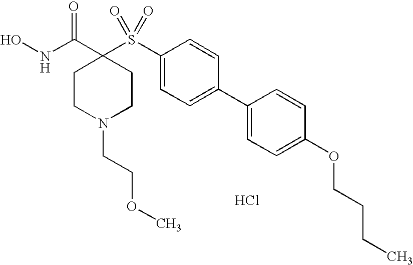 | 1770 | 0.093 | 0.150 | 0.018 | 171 | 491.2216 | 491.2214 |
| 120 | 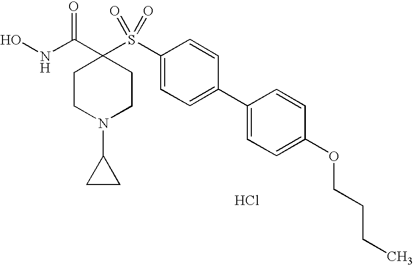 | 2460 | 0.191 | 0.336 | 0.082 | 311 | 473.211 | 473.2119 |
| 121 | 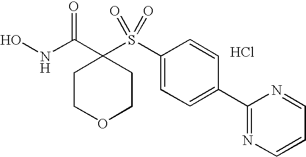 | 1600 | 46.8 | 57.9 | 4.86 | 1880 | 364.0962 | 364.0921 |
| 122 | 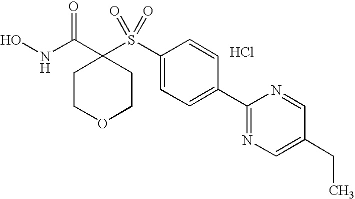 | 3580 | 5.67 | 4.84 | 1.56 | 315 | 392.1275 | 392.1265 |
| 123 | 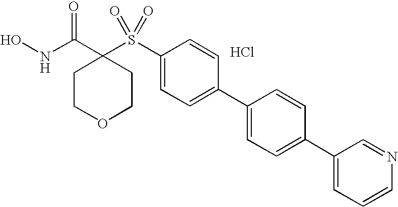 | 260 | 0.123 | 0.392 | 0.0308 | 418 | 526.1117 | 526.1103 |
| 124 | 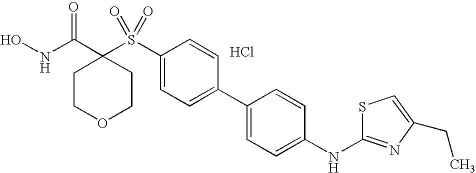 | >2500 | 32.9 | 191 | 3.3 | 6230 | 488.1308 | 488.1276 |
| 125 | 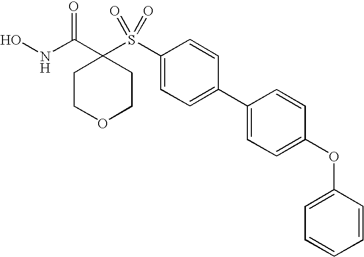 | 3950 | 3.83 | 10 | 0.718 | 453 | 439.1322 | 439.1324 |
| 126 | 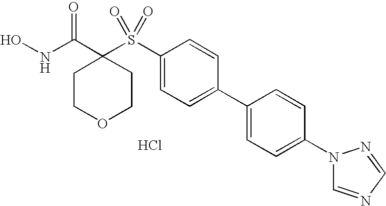 | 1239 | 0.18 | 1.92 | 0.048 | 905 | 429.1227 | 429.1241 |
| 127 | 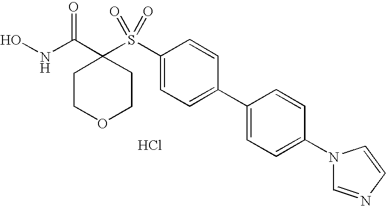 | 1012 | 0.147 | 1.89 | 0.03 | 414 | 428.1275 | 428.1277 |
| 128 | 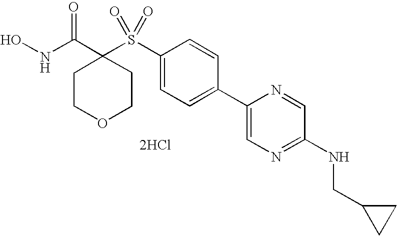 | >2500 | 5.08 | 4.36 | 3.40 | >2500 | 433.154 | 433.1577 |
| 129 | 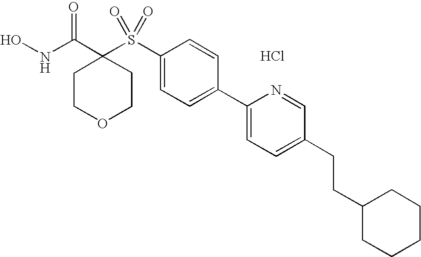 | 4410 | 0.940 | 8.56 | 0.383 | 745 | 473.2105 | 473.2119 |
| 130 | 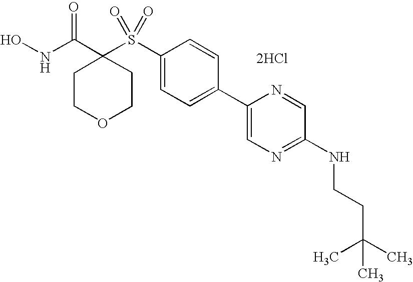 | >1250 | 14.2 | 12.8 | 1.27 | >1250 | 463.201 | 463.2025 |
| 131 | 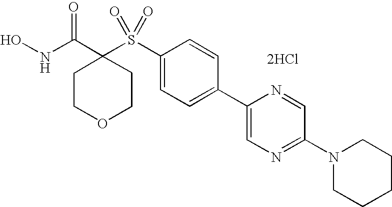 | >10000 | 0.890 | 5.30 | 2.04 | 6240 | 447.1697 | 447.1665 |
| 132 | 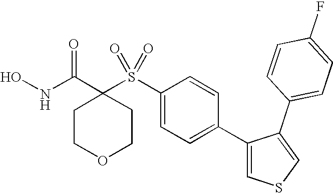 | >10000 | 153 | 298 | 71 | 3170 | 479.1105 | 479.1083 |
| 133 | 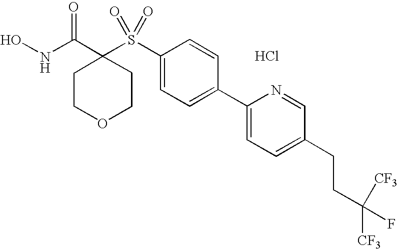 | 7400 | 11.9 | 35.2 | 2.06 | 4150 | 559.1138 | 559.1108 |
| 134 | 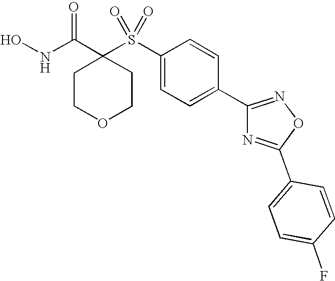 | 5910 | 1.13 | 5.25 | 1.76 | 67.1 | 448.0979 | 448.0968 |
| 135 | 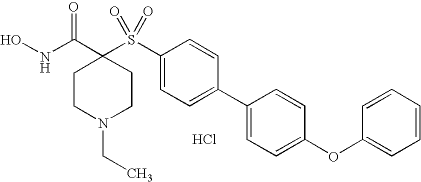 | >10000 | 4.22 | 9.95 | 0.742 | 1010 | 481.1797 | 481.1795 |
| 136 | 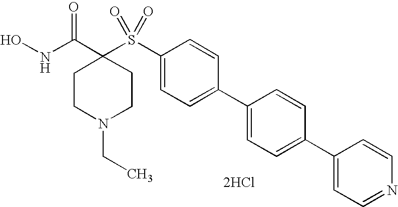 | 1070 | 0.074 | 0.418 | 0.021 | 308 | | |
| 137 | 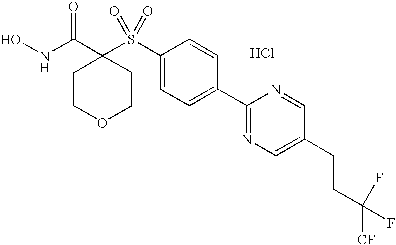 | >10000 | 2.72 | 1.15 | 0.17 | 2870 | 510.1125 | 510.1122 |
| 138 | 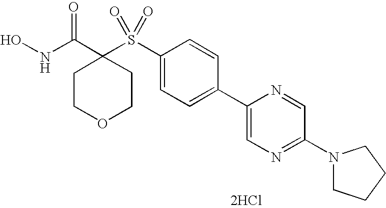 | >10000 | 0.218 | 1.04 | 0.571 | 1110 | 433.1546 | 433.154 |
| 139 | 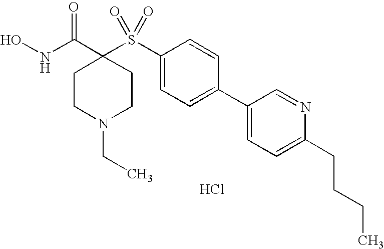 | >10000 | 9 | 6.23 | 5.22 | >10000 | 446.2114 | 446.2149 |
| 140 | 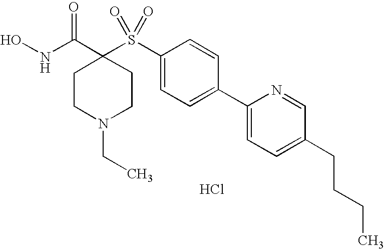 | >10000 | 0.176 | 0.127 | 0.058 | 604 | 446.2114 | 446.2133 |
| 141 | 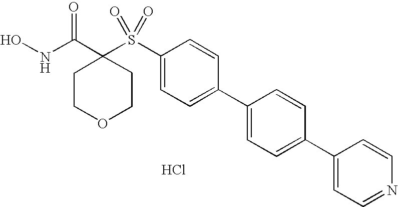 | 257 | 0.041 | 0.198 | 0.012 | 79.4 | 437.1166 | 437.117 |
| 142 | 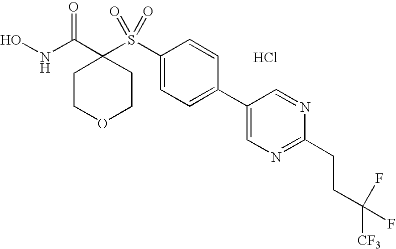 | >10000 | 53.7 | 76.6 | 22.5 | >10000 | 510.1117 | 510.10 |
| 143 | 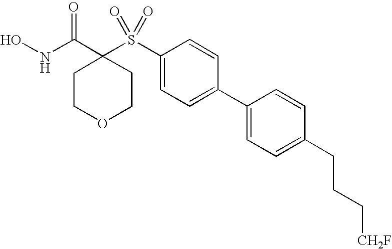 | 1600 | 0.13 | 0.24 | 0.05 | 266 | 454.1494 | 454.1468 |
| 144 | 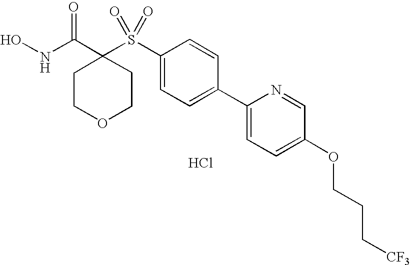 | 481 | 0.037 | 1.13 | 0.007 | 187 | 489.1302 | 489.1308 |
| 145 | 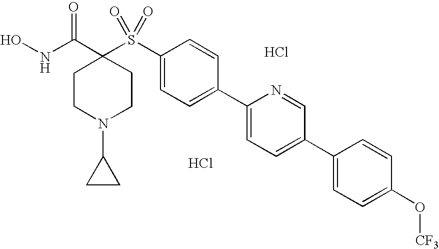 | 3390 | 0.72 | 6.62 | 0.26 | 560 | 562.1624 | 562.1638 |
| 146 | 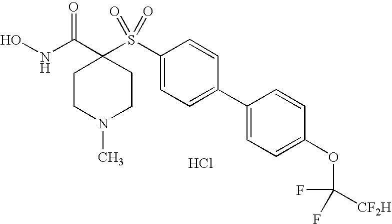 | 1240 | 0.30 | 0.48 | 0.15 | 1050 | 491.1264 | 491.1248 |
| 147 | 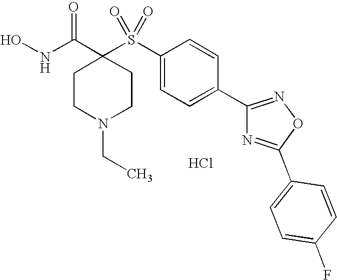 | >10000 | 1.75 | 7.09 | 2.59 | 139 | 475.1452 | 475.1442 |
| 148 | 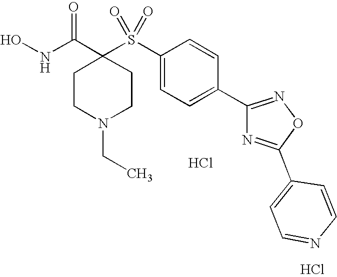 | >10000 | 7.26 | 39.3 | 5.7 | 1020 | 458.1498 | 458.1482 |
| 149 | 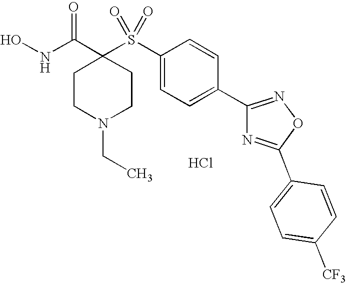 | >10000 | 0.76 | 5.18 | 0.45 | 117 | 525.142 | 525.1409 |
| 150 | 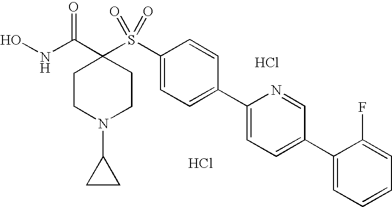 | 1500 | 0.37 | 0.47 | 0.12 | 307 | 496.1706 | 496.1714 |
| 151 | 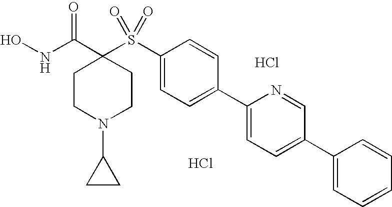 | 391 | 0.15 | 0.09 | 0.05 | 120 | 478.1801 | 478.1817 |
| 152 | 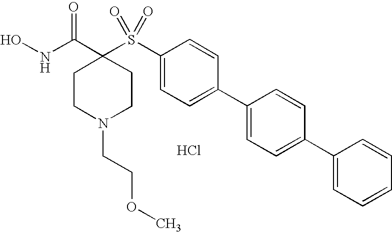 | 842 | 0.10 | 0.09 | 0.05 | 144 | 495.1954 | 495.1949 |
| 153 | 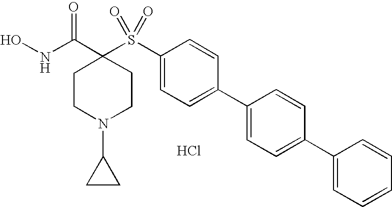 | 1310 | 0.26 | 0.18 | 0.15 | 205 | 477.1848 | 477.1847 |
| 154 | 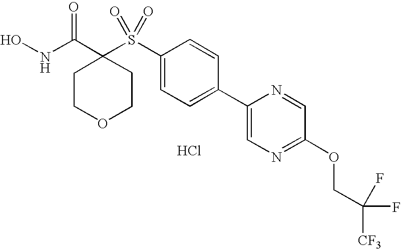 | 1840 | 0.13 | 0.34 | 0.12 | 493 | 512.0909 | 512.092 |
| 155 | 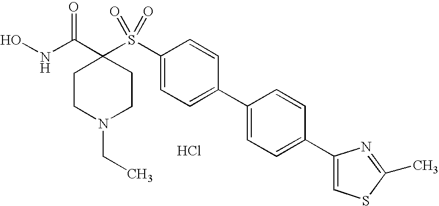 | 3860 | 0.66 | 0.44 | 0.17 | 283 | 486.1516 | 486.152 |
| 156 | 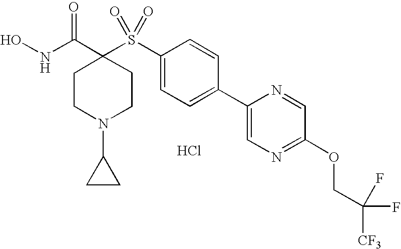 | 7470 | 0.39 | 0.48 | 0.31 | 1140 | 551.1382 | 551.1421 |
| 157 | 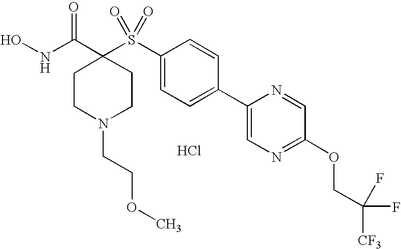 | 9580 | 0.37 | 0.42 | 0.26 | 1220 | 569.1488 | 569.146 |
| 158 | 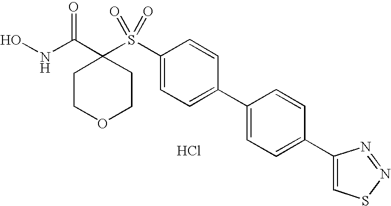 | 61.9 | 0.06 | 0.03 | 0.04 | 101 | 482.9811 | 482.9803 |
| 159 | 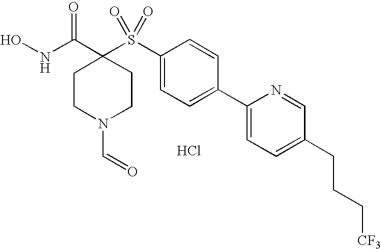 | 3480 | 0.10 | 0.38 | 0.02 | 599 | 500.1462 | 500.1439 |
| 160 | 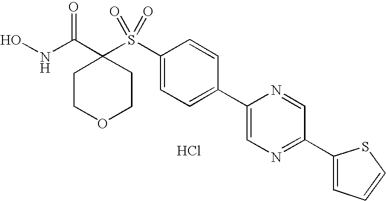 | >10000 | 2.01 | 3.16 | 0.70 | >10000 | 446.0830 | 446.083 |
| 161 | 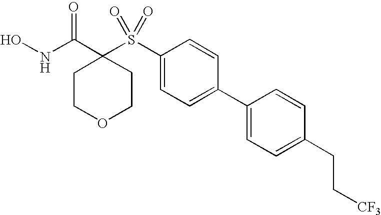 | 3350 | 0.15 | 0.21 | 0.07 | 303 | 458.4721 | 458.475 |
| 162 | 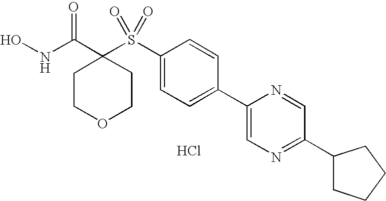 | >10000 | 3.11 | 34.0 | 0.950 | 7350 | 432.1588 | 432.1565 |
| 163 | 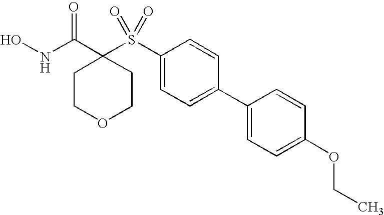 | 327 | 0.020 | 0.030 | 0.011 | 56.7 | 406.4546 | 406.4532 |
| 164 | 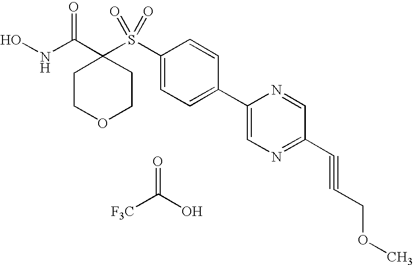 | 2490 | 0.6 | 0.57 | 0.09 | 152 | 432.1224 | 432.1231 |
| 165 | 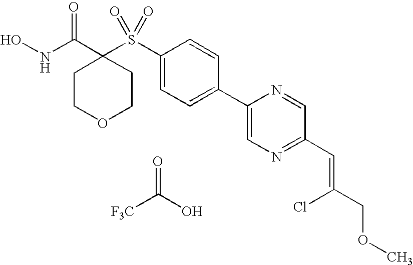 | 8950 | 2.46 | 267 | 0.84 | 1110 | 468.0991 | 468.0991 |
| 166 | 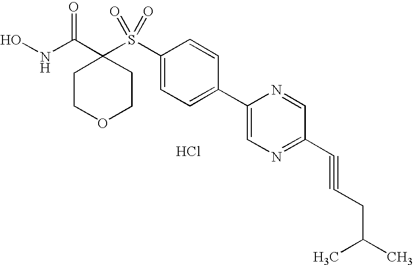 | 1660 | 0.40 | 0.10 | 0.03 | 264 | 444.1588 | 444.161 |
| 167 | 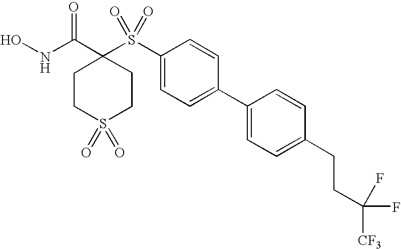 | >10000 | 1.9 | 12.5 | 0.74 | 3590 | 555.9848 | 555.9842 |
| 168 | 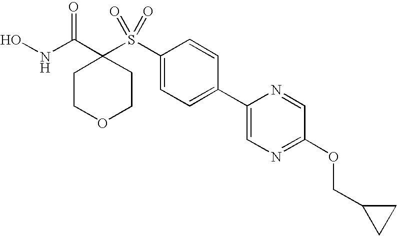 | 1920 | 0.08 | 0.19 | 0.06 | 46.2 | 434.138 | 434.1382 |
| 169 | 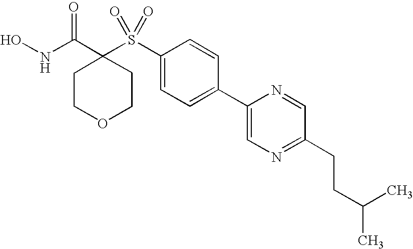 | >10000 | 1.09 | 0.59 | 0.24 | 2520 | 434.1744 | 434.1724 |
| 170 | 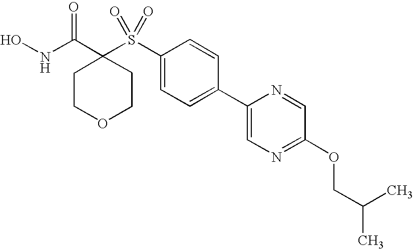 | 693 | 0.09 | 0.08 | 0.08 | 234 | 436.1537 | 436.153 |
| 171 | 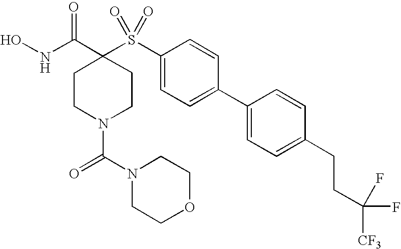 | 6370 | 0.24 | 0.62 | 0.23 | 633 | 620.1848 | 620.184 |
| 172 | 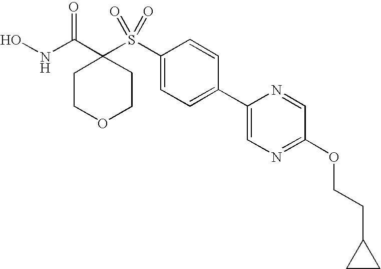 | 1360 | 0.07 | 0.06 | 0.03 | 222 | 448.1537 | 448.1529 |
| 173 | 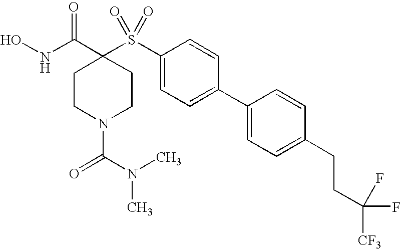 | 4610 | 0.35 | 0.67 | 0.32 | 441 | 578.1743 | 578.1757 |
| 174 | 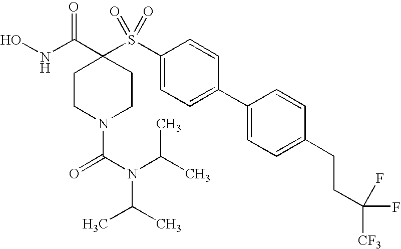 | >10000 | 1.24 | 3.77 | 1.92 | 1180 | 634.6218 | 634.6049 |
| 175 | 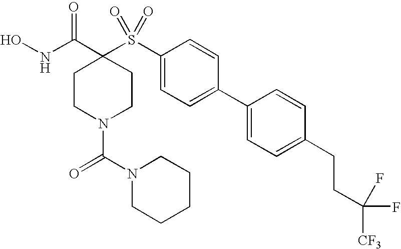 | >10000 | 0.42 | 1.35 | 0.71 | >10000 | 618.2056 | 618.2058 |
| 176 | 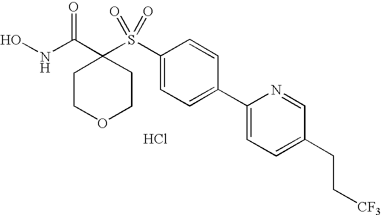 | 1200 | 0.04 | 0.04 | 0.02 | 111 | 459.1196 | 459.1183 |
| 177 | 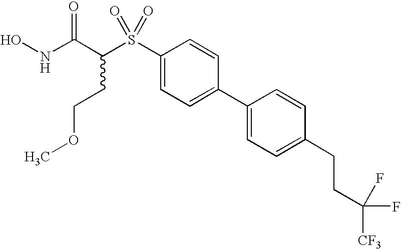 | >10000 | 1.57 | 5.36 | 0.82 | 5860 | 496.1212 | 496.1219 |
| 178 | 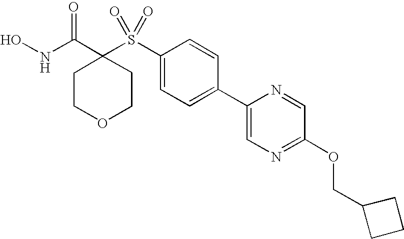 | 1100 | 0.10 | 0.14 | 0.08 | 326 | 448.1537 | 448.1529 |
| 179 | 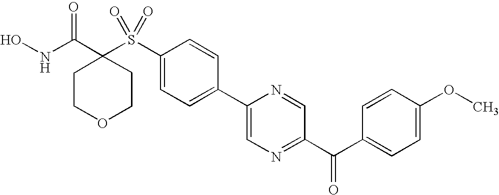 | >10000 | 2.99 | 38.6 | 0.973 | 218 | 498.1320 | 498.1333 |
| 180 | 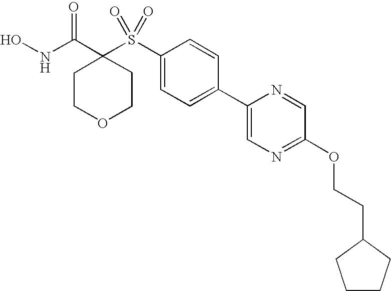 | >10000 | 0.295 | 1.03 | 0.15 | 1140 | 476.1850 | 476.1832 |
| 181 | 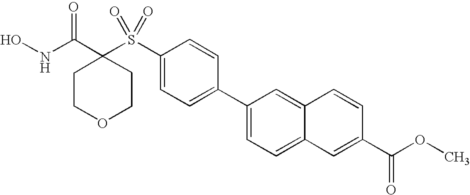 | 465 | 0.272 | 0.264 | 0.09 | 623 | 470.1273 | 470.1277 |
| 182 | 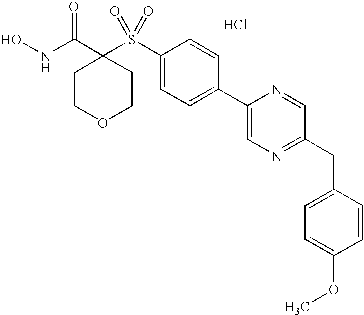 | >10000 | 114 | 246 | 7.07 | >10000 | | |
| 183 | 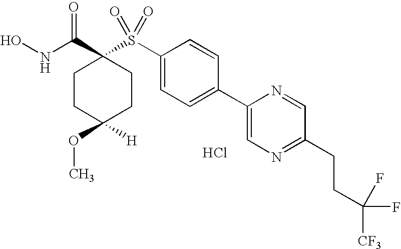 | >10000 | 0.35 | 0.14 | 0.13 | 798 | 538.1430 | 538.1415 |
| 184 | 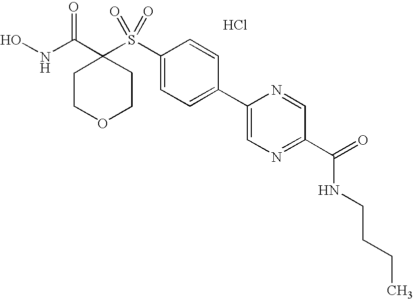 | 533 | 0.80 | 13.1 | 0.21 | 1290 | 463.1646 | 463.1664 |
| 185 | 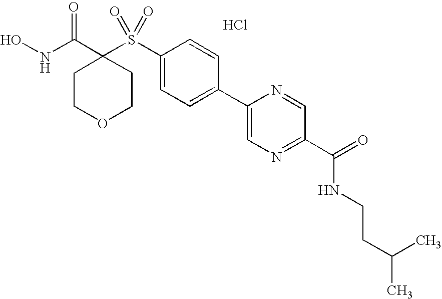 | 501 | 0.91 | 13.5 | 0.19 | 2050 | 477.1802 | 477.1816 |
| 186 | 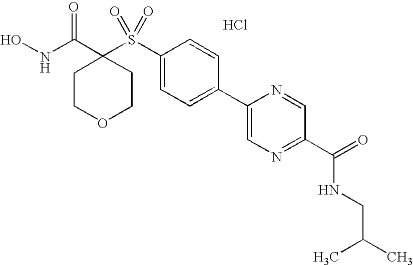 | 1100 | 2.33 | 21.7 | 0.55 | 4610 | 463.1646 | 463.1634 |
| 187 | 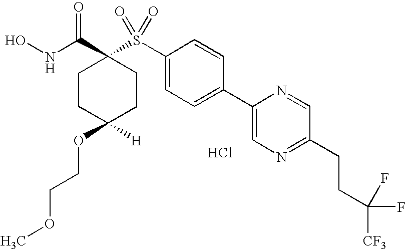 | 7020 | 0.32 | 0.16 | 0.10 | 625 | 582.1692 | 582.1714 |
| 188 | 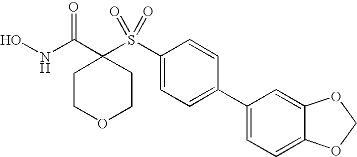 | 81.2 | 0.13 | 4.23 | 0.08 | 27.1 | 406 | 406 |
| 189 | 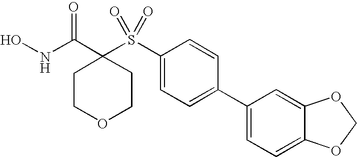 | 29.9 | 0.12 | 0.39 | 0.04 | 13.3 | 402 | 402 |
| 190 | 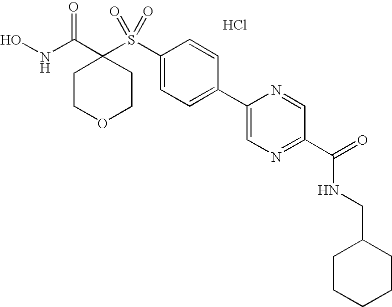 | 1310 | 1.7 | 32.3 | 0.28 | 4700 | 503.1959 | 503.1974 |
| 191 | 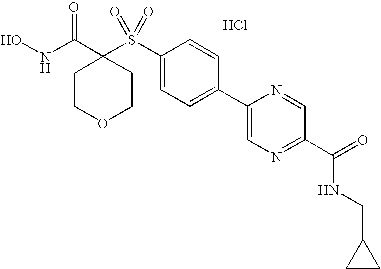 | 200 | 0.75 | 1.43 | 0.24 | 930 | 461.1489 | 461.1481 |
| 192 | 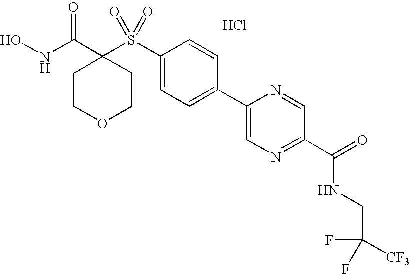 | 1540 | 0.89 | 41.1 | 0.22 | 5920 | 539.1018 | 539.1004 |
| 193 | 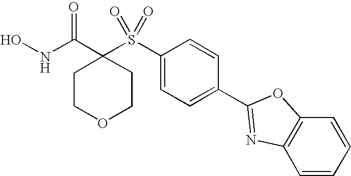 | 1490 | 1.34 | 11.5 | 1.69 | 96.7 | 403 | 403 |
| 194 | 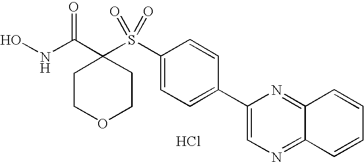 | >10000 | 176 | 1260 | 319 | >10000 | 414.1124 | 414.1125 |
| 195 | 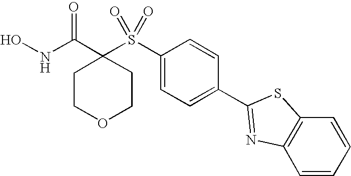 | >10000 | 5.74 | 32.2 | 7.2 | 1790 | 419 | 419 |
| 196 | 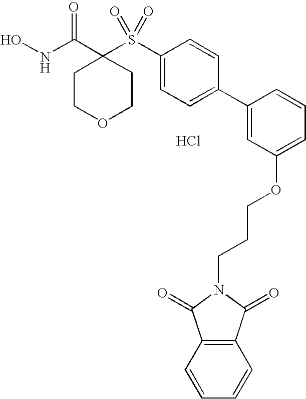 | >10000 | 947 | 430 | 13.5 | >10000 | 565.1645 | 565.1652 |
| 197 | 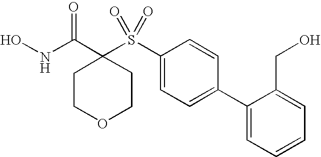 | >10000 | 6200 | >10000 | 1080 | >10000 | | |
| 198 | 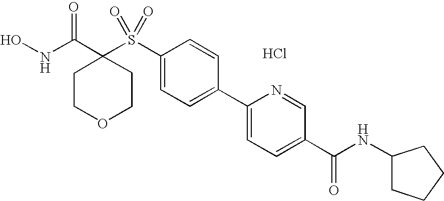 | 552 | 2.16 | 28.4 | 0.323 | 5120 | | |
| 199 | 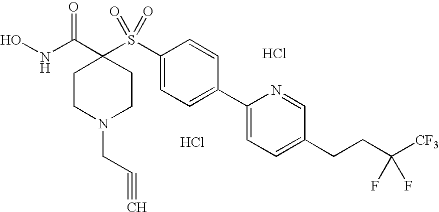 | 4510 | 0.085 | 0.147 | 0.086 | 586 | | |
| 200 | 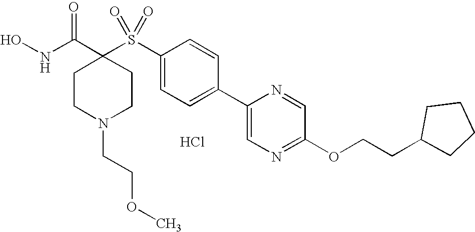 | >10000 | 0.404 | 0.648 | 0.144 | 942 | | |
| 201 | 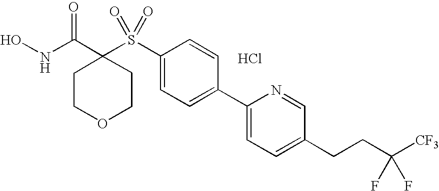 | 980 | 0.045 | 0.097 | 0.02 | 221 | | |
| 202 | 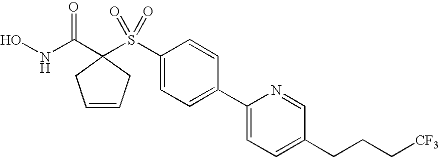 | 4060 | 0.32 | 0.578 | 0.076 | 1260 | | |
| 203 | 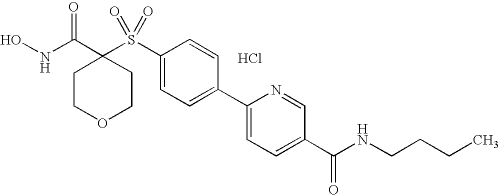 | 1140 | 1.77 | 61.4 | 0.375 | 7920 | | |
| 204 | 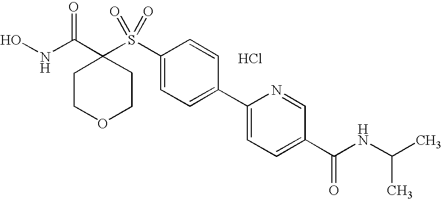 | 751 | 4.32 | 11.8 | 0.751 | 6370 | | |
| 205 | 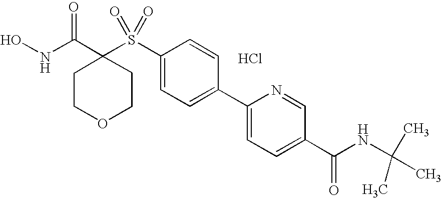 | 1070 | 15.1 | 21.8 | 1.19 | 7630 | | |
| 206 | 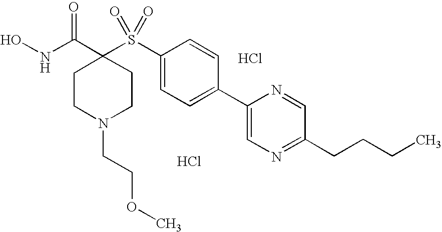 | >10000 | 2.98 | 1.8 | 0.41 | >10000 | | |
| 207 | 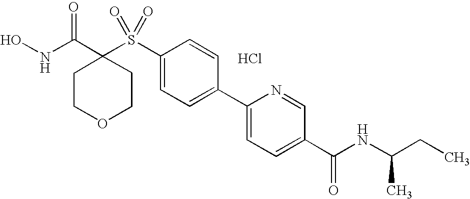 | 1380 | 4.99 | 78 | 0.729 | >10000 | | |
| 208 | 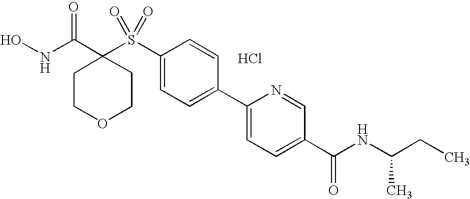 | 1260 | 10.6 | 39.4 | 0.715 | >10000 | | |
| 209 | 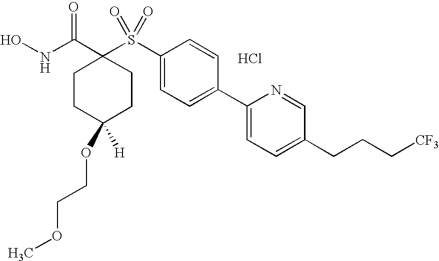 | 1280 | 0.081 | 0.138 | 0.029 | 272 | | |
| 210 | 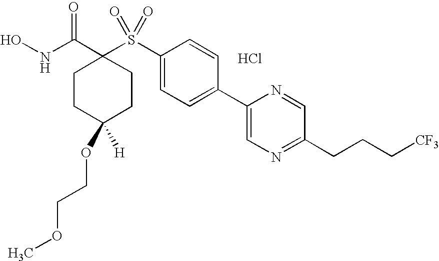 | >10000 | 1.01 | 0.75 | 0.151 | 3430 | | |
| 211 | 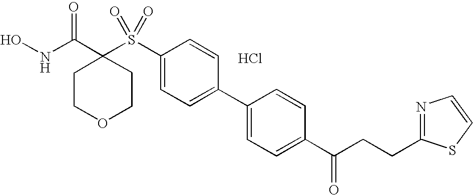 | 975 | 0.246 | 9.78 | 0.056 | 773 | | |
| 212 | 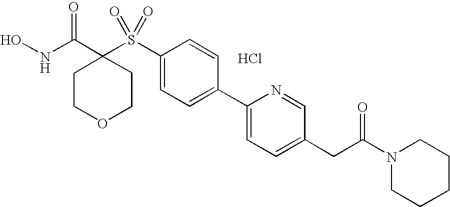 | >10000 | 34.7 | 437 | 0.679 | >10000 | | |
| 213 | 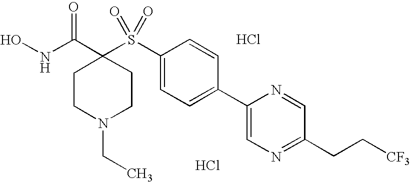 | >10000 | 3.57 | 4.8 | 1.41 | >10000 | | |
| 214 | 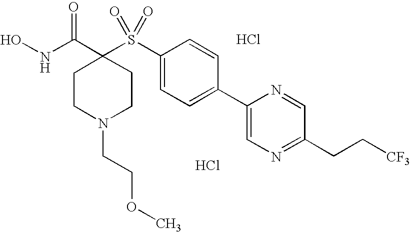 | >10000 | 2.09 | 2.05 | 0.661 | 6980 | | |
| 215 | 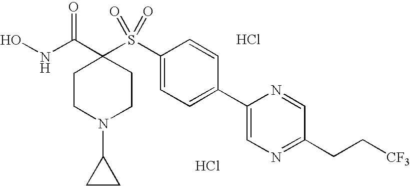 | >10000 | 1.15 | 1 | 0.356 | 5070 | | |
| 216 | 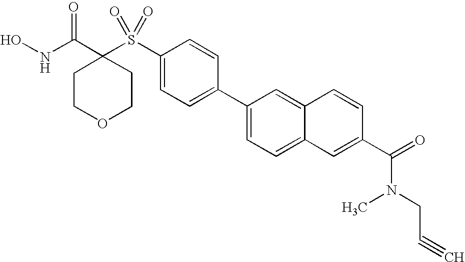 | 1860 | 2.51 | 13.3 | 0.572 | 2550 | | |
| 217 | 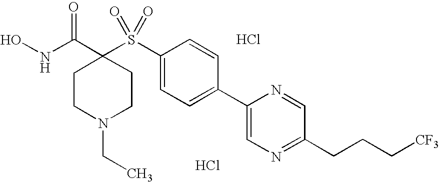 | >10000 | 9.06 | 5.22 | 1.20 | >10000 | | |
| 218 | 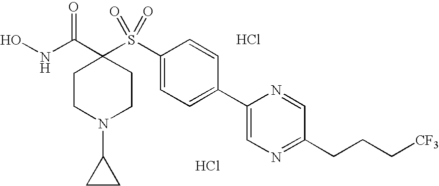 | >10000 | 1.93 | 0.938 | 0.267 | >10000 | | |
| 219 | 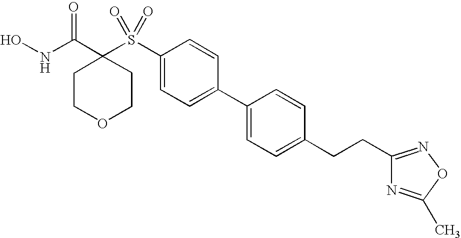 | 1700 | 0.588 | 9.25 | 0.111 | 1760 | | |
| 220 | 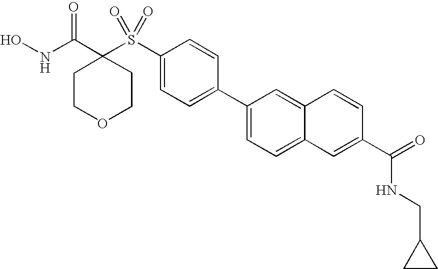 | 251 | 0.539 | 50.5 | 0.259 | >2500 | | |
| 221 | 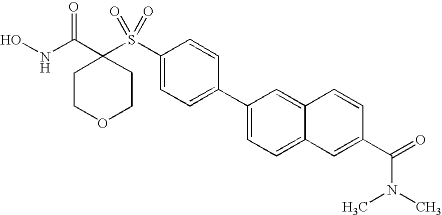 | 1860 | 5.61 | 16.7 | 0.736 | 6850 | | |
| 222 | 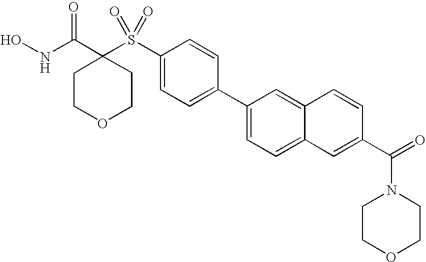 | 1440 | 7.8 | 29.5 | 0.612 | >10000 | | |
| 223 | 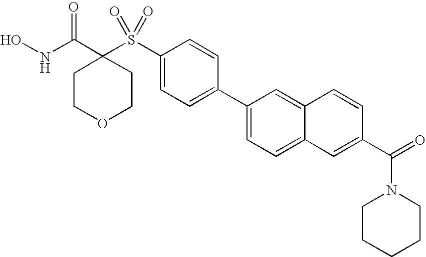 | 1500 | 11 | 34.3 | 0.970 | >10000 | | |

### Example 224

### In Vivo Angiogenesis Assay

The study of angiogenesis depends on a reliable and reproducible model for the stimulation and inhibition of a neovascular response. The corneal micropocket assay provides such a model of angiogenesis in the cornea of a mouse. See, A Model of Angiogenesis in the Mouse Cornea; Kenyon, B M, et al., Investigative Ophthalmology & Visual Science, July 1996, Vol. 37, No. 8.

In this assay, uniformly sized Hydron™ pellets containing bFGF and sucralfate are prepared and surgically implanted into the stroma mouse cornea adjacent to the temporal limbus. The pellets are formed by making a suspension of 20 μL sterile saline containing 10 μg recombinant bFGF, 10 mg of sucralfate and 10 μL of 12 percent Hydron™ in ethanol. The slurry is then deposited on a 10×10 mm piece of sterile nylon mesh. After drying, the nylon fibers of the mesh are separated to release the pellets.

The corneal pocket is made by anesthetizing a 7 week old C57B1/6 female mouse, then proptosing the eye with a jeweler's forceps. Using a dissecting microscope, a central, intrastromal linear keratotomy of approximately 0.6 mm in length is performed with a #15 surgical blade, parallel to the insertion of the lateral rectus muscle. Using a modified cataract knife, a lamellar micropocket is dissected toward the temporal limbus. The pocket is extended to within 1.0 mm of the temporal limbus. A single pellet is placed on the corneal surface at the base of the pocket with a jeweler's forceps. The pellet is then advanced to the temporal end of the pocket. Antibiotic ointment is then applied to the eye.

Mice are dosed on a daily basis for the duration of the assay. Dosing of the animals is based on bioavailability and overall potency of the compound. An exemplary dose is 10 or 50 mg/kg (mpk) bid, po. Neovascularization of the corneal stroma is permitted to continue under the influence of the assayed compound for 2 days. At that point, the degree of angiogenic inhibition is scored by viewing the neovascular progression with a slit lamp microscope.

The mice are anesthetized and the studied eye is once again proptosed. The maximum vessel length of neovascularization, extending from the limbal vascular plexus toward the pellet is measured. In addition, the contiguous circumferential zone of neovascularization is measured as clock hours, where 30 degrees of arc equals one clock hour. The area of angiogenesis is calculated as follows.
area=(0.4×clock hours×3.14×vessel length (in mm))/2

Five to six mice should be utilized for each compound in each study. The studied mice are thereafter compared to control mice and the difference in the area of neovascularization is recorded as an averaged value. A contemplated compound typically exhibits about 25 to about 75 percent inhibition, whereas the vehicle control exhibits zero percent inhibition.

### Example 225

### Tumor Necrosis Factor Assays

Cell Culture.

The cells used in the assay are the human monocytic line U-937 (ATCC CRL-1593). The cells are grown in RPMI w/10% FCS and PSG supplement (R-10) and are not permitted to overgrow. The assay is carried out as follows:

1. Count, then harvest cells by centrifugation. Resuspend the pellet in R-10 supplement to a concentration of 1.540×10⁶ cells/mL.

2. Add test compound in 65 uL R-10 to the appropriate wells of a 96-well flat bottom tissue culture plate. The initial dilution from a DMSO stock (100 mM compound) provides a 400 uM solution, from which five additional three-fold serial dilutions are made. Each dilution of 65 ul (in triplicate) yields final compound test concentrations of 100 μM, 33.3 μM, 11.1 μM, 3.7 μM, 1.2 μM and 0.4 μM.

3. The counted, washed and resuspended cells (200,000 cells/well) in 130 μL are added to the wells.

4. Incubation is for 45 min to 1 hr at 37° C. in 5% CO₂ in a water saturated container.

5. R-10 (65 uL) containing 160 ng/mL PMA (Sigma) is added to each well.

6. The test system is incubated at 37° C. in 5% CO₂ overnight (18-20 hr) under 100% humidity.

7. Supernatant, 150 μL, is carefully removed from each well for use in the ELISA assay.

8. For toxicity, a 50 μL aliquot of working solution containing 5 mL R-10, 5 mL MTS solution [CellTiter 96 AQueous One Solution Cell Proliferation Assay Cat.#G358/0,1 (Promega Biotech)] and 250 ul PMS solution are added to each well containing the remaining supernatant and cells and the cells incubated at 37° C. in 5% CO₂ until the color develops. The system is excited at 570 nm and read at 630 nm.

TNF Receptor II ELISA Assay

1. Plate 100 μL/well 2 ug/mL mouse anti-human TNFrII antibody (R&D Systems #MAB226) in 1× PBS (pH 7.1, Gibco) on NUNC-Immuno Maxisorb plate. Incubate the plate at 4° C. overnight (about 18-20 hr).

2. Wash the plate with PBS-Tween (1× PBS w/0.05% Tween).

3. Add 200 μL 5% BSA in PBS and block at 37° C. in a water saturated atmosphere for 2 hr.

4. Wash the plate with PBS-Tween.

5. Add sample and controls (100 ul of each) to each well. The standards are 0, 50, 100, 200, 300 and 500 pg recombinant human TNFrII (R&D Systems #226-B2) in 100 μL 0.5% BSA in PBS. The assay is linear to between 400-500 pg of standard.

6. Incubate at 37° C. in a saturated atmosphere for 1.5 hr.

7. Wash the plate with PBS-Tween.

8. Add 100 μL goat anti-human TNFrII polyclonal (1.5 μg/mL R&D Systems #AB226-PB in 0.5% BSA in PBS).

9. Incubate at 37° C. in a saturated atmosphere for 1 hr.

10. Wash the plate with PBS-Tween.

11. Add 100 μL anti-goat IgG-peroxidase (1:50,000 in 0.5% BSA in PBS, Sigma #A5420).

12. Incubate at 37° C. in a saturated atmosphere for 1 hr.

13. Wash the plate with PBS-Tween.

14. Add 10 μL KPL TMB developer, develop at room temperature (usually about 10 min), then terminate with phosphoric acid and excite at 450 nm and read at 570 nm.

TNFα ELISA Assay.

Coat Immulon® 2 plates with 0.1 mL/well of 1 ug/L Genzyme mAb in 0.1 M NaHCO3 pH 8.0 buffer overnight (about 18-20 hr) at 4° C., wrapped tightly in Saran® wrap.

Flick out coating solution and block plates with 0.3 mL/well blocking buffer overnight at 4° C., wrapped in Saran® wrap.

Wash wells thoroughly 4× with wash buffer and completely remove all wash buffer. Add 0.1 mL/well of either samples or rhTNFα standards. Dilute samples if necessary in appropriate diluant (e.g. tissue culture medium). Dilute standard in same diluant. Standards and samples should be in triplicates.

Incubate at 37° C. for 1 hr in humified container.

Wash plates as above. Add 0.1 mL/well of 1:200 dilution of Genzyme rabbit anti-hTNFα.

Repeat incubation.

Repeat wash. Add 0.1 mL/well of 1 μg/mL Jackson goat anti-rabbit IgG (H+L)-peroxidase.

Incubate at 37° C. for 30 min.

Repeat wash. Add 0.1 mL/well of peroxide-ABTS solution.

Incubate at room temperature for 5-20 min.

Read OD at 405 nm.

Reagents are:
Genzyme mouse anti-human TNF monoclonal (Cat.# 80-3399-01)
Genzyme rabbit anti-human TNF polyclonal (Cat.#IP-300)
Genzyme recombinant human TNF (Cat.#TNF-H).
Jackson Immunoresearch peroxide-conjugated goat anti-rabbit IgG (H+L) (Cat.#111-035-144).
Kirkegaard/Perry peroxide ABTS solution (Cat#50-66-01).
Immulon 2 96-well microtiter plates.
Blocking solution is 1 mg/mL gelatin in PBS with 1× thimerasol.
Wash buffer is 0.5 mL Tween® 20 in 1 liter of PBS.

### Example 226

### In Vitro Aggrecanase Inhibition Analysis

Assays for measuring the potency (IC₅₀) of a compound toward inhibiting aggrecanase are known in the art.

One such assay, for example, is reported in European Patent Application Publ. No. EP 1 081 137 A1. In that assay, primary porcine chondrocytes from articular joint cartilage are isolated by sequential trypsin and collagenase digestion followed by collagenase digestion overnight and are plated at 2×10⁵ cells per well into 48 well plates with 5 μCi/ml³⁵S (1000 Ci/mmol) sulphur in type 1 collagen coated plates. Cells are allowed to incorporate label into their proteoglycan matrix (approximately 1 week) at 37° C. under an atmosphere of 5% CO₂. The night before initiating the assay, chondrocyte monolayers are washed 2 times in DMEM/1% PSF/G and then allowed to incubate in fresh DMEM/1% FBS overnight. The next morning, chondrocytes are washed once in DMEM/1% PSF/G. The final wash is allowed to sit on the plates in the incubator while making dilutions. Media and dilutions are made as described in the following Table 3:

TABLE 3

| control media | DMEM alone |
|---|---|
| IL-1 media | DMEM + IL-1 (5 ng/ml) |
| drug dilutions | Make all compound stocks at 10 mM in DMSO. |
| | Make a 100 μM stock of each compound in DMEM in |
| | 96-well plate. Store in freezer overnight. |
| | The next day, perform serial dilutions in DMEM with |
| | IL-1 to 5 μM, 500 nM, and 50 nM. |
| | Aspirate final wash from wells and add 50 μM of |
| | compound from above dilutions to 450 μL of IL-1 |
| | media in appropriate wells of the 48 well plates. |
| | Final compound concentrations equal 500 nM, 50 nM, |
| | and 5 nM. |
| | All samples completed in triplicate with control and IL-1 |
| | alone on each plate. |

Plates are labeled and only the interior 24 wells of the plate are used. On one of the plates, several columns are designated as IL-1 (no drug) and control (no IL-1, no drug). These control columns are periodically counted to monitor 35S-proteoglycan release. Control and IL-1 media are added to wells (450 μL) followed by compound (50 μL) so as to initiate the assay. Plates are incubated at 37° C. with 5% CO₂ atmosphere. At 40-50% release (when CPM from IL-1 media is 4-5 times control media) as assessed by liquid scintillation counting (LSC) of media samples, the assay is terminated (about 9 to about 12 hours). Media is removed from all wells and placed into scintillation tubes. Scintillate is added and radioactive counts are acquired (LSC). To solubilize cell layers, 500 μL of papain digestion buffer (0.2 M Tris, pH 7.0, 5 mM DTT, and 1 mg/ml papain) is added to each well. Plates with digestion solution are incubated at 60° C. overnight. The cell layer is removed from the plates the next day and placed in scintillation tubes. Scintillate is then added, and samples counted (LSC). The percent of released counts from the total present in each well is determined. Averages of the triplicates are made with control background subtracted from each well. The percent of compound inhibition is based on IL-1 samples as 0% inhibition (100% of total counts).

Another assay for measuring aggrecanase inhibition is reported in WIPO Int'l Publ. No. WO 00/59874. That assay reportedly uses active aggrecanase accumulated in media from stimulated bovine cartilage (BNC) or related cartilage sources and purified cartilage aggrecan monomer or a fragment thereof as a substrate. Aggrecanase is generated by stimulation of cartilage slices with interleukin-1 (IL-1), tumor necrosis factor alpha (TNF-α), or other stimuli. To accumulate BNC aggrecanase in culture media, cartilage reportedly is first depleted of endogenous aggrecan by stimulation with 500 ng/ml human recombinant IL-β for 6 days with media changes every 2 days. Cartilage is then stimulated for an additional 8 days without media change to allow accumulation of soluble, active aggrecanase in the culture media. To decrease the amounts of matrix metalloproteinases released into the media during aggrecanase accumulation, agents which inhibit MMp-1, -2, -3, and -9 biosynthesis are included during stimulation. This BNC conditioned media containing aggrecanase activity is then used as the source of aggrecanase for the assay. Aggrecanase enzymatic activity is detected by monitoring production of aggrecan fragments produced exclusively by cleavage at the Glu373-Ala374 bond within the aggrecan core protein by Western analysis using the monoclonal antibody, BC-3 (Hughes, et al., Biochem J, 306:799-804 (1995)). This antibody reportedly recognizes aggrecan fragments with the N-terminus, 374ARGSVIL, generated upon cleavage by aggrecanase. The BC-3 antibody reportedly recognizes this neoepitope only when it is at the N-terminus and not when it is present internally within aggrecan fragments or within the aggrecan protein core. Only products produced upon cleavage by aggrecanase reportedly are detected. Kinetic studies using this assay reportedly yield a Km of 1.5±0.35 μM for aggrecanase. To evaluate inhibition of aggrecanase, compounds are prepared as 10 mM stocks in DMSO, water, or other solvents and diluted to appropriate concentrations in water. Drug (50 μL) is added to 50 μL of aggrecanase-containing media and 50 μL of 2 mg/ml aggrecan substrate and brought to a final volume of 200 μL in 0.2 M Tris, pH 7.6, containing 0.4 M NaCl and 40 mM CaCl₂. The assay is run for 4 hr at 37° C., quenched with 20 mM EDTA, and analyzed for aggrecanase-generated products. A sample containing enzyme and substrate without drug is included as a positive control and enzyme incubated in the absence of substrate serves as a measure of background. Removal of the glycosaminoglycan side chains from aggrecan reportedly is necessary for the BC-3 antibody to recognize the ARGSVIL epitope on the core protein. Therefore, for analysis of aggrecan fragments generated by cleavage at the Glu373-Ala374 site, proteoglycans and proteoglycan fragments are enzymatically deglycosylated with chondroitinase ABC (0.1 units/10 μg GAG) for 2 hr at 37° C. and then with keratanase (0.1 units/10 μg GAG) and keratanase II (0.002 units/10 μg GAG) for 2 hr at 37° C. in buffer containing 50 mM sodium acetate, 0.1 M Tris/HCl, pH 6.5. After digestion, aggrecan in the samples is precipitated with 5 volumes of acetone and resuspended in 30 μL of Tris glycine SDS sample buffer (Novex) containing 2.5% beta mercaptoethanol. Samples are loaded and then separated by SDS-PAGE under reducing conditions with 4-12% gradient gels, transferred to nitrocellulose and immunolocated with 1:500 dilution of antibody BC3. Subsequently, membranes are incubated with a 1:5000 dilution of goat anti-mouse IgG alkaline phosphatase second antibody and aggrecan catabolites visualized by incubation with appropriate substrate for 10-30 minutes to achieve optimal color development. Blots are quantitated by scanning densitometry and inhibition of aggrecanase determined by comparing the amount of product produced in the presence versus absence of compound.

The above detailed description of preferred embodiments is intended only to acquaint others skilled in the art with the invention, its principles, and its practical application so that others skilled in the art may adapt and apply the invention in its numerous forms, as they may be best suited to the requirements of a particular use. This invention, therefore, is not limited to the above embodiments, and may be variously modified.

We claim:

1. A compound or a salt thereof, wherein:
the compound corresponds in structure to Formula (1-1): 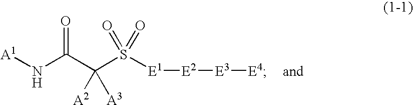
A¹ is selected from the group consisting of hydrogen, hydroxy, carbocyclyloxy, and heterocyclyloxy; and
as to A² and A³:
A² and A³, together with the carbon to which they are bonded, form heterocyclyl or carbocyclyl, wherein:
the heterocyclyl or carbocyclyl optionally is substituted with up to 3 independently selected Rx substituents, and
the heterocyclyl or carbocyclyl optionally is substituted with two substituents such that the two substituents, together with the atom(s) to which they are bonded, form a carbocyclyl or heterocyclyl, wherein:
the optional heterocyclyl or carbocyclyl is, in turn,
optionally substituted with up to 3 independently selected R^{x} substituents, or
A² and A³ are independently selected from the group consisting of hydrogen, alkyl, alkoxyalkyl, alkylthioalkyl, alkenyl, alkynyl, carbocyclyl, carbocyclylalkyl, carbocyclylalkenyl, carbocyclylalkynyl, carbocyclyloxyalkyl, carbocyclylalkoxyalkyl, carbocyclylalkylthio, carbocyclylthioalkyl, carbocyclylalkylthioalkyl, heterocyclyl, heterocyclylalkyl, heterocyclylalkenyl, heterocyclylalkynyl, heterocyclyloxyalkyl, heterocyclylalkoxyalkyl, heterocyclylalkylthio, heterocyclylthioalkyl, and heterocyclylalkylthioalkyl, wherein:
any member of such group optionally is substituted with up to 3 independently selected R^{x} substituents, and
any member of such group optionally is substituted with two substituents such that the two substituents, together with the atom(s) to which they are bonded, form a carbocyclyl or heterocyclyl, wherein:
the heterocyclyl and carbocyclyl optionally are substituted with up to 3 independently selected R^{x} substituents; and
E¹ is aryl optionally substituted with one or more independently selected R^{x} substituents; and
E² is selected from the group consisting of aryl and heteroaryl, wherein:
the aryl or heteroaryl optionally substituted with one or more independently selected R^{x} substituents; and
E³is selected from the group consisting of —O—, —C(O)—, —C(O)—O—, —O—C(O)—, —N(R^{b})—, —C(O)—N(R^{b})—, —N(R^{b})—C(O)—, —C(O)—N(R^{b})—N(R^{b})—C(O)—, —N(R^{b})—C(O)—N(R^{b})—, —S—, —S(O)—, —S(O)₂—, —N(R^{b})—S(O)₂—, —S(O)₂—N(R^{b})—, —O—S(O)₂—, —S(O)₂—O—, —C(NH)—, —C(NOH)—, —N(R^{b})—C(NH)—, —N(R^{b})—C(NOH)—, —C(NH)—N(R^{b})—, —C(NOH)—N(R^{b})—, alkyl, alkenyl, carbonylalkyl, alkylcarbonyl, and a bond, wherein:
any alkyl or alkenyl portion of a substituent in such group optionally is substituted with one or more independently selected R^{c} substituents; and
E⁴ is selected from the group consisting of alkyl, alkenyl, alkynyl, alkoxyalkyl, alkoxyalkoxyalkyl, alkylthioalkyl, alkylthioalkylthioalkyl, alkylthioalkoxyalkyl, alkoxyalkylthioalkyl, aminoalkyl, carbocyclyl, carbocyclylalkyl, carbocyclylalkoxyalkyl, heterocyclyl, heterocyclylalkyl, and heterocyclylalkoxyalkyl, wherein any such group:
comprises at least two carbon atoms, and
is substituted with one or more independently-selected halogen, and
is optionally substituted with one or more independently selected R^{d} substituents; and
each R^{x} is independently selected from the group consisting of halogen, cyano, hydroxy, nitro, nitroso, oxo, alkyl, alkenyl, alkynyl, alkoxy, alkoxyalkoxy, R^{b}-oxyalkyl, alkenyloxy, alkynyloxy, alkylthio, R^{b}R^{b}-amino, R^{b}R^{b}-aminoalkyl, R^{b}R^{b}-aminoalkoxy, R^{b}R^{b}-aminoalkyl(R^{b})amino, carbocyclyl, carbocyclylalkyl, carbocyclyloxy, carbocyclyloxyalkoxy, carbocyclylthio, heterocyclyl, heterocyclylalkyl, heterocyclyloxy, heterocyclyloxyalkoxy, heterocyclylthio, alkyliminocarbonyl, alkylthioalkyl, alkylsulfonylalkyl, alkylsulfoxidoalkyl, alkylthioalkenyl, alkylsulfoxidoalkenyl, alkylsulfonylalkenyl, carbocyclylalkoxyalkyl, carbocyclyliminocarbonyl, carbocyclylthioalkyl, carbocyclylsulfoxidoalkyl, carbocyclylsulfonylalkyl, carbocyclylthioalkenyl, carbocyclylsulfoxidoalkenyl, carbocyclylsulfonylalkenyl, heterocyclylalkoxyalkyl, heterocyclylthioalkyl, heterocyclylsulfoxidoalkyl, heterocyclylsulfonylalkyl, heterocyclylthioalkenyl, heterocyclylsulfoxidoalkenyl, heterocyclylsulfonylalkenyl, heterocyclyliminocarbonyl, aminosulfonylalkyl, and —R^{x1}—Rx², wherein:
any member of such group optionally is substituted with one or more substituents independently selected from the group consisting of halogen, hydroxy, cyano, amino, carboxy, thiol, sulfo, nitro, nitroso, oxo, thioxo, imino, alkyl, alkoxy, alkoxyalkyl, and alkoxyalkoxy, wherein:
the alkyl, alkoxy, alkoxyalkyl, and alkoxyalkoxy optionally are substituted with one or more substituents independently selected from the group consisting of halogen and hydroxy, and
the amino optionally is substituted with up to 2 independently selected alkyl; and
each R^{x1} is independently selected from the group consisting of —C(O)—, —C(S)—, —C(NR^{y})—, and —S(O)₂—; and
each R^{y} is independently selected from the group consisting of hydrogen and hydroxy; and
each R^{x2} is independently selected from the group consisting of hydrogen, hydroxy, alkyl, alkenyl, alkynyl, alkoxy, alkoxyalkyl, alkoxyalkoxy, R^{b}-oxyalkyl, alkenyloxy, alkynyloxy, R^{b}R^{b}-amino, R^{b}R^{b}-aminoalkyl, R^{b}R^{b}-aminoalkoxy, R^{b}R^{b}-aminoalkyl(R^{b})amino, carbocyclyl, carbocyclylalkyl, carbocyclyloxy, carbocyclyloxyalkoxy, heterocyclyl, heterocyclylalkyl, heterocyclyloxy, and heterocyclyloxyalkoxy, wherein:
any member of such group optionally is substituted with one or more substituents independently selected from the group consisting of halogen, hydroxy, cyano, carboxy, thiol, sulfo, nitro, nitroso, oxo, thioxo, imino, alkyl, alkoxy, alkoxyalkyl, and alkoxyalkoxy, wherein:
the alkyl, alkoxy, alkoxyalkyl, and alkoxyalkoxy optionally are substituted with one or more substituents independently selected from the group consisting of halogen and hydroxy; and
each R^{b} is independently selected from the group consisting of hydrogen, hydroxy, alkyl, alkenyl, alkynyl, alkoxy, alkoxyalkyl, bisalkoxyalkyl, alkylthioalkyl, alkylthioalkenyl, alkylsulfoxidoalkyl, alkylsulfonyl, alkylsulfonylalkyl, carbocyclyl, carbocyclylalkyl, carbocyclyloxyalkyl, carbocyclylalkoxyalkyl, carbocyclylthioalkyl, carbocyclylthioalkenyl, carbocyclylsulfoxidoalkyl, carbocyclylsulfonyl, carbocyclylsulfonylalkyl, heterocyclyl, heterocyclylalkyl, heterocyclyloxyalkyl, heterocyclylalkoxyalkyl, heterocyclylthioalkyl, heterocyclylsulfoxidoalkyl, heterocyclylsulfonyl, heterocyclylsulfonylalkyl, aminoalkyl, aminosulfonyl, aminoalkylsulfonyl, and alkoxyalkylaminoalkyl, wherein:
any member of such group optionally is substituted with one or more substituents independently selected from the group consisting of halogen, hydroxy, cyano, carboxy, thiol, sulfo, nitro, nitroso, oxo, thioxo, imino, alkyl, alkylcarbonyl, carbocyclyl, and carbocyclylalkyl; and
each R^{c} is independently selected from the group consisting of halogen, hydroxy, cyano, carboxy, —C(H)(NH), —C(H)(NOH), thiol, sulfo, nitro, nitroso, oxo, thioxo, imino, amino, alkyl, alkoxy, alkenyl, alkynyl, alkoxyalkyl, mono-alkylamino, di-alkylamino, alkylthio, carbocyclyl, carbocyclylalkyl, heterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, imino, aminocarbonyl, amino, alkyl, and carbocyclylalkyl; and
each R^{d} is independently selected from the group consisting of halogen, hydroxy, cyano, sulfo, nitro, nitroso, oxo, thioxo, imino, alkyl, alkoxy, alkoxyalkyl, —N(R^{e})(R^{e}), —C(O)(R^{g}), —S—Re, —S(O)₂—R^{e}, carbocyclyl, alkylcarbocyclyl, carbocyclylalkyl, heterocyclyl, alkylheterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, imino, aminocarbonyl, and amino; and
each R^{e} is independently selected from the group consisting of hydrogen alkyl, carbocyclyl, carbocyclylalkyl, heterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, imino, aminocarbonyl, and amino; and
each R^{g} is independently selected from the group consisting of hydrogen, alkyl, —O—R^{h}, —N(R^{h})(R^{h}), carbocyclylalkyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, imino, aminocarbonyl, and amino; and
each R^{h} is independently selected from the group consisting of hydrogen, alkyl, carbocyclyl, carbocyclylalkyl, heterocyclyl, and heterocyclylalkyl, wherein:
any member of such group optionally is substituted with one or more substituents independently selected from the group consisting of halogen, hydroxy, cyano, carboxy, thiol, sulfo, nitro, nitroso, oxo, thioxo, imino, aminocarbonyl, and amino.
